(19) United States
(12) Patent Application Publication
(10) Pub. No.: US 2007/0155737 A1
(43) Pub. Date: Jul. 5, 2007

(54) Heterobicyclic metalloprotease inhibitors

(75) Inventors: Brian M. Gallagher JR., Merrimac, MA (US); Joshua Van Veldhuizen, Seattle, WA (US); Xinyuan Wu, Newton, MA (US); Hongbo Deng, Southborough, MA (US); Irving Sucholeiki, Winchester, MA (US); Matthias Hochguertel, Schriesheim (DE); Harald Bluhm, Dossenheim (DE); Arthur G. Taveras, Southborough, MA (US)

Correspondence Address: HOFFMANN & BARON, LLP, 6900 JERICHO TURNPIKE, SYOSSET, NY, 11791, US

(73) Assignee: Alantos Pharmaceuticals, Inc.

(21) Appl. No.: 11/602,116

(22) Filed: Nov. 20, 2006

Related U.S. Application Data
(63) Continuation-in-part of application No. 11/440,087, filed on May 22, 2006.
(60) Provisional application No. 60/734,991, filed on Nov. 9, 2005.
(60) Provisional application No. 60/706,465, filed on Aug. 8, 2005.
(60) Provisional application No. 60/683,470, filed on May 20, 2005.

(51) Int. Cl. A61K 31/5377; C07D 491/04; C07D 487/04; A61K 31/538; A61K 31/519

(52) U.S. Cl. 514/230.5; 544/254; 544/255; 544/256; 544/263; 544/281; 544/114; 544/105; 514/234.5; 514/259.1; 514/259.2; 514/259.3; 514/259.31

(57) ABSTRACT

The present invention relates generally to amide group containing pharmaceutical agents, and in particular, to amide containing heterobicyclic metalloprotease inhibitor compounds. More particularly, the present invention provides a new class of heterobicyclic ADAMTS-4 inhibiting compounds.

### CROSS REFERENCE TO RELATED APPLICATIONS

This application is a continuation in part of U.S. application Ser. No. 11/440,087, filed May 22, 2006, which claims the benefit of U.S. Provisional Application No. 60/734,991, filed Nov. 9, 2005, U.S. Provisional Application No. 60/706,465, filed Aug. 8, 2005, and U.S. Provisional Application No. 60/683,470, filed May 20, 2005, the contents of each of which are hereby incorporated by reference.

### FIELD OF THE INVENTION

The present invention relates generally to amide containing heterobicyclic metalloprotease inhibiting compounds, and more particularly to heterobicyclic ADAMTS-4 inhibiting compounds.

### BACKGROUND OF THE INVENTION

Aggrecanases (ADAMTS=a disintegrin and metalloproteinase with thrombospondin motif) and matrix metalloproteinases (MMPs) are a family of structurally related zinc-containing enzymes that have been reported to mediate the breakdown of connective tissue in normal physiological processes such as embryonic development, reproduction, and tissue remodelling. Over-expression of aggrecanases and MMPs or an imbalance between extracellular matrix synthesis and degradation has been suggested as factors in inflammatory, malignant and degenerative disease processes. Aggrecanases and MMPs are, therefore, targets for therapeutic inhibitors in several inflammatory, malignant and degenerative diseases such as rheumatoid arthritis, osteoarthritis, osteoporosis, periodontitis, multiple sclerosis, gingivitis, corneal epidermal and gastric ulceration, atherosclerosis, neointimal proliferation (which leads to restenosis and ischemic heart failure) and tumor metastasis.

The ADAMTSs are a group of proteases that are encoded in 19 ADAMTS genes in humans. The ADAMTSs are extracellular, multidomain enzymes whose functions include collagen processing, cleavage of the matrix proteoglycans, inhibition of angiogenesis and blood coagulation homoeostasis (Biochem. J. 2005, 386, 15-27; Arthritis Res. Ther. 2005, 7, 160-169; Curr. Med. Chem. Anti-Inflammatory Anti-Allergy Agents 2005, 4, 251-264).

The mammalian MMP family has been reported to include at least 20 enzymes, (Chem. Rev. 1999, 99, 2735-2776). Collagenase-3 (MMP-13) is among three collagenases that have been identified. Based on identification of domain structures for individual members of the MMP family, it has been determined that the catalytic domain of the MMPs contains two zinc atoms; one of these zinc atoms performs a catalytic function and is coordinated with three histidines contained within the conserved amino acid sequence of the catalytic domain. MMP-13 is over-expressed in rheumatoid arthritis, osteoarthritis, abdominal aortic aneurysm, breast carcinoma, squamous cell carcinomas of the head and neck, and vulvar squamous cell carcinoma. The principal substrates of MMP-13 are fibrillar collagens (types I, II, III) and gelatins, proteoglycans, cytokines and other components of ECM (extracellular matrix).

The activation of the MMPs involves the removal of a propeptide, which features an unpaired cysteine residue complexes the catalytic zinc (II) ion. X-ray crystal structures of the complex between MMP-3 catalytic domain and TIMP-1 and MMP-14 catalytic domain and TIMP-2 also reveal ligation of the catalytic zinc (II) ion by the thiol of a cysteine residue. The difficulty in developing effective aggrecanase and MMP inhibiting compounds comprises several factors, including choice of selective versus broad-spectrum aggrecanase and MMP inhibitors and rendering such compounds bioavailable via an oral route of administration.

### SUMMARY OF THE INVENTION

The present invention relates to a new class of heterobicyclic amide containing pharmaceutical agents which inhibits metalloproteases. In particular, the present invention provides a new class of metalloprotease inhibiting compounds that exhibit potent ADAMTS-4 inhibiting activity and/or activity towards MMP-3, MMP-8, MMP-12, MMP-13, and ADAMTS-5.

The present invention provides several new classes of amide containing heterobicyclic metalloprotease compounds, of which some are represented by the following general formulas: 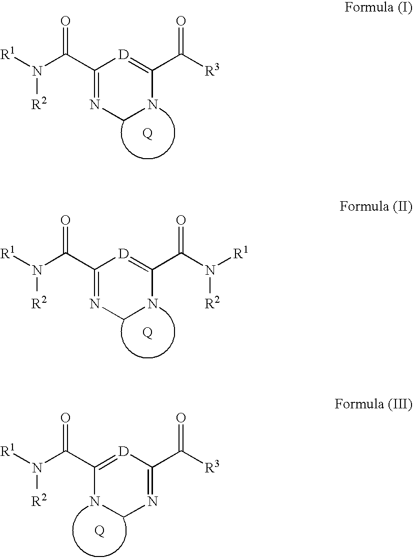
wherein all variables in the preceding Formulas (I) to (III) are as defined hereinbelow.

The heterobicyclic metalloprotease inhibiting compounds of the present invention may be used in the treatment of metalloprotease mediated diseases, such as rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, cardiovascular disease, reperfusion injury, trauma, chemical exposure or oxidative damage to tissues, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, bum therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, wheeze.

In particular, the heterobicyclic metalloprotease inhibiting compounds of the present invention may be used in the treatment of ADAMTS-4 mediated osteoarthritis and may be used for other ADAMTS-4 mediated symptoms, inflammatory, malignant and degenerative diseases characterized by excessive extracellular matrix degradation and/or remodelling, such as cancer, and chronic inflammatory diseases such as arthritis, rheumatoid arthritis, osteoartritis, atherosclerosis, abdominal aortic aneurysm, inflammation, multiple sclerosis, and chronic obstructive pulmonary disease, and pain, such as inflammatory pain, bone pain and joint pain.

The present invention also provides heterobicyclic metalloprotease inhibiting compounds that are useful as active ingredients in pharmaceutical compositions for treatment or prevention of metalloprotease—especially ADAMTS-4—mediated diseases. The present invention also contemplates use of such compounds in pharmaceutical compositions for oral or parenteral administration, comprising one or more of the heterobicyclic metalloprotease inhibiting compounds disclosed herein.

The present invention further provides methods of inhibiting metalloproteases, by administering formulations, including, but not limited to, oral, rectal, topical, intravenous, parenteral (including, but not limited to, intramuscular, intravenous), ocular (ophthalmic), transdermal, inhalative (including, but not limited to, pulmonary, aerosol inhalation), nasal, sublingual, subcutaneous or intraarticular formulations, comprising the heterobicyclic metalloprotease inhibiting compounds by standard methods known in medical practice, for the treatment of diseases or symptoms arising from or associated with metalloprotease, especially ADAMTS-4, including prophylactic and therapeutic treatment. Although the most suitable route in any given case will depend on the nature and severity of the conditions being treated and on the nature of the active ingredient. The compounds from this invention are conveniently presented in unit dosage form and prepared by any of the methods well-known in the art of pharmacy.

The heterobicyclic metalloprotease inhibiting compounds of the present invention may be used in combination with a disease modifying antirheumatic drug, a nonsteroidal anti-inflammatory drug, a COX-2 selective inhibitor, a COX-1 inhibitor, an immunosuppressive, a steroid, a biological response modifier or other anti-inflammatory agents or therapeutics useful for the treatment of chemokines mediated diseases.

### DETAILED DESCRIPTION OF THE INVENTION

The terms “alkyl” or “alk”, as used herein alone or as part of another group, denote optionally substituted, straight and branched chain saturated hydrocarbon groups, preferably having 1 to 10 carbons in the normal chain, most preferably lower alkyl groups. Exemplary unsubstituted such groups include methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl and the like. Exemplary substituents may include, but are not limited to, one or more of the following groups: halo, alkoxy, alkylthio, alkenyl, alkynyl, aryl (e.g., to form a benzyl group), cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The terms “lower alk” or “lower alkyl” as used herein, denote such optionally substituted groups as described above for alkyl having 1 to 4 carbon atoms in the normal chain.

The term “alkoxy” denotes an alkyl group as described above bonded through an oxygen linkage (—O—).

The term “alkenyl”, as used herein alone or as part of another group, denotes optionally substituted, straight and branched chain hydrocarbon groups containing at least one carbon to carbon double bond in the chain, and preferably having 2 to 10 carbons in the normal chain. Exemplary unsubstituted such groups include ethenyl, propenyl, isobutenyl, butenyl, pentenyl, hexenyl, heptenyl, octenyl, nonenyl, decenyl, and the like. Exemplary substituents may include, but are not limited to, one or more of the following groups: halo, alkoxy, alkylthio, alkyl, alkynyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “alkynyl”, as used herein alone or as part of another group, denotes optionally substituted, straight and branched chain hydrocarbon groups containing at least one carbon to carbon triple bond in the chain, and preferably having 2 to 10 carbons in the normal chain. Exemplary unsubstituted such groups include, but are not limited to, ethynyl, propynyl, butynyl, pentynyl, hexynyl, heptynyl, octynyl, nonynyl, decynyl, and the like. Exemplary substituents may include, but are not limited to, one or more of the following groups: halo, alkoxy, alkylthio, alkyl, alkenyl, aryl, cycloalkyl, cycloalkenyl, hydroxy or protected hydroxy, carboxyl (—COOH), alkyloxycarbonyl, alkylcarbonyloxy, alkylcarbonyl, carbamoyl (NH₂—CO—), substituted carbamoyl ((R¹⁰)(R¹¹)N—CO— wherein R¹⁰ or R¹¹ are as defined below, except that at least one of R¹⁰ or R¹¹ is not hydrogen), amino, heterocyclo, mono- or dialkylamino, or thiol (—SH).

The term “cycloalkyl”, as used herein alone or as part of another group, denotes optionally substituted, saturated cyclic hydrocarbon ring systems, containing one ring with 3 to 9 carbons. Exemplary unsubstituted such groups include, but are not limited to, cyclopropyl, cyclobutyl, cyclopentyl, cyclohexyl, cycloheptyl, cyclooctyl, cyclodecyl, and cyclododecyl. Exemplary substituents include, but are not limited to, one or more alkyl groups as described above, or one or more groups described above as alkyl substituents.

The term “bicycloalkyl”, as used herein alone or as part of another group, denotes optionally substituted, saturated cyclic bridged hydrocarbon ring systems, desirably containing 2 or 3 rings and 3 to 9 carbons per ring. Exemplary unsubstituted such groups include, but are not limited to, adamantyl, bicyclo[2.2.2]octane, bicyclo[2.2.1]heptane and cubane. Exemplary substituents include, but are not limited to, one or more alkyl groups as described above, or one or more groups described above as alkyl substituents.

The term “spiroalkyl”, as used herein alone or as part of another group, denotes optionally substituted, saturated hydrocarbon ring systems, wherein two rings of 3 to 9 carbons per ring are bridged via one carbon atom. Exemplary unsubstituted such groups include, but are not limited to, spiro[3.5]nonane, spiro[4.5]decane or spiro[2.5]octane. Exemplary substituents include, but are not limited to, one or more alkyl groups as described above, or one or more groups described above as alkyl substituents.

The term “spiroheteroalkyl”, as used herein alone or as part of another group, denotes optionally substituted, saturated hydrocarbon ring systems, wherein two rings of 3 to 9 carbons per ring are bridged via one carbon atom and at least one carbon atom is replaced by a heteroatom independently selected from N, O and S. The nitrogen and sulfur heteroatoms may optionally be oxidized. Exemplary unsubstituted such groups include, but are not limited to, 1,3-diaza-spiro[4.5]decane-2,4-dione. Exemplary substituents include, but are not limited to, one or more alkyl groups as described above, or one or more groups described above as alkyl substituents.

The terms “ar” or “aryl”, as used herein alone or as part of another group, denote optionally substituted, homocyclic aromatic groups, preferably containing 1 or 2 rings and 6 to 12 ring carbons. Exemplary unsubstituted such groups include, but are not limited to, phenyl, biphenyl, and naphthyl. Exemplary substituents include, but are not limited to, one or more nitro groups, alkyl groups as described above or groups described above as alkyl substituents.

The term “heterocycle” or “heterocyclic system” denotes a heterocyclyl, heterocyclenyl, or heteroaryl group as described herein, which contains carbon atoms and from 1 to 4 heteroatoms independently selected from N, O and S and including any bicyclic or tricyclic group in which any of the above-defined heterocyclic rings is fused to one or more heterocycle, aryl or cycloalkyl groups. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom.

Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolinyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isatinoyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, oxindolyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl.

Further examples of heterocycles include, but not are not limited to, “heterobicycloalkyl” groups such as 7-oxa-bicyclo[2.2.1]heptane, 7-aza-bicyclo[2.2.1]heptane, and 1-aza-bicyclo[2.2.2]octane.

“Heterocyclenyl” denotes a non-aromatic monocyclic or multicyclic hydrocarbon ring system of about 3 to about 10 atoms, desirably about 4 to about 8 atoms, in which one or more of the carbon atoms in the ring system is/are hetero element(s) other than carbon, for example nitrogen, oxygen or sulfur atoms, and which contains at least one carbon-carbon double bond or carbon-nitrogen double bond. Ring sizes of rings of the ring system may include 5 to 6 ring atoms. The designation of the aza, oxa or thia as a prefix before heterocyclenyl define that at least a nitrogen, oxygen or sulfur atom is present respectively as a ring atom. The heterocyclenyl may be optionally substituted by one or more substituents as defined herein. The nitrogen or sulphur atom of the heterocyclenyl may also be optionally oxidized to the corresponding N-oxide, S-oxide or S,S-dioxide. “Heterocyclenyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960), the contents all of which are incorporated by reference herein. Exemplary monocyclic azaheterocyclenyl groups include, but are not limited to, 1,2,3,4-tetrahydrohydropyridine, 1,2-dihydropyridyl, 1,4-dihydropyridyl, 1,2,3,6-tetrahydropyridine, 1,4,5,6-tetrahydropyrimidine, 2-pyrrolinyl, 3-pyrrolinyl, 2-imidazolinyl, 2-pyrazolinyl, and the like. Exemplary oxaheterocyclenyl groups include, but are not limited to, 3,4-dihydro-2H-pyran, dihydrofuranyl, and fluorodihydrofuranyl. An exemplary multicyclic oxaheterocyclenyl group is 7-oxabicyclo[2.2.1]heptenyl. “Heterocyclyl,” or “heterocycloalkyl,” denotes a non-aromatic saturated monocyclic or multicyclic ring system of about 3 to about 10 carbon atoms, desirably 4 to 8 carbon atoms, in which one or more of the carbon atoms in the ring system is/are hetero element(s) other than carbon, for example nitrogen, oxygen or sulfur. Ring sizes of rings of the ring system may include 5 to 6 ring atoms. The designation of the aza, oxa or thia as a prefix before heterocyclyl define that at least a nitrogen, oxygen or sulfur atom is present respectively as a ring atom. The heterocyclyl may be optionally substituted by one or more substituents which may be the same or different, and are as defined herein. The nitrogen or sulphur atom of the heterocyclyl may also be optionally oxidized to the corresponding N-oxide, S-oxide or S,S-dioxide.

“Heterocyclyl” as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary monocyclic heterocyclyl rings include, but are not limited to, piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

“Heteroaryl” denotes an aromatic monocyclic or multicyclic ring system of about 5 to about 10 atoms, in which one or more of the atoms in the ring system is/are hetero element(s) other than carbon, for example nitrogen, oxygen or sulfur. Ring sizes of rings of the ring system include 5 to 6 ring atoms. The “heteroaryl” may also be substituted by one or more substituents which may be the same or different, and are as defined herein. The designation of the aza, oxa or thia as a prefix before heteroaryl define that at least a nitrogen, oxygen or sulfur atom is present respectively as a ring atom. A nitrogen atom of a heteroaryl may be optionally oxidized to the corresponding N-oxide. Heteroaryl as used herein includes by way of example and not limitation those described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and “J. Am. Chem. Soc.”, 82:5566 (1960). Exemplary heteroaryl and substituted heteroaryl groups include, but are not limited to, pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidyl, pyrrolopyridyl, imidazopyridyl, benzoazaindole, 1,2,3-triazinyl, 1,2,4-triazinyl, 1,3,5-triazinyl, benzthiazolyl, dioxolyl, furanyl, imidazolyl, indolyl, indolizinyl, isoxazolyl, isoquinolinyl, isothiazolyl, , oxadiazolyl, oxazinyl, oxiranyl, piperazinyl, piperidinyl, pyranyl, pyrazinyl, pyridazinyl, pyrazolyl, pyridyl, pyrimidinyl, pyrrolyl, pyrrolidinyl, quinazolinyl, quinolinyl, tetrazinyl, tetrazolyl, 1,3,4-thiadiazolyl, 1,2,3-thiadiazolyl, 1,2,4-thiadiazolyl, 1,2,5-thiadiazolyl, thiatriazolyl, thiazinyl, thiazolyl, thienyl, 5-thioxo-1,2,4-diazolyl, thiomorpholino, thiophenyl, thiopyranyl, triazolyl and triazolonyl.

The phrase “fused” means, that the group, mentioned before “fused” is connected via two adjacent atoms to the ring system mentioned after “fused” to form a bicyclic system. For example, “heterocycloalkyl fused aryl” includes, but is not limited to, 2,3-dihydro-benzo[1,4]dioxine, 4H-benzo[1,4]oxazin-3-one, 3H-Benzooxazol-2-one and 3,4-dihydro-2H-benzof[f][1,4]oxazepin-5-one.

The term “amino” denotes the radical —NH₂ wherein one or both of the hydrogen atoms may be replaced by an optionally substituted hydrocarbon group. Exemplary amino groups include, but are not limited to, n-butylamino, tert-butylamino, methylpropylamino and ethyldimethylamino.

The term “cycloalkylalkyl” denotes a cycloalkyl-alkyl group wherein a cycloalkyl as described above is bonded through an alkyl, as defined above. Cycloalkylalkyl groups may contain a lower alkyl moiety. Exemplary cycloalkylalkyl groups include, but are not limited to, cyclopropylmethyl, cyclopentylmethyl, cyclohexylmethyl, cyclopropylethyl, cyclopentylethyl, cyclohexylpropyl, cyclopropylpropyl, cyclopentylpropyl, and cyclohexylpropyl.

The term “arylalkyl” denotes an aryl group as described above bonded through an alkyl, as defined above.

The term “heteroarylalkyl” denotes a heteroaryl group as described above bonded through an alkyl, as defined above.

The term “heterocyclylalkyl,” or “heterocycloalkylalkyl,” denotes a heterocyclyl group as described above bonded through an alkyl, as defined above.

The terms “halogen”, “halo”, or “hal”, as used herein alone or as part of another group, denote chlorine, bromine, fluorine, and iodine.

The term “haloalkyl” denotes a halo group as described above bonded though an alkyl, as defined above. Fluoroalkyl is an exemplary group.

The term “aminoalkyl” denotes an amino group as defined above bonded through an alkyl, as defined above.

The phrase “bicyclic fused ring system wherein at least one ring is partially saturated” denotes an 8- to 13-membered fused bicyclic ring group in which at least one of the rings is non-aromatic. The ring group has carbon atoms and optionally 1-4 heteroatoms independently selected from N, O and S. Illustrative examples include, but are not limited to, indanyl, tetrahydronaphthyl, tetrahydroquinolyl and benzocycloheptyl.

The phrase “tricyclic fused ring system wherein at least one ring is partially saturated” denotes a 9- to 18-membered fused tricyclic ring group in which at least one of the rings is non-aromatic. The ring group has carbon atoms and optionally 1-7 heteroatoms independently selected from N, O and S. Illustrative examples include, but are not limited to, fluorene, 10,11-dihydro-5H-dibenzo[a,d]cycloheptene and 2,2a,7,7a-tetrahydro-1H-cyclobuta[a]indene.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Examples therefore may be, but are not limited to, sodium, potassium, choline, lysine, arginine or N-methyl-glucamine salts, and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric and the like; and the salts prepared from organic acids such as, but not limited to, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts are found in Remington's Pharmaceutical Sciences, 18th ed., Mack Publishing Company, Easton, Penn., 1990, p. 1445, the disclosure of which is hereby incorporated by reference.

The phrase “pharmaceutically acceptable” denotes those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The phrase “pharmaceutically acceptable carrier” denotes media generally accepted in the art for the delivery of biologically active agents to mammals, e.g., humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Non-limiting examples of a pharmaceutically acceptable carrier are hyaluronic acid and salts thereof, and microspheres (including, but not limited to poly(D,L)-lactide-co-glycolic acid copolymer (PLGA), poly(L-lactic acid) (PLA), poly(caprolactone (PCL) and bovine serum albumin (BSA)). Descriptions of suitable pharmaceutically acceptable carriers, and factors involved in their selection, are found in a variety of readily available sources, e.g., Remington's Pharmaceutical Sciences, 17th ed., Mack Publishing Company, Easton, Pa., 1985, the contents of which are incorporated herein by reference.

Pharmaceutically acceptable carriers particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

The compositions of the invention may also be formulated as suspensions including a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Carriers suitable for use in connection with suspensions include suspending agents, such as sodium carboxymethylcellulose, methylcellulose, hydroxypropyl methylcelluose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

Cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. The amount of solubility enhancer employed will depend on the amount of the compound of the present invention in the composition.

The term “formulation” denotes a product comprising the active ingredient(s) and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing a compound of the present invention and a pharmaceutical carrier.

The term “N-oxide” denotes compounds that can be obtained in a known manner by reacting a compound of the present invention including a nitrogen atom (such as in a pyridyl group) with hydrogen peroxide or a peracid, such as 3-chloroperoxy-benzoic acid, in an inert solvent, such as dichloromethane, at a temperature between about −10-80° C., desirably about 0° C.

The term “polymorph” denotes a form of a chemical compound in a particular crystalline arrangement. Certain polymorphs may exhibit enhanced thermodynamic stability and may be more suitable than other polymorphic forms for inclusion in pharmaceutical formulations.

The compounds of the invention can contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures.

The term “racemic mixture” denotes a mixture that is about 50% of one enantiomer and about 50% of the corresponding enantiomer relative to all chiral centers in the molecule. Thus, the invention encompasses all enantiomerically-pure, enantiomerically-enriched, and racemic mixtures of compounds of Formulas (I) through (VI).

Enantiomeric and stereoisomeric mixtures of compounds of the invention can be resolved into their component enantiomers or stereoisomers by well-known methods. Examples include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, Ind., 1972); Stereochemistry of Organic Compounds, Ernest L. Eliel, Samuel H. Wilen and Lewis N. Manda (1994 John Wiley & Sons, Inc.), and Stereoselective Synthesis A Practical Approach, Mihaly Nogradi (1995 VCH Publishers, Inc., NY, N.Y.). Enantiomers and stereoisomers can also be obtained from stereomerically- or enantiomerically-pure intermediates, reagents, and catalysts by well-known asymmetric synthetic methods.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

Unless moieties of a compound of the present invention are defined as being unsubstituted, the moieties of the compound may be substituted. In addition to any substituents provided above, the moieties of the compounds of the present invention may be optionally substituted with one or more groups independently selected from:

C₁-C₄ alkyl;

C₂-C₄ alkenyl;

C₂-C₄ alkynyl;

CF₃;

halo;

OH;

O—(C₁-C₄ alkyl);

OCH₂F;

OCHF₂;

OCF₃;

ONO₂;

OC(O)—(C₁-C₄ alkyl);

OC(O)—(C₁-C₄ alkyl);

OC(O)NH—(C₁-C₄ alkyl);

OC(O)N(C₁-C₄ alkyl)₂;

OC(S)NH—(C₁-C₄ alkyl);

OC(S)N(C₁-C₄ alkyl)₂,

SH;

S—(C₁-C₄ alkyl);

S(O)—(C₁-C₄ alkyl);

S(O)₂-(C₁-C₄ alkyl);

SC(O)—(C₁-C₄ alkyl);

SC(O)O—(C₁-C₄ alkyl);

NH₂;

N(H)—(C₁-C₄ alkyl);

N(C₁-C₄ alkyl)₂;

N(H)C(O)—(C₁-C₄ alkyl);

N(CH₃)C(O)—(C₁-C₄ alkyl);

N(H)C(O)—CF₃;

N(CH₃)C(O)—CF₃;

N(H)C(S)—(C₁-C₄ alkyl);

N(CH₃)C(S)—(C₁-C₄ alkyl);

N(H)S(O)₂-(C₁-C₄ alkyl);

N(H)C(O)NH₂;

N(H)C(O)NH—(C₁-C₄ alkyl);

N(CH₃)C(O)NH—(C₁-C₄ alkyl);

N(H)C(O)N(C₁-C₄ alkyl)₂;

N(CH₃)C(O)N(C₁-C₄ alkyl)₂;

N(H)S(O)₂NH₂);

N(H)S(O)₂NH—(C₁-C₄ alkyl);

N(CH₃)S(O)₂NH—(C₁-C₄ alkyl);

N(H)S(O)₂N(C₁-C₄ alkyl)₂;

N(CH₃)S(O)₂N(C₁-C₄ alkyl)₂;

N(H)C(O)O—(C₁-C₄ alkyl);

N(CH₃)C(O)O—(C₁-C₄ alkyl);

N(H)S(O)₂O—(C₁-C₄ alkyl);

N(CH₃)S(O)₂O—(C₁-C₄ alkyl);

N(CH₃)C(S)NH—(C₁-C₄ alkyl);

N(CH₃)C(S)N(C₁-C₄ alkyl)₂;

N(CH₃)C(S)O—(C₁-C₄ alkyl);

N(H)C(S)NH₂;

NO₂;

CO₂H;

CO₂—(C₁-C₄ alkyl);

C(O)N(H)OH;

C(O)N(CH₃)OH:

C(O)N(CH₃)OH;

C(O)N(CH₃)O—(C₁-C₄ alkyl);

C(O)N(H)—(C₁-C₄ alkyl);

C(O)N(C₁-C₄ alkyl)₂;

C(S)N(H)—(C₁-C₄ alkyl);

C(S)N(C₁-C₄alkyl)₂;

C(NH)N(H)—(C₁-C₄ alkyl);

C(NH)N(C₁-C₄ alkyl)₂;

C(NCH₃)N(H)—(C₁-C₄ alkyl);

C(NCH₃)N(C₁-C₄ alkyl)₂;

C(O)—(C₁-C₄ alkyl);

C(NH)—(C₁-C₄ alkyl);

C(NCH₃)—(C₁-C₄ alkyl);

C(NOH)—(C₁-C₄ alkyl);

C(NOCH₃)—(C₁-C₄ alkyl);

CN;

CHO;

CH₂OH;

CH₂O—(C₁-C₄ alkyl);

CH₂NH₂;

CH₂N(H)—(C₁-C₄ alkyl);

CH₂N(C₁-C₄ alkyl)₂;

aryl;

heteroaryl;

cycloalkyl; and

heterocyclyl.

In some cases, a ring substituent may be shown as being connected to the ring by a bond extending from the center of the ring. The number of such substituents present on a ring is indicated in subscript by a number. Moreover, the substituent may be present on any available ring atom, the available ring atom being any ring atom which bears a hydrogen which the ring substituent may replace. For illustrative purposes, if variable R^{X} were defined as being:

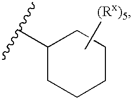

this would indicate a cyclohexyl ring bearing five R^{X} substituents. The R^{X} substituents may be bonded to any available ring atom. For example, among the configurations encompassed by this are configurations such as:

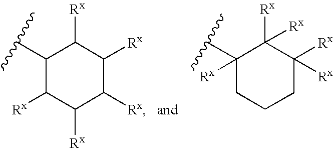

These configurations are illustrative and are not meant to limit the scope of the invention in any way.

In one embodiment of the present invention, the amide containing heterobicyclic metalloprotease compounds may be represented by the general Formula (I):

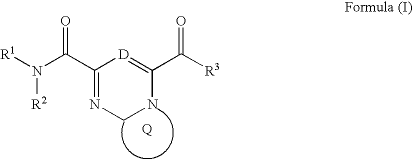

wherein:

R¹ is selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,

wherein R¹ is optionally substituted one or more times, or

wherein R¹ is optionally substituted one or more times by R⁹, or

wherein R¹ is optionally substituted by one R¹⁶ group and optionally substituted by one or more R⁹ groups;

R² is selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R³ is NR²⁰R²¹;

R⁴ in each occurrence is independently selected from the group consisting of R₁₀, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,

wherein each R⁴ group is optionally substituted one or more times, or

wherein each R⁴ group is optionally substituted by one or more R¹⁴ groups;

R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;

R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰-(C₀-C₆)-alkyl-aryl, S(O)₂NR¹⁰-(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,

wherein each R⁹ group is optionally substituted, or

wherein each R⁹ group is optionally substituted by one or more R¹⁴ groups;

R¹⁰ and R¹¹ in each occurrence are independently selected from the group consisting of hydrogen, alkyl, alkenyl, alkynyl, cycloalkyl, cycloalkylalkyl, heterocycloalkyl, bicycloalkyl, heterobicycloalkyl, spiroalkyl, spiroheteroalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, bicycloalkyl, heterobicycloalkyl, spiroalkyl, spiroheteroalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted one or more times;

R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;

R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii):

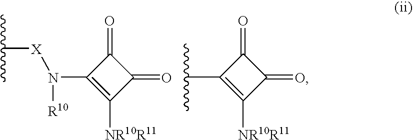

wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;

R²⁰ is selected from the group consisting of hydrogen and alkyl, wherein alkyl is optionally substituted one or more times;

R²¹ is a bicyclic or tricyclic fused ring system, wherein at least one ring is partially saturated, and

wherein R²¹ is optionally substituted one or more times, or

wherein R²¹ is optionally substituted by one or more R⁹ groups;

R²² is selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰NR¹⁰R¹¹, NR¹⁰N═CR¹⁰R¹¹, NR¹⁰SO₂R¹¹, C(O)OR¹⁰, C(O)NR¹⁰R¹¹, SO₂R¹⁰, SO₂NR¹⁰R¹¹ and NR¹⁰R¹¹ and fluoroalkyl, wherein alkyl, cycloalkyl, alkoxy, alkenyl, alkynyl, and fluoroalkyl are optionally substituted one or more times;

R³⁰ is selected from the group consisting of alkyl and (C₀-C₆)-alkyl-aryl, wherein alkyl and aryl are optionally substituted;

R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;

R⁸⁰ and R⁸¹ in each occurrence are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;

E is selected from the group consisting of a bond, CR¹⁰R¹¹, O, NR⁵, S, S═O, S(═O)₂, C(═O), N(R¹⁰) (C═O), (C═O)N(R¹⁰), N(R¹⁰)S(═O)₂, S(═O)₂N(R¹⁰), C═N—OR¹¹, —C(R¹⁰R¹¹)C(R¹⁰R¹¹)—, —CH₂—W¹— and

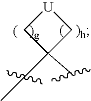

Q is a 5- or 6-membered ring selected from the group consisting of aryl and heteroaryl, wherein aryl and heteroaryl are optionally substituted one or more times with R⁴;

D is a member selected from the group consisting of CR²² and N;

U is selected from the group consisting of C(R⁵R¹⁰), NR⁵, O, S, S═O and S(═O)₂;

W¹ is selected from the group consisting of O, NR⁵, S, S═O, S(═O)₂, N(R¹⁰)(C═O), N(R¹⁰)S(═O)₂ and S(═O)₂N(R¹⁰);

X is selected from the group consisting of a bond and (CR¹⁰R¹¹)_{w}E(CR¹⁰R¹¹)_{w};

g and h are independently selected from 0-2;

w is independently selected from 0-4;

x is selected from 0 to 2;

y is selected from 1 and 2; and

N-oxides, pharmaceutically acceptable salts, prodrugs, formulation, polymorphs, racemic mixtures and stereoisomers thereof.

In another embodiment, compounds of Formula (I) may be selected from: 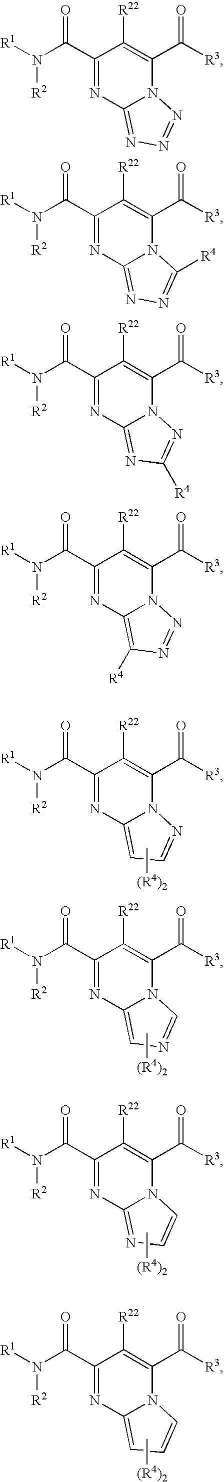 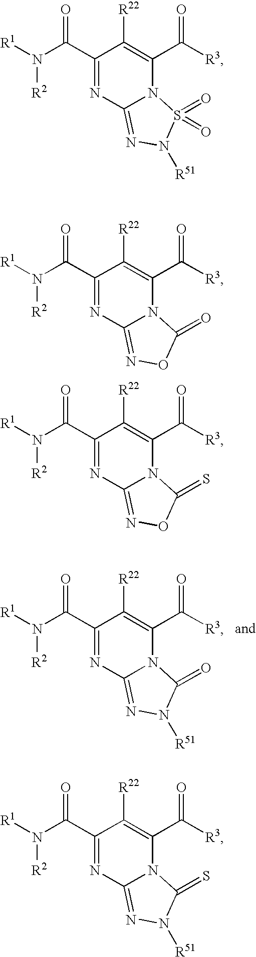
wherein:

R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

In still another embodiment, compounds of Formula (I) may be selected from:

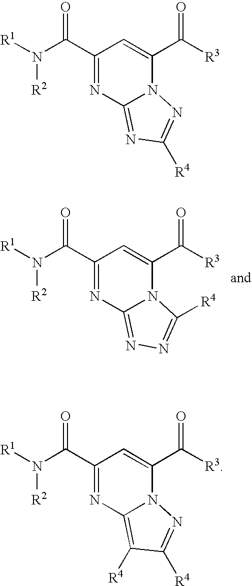

In yet another embodiment, compounds of Formula (I) may be selected from:

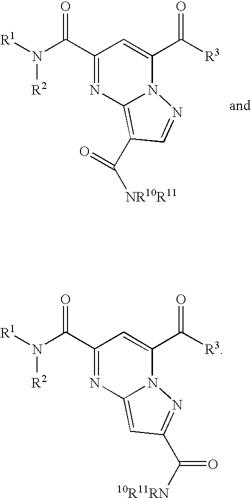

In yet another embodiment, compounds of Formula (I) may be selected from: 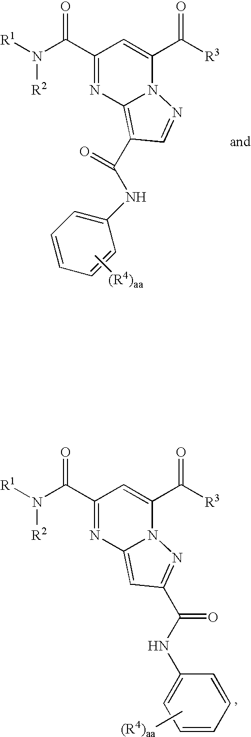
wherein: aa is selected from 0-5.

In some embodiments, R³ of the compounds of Formula (I) may be selected from: 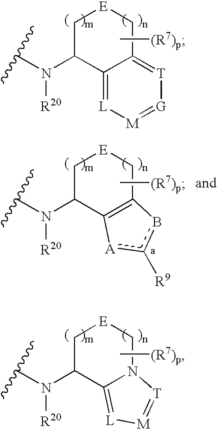
wherein:

R⁷is independently selected from the group consisting of hydrogen, alkyl, cycloalkyl, halo, R⁴ and NR¹⁰R¹¹, or optionally two R⁷ groups together at the same carbon atom form ═O, ═S or ═NR¹⁰;

A and B are independently selected from the group consisting of CR⁹, CR⁹R¹⁰, NR¹⁰, N, O and S(O)ₓ;

G, L, M and T are independently selected from the group consisting of CR⁹ and N;

m and n are independently selected from 0-3, provided that:

when E is present, m and n are not both 3;

when E is —CH₂—W¹—, m and n are not 3; and

when E is a bond, m and n are not 0; and

p is selected from 0-6;

wherein the dotted line represents a double bond between one of: carbon “a” and A, or carbon “a” and B.

In some embodiments, R³ of Formula (I) may be selected from:

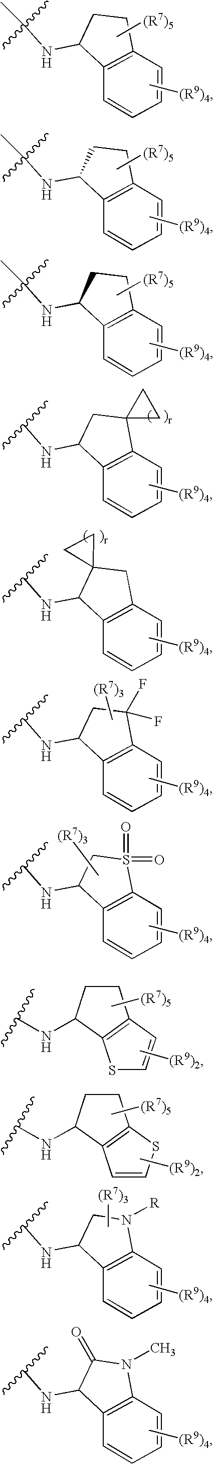

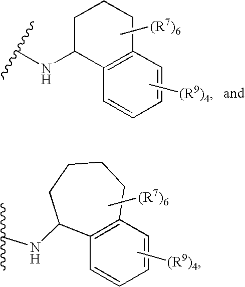

wherein:

R is selected from the group consisting of C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and

r is selected from 1-6.

In yet a further embodiment, R³ of Formula (I) may be selected from:

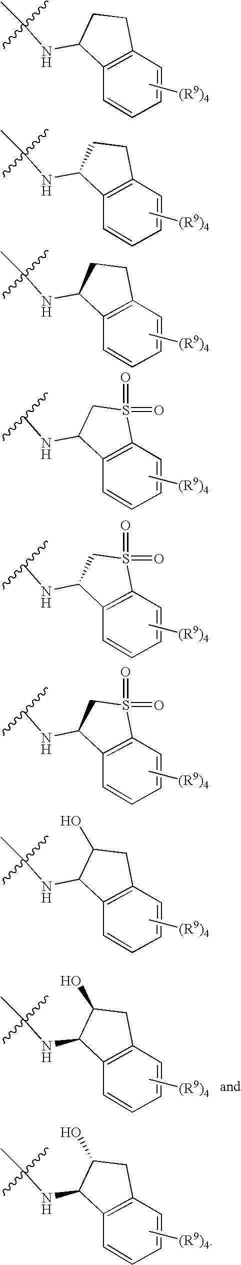

In another embodiment, R⁹ may be selected from:

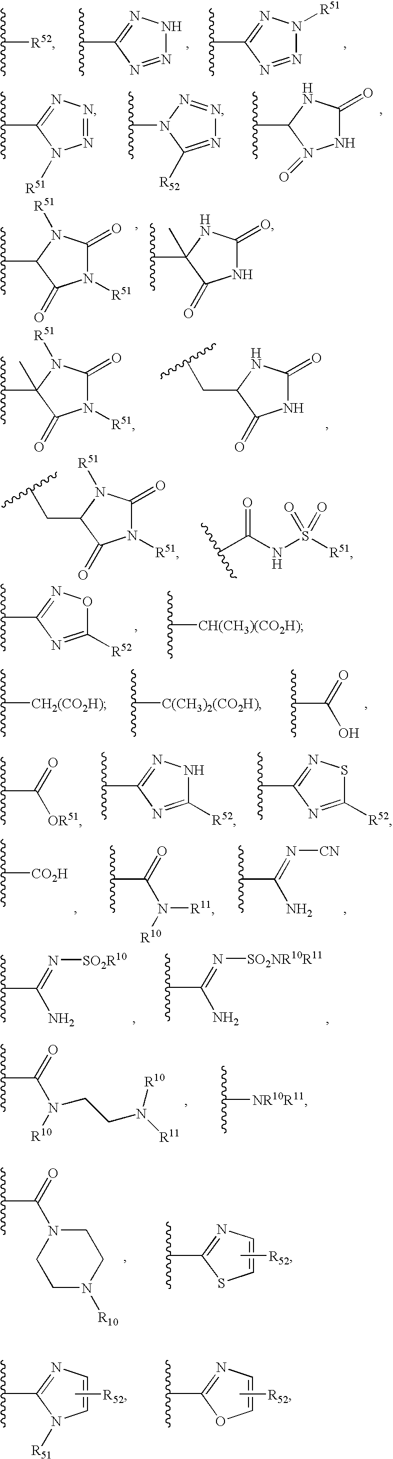

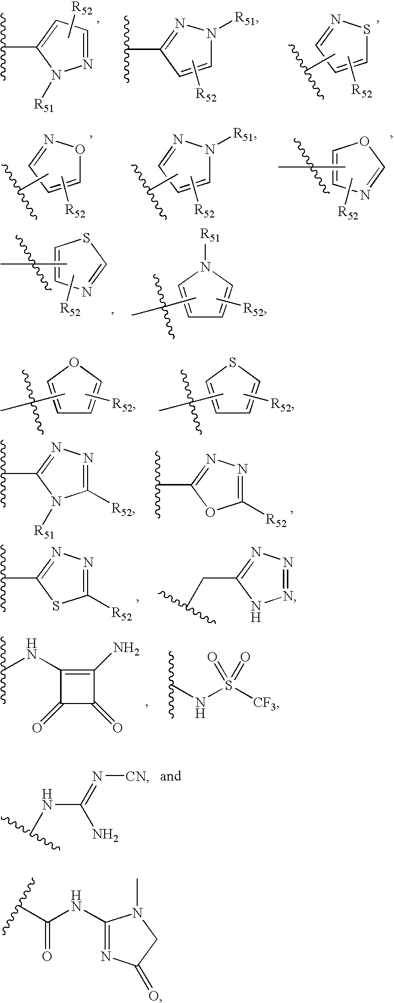

wherein:

R⁵² is selected from the group consisting of hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times.

In yet a further embodiment, R³ of the structures of Formula (I) may be:

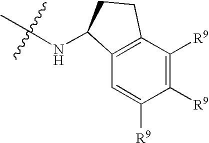

In still a further embodiment, R³ of Formula (I) may be selected from:

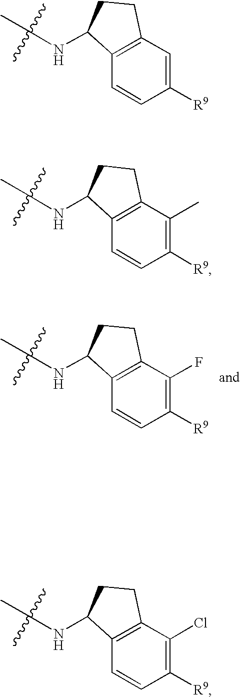

wherein:

R⁹ is selected from the group consisting of hydrogen, fluoro, halo, CN, alkyl, CO₂H,

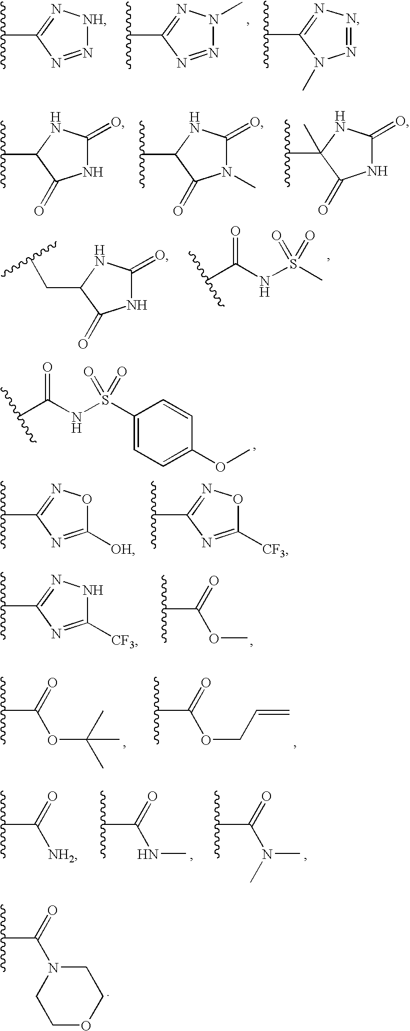

In some embodiments, R¹ of Formula (1) may be selected from:

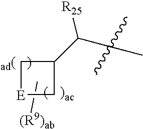

wherein:

ab is selected from the integer (2×ac)+(2×ad)+1;

ac is selected from 1-5;

ad is selected from 0-5;

optionally two R⁹ groups together at the same carbon atom form ═O, ═S or ═NR¹⁰; and

R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times.

In another embodiment, R¹ of Formula (I) may be selected from:

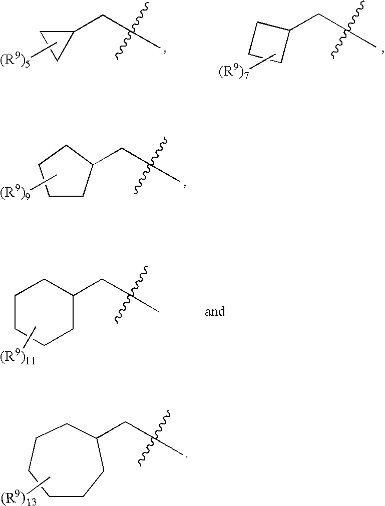

In yet another embodiment, R¹ of Formula (I) may be selected from:

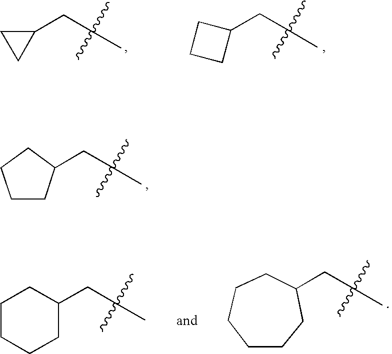

In some embodiments, R¹ of Formula (I) may be selected from:

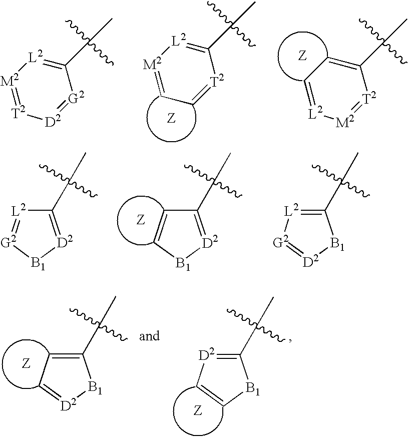

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

B₁ is selected from the group consisting of NR¹⁰, O and S(O)ₓ;

D², G², L², M² and T² are independently selected from the group consisting of CR⁹, CR¹⁸ and N; and

Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

In another embodiment, R¹ of Formula (I) may be selected from:

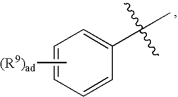

wherein:

ad is selected from 0-5.

In yet another embodiment, R¹ of Formula (I) may be selected from:

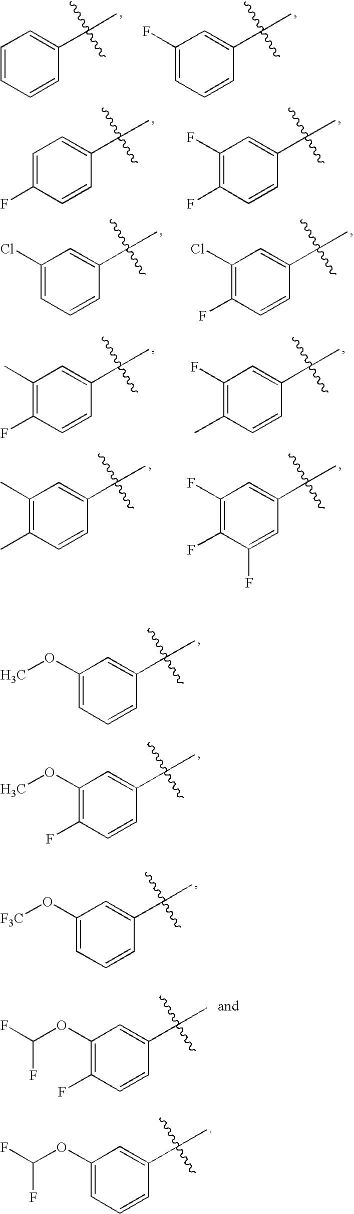

In another embodiment, R¹ of Formula (I) may be selected from:

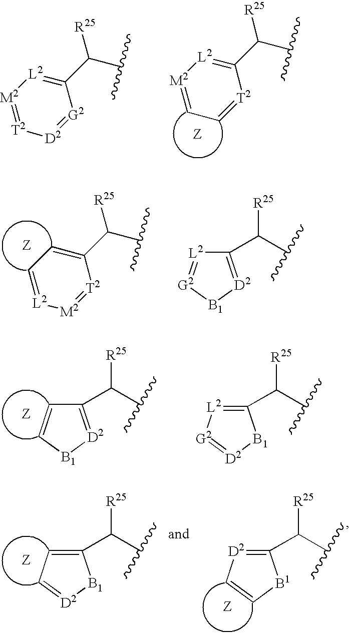

wherein:

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

B, is selected from the group consisting of NR¹⁰, O and S(O)ₓ;

D², G², L², M² and T² are independently selected from the group consisting of CR⁹, CR¹⁸ and N; and

Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

In yet another embodiment, R¹ of Formula (I) may be selected from:

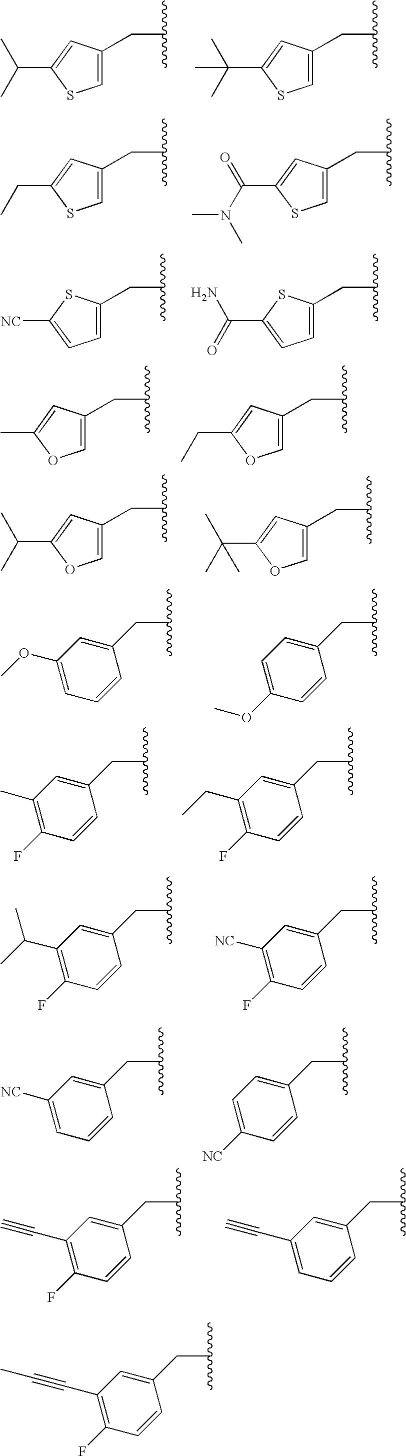

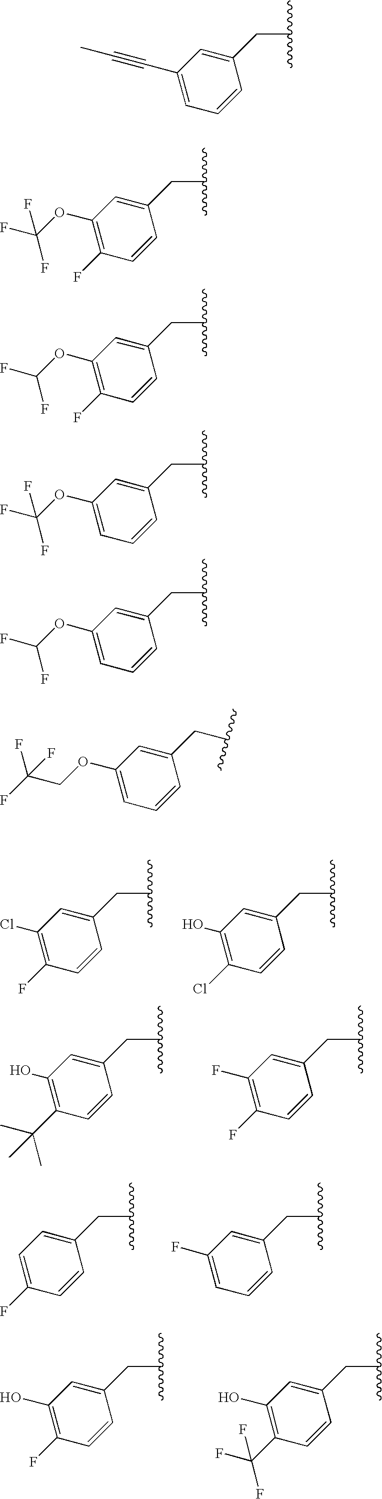

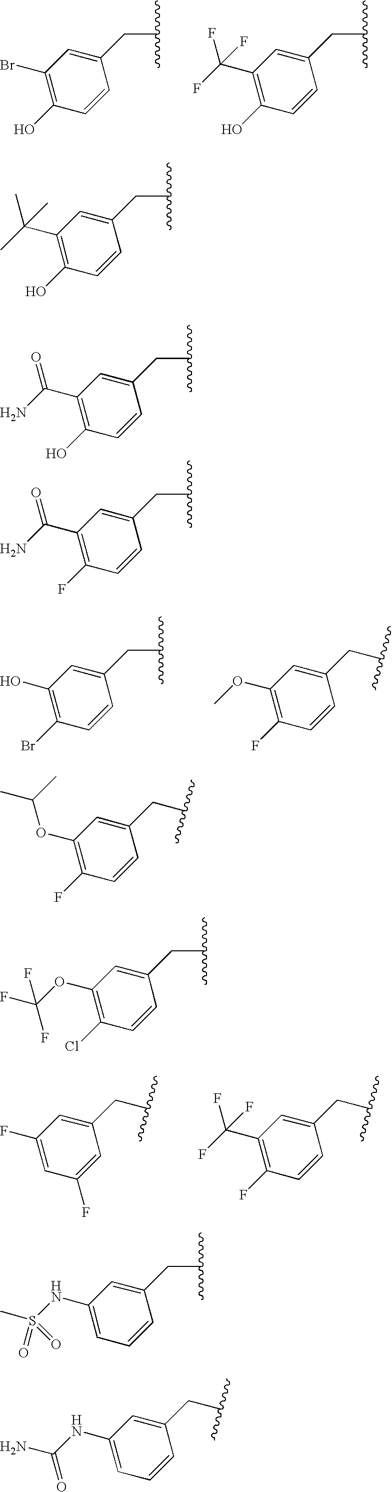

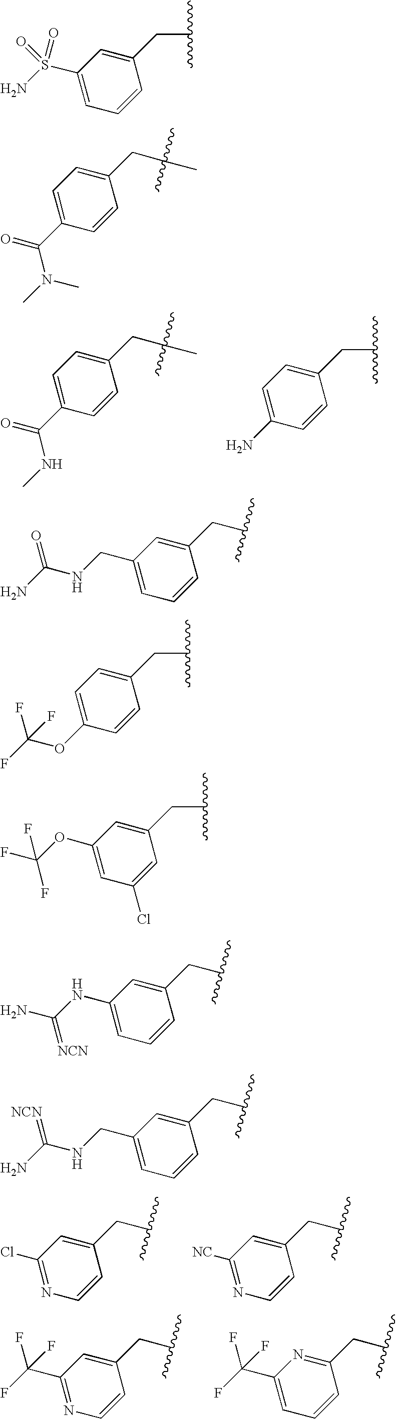

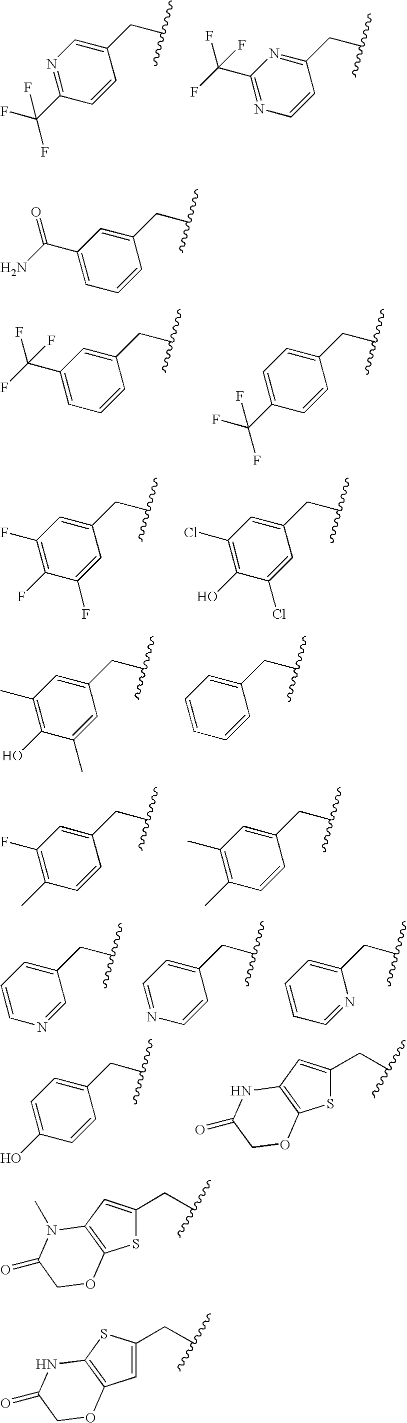

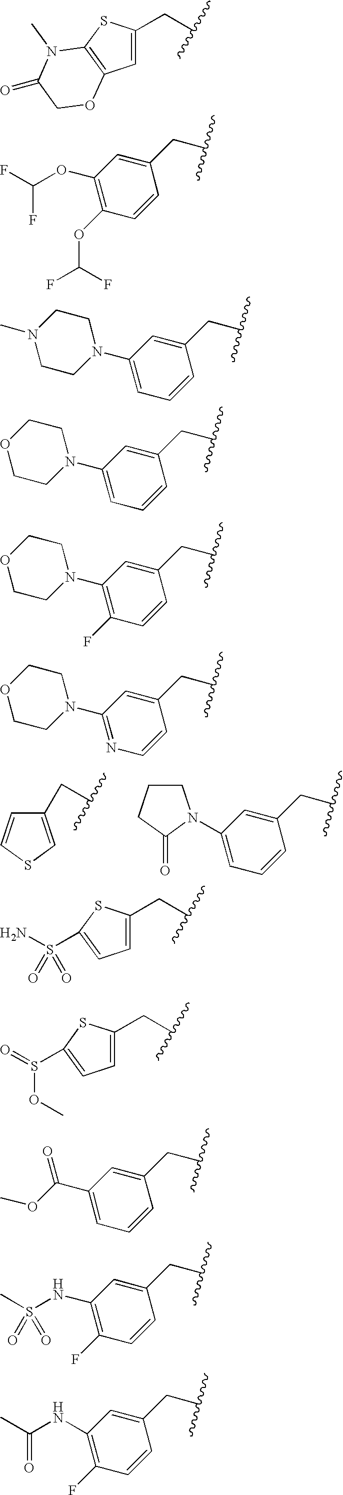

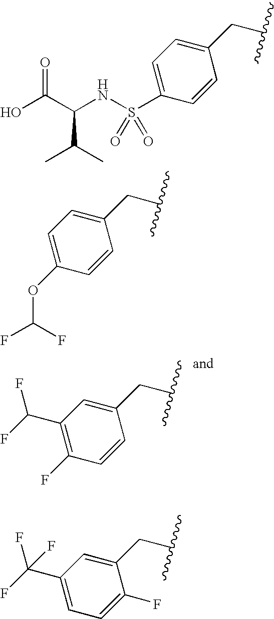

In still another embodiment, R¹ of Formula (I) may be selected from:

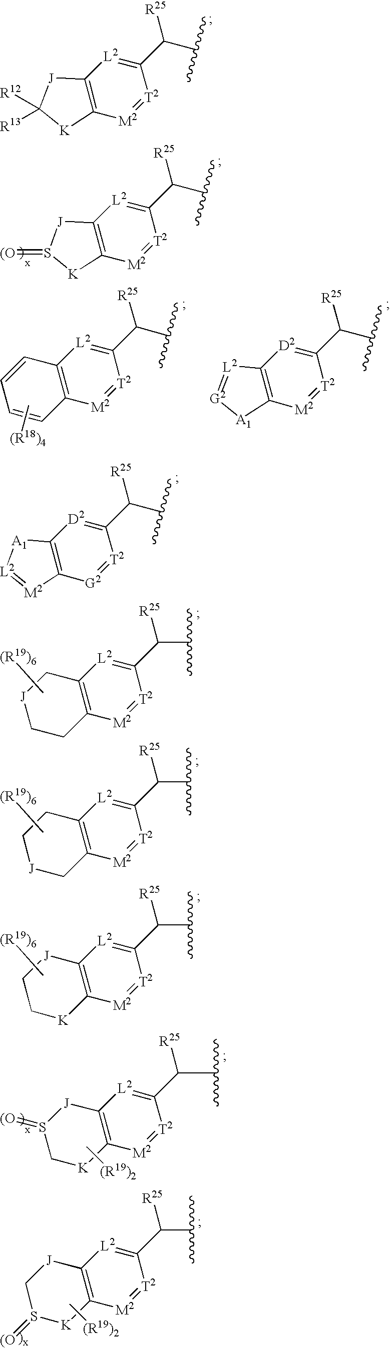

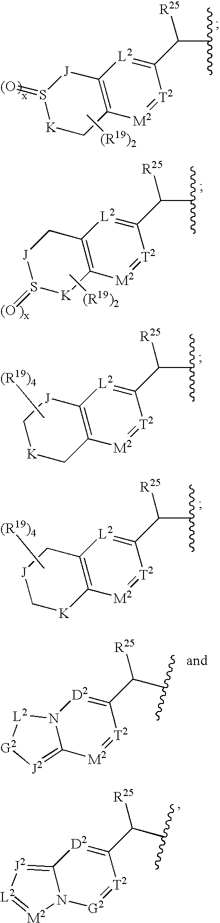

wherein:

R¹² and R¹³ are independently selected from the group consisting of hydrogen, alkyl and halo, wherein alkyl is optionally substituted one or more times, or optionally R¹² and R¹³ together form ═O, ═S or ═NR¹⁰;

R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;

R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;

R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;

J and K are independently selected from the group consisting of CR¹⁰R¹⁸, NR¹⁰, O and S(O)ₓ;

A₁ is selected from the group consisting of NR¹⁰, O and S(O)ₓ; and

D², G², J², L², M² and T² are independently selected from the group consisting of CR⁹, CR¹⁸ and N.

In a further embodiment, R¹ of Formula (I) may be selected from:

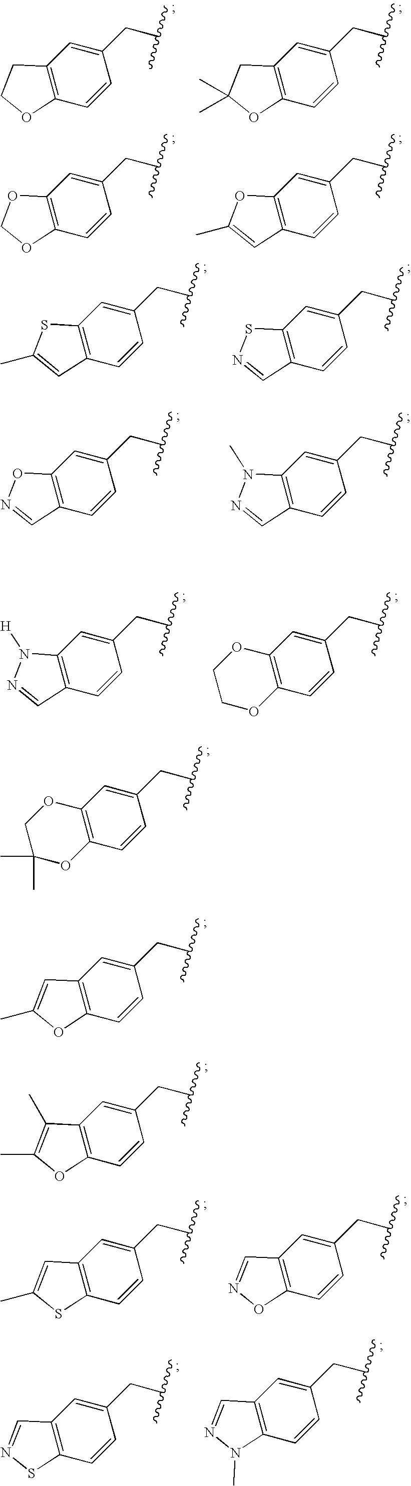

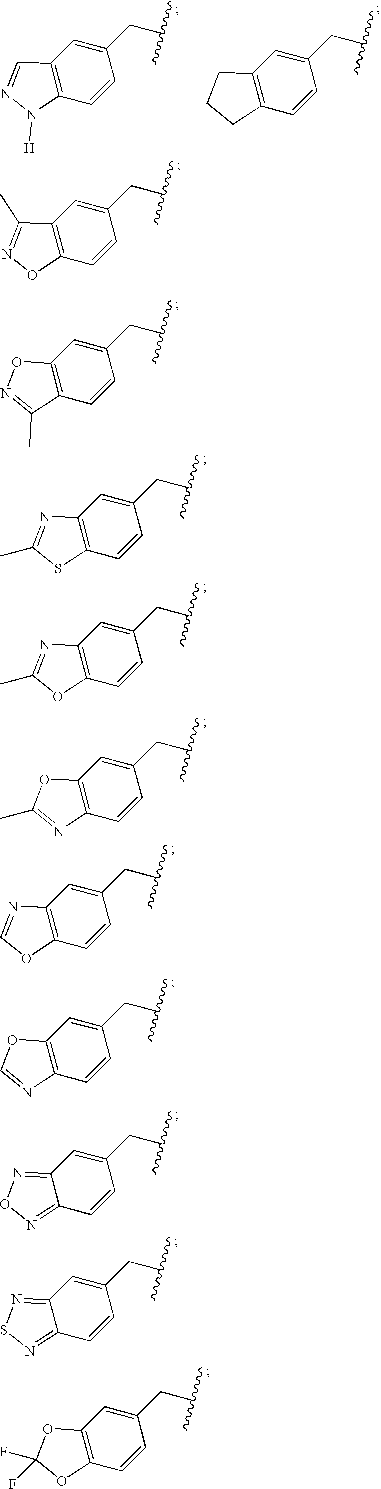

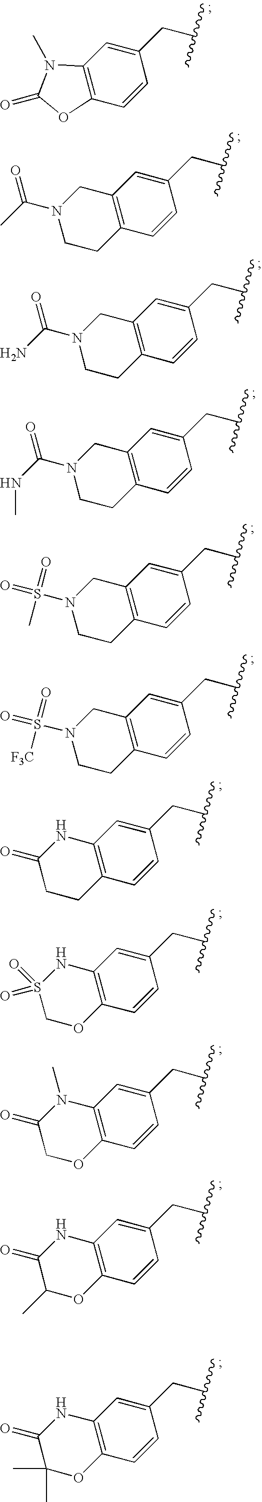

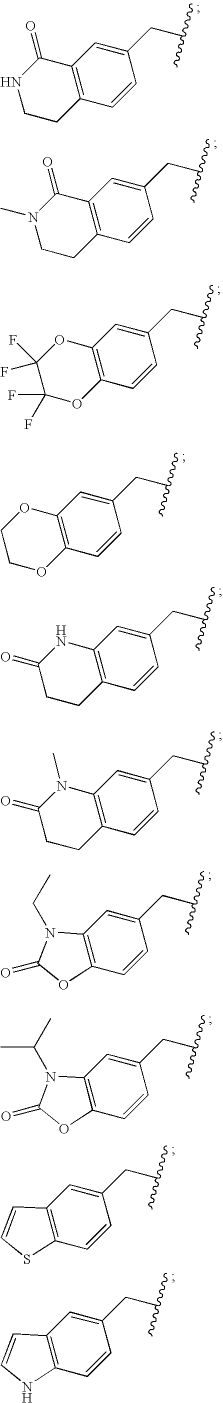

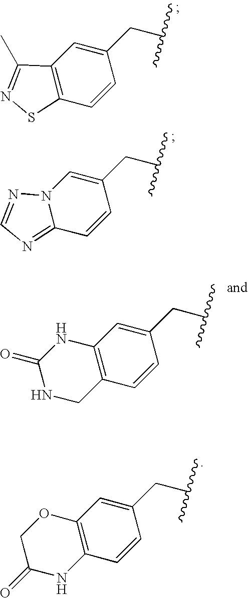

In yet another embodiment, the amide containing heterobicyclic metailoprotease compounds may be represented by the general Formula (II):

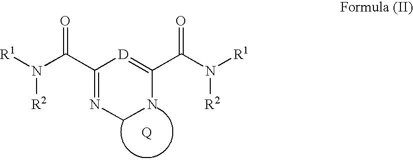

and N-oxides, pharmaceutically acceptable salts, prodrugs, formulation, polymorphs, racemic mixtures and stereoisomers thereof,

wherein:

R¹ in each occurrence may be the same or different and is as defined hereinabove;

R² in each occurrence may be the same or different and is as defined hereinabove; and

all remaining variables are as defined hereinabove.

In still another embodiment, the compound of Formula (II) may be selected from:

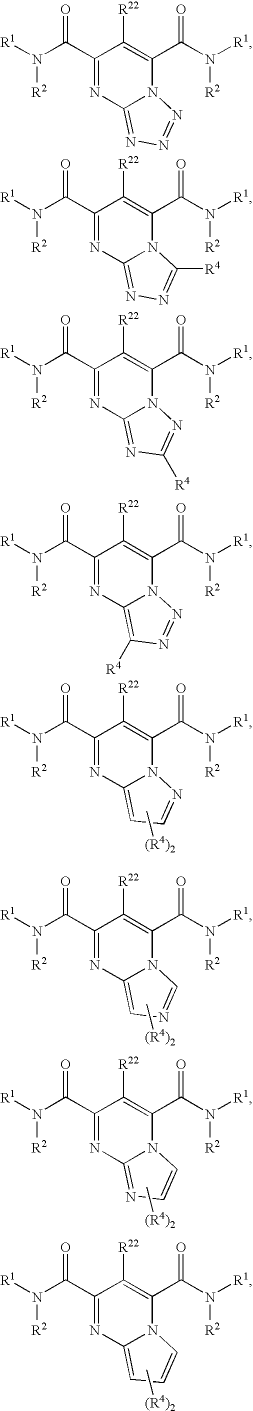

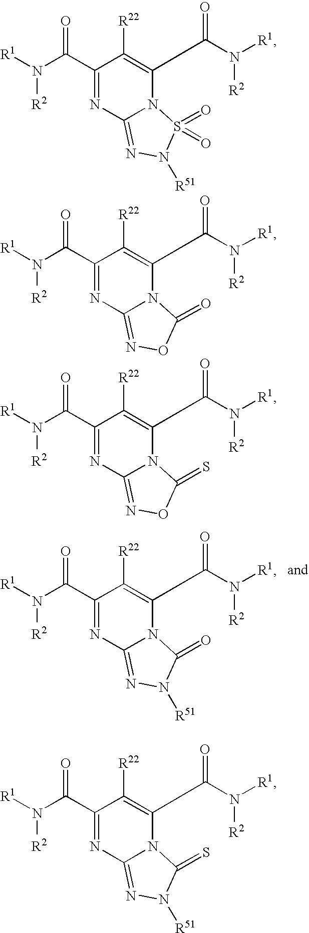

wherein all variables are as defined hereinabove.

In a further embodiment, the compound of Formula (II) may be selected from:

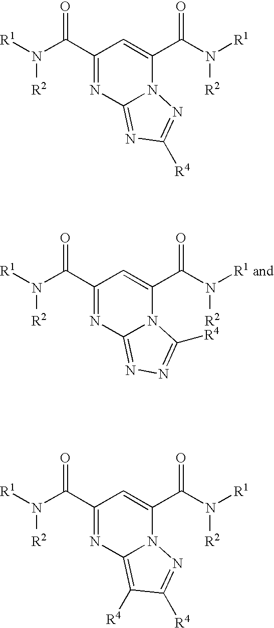

wherein all variables are as defined hereinabove.

In yet a further embodiment, the compound of Formula (II) may be selected from:

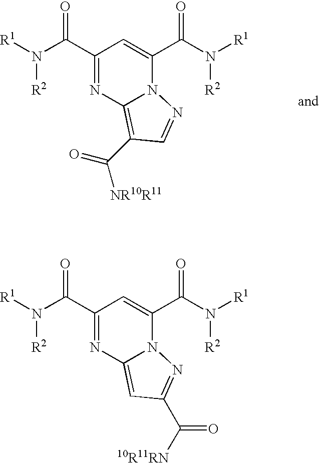

wherein all variables are as defined hereinabove.

In yet a further embodiment, the compound of Formula (II) may be selected from:

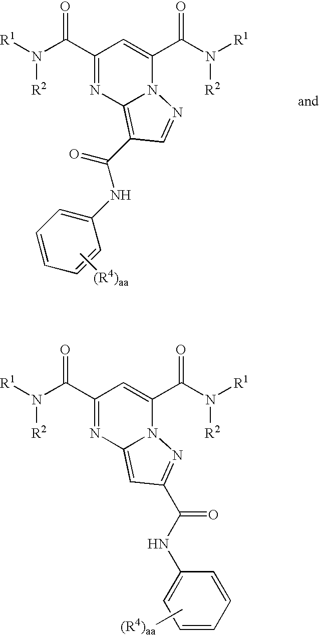

wherein all variables are as defined hereinabove.

In some embodiments, R¹ of Formula (II) may be selected from:

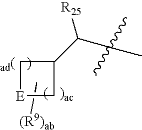

wherein all variables are as defined hereinabove.

In another embodiment, R¹ of Formula (II) may be selected from:

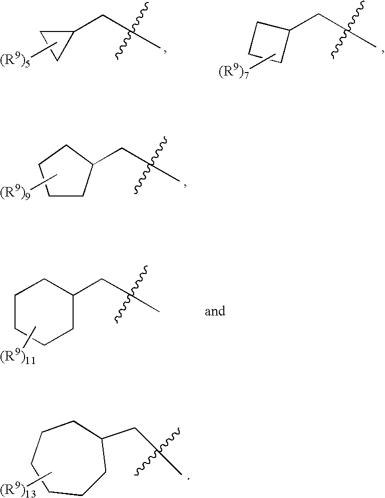

In yet another embodiment, R¹ of Formula (II) may be selected from:

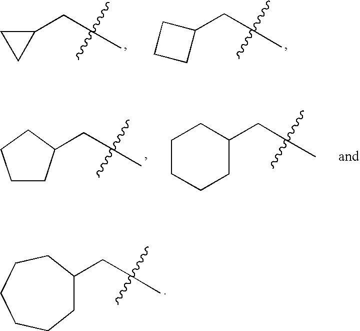

In some embodiments, R¹ of Formula (II) may be selected from:

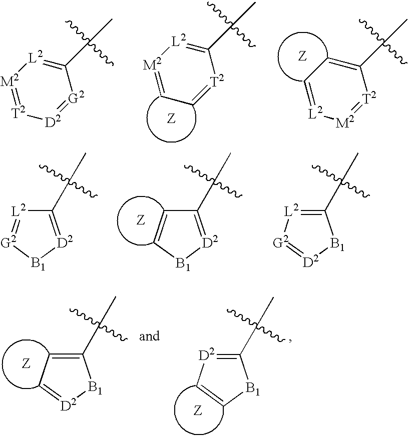

wherein all variables are as defined hereinabove.

In another embodiment, R¹ of Formula (II) may be selected from:

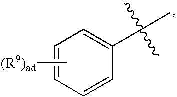

wherein all variables are as defined hereinabove.

In yet another embodiment, R¹ of Formula (II) may be selected from:

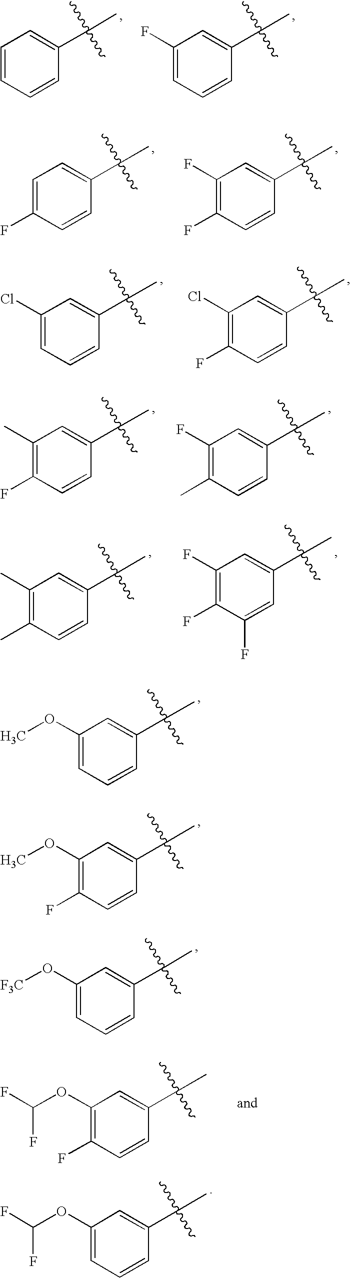

In still a further embodiment, at least one R¹ of Formula (I) may be selected from:

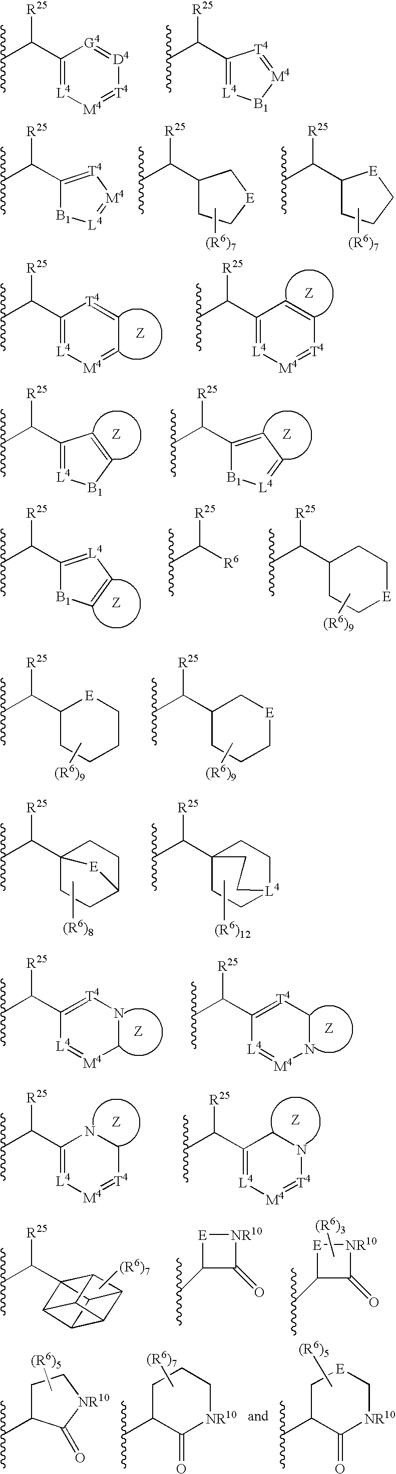

wherein:

R⁶ is independently selected from the group consisting of R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alhkyl-NO₂, (C₀-C₆)-alkyl-CN, (C₀-C₆)-alkyl-S(O)_{y}OR¹⁰, (C₀-C₆)-alkyl-P(O)₂OH, (C₀-C6)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰—alkyl, S(O)₂—(C₀-C₆)-alkyl-aryl, S(O)₂—(C₀-C₆)-alkyl-heteroaryl, (C₀-C₆)-alkyl-C(O)-NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl, wherein each R⁶ group is optionally substituted by one or more R¹⁴ groups;

D⁴, G⁴, L⁴, M⁴, and T¹ are independently selected from CR⁶ and N; and

all remaining variables are as defined hereinabove.

In another embodiment, at least one R¹ of Formula (II) may be selected from:

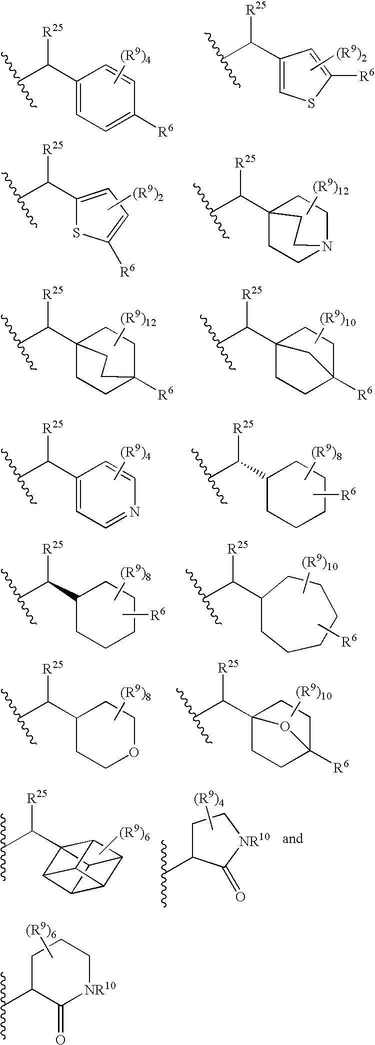

In yet another embodiment, R⁶ is selected from the group consisting of hydrogen, halo, CN, OH, CH₂OH, CF₃, CHF₂, OCF₃, OCHF₂, COCH₃, SO₂CH₃, SO₂CF₃, SO₂NH₂, SO₂NHCH₃, SO₂N(CH₃)₂, NH₂, NHCOCH₃, N(COCH₃)₂, NHCONH₂, NHSO₂CH₃, alkoxy alkyl, cycloalkyl, heterocycloalkyl, bicycloalkyl, CO₂H,

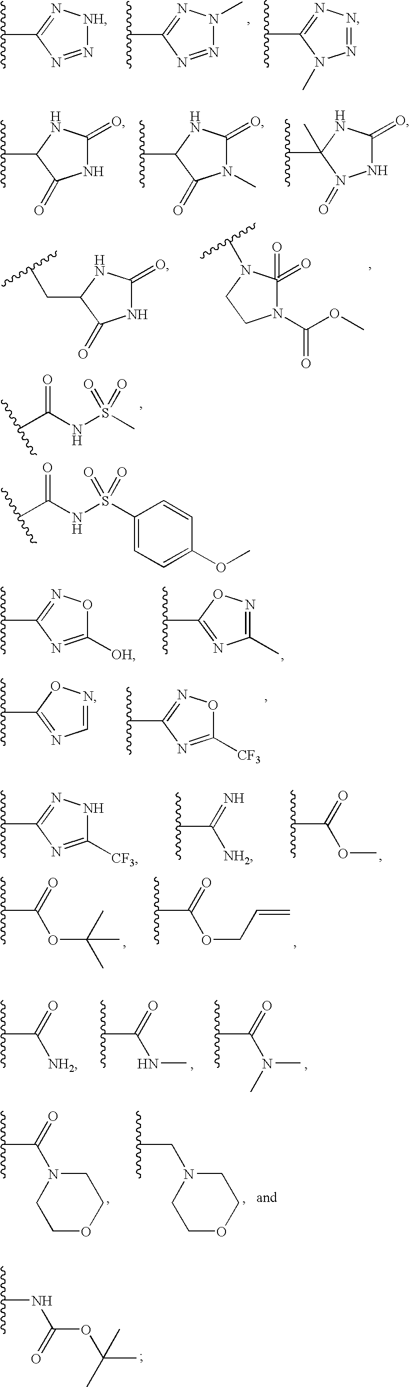

R⁹ is independently selected from the group consisting of hydrogen, fluoro, chloro, CH₃, CF₃, CHF₂, OCF₃, and OCHF₂;

R₂₅ is selected from the group consisting of hydrogen, CH₃, COOCH₃, COOH, and CONH₂.

In yet another embodiment, at least one R¹ of Formula (II) may be selected from:

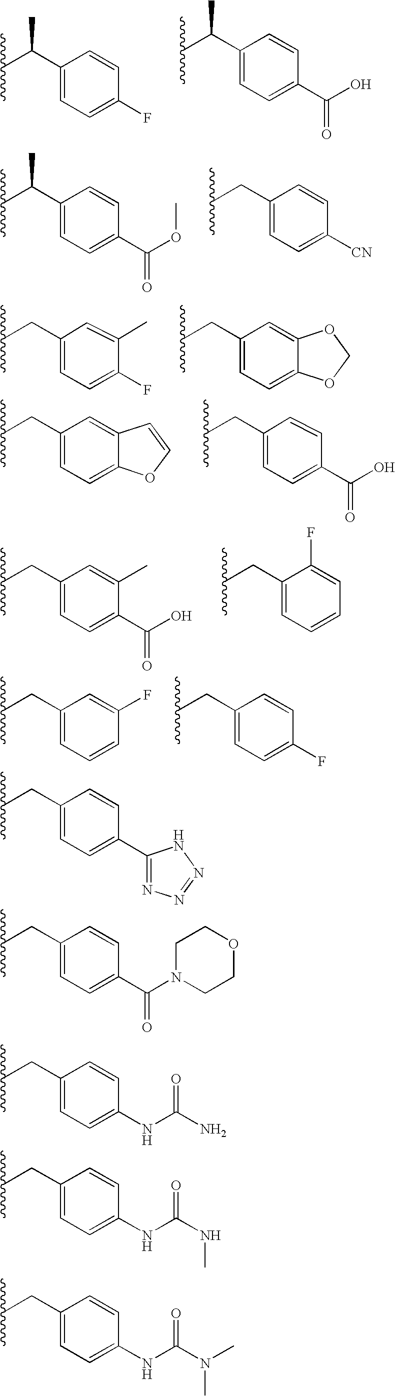

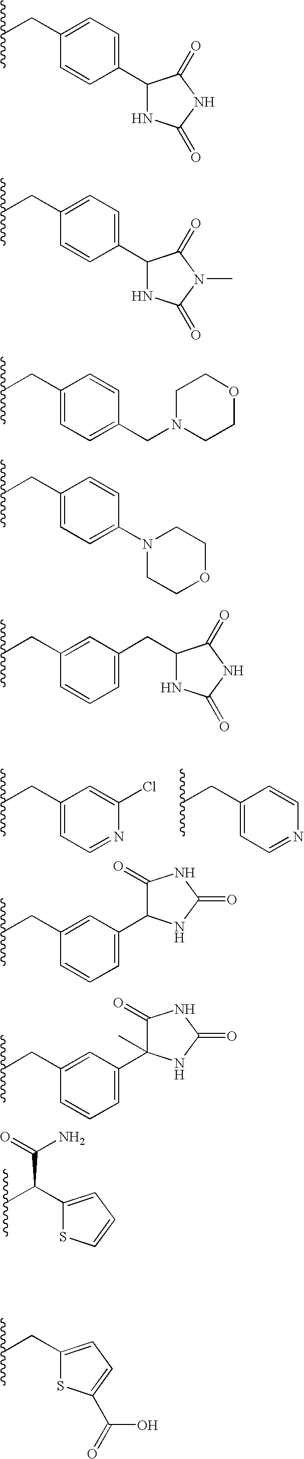

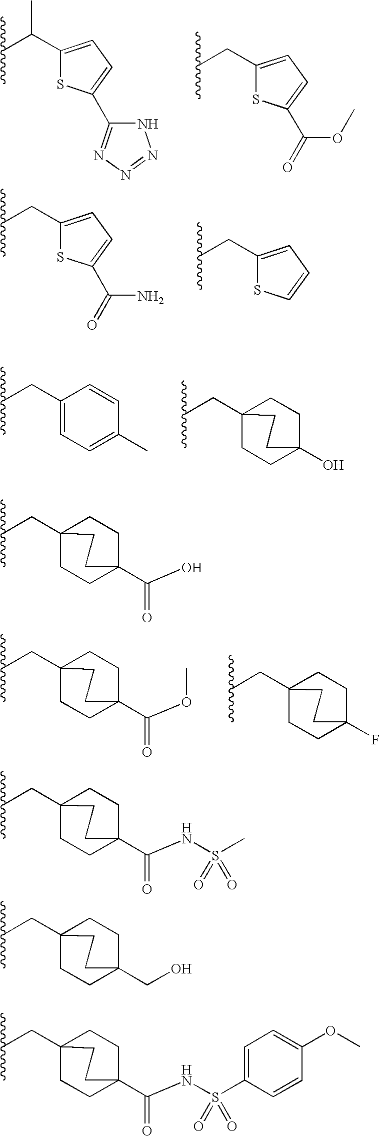

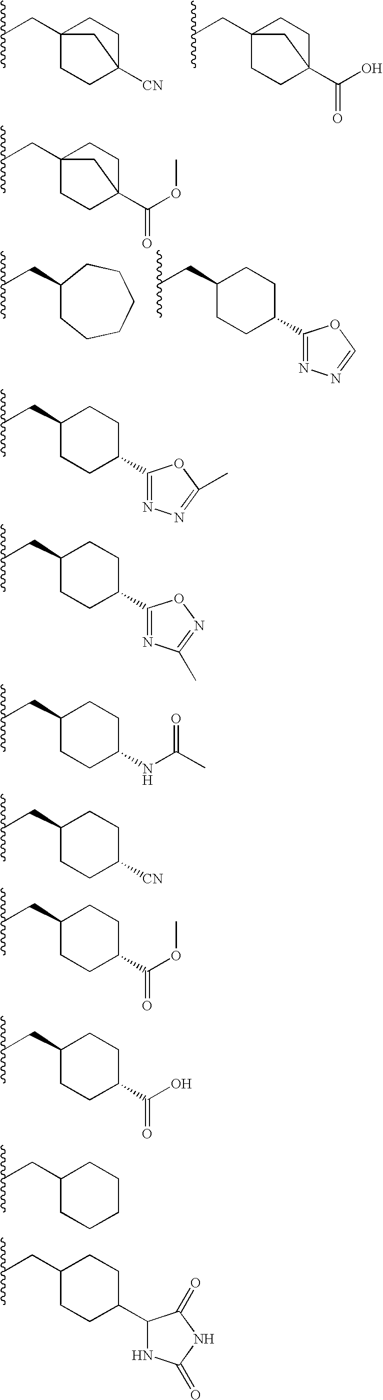

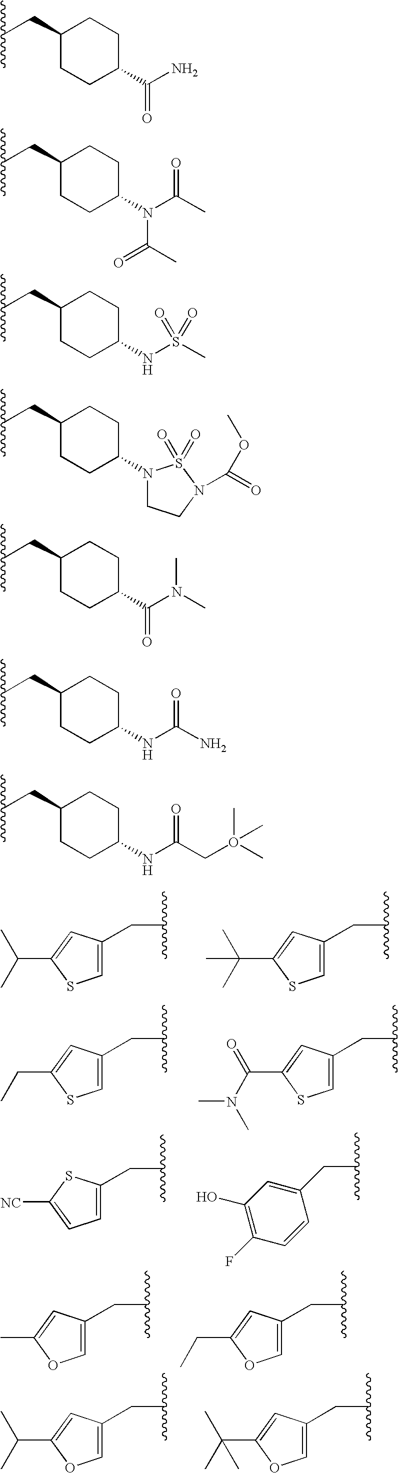

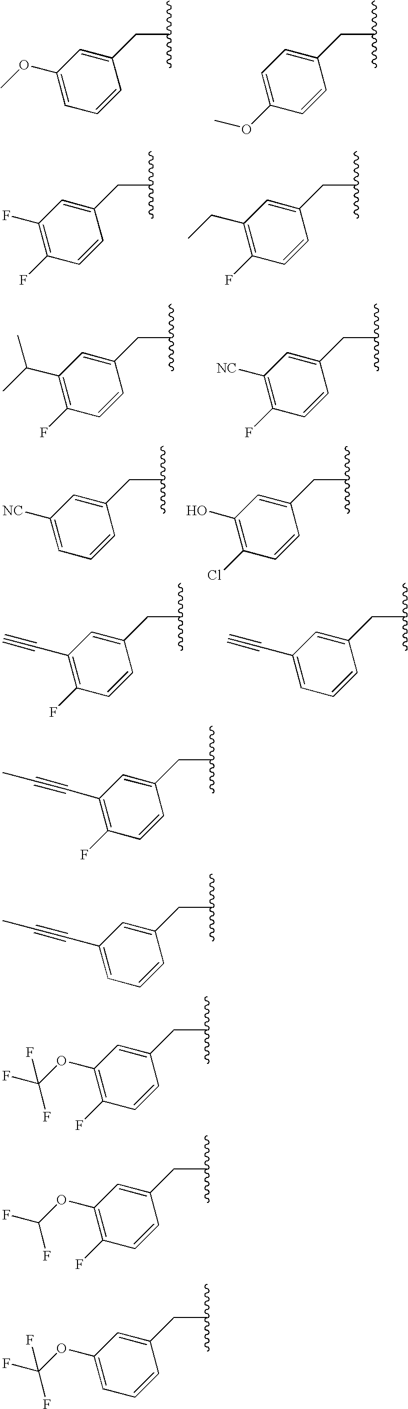

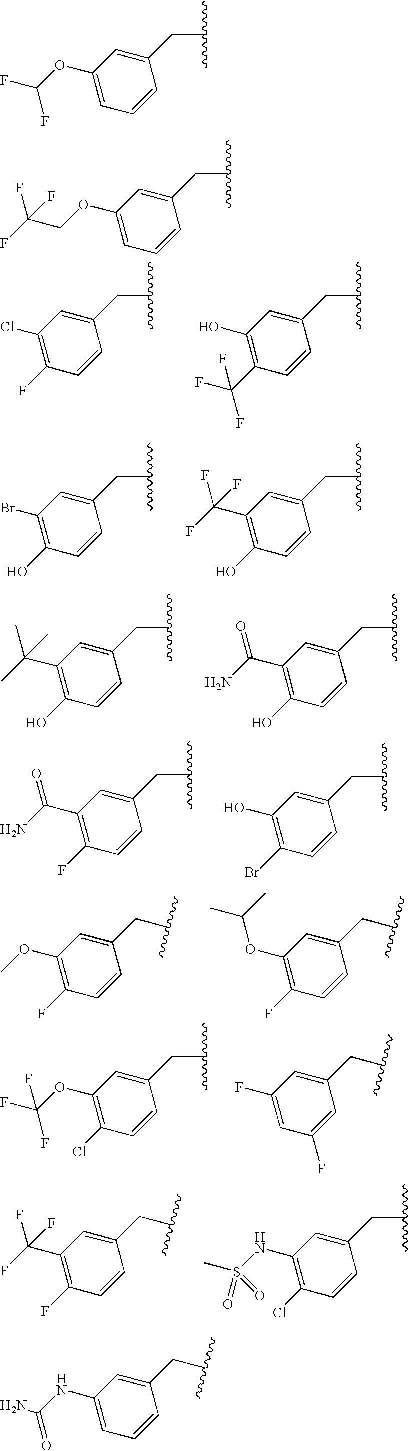

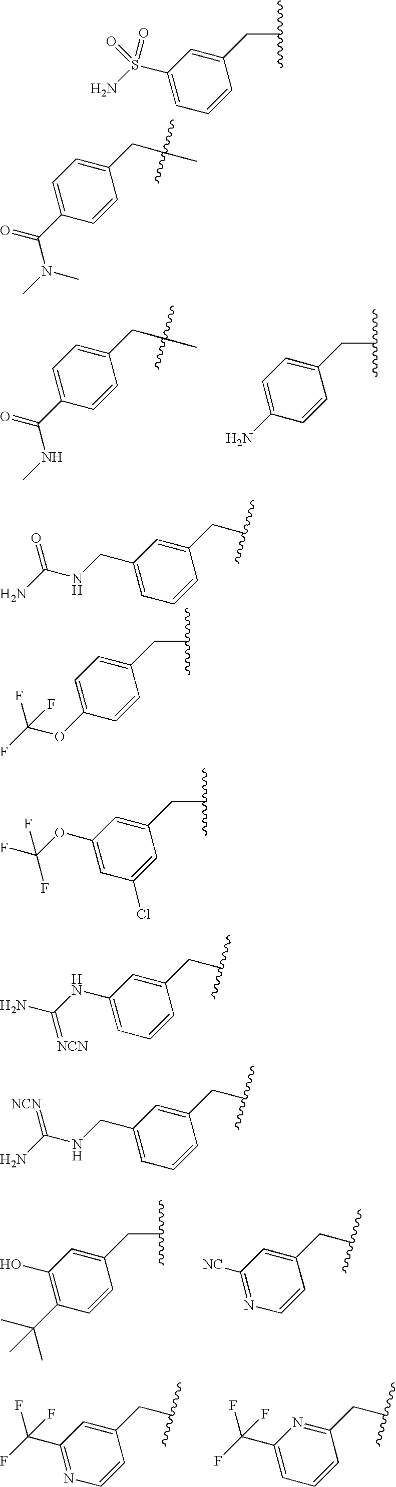

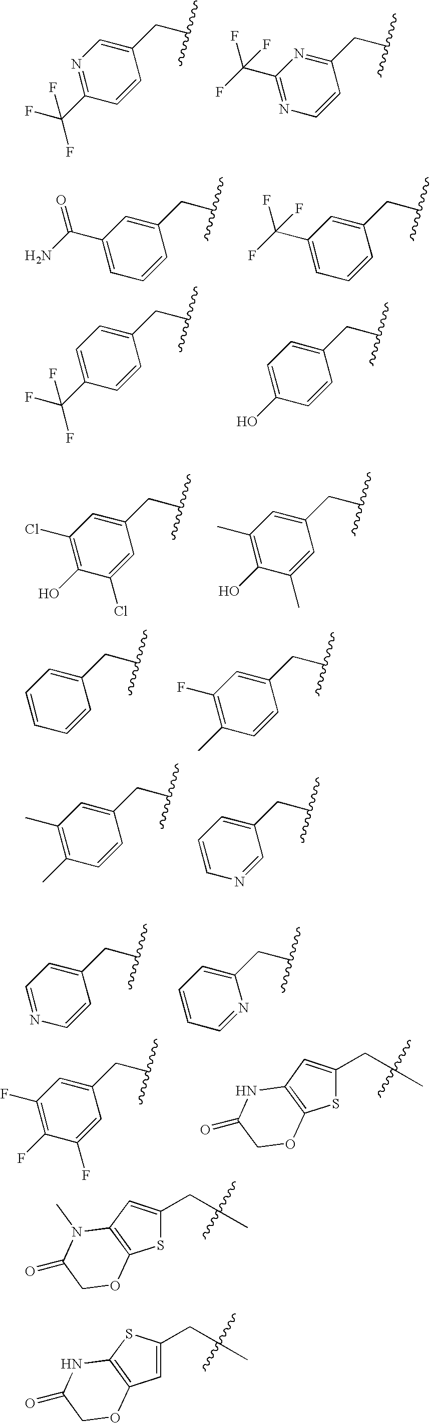

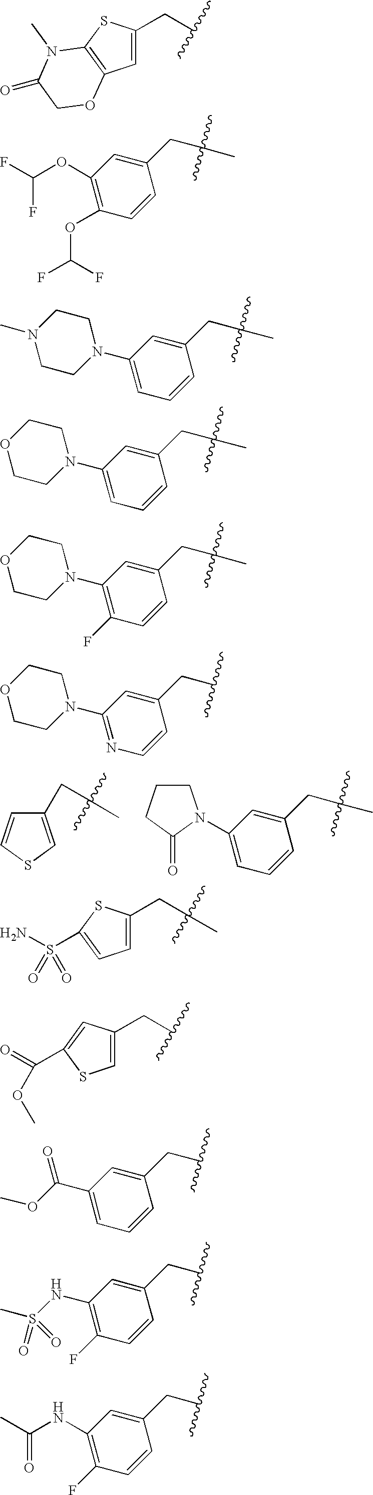

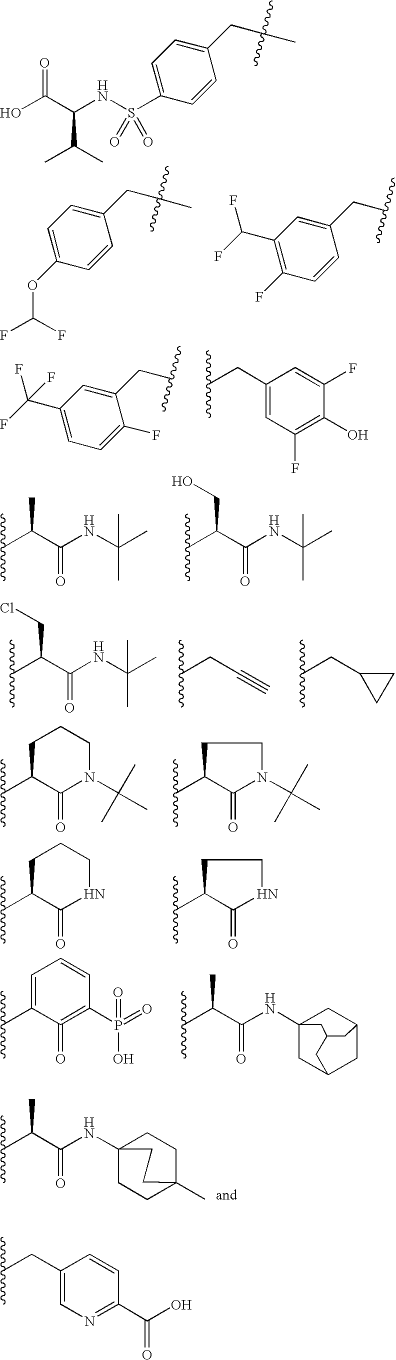

In still another embodiment, at least one R¹ of Formula (II) may be selected from: 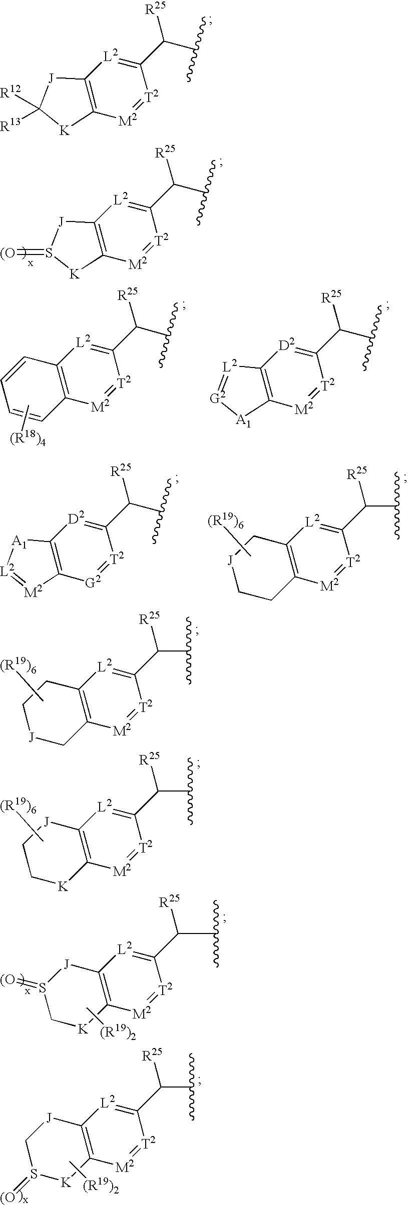 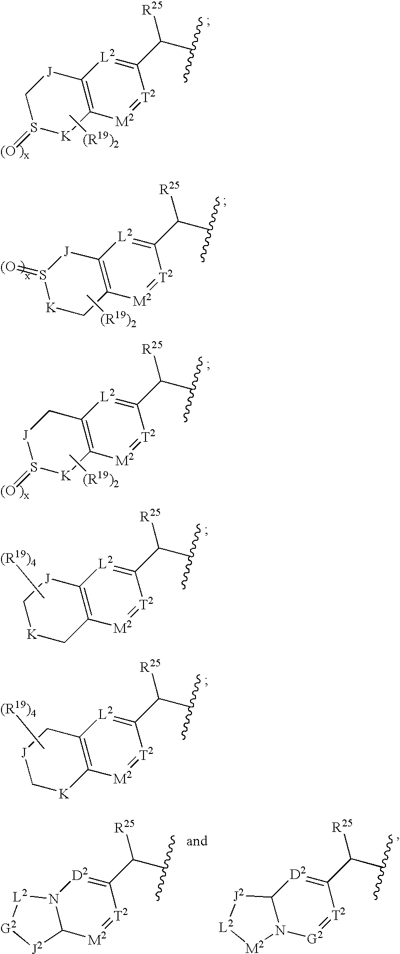
wherein all variables are as defined hereinabove.

In a further embodiment, at least one R¹ of Formula (II) may be selected from:

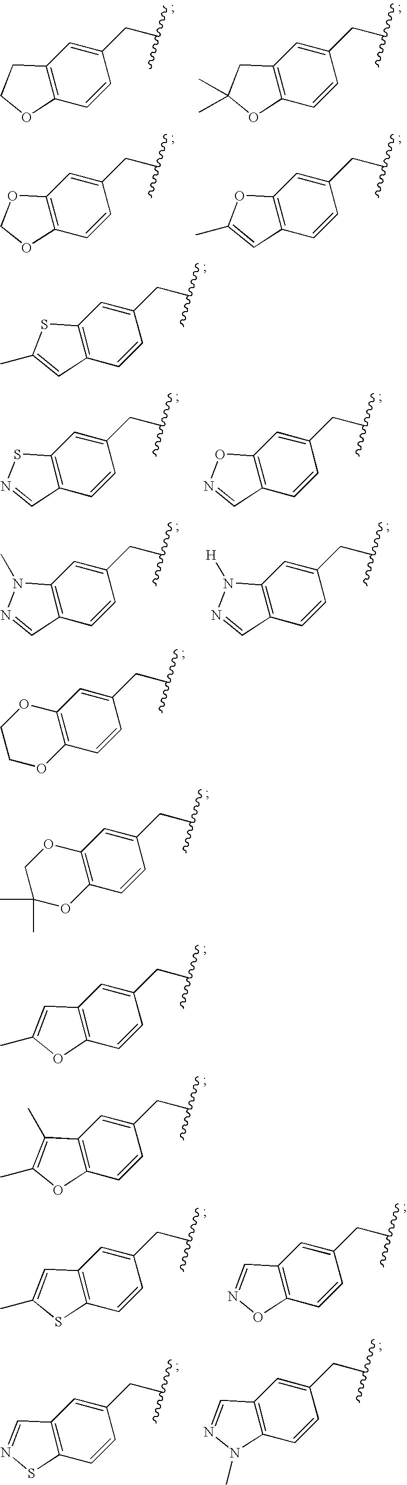

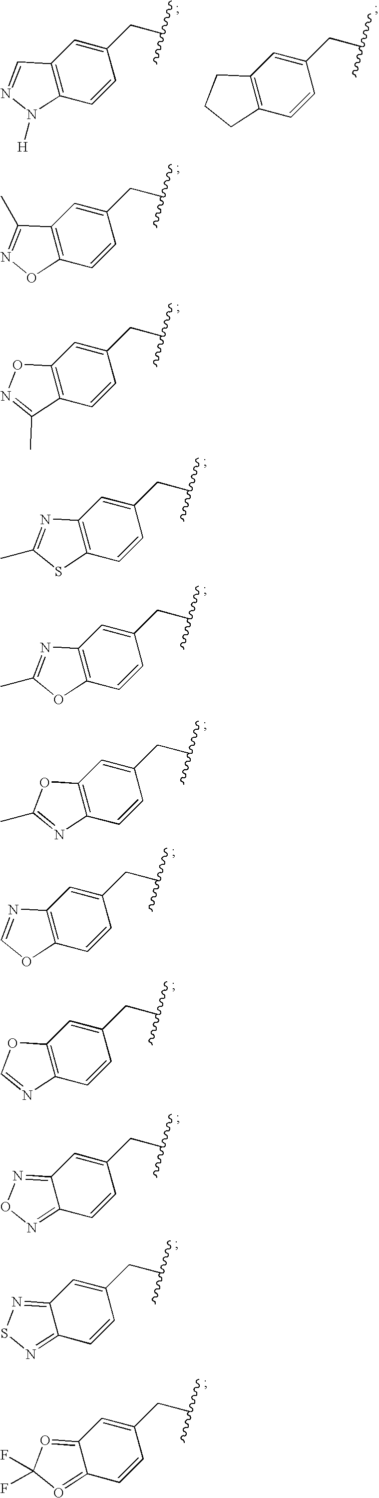

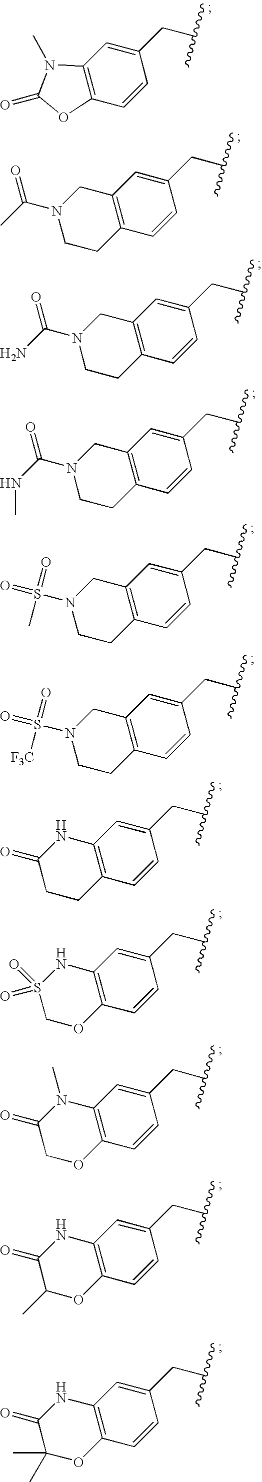

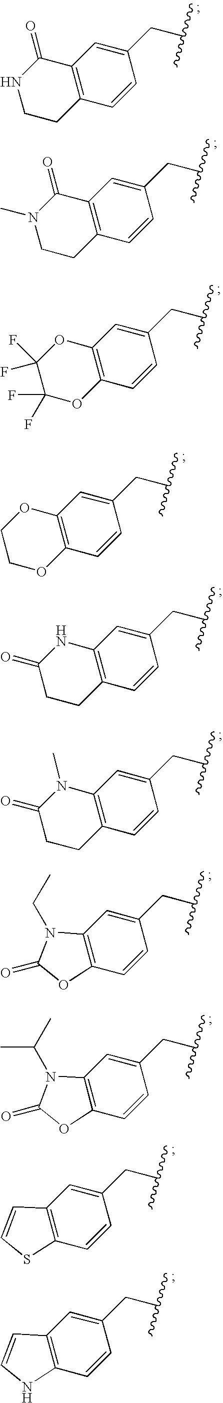

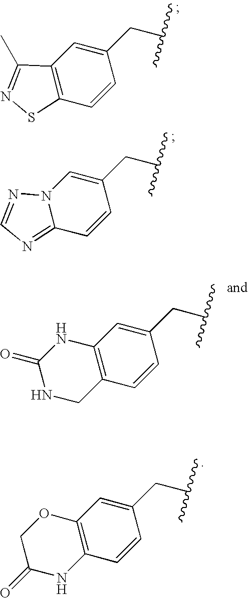

In another embodiment of the present invention, the amide containing heterobicyclic metalloprotease compounds may be represented by the general Formula (II): 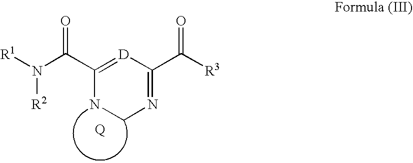
and N-oxides, pharmaceutically acceptable salts, prodrugs, formulation, polymorphs, racemic mixtures and stereoisomers thereof,
wherein all variables are as defined hereinabove.

In yet another embodiment, the compounds of Formula (III) may be selected from: 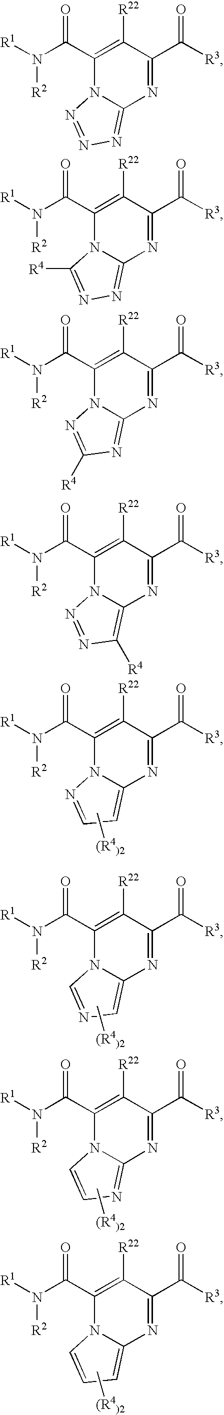 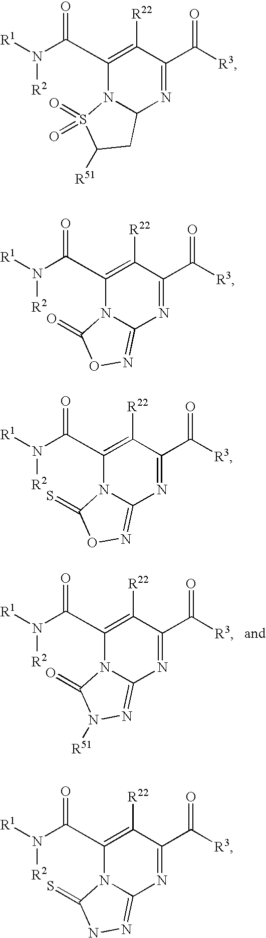
wherein all variables are as defined hereinabove.

In still another embodiment, the compounds of Formula (III) may be selected from:

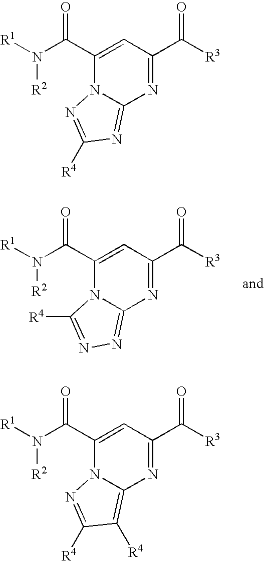

In a further embodiment, the compounds of Formula (III) may be selected from:

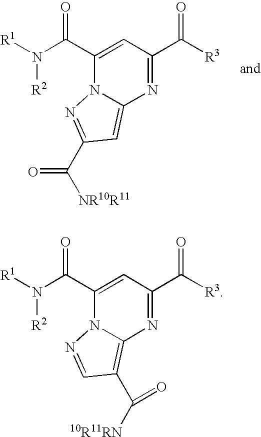

In a further embodiment, the compounds of Formula (III) may be selected from:

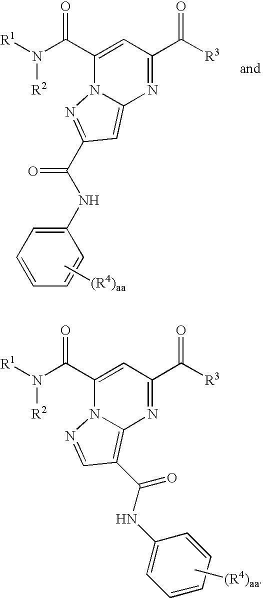

In yet a further embodiment, R³ of Formula (III) may be selected from: 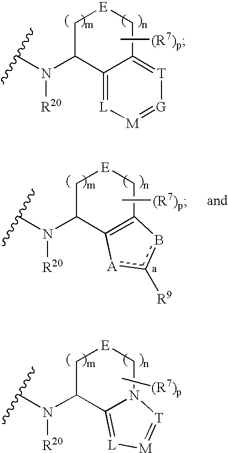
wherein all variables are as defined hereinabove.

In still a further embodiment, R³ of Formula (III) may be selected from: 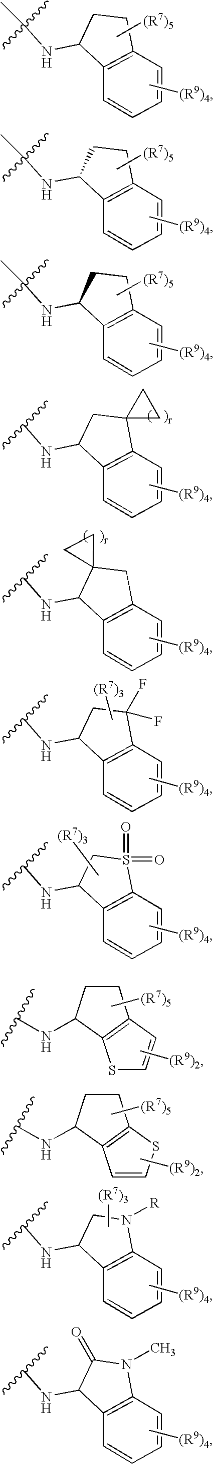 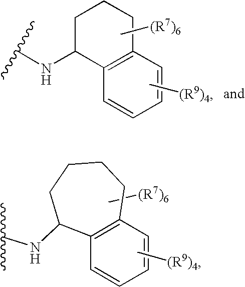
wherein all variables are as defined hereinabove.

In one embodiment, R³ of Formula (III) may be selected from:

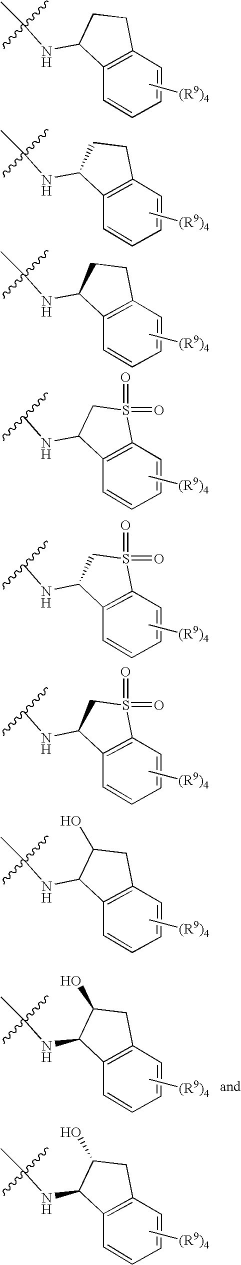

In one embodiment, R⁹ may be selected from: 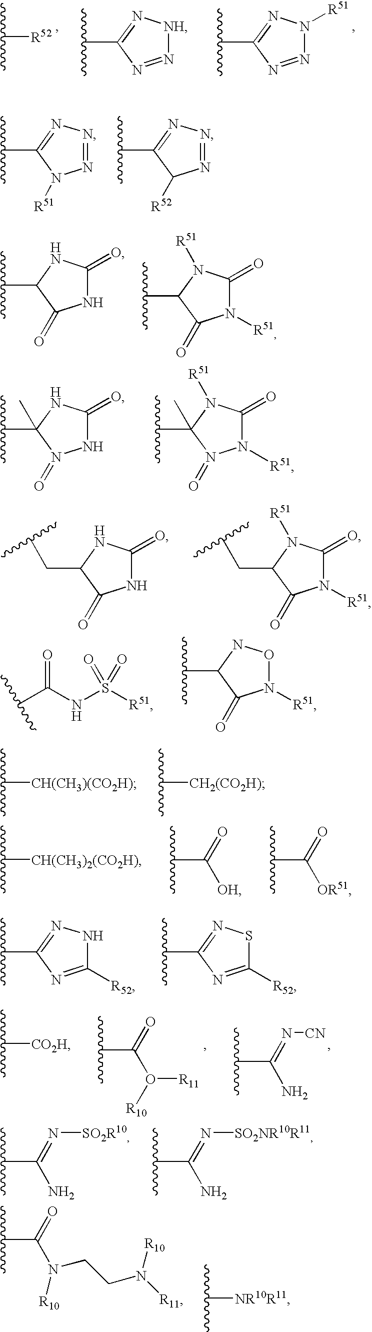 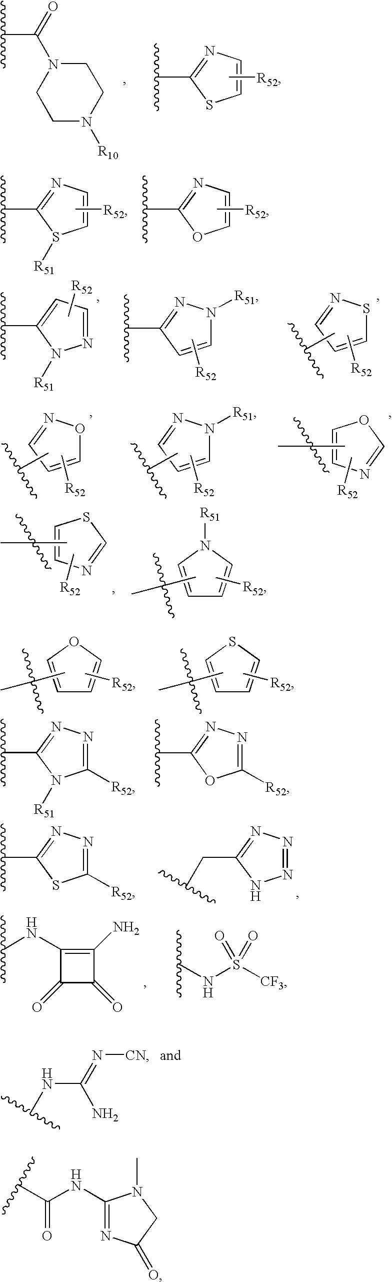
wherein all variables are as defined hereinabove.

In yet another embodiment, R³ of Formula (III) may be:

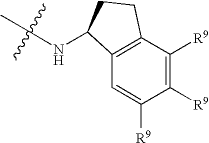

In yet another embodiment, R³ of Formula (III) may be: 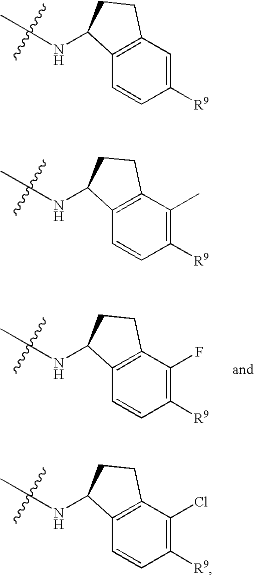
wherein:
R⁹ is selected from the group consisting of hydrogen, fluoro, halo, CN, alkyl, CO₂H,

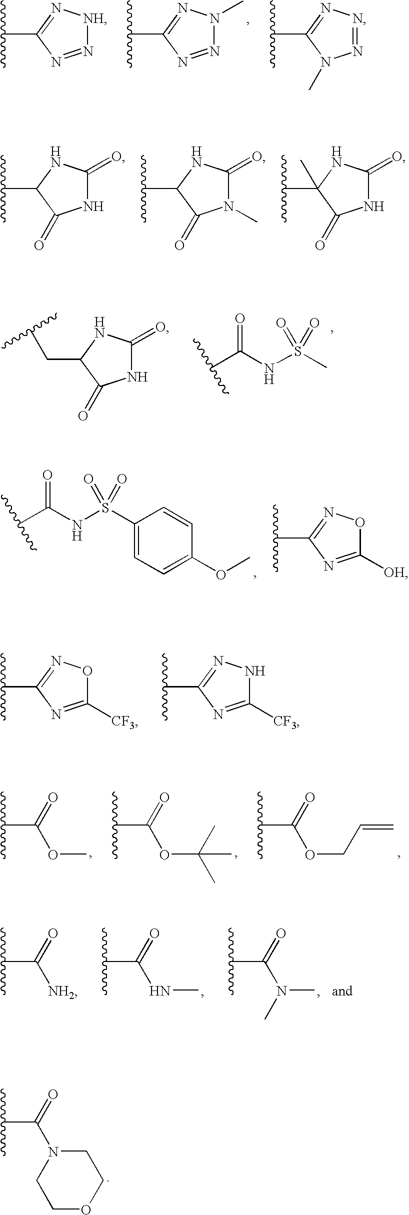

In some embodiments, R¹ of Formula (III) may be selected from: 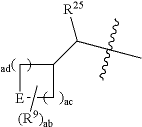
wherein all variables are as defined hereinabove.

In another embodiment, R¹ of Formula (III) may be selected from:

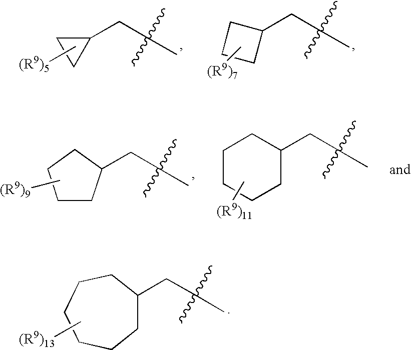

In yet another embodiment, R¹ of Formula (III) may be selected from:

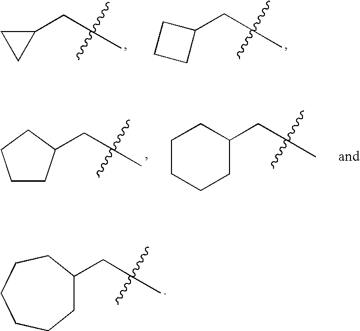

In some embodiments, R¹ of Formula (III) may be selected from: 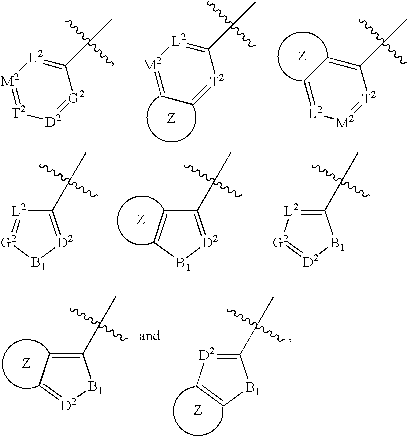
wherein all variables are as defined hereinabove.

In another embodiment, R¹ of Formula (III) may be selected from: 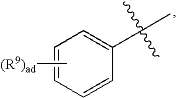
wherein all variables are as defined hereinabove.

In yet another embodiment, R¹ of Formula (III) may be selected from:

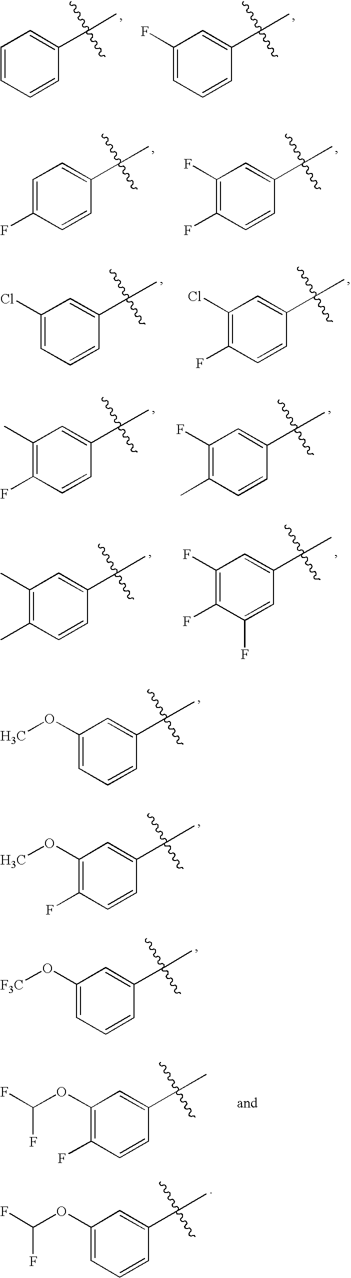

In still another embodiment, R¹ of the structures of Formula (III) may be selected from: 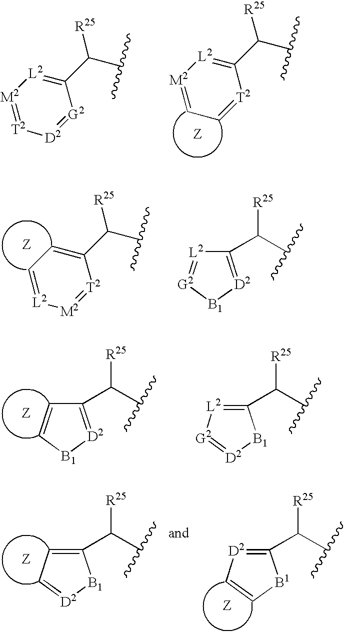
wherein all variables are as defined hereinabove.

In a further embodiment, R¹ of Formula (III) may be selected from:

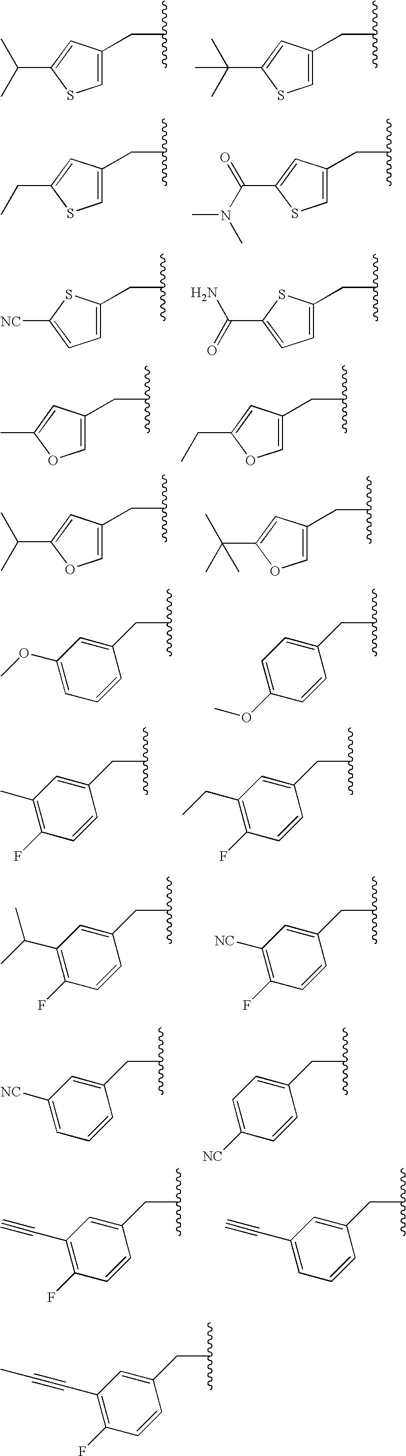

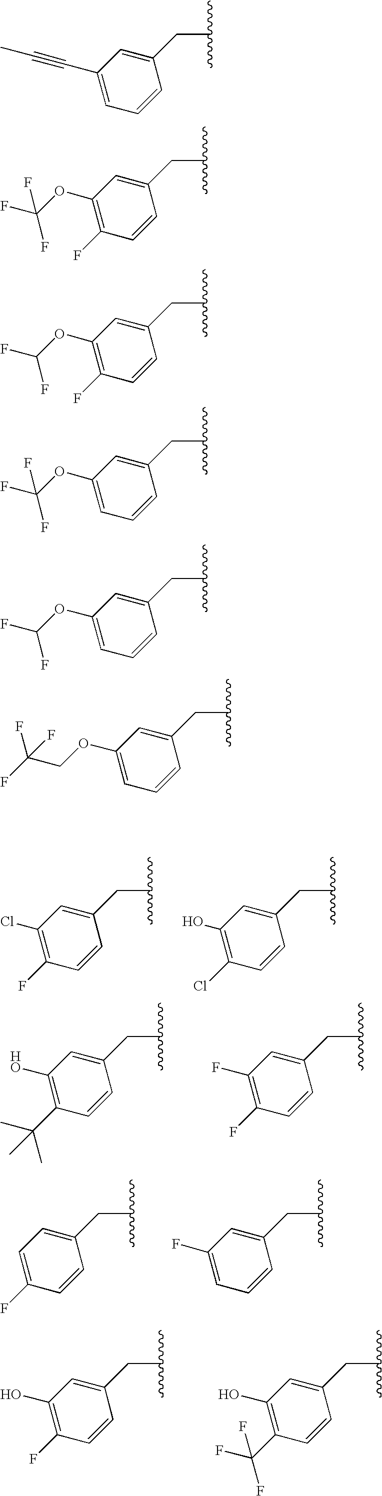

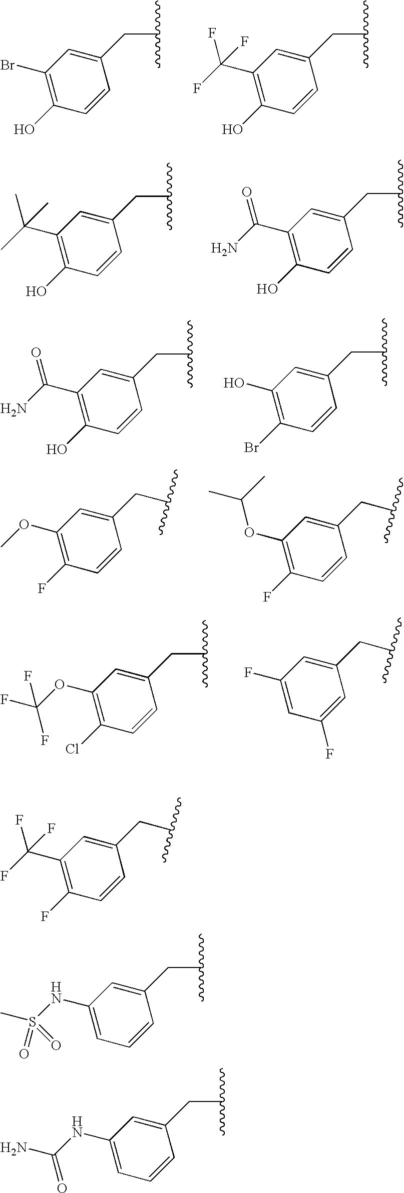

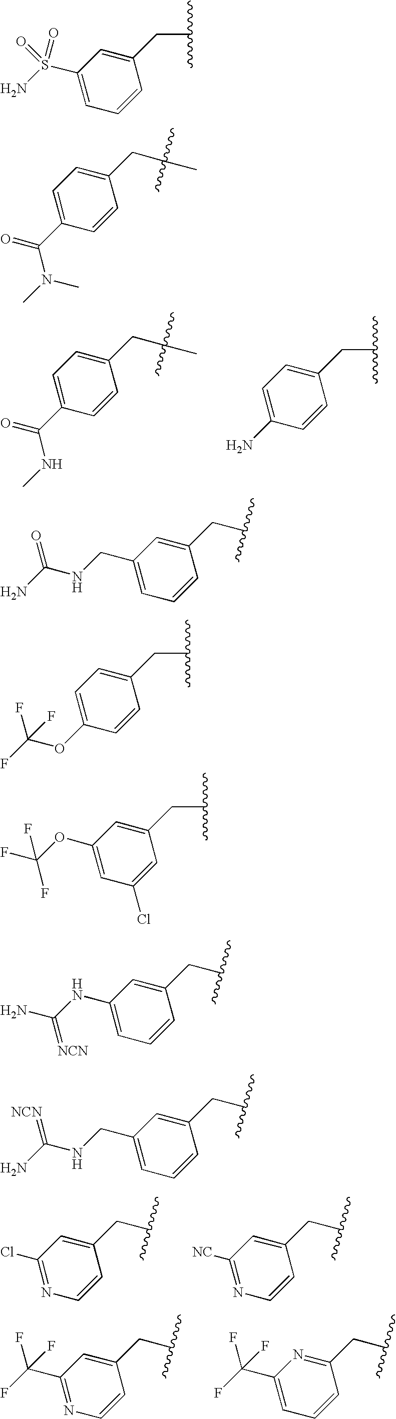

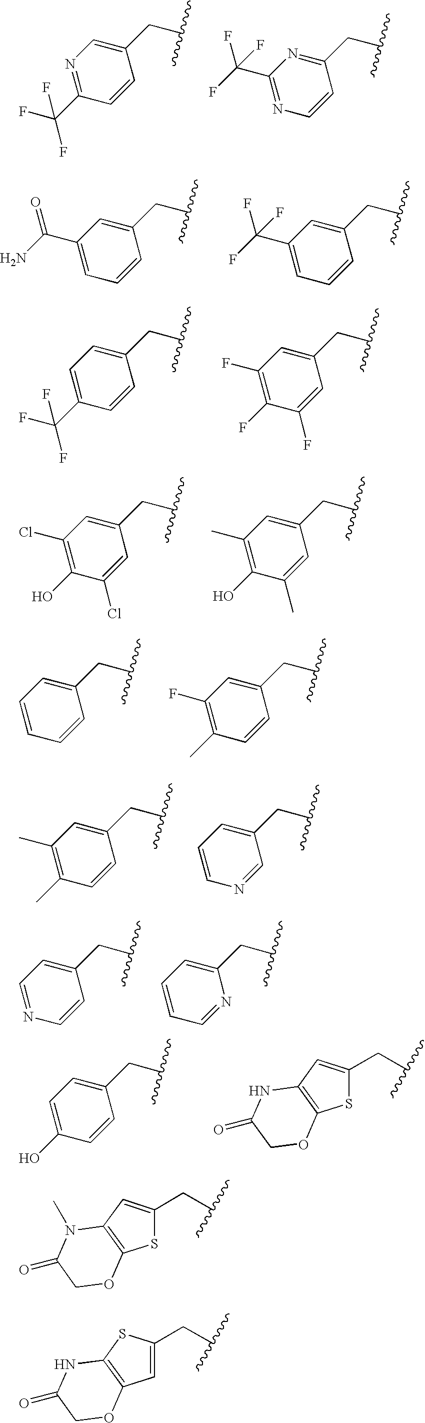

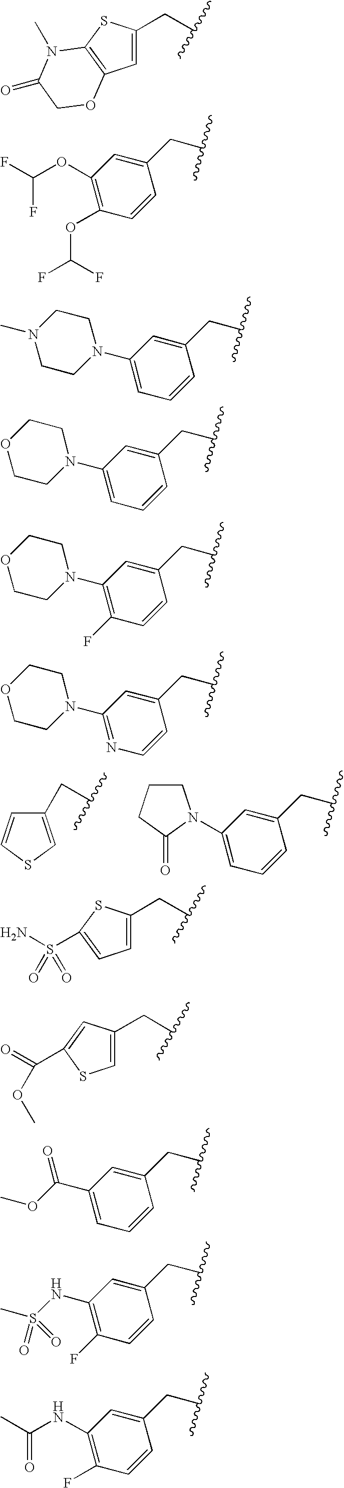

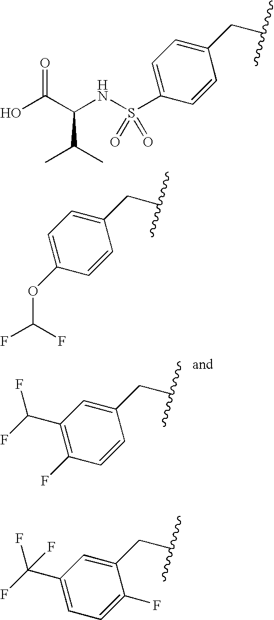

In yet a further embodiment, R¹ of Formula (III) may be selected from: 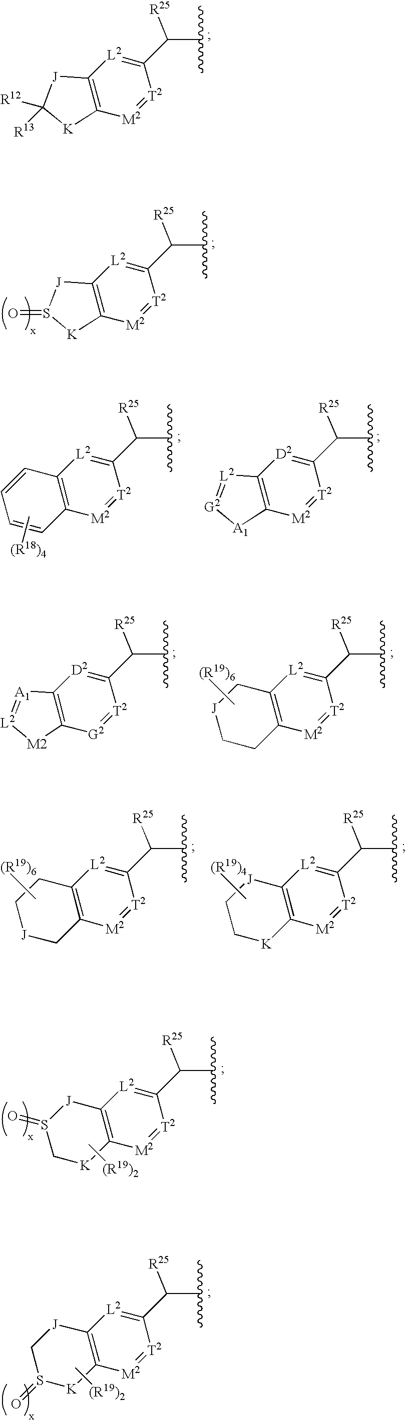 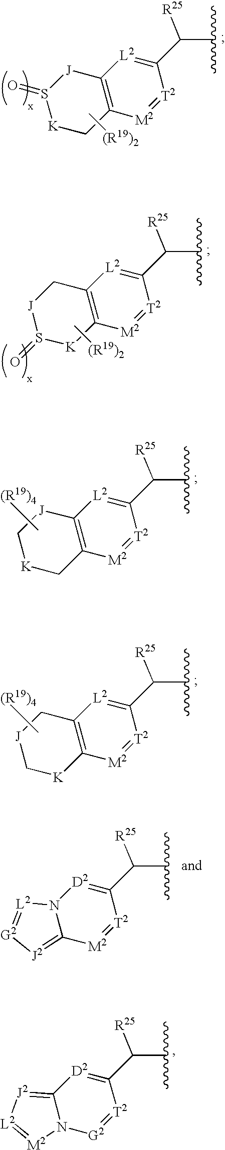
wherein all variables are as defined hereinabove.

In still a further embodiment, R¹ of Formula (III) may be selected from:

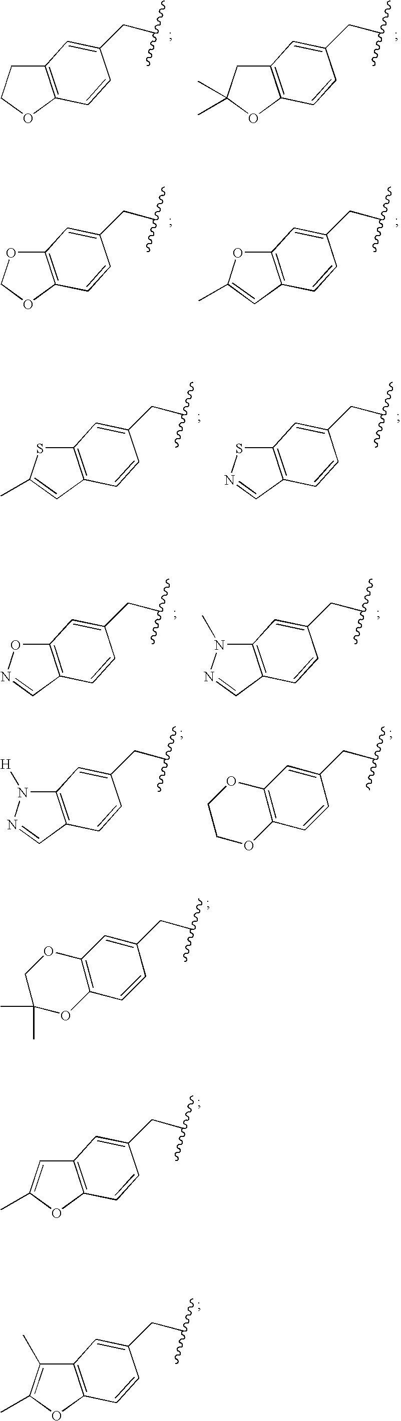

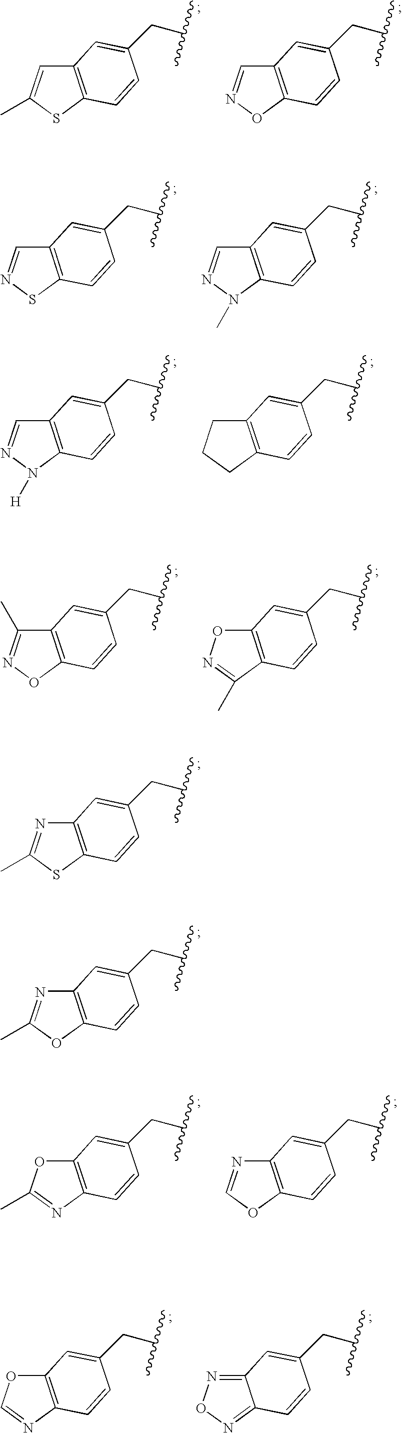

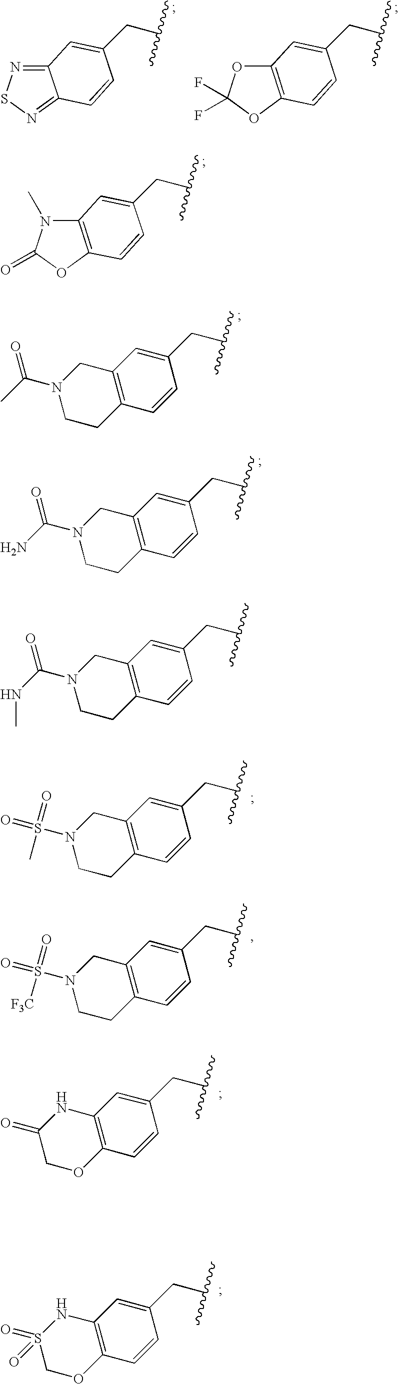

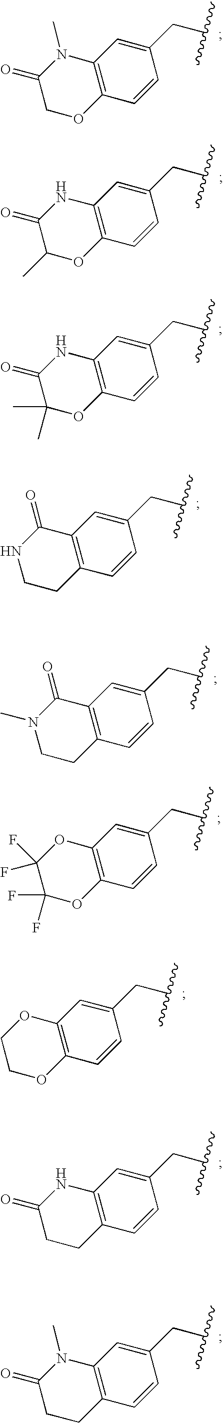

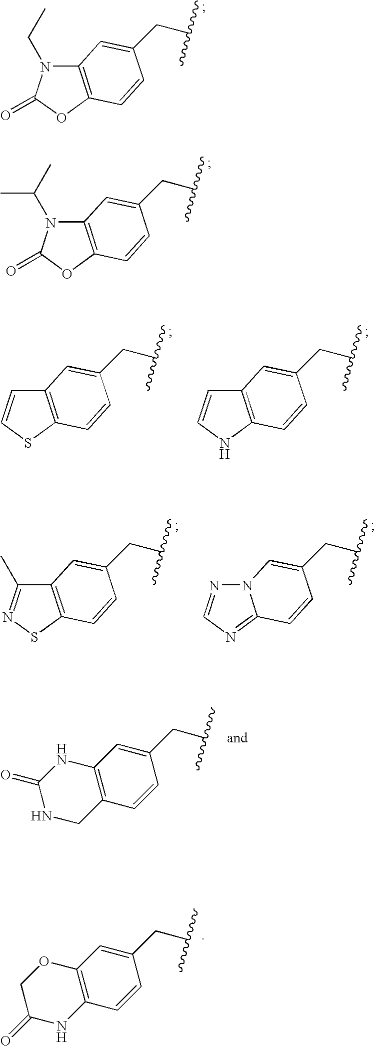

In still another embodiment, the present invention provides a compound selected from: 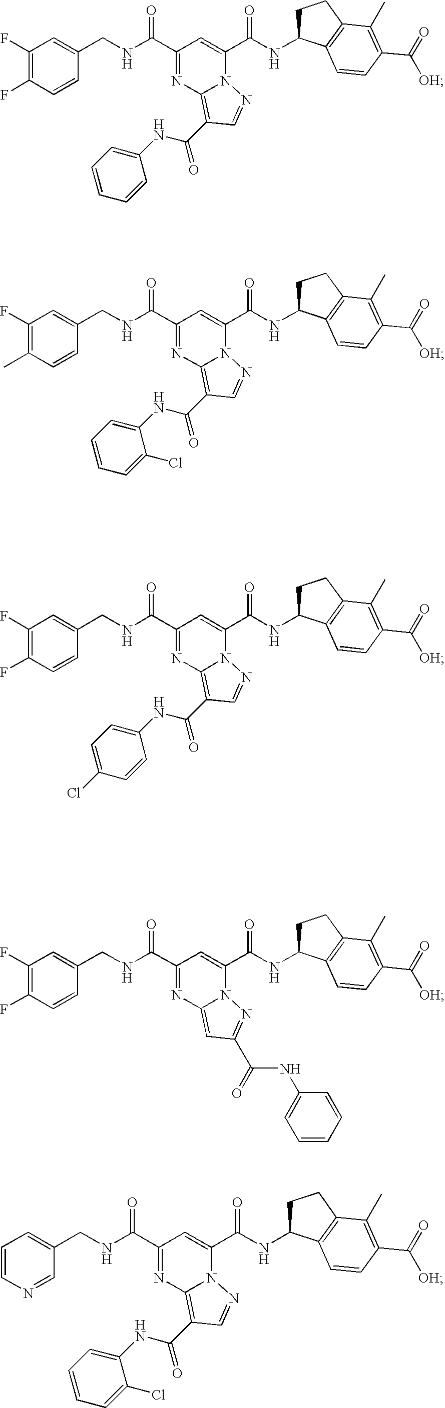 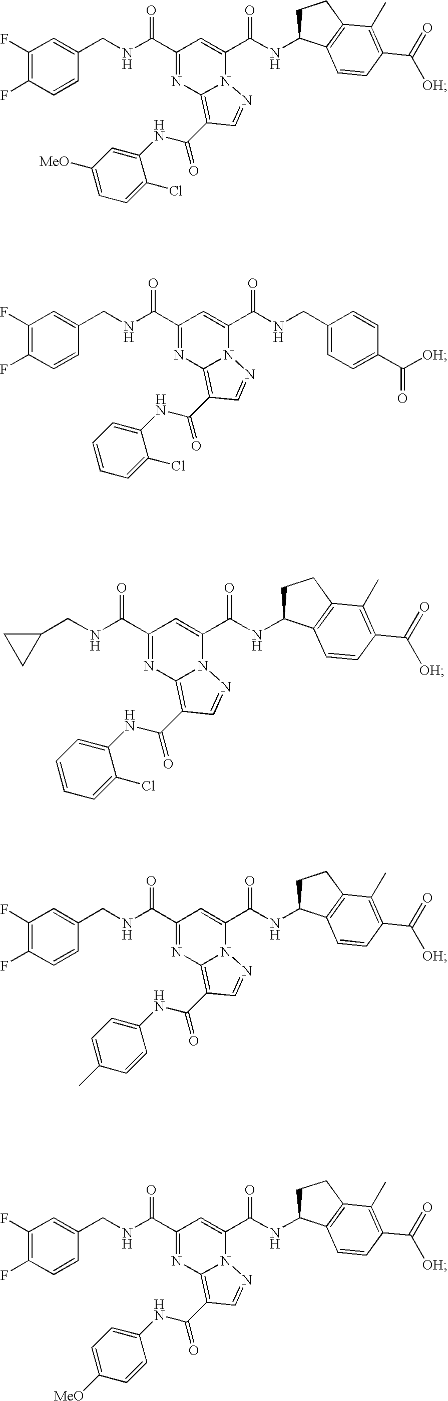 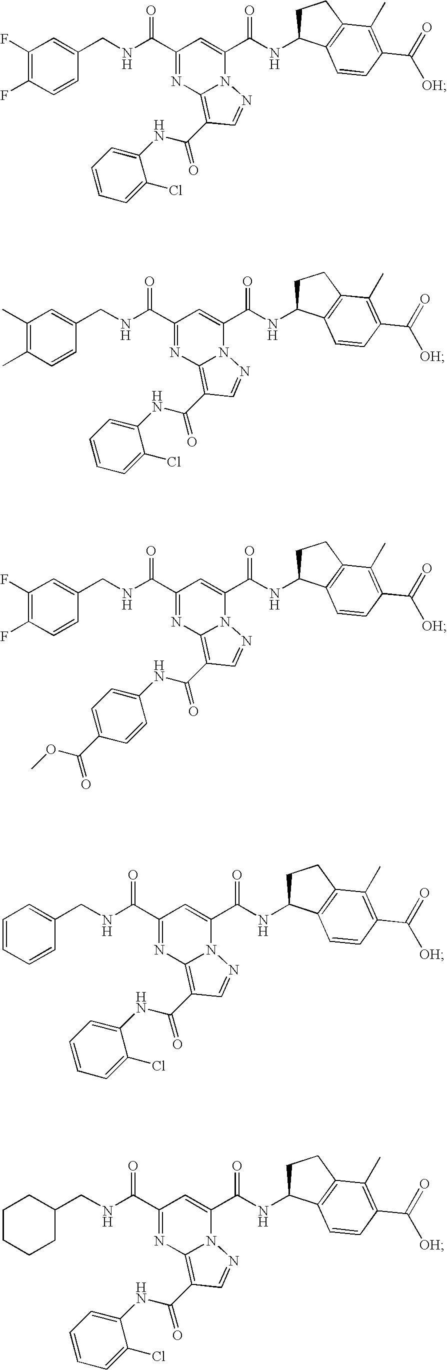 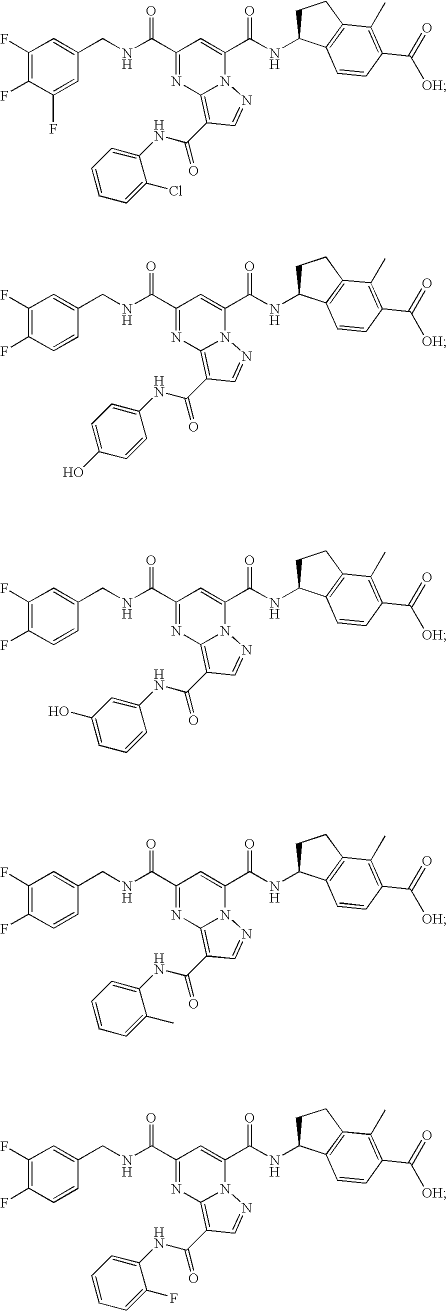 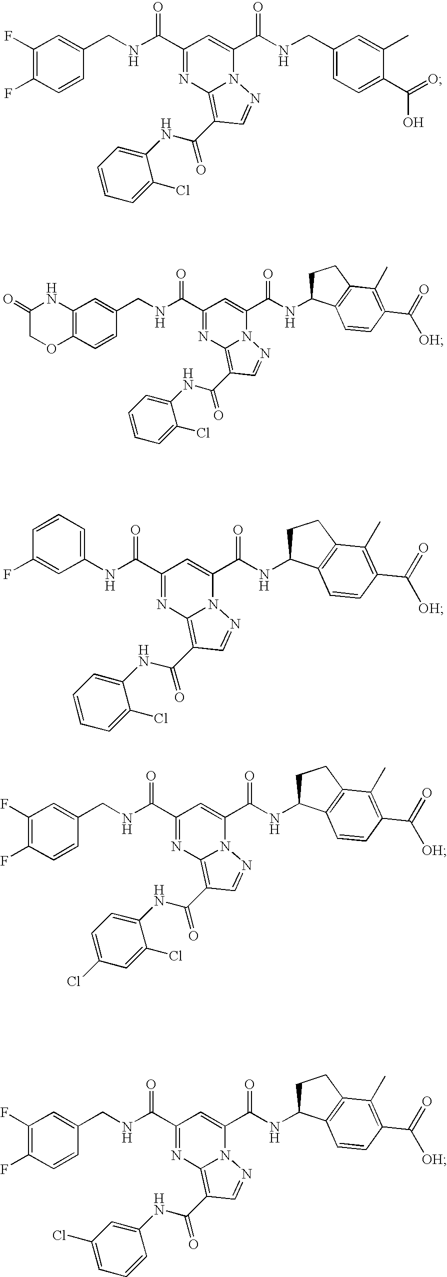 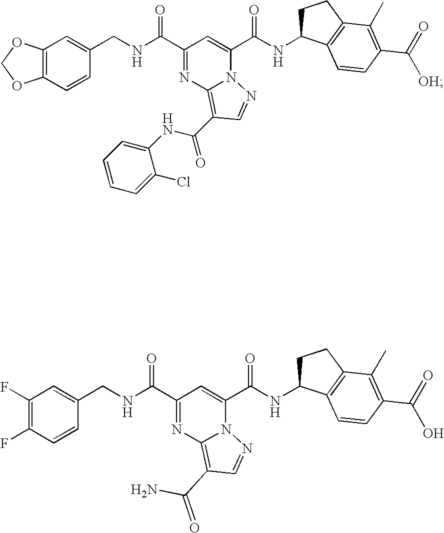
or a pharmaceutically acceptable salt thereof.

In a further embodiment, the present invention provides a compound selected from: 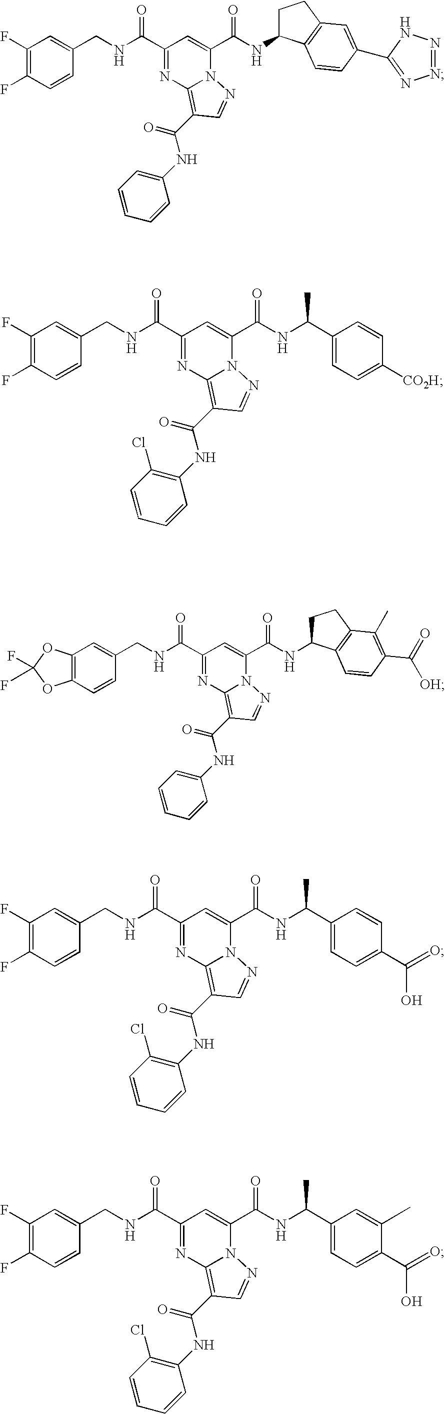 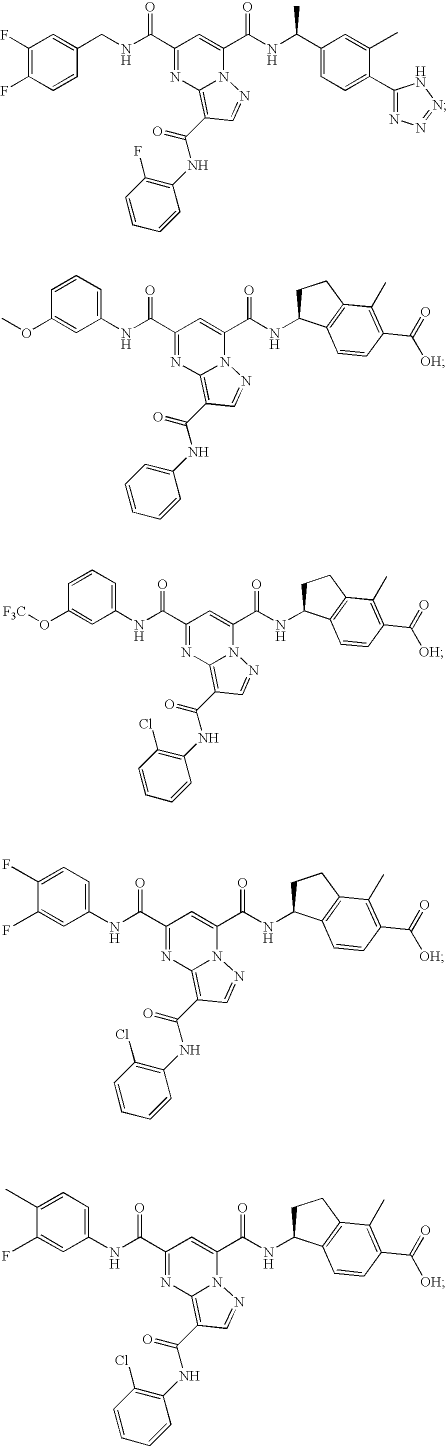 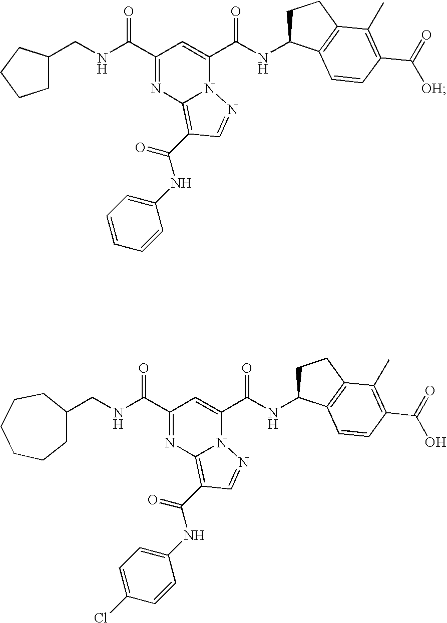
or a pharmaceutically acceptable salt thereof.

In one embodiment, the present invention provides a compound having the structure: 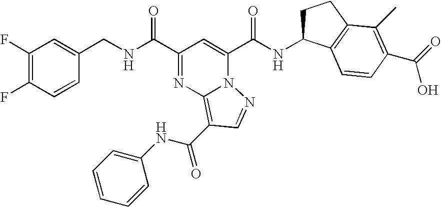
or a pharmaceutially acceptable salt thereof.

In another embodiment, the present invention provides a compound having the structure: 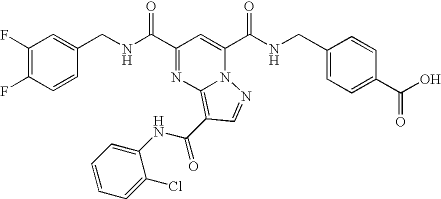
or a pharmaceutically acceptable salt thereof.

In yet another embodiment, the present invention provides a compound having the structure: 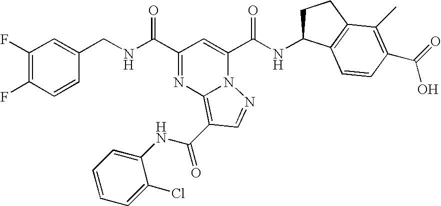
or a pharmaceutically acceptable salt thereof.

In still another embodiment, the present invention provides a compound having the structure: 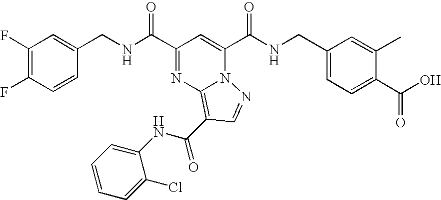
or a pharmaceutically acceptable salt thereof.

In a further embodiment, the present invention provides a compound having the structure: 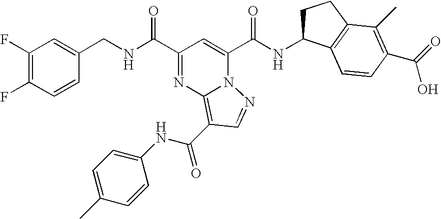
or a pharmaceutically acceptable salt thereof.

In yet a further embodiment, the present invention provides a compound having the structure: 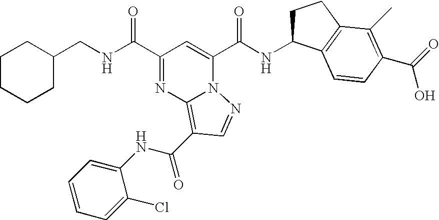
or a pharmaceutically acceptable salt thereof.

In still a further embodiment, the present invention provides a compound having the structure: 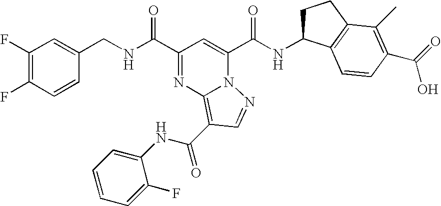
or a pharmaceutically acceptable salt thereof.

In another embodiment, the present invention provides a compound having the structure: 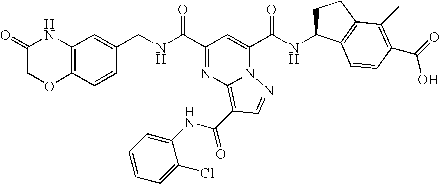
or a pharmaceutically acceptable salt thereof.

In yet another embodiment, the present invention provides a compound having the structure: 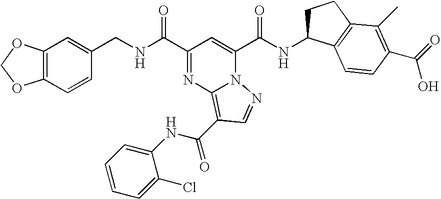
or a pharmaceutically acceptable salt thereof.

In still another embodiment, the present invention provides a compound having the structure: 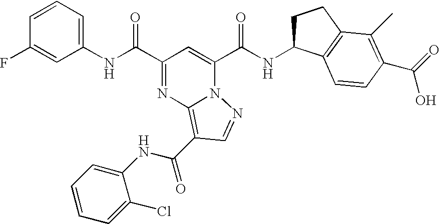
or a pharmaceutically acceptable salt thereof.

In still another embodiment, the present invention provides a compound having the structure: 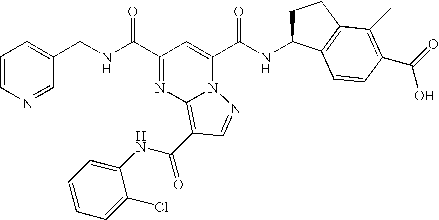
or a pharmaceutically acceptable salt thereof.

In still another embodiment, the present invention provides a compound having the structure: 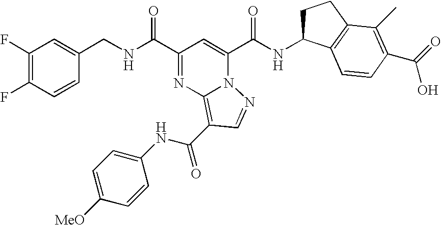
or pharmaceutically acceptable salt thereof.

In still another embodiment, the present invention provides a compound having the structure: 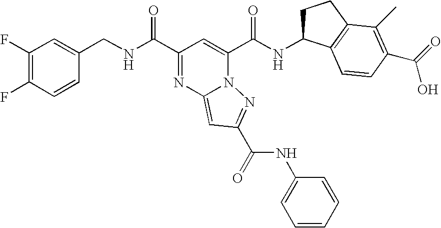
or a pharmaceutically acceptable salt thereof.

In still another embodiment, the present invention provides a compound having the structure: 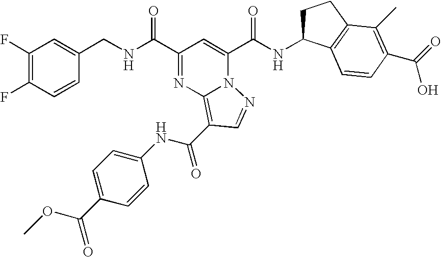
or a pharmaceutically acceptable salt thereof.

The present invention is also directed to pharmaceutical compositions which include any of the amide containing heterobicyclic metalloproteases of the invention described hereinabove. In accordance therewith, some embodiments of the present invention provide a pharmaceutical composition which may include an effective amount of an amide containing heterobicyclic metalloprotease compound of the present invention and a pharmaceutically acceptable carrier.

In one embodiment, the present invention provides a pharmaceutical composition including an effective amount of the compound of Formula (I) and N-oxides, pharmaceutically acceptable salts, prodrugs, formulation, polymorphs, racemic mixtures and stereoisomers thereof, and a pharmaceutically acceptable carrier.

In yet another embodiment, the present invention provides a pharmaceutical composition including an effective amount of the compound of Formula (II) and N-oxides, pharmaceutically acceptable salts, prodrugs, formulation, polymorphs, racemic mixtures and stereoisomers thereof, and a pharmaceutically acceptable carrier.

In another embodiment, the present invention provides a pharmaceutical composition including an effective amount of the compound of Formula (III) and N-oxides, pharmaceutically acceptable salts, prodrugs, formulation, polymorphs, racemic mixtures and stereoisomers thereof, and a pharmaceutically acceptable carrier.

The present invention is also directed to methods of inhibiting metalloproteases and methods of treating diseases or symptoms mediated by a metalloprotease enzyme, particularly ADAMTS-4 enzyme. Such methods include administering a heterobicyclic metalloprotease inhibiting compound of the present invention, or a pharmaceutically acceptable salt thereof. Examples of diseases or symptoms mediated by an ADAMTS-4 mediated enzyme include, but are not limited to, rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, cardiovascular disease, reperfusion injury, trauma, chemical exposure or oxidative damage to tissues, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, burn therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheeze.

In one embodiment, the present invention provides a method of inhibiting ADAMTS-4, which includes administering to a subject in need of such treatment a compound of Formula (I) and N-oxides, pharmaceutically acceptable salts, prodrugs, formulation, polymorphs, racemic mixtures and stereoisomers thereof.

In another embodiment, the present invention provides a method of inhibiting ADAMTS-4, which includes administering to a subject in need of such treatment a compound of Formula (II) and N-oxides, pharmaceutically acceptable salts, prodrugs, formulation, polymorphs, racemic mixtures and stereoisomers thereof.

In yet another embodiment, the present invention provides a method of inhibiting ADAMTS-4, which includes administering to a subject in need of such treatment a compound of Formula (III) and N-oxides, pharmaceutically acceptable salts, prodrugs, formulation, polymorphs, racemic mixtures and stereoisomers thereof.

In still a further embodiment, the present invention provides a method of treating an ADAMTS-4 mediated disease, which includes administering to a subject in need of such treatment an effective amount of a compound of Formula (I) and N-oxides, pharmaceutically acceptable salts, prodrugs, formulation, polymorphs, racemic mixtures and stereoisomers thereof.

In one embodiment, the present invention provides a method of treating an ADAMTS-4 mediated disease, which includes administering to a subject in need of such treatment an effective amount of a compound of Formula (II) and N-oxides, pharmaceutically acceptable salts, prodrugs, formulation, polymorphs, racemic mixtures and stereoisomers thereof.

In another embodiment, the present invention provides a method of treating an ADAMTS-4 mediated disease, which includes administering to a subject in need of such treatment an effective amount of a compound of Formula (III) and N-oxides, pharmaceutically acceptable salts, prodrugs, formulation, polymorphs, racemic mixtures and stereoisomers thereof.

Illustrative of the diseases which may be treated with such methods are: rheumatoid arthritis, osteoarthritis, abdominal aortic aneurysm, cancer (e.g. but not limited to melanoma, gastric carcinoma or non-small cell lung carcinoma), inflammation, atherosclerosis, multiple sclerosis, chronic obstructive pulmonary disease, ocular diseases (e.g. but not limited to ocular inflammation, retinopathy of prematurity, macular degeneration with the wet type preferred and corneal neovascularization), neurologic diseases, psychiatric diseases, thrombosis, bacterial infection, Parkinson's disease, fatigue, tremor, diabetic retinopathy, vascular diseases of the retina, aging, dementia, cardiomyopathy, renal tubular impairment, diabetes, psychosis, dyskinesia, pigmentary abnormalities, deafness, inflammatory and fibrotic syndromes, intestinal bowel syndrome, allergies, Alzheimers disease, arterial plaque formation, oncology, periodontal, viral infection, stroke, cardiovascular disease, reperfusion injury, trauma, chemical exposure or oxidative damage to tissues, wound healing, hemorroid, skin beautifying, pain, inflammatory pain, bone pain and joint pain, acne, acute alcoholic hepatitis, acute inflammation, acute pancreatitis, acute respiratory distress syndrome, adult respiratory disease, airflow obstruction, airway hyperresponsiveness, alcoholic liver disease, allograft rejections, angiogenesis, angiogenic ocular disease, arthritis, asthma, atopic dermatitis, bronchiectasis, bronchiolitis, bronchiolitis obliterans, bum therapy, cardiac and renal reperfusion injury, celiac disease, cerebral and cardiac ischemia, CNS tumors, CNS vasculitis, colds, contusions, cor pulmonae, cough, Crohn's disease, chronic bronchitis, chronic inflammation, chronic pancreatitis, chronic sinusitis, crystal induced arthritis, cystic fibrosis, delayted type hypersensitivity reaction, duodenal ulcers, dyspnea, early transplantation rejection, emphysema, encephalitis, endotoxic shock, esophagitis, gastric ulcers, gingivitis, glomerulonephritis, glossitis, gout, graft vs. host reaction, gram negative sepsis, granulocytic ehrlichiosis, hepatitis viruses, herpes, herpes viruses, HIV, hypercapnea, hyperinflation, hyperoxia-induced inflammation, hypoxia, hypersensitivity, hypoxemia, inflammatory bowel disease, interstitial pneumonitis, ischemia reperfusion injury, kaposi's sarcoma associated virus, lupus, malaria, meningitis, multi-organ dysfunction, necrotizing enterocolitis, osteoporosis, periodontitis, peritonitis associated with continous ambulatory peritoneal dialysis (CAPD), pre-term labor, polymyositis, post surgical trauma, pruritis, psoriasis, psoriatic arthritis, pulmatory fibrosis, pulmatory hypertension, renal reperfusion injury, respiratory viruses, restinosis, right ventricular hypertrophy, sarcoidosis, septic shock, small airway disease, sprains, strains, subarachnoid hemorrhage, surgical lung volume reduction, thrombosis, toxic shock syndrome, transplant reperfusion injury, traumatic brain injury, ulcerative colitis, vasculitis, ventilation-perfusion mismatching, and wheezing.

In some embodiments of the present invention, the heterobicyclic metalloprotease inhibiting compounds defined above are used in the manufacture of a medicament for the treatment of a disease or symptom mediated by an metalloprotease enzyme, particularly an ADAMTS-4 enzyme.

In some embodiments, the heterobicyclic metalloprotease inhibiting compounds defined above may be used in combination with a drug, active, or therapeutic agent such as, but not limited to: (a) a disease modifying antirheumatic drug, such as, but not limited to, methotrexate, azathioptrineluflunomide, penicillamine, gold salts, mycophenolate, mofetil, and cyclophosphamide; (b) a nonsteroidal anti-inflammatory drug, such as, but not limited to, piroxicam, ketoprofen, naproxen, indomethacin, and ibuprofen; (c) a COX-2 selective inhibitor, such as, but not limited to, rofecoxib, celecoxib, and valdecoxib; (d) a COX-1 inhibitor, such as, but not limited to, piroxicam; (e) an immunosuppressive, such as, but not limited to, methotrexate, cyclosporin, leflunimide, tacrolimus, rapamycin, and sulfasalazine; (f) a steroid, such as, but not limited to, p-methasone, prednisone, cortisone, prednisolone, and dexamethasone; (g) a biological response modifier, such as, but not limited to, anti-TNF antibodies, TNF-α antagonists, IL-1 antagonists, anti- CD40, anti-CD28, IL-10, and anti-adhesion molecules; and (h) other anti-inflammatory agents or therapeutics useful for the treatment of chemokine mediated diseases, such as, but not limited to, p38 kinase inhibitors, PDE4 inhibitors, TACE inhibitors, chemokine receptor antagonists, thalidomide, leukotriene inhibitors, and other small molecule inhibitors of pro-inflammatory cytokine production.

In one embodiment, the present invention provides a pharmaceutical composition which includes:

an effective amount of a compound of Formula (I) and N-oxides, pharmaceutically acceptable salts, prodrugs, formulation, polymorphs, racemic mixtures and stereoisomers thereof;

a pharmaceutically acceptable carrier; and

a member selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

In another embodiment, the present invention provides a pharmaceutical composition which includes:

an effective amount of a compound of Formula (II) and N-oxides, pharmaceutically acceptable salts, prodrugs, formulation, polymorphs, racemic mixtures and stereoisomers thereof;

a pharmaceutically acceptable carrier; and

a member selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

In still another embodiment, the present invention provides a pharmaceutical composition which includes:

an effective amount of a compound of Formula (III) and N-oxides, pharmaceutically acceptable salts, prodrugs, formulation, polymorphs, racemic mixtures and stereoisomers thereof;

a pharmaceutically acceptable carrier; and

a member selected from: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

Biological Activity

The inhibiting activity towards different metalloproteases of the heterobicyclic metalloprotease inhibiting compounds of the present invention may be measured using any suitable assay known in the art. A standard in vitro assay for measuring the metalloprotease inhibiting activity is described in Examples 1700 to 1705. The heterobicyclic metalloprotease inhibiting compounds show activity towards ADAMTS-4, MMP-3, MMP-8, MMP-12, MMP-13 and/or ADAMTS-5.

Some heterobicyclic metalloprotease inhibiting compounds of the invention have an ADAMTS-4 inhibition activity (IC₅₀ ADAMTS-4) ranging from below 300 nM to about 20 μM. Table 1 lists typical examples of heterobicyclic metalloprotease inhibiting compounds of the invention that have ADAMTS-4 inhibitory activity lower than 1 μM (Group A) and from 1 μM to 20 μM (Group B).

TABLE 1
Summary of ADAMTS-4 Activity for Compounds

| Group | Ex. # |
|---|---|
| A | 4, 5, 7, 11, 19, 20, 28, 34, 38, 39, 41 |
| B | 9, 10, 12, 16, 21, 22, 23, 27, 31, 32, 33, 36, 37, 43, 48, 51 |

Some heterobicyclic metalloprotease inhibiting compounds of the invention have an MMP-13 inhibition activity (IC₅₀ MMP-13) ranging from below 300 nM to about 20 μM. Table 2 lists typical examples of heterobicyclic metalloprotease inhibiting compounds of the invention that have MMP-13 inhibitory activity lower than 1 μM (Group A).

TABLE 2
Summary of MMP-13 Activity for Compounds

| Group | Ex. # |
|---|---|
| A | 12, 19, 20 |

The synthesis of metalloprotease inhibiting compounds of the invention and their biological activity assay are described in the following examples which are not intended to be limiting in any way.

Schemes

Provided below are schemes according to which compounds of the present invention may be prepared. In schemes described herein, each of R^{A}R^{B} and R^{C}R^{D} may be the same or different, and each may independently be selected from R¹R² and R²⁰R²¹ as defined hereinabove. Each of X^{a}, Y^{a}, and Z^{a} shown in the schemes below may be the same or different, and each may independently be selected from N and CR⁴. X^{b} shown in the schemes below in each occurrence may be the same or different and is independently selected from O, S, and NR⁵¹. Y^{b} shown in the schemes below in each occurrence may be the same and is independently selected from CR⁴ and N.

In some embodiments the compounds of Formula (I)-(III) are synthesized by the general methods shown in Scheme 1 to Scheme 3.

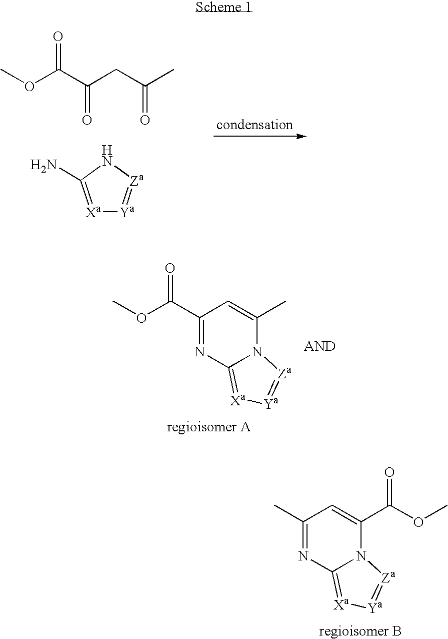

Methyl acetopyruvate is condensed (e.g. MeOH/reflux, aqueous HCl/100° C. or glacial AcOH/95° C.) with an amino substituted 5-membered heterocycle (e.g. 1H-pyrazol-5-amine) to afford a bicyclic ring system as a separable mixture of regioisomer A and regioisomer B (Scheme 1).

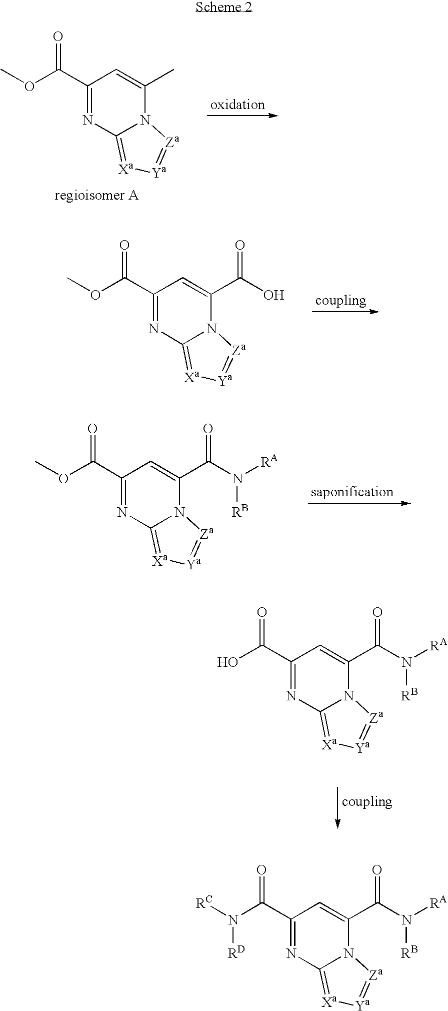

The regioisomer A of the bicyclic ring system from Scheme 1 (e.g. 7-methyl-pyrazolo[1,5-α]pyrimidine-5-carboxylic acid methyl ester) is oxidized (e.g. selenium dioxide/120-130° C. and then oxone®/room temperature) to afford the corresponding carboxylic acid (Scheme 2). Activated acid coupling (e.g. oxalyl chloride, PyBOP, PyBrOP, EDCI/HOAt or HATU/HOAt) with R^{A}R^{B}NH (e.g. 4-fluoro-3-methyl-benzylamine) in a suitable solvent gives the desired amide after purification. Saponification (e.g. aqueous LiOH/dioxane, NaOH/MeOH or TMSnOH/80° C.) and further activated acid coupling (e.g. oxalyl chloride, PyBOP, PyBrOP, EDCI/HOAt, HATU/HOAt, N-cyclohexyl-carbodiimide-N′-methyl-polystyrene or polystyrene-IIDQ) with R^{C}R^{D}NH gives the desired bicyclic bisamide inhibitor after purification. If necessary, the R group can be further manipulated (e.g. saponification of a COOMe group in R).

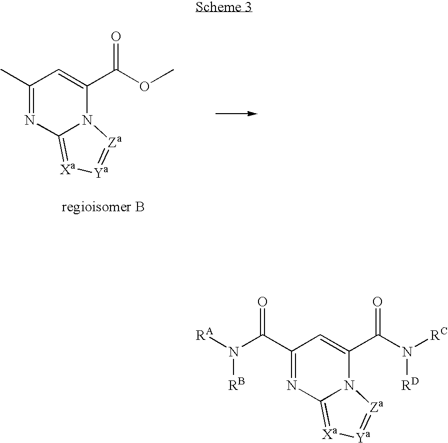

The regioisomer B of the bicyclic ring system from Scheme 1 (e.g. 5-methyl-pyrazolo[1,5-α]pyrimidine-7-carboxylic acid methyl ester) is treated similarly as shown in Scheme 2 to give the desired bicyclic bisamide inhibitor after purification (Scheme 3). If necessary, the R group can be further manipulated (e.g. saponification of a COOMe group in R).

In some embodiments the compounds of Formula (I)-(III) are synthesized by the general methods shown in Scheme 4 to Scheme 8.

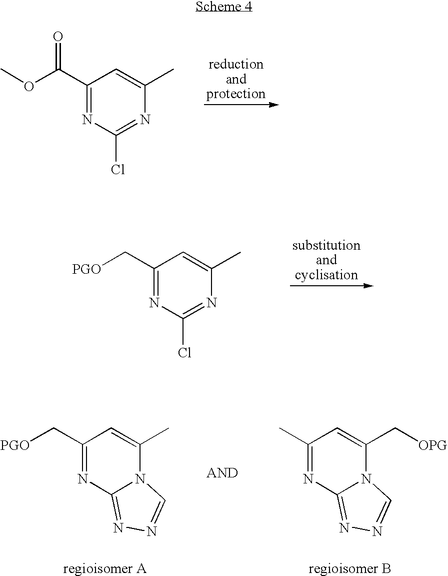

2-Chloro-6-methyl-pyrimidine-4-carboxylic acid methyl ester is reduced (e.g. NaBH₄/MeOH) to the corresponding alcohol and protected with a suitable protecting group [PG, e.g. (2-methoxyethoxy)methyl] (Scheme 4). The obtained intermediate is stirred with hydrazine hydrate at 70° C. to afford the corresponding hydrazino pyrimidine after concentration. Cyclization with a suitable reagent (e.g. triethylortho formate) gives the protected hydroxymethyl substituted bicyclic ring system as a separable mixture of regioisomer A and regioisomer B.

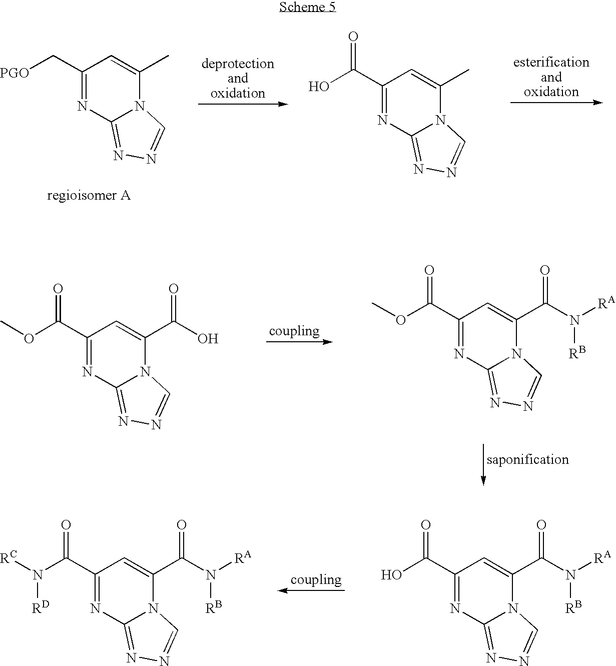

The regioisomer A of the protected hydroxymethyl substituted bicyclic ring system from Scheme 4 (e.g. 7-(2-methoxy-ethoxymethoxymethyl)-5-methyl-[1,2,4]triazolo[4,3-α]pyrimidine) is deprotected (e.g. HCl/THF) and then oxidized (e.g. KMnO₄ in aqueous Na₂CO₃/50° C.) to afford the corresponding carboxy substituted bicyclic ring system (Scheme 5). Esterifcation (e.g. thionyl chloride/MeOH) and oxidation (e.g. selenium dioxide/70° C.) of this intermediate gives the corresponding carboxylic acid. Activated acid coupling (e.g. oxalyl chloride, PyBOP, PyBrOP, EDCI(HOAt or HATU/HOAt) with R^{A}R^{B}NH (e.g. 4-fluoro-3-methyl-benzylamine) in a suitable solvent gives the desired amide after purification. Saponification (e.g. aqueous LiOH/dioxane, NaOH/MeOH or TMSnOH/80° C.) and further activated acid coupling (e.g. oxalyl chloride, PyBOP, PyBrOP, EDCI/HOAt, HATU/HOAt) with R^{C}R^{D}NH gives the desired bicyclic bisamide inhibitor after purification. If necessary, the R group can be further manipulated (e.g. saponification of a COOMe group in R).

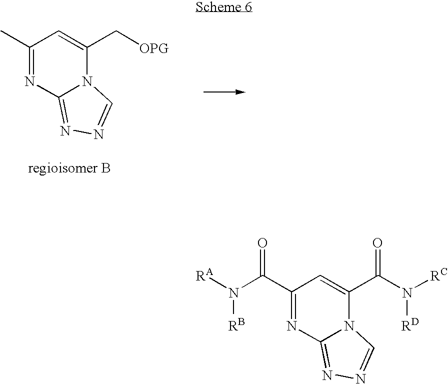

The regioisomer B of the protected hydroxymethyl substituted bicyclic ring system from Scheme 4 (e.g. 5-(2-methoxy-ethoxymethoxymethyl)-7-methyl-[1,2,4]triazolo[4,3-α]pyrimidine) is treated similarly as shown in Scheme 5 to give the desired bicyclic bisamide inhibitor after purification (Scheme 6). If necessary, the R group can be further manipulated (e.g. saponification of a COOMe group in R).

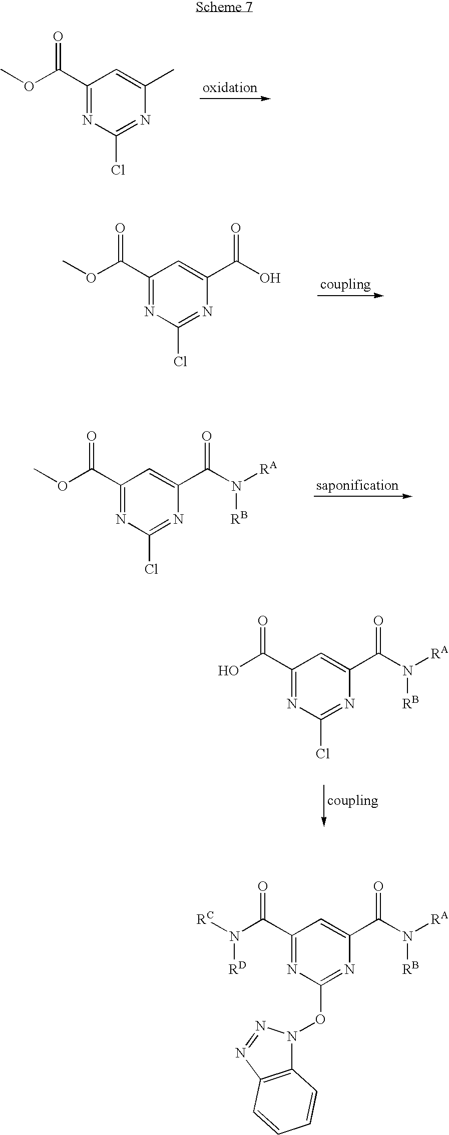

2-Chloro-6-methyl-pyrimidine-4-carboxylic acid methyl ester is oxidized (e.g. selenium dioxide/105° C.) to the corresponding carboxylic acid (Scheme 7). Activated acid coupling (e.g. oxalyl chloride) with R^{A}R^{B}NH (e.g. 4-fluoro-3-methyl-benzylamine) in a suitable solvent gives the desired amide after purification. Saponification (e.g. aqueous LiOH/THF) and further activated acid coupling (e.g. PYBOP) with R^{C}R^{D}NH (e.g. 4-aminomethyl-benzoic acid methyl ester) gives the corresponding benzotriazol-1-yloxy substituted pyrimidine bisamide.

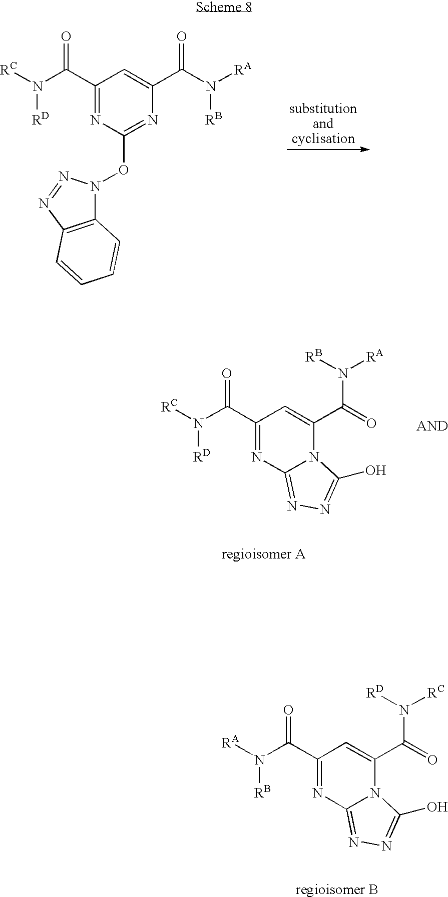

A benzotriazol-1-yloxy substituted pyrimidine bisamide from Scheme 7 (e.g. 4-({[2-(benzotriazol-1-yloxy)-6-(4-fluoro-3-methyl-benzylcarbamoyl)-pyrimidine-4-carbonyl]-amino}-methyl)-benzoic acid methyl ester) is stirred with hydrazine hydrate at room temperature to afford the corresponding hydrazino pyrimidine bisamide after concentration (Scheme 8). Cyclization with a suitable reagent (e.g. phosgene) gives the corresponding bicyclic bisamide inhibitor as a mixture of regioisomer A and regioisomer B. If necessary, the R group can be further manipulated (e.g. saponification of a COOMe group in R)

### EXAMPLES AND METHODS

All reagents and solvents were obtained from commercial sources and used without further purification. Proton (¹H) spectra were recorded on a 400 MHz NMR spectrometer in deuterated solvents. Flash chromatography was performed using Merck silica gel, grade 60, 70-230 mesh using suitable organic solvents as indicated in specific examples. Thin layer chromatography (TLC) was carried out on silica gel plates with UV detection.

### Preparative Example 1

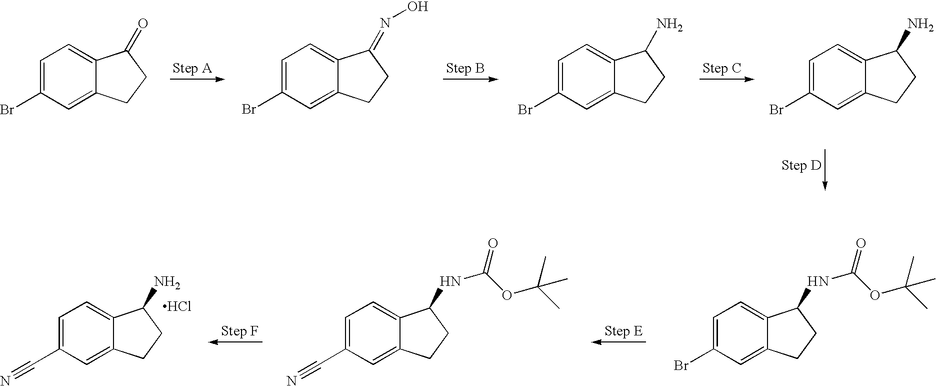 Step A

A mixture of commercially available 5-bromo-indan-1-one (1.76 g), hydroxylamine hydrochloride (636 mg) and sodium acetate (751 mg) in methanol (40 mL) was allowed to stir for 16 h at room temperature. Water (100 mL) was added and the resulting precipitate was filtered and washed with water (3×20 mL) to afford the title compound (1.88 g; >99%) as a colourless solid. [MH]⁺=226/228.

Step B

To a solution of the title compound from Step A above (1.88 g) in diethyl ether (20 mL) at −78° C. under an atmosphere of argon was slowly added a 1M solution of lithium aluminum hydride in diethyl ether (42.4 mL). The mixture was heated to reflux (40° C.) and allowed to stir for 5 h. The mixture was cooled to 0° C. and water (1.6 mL), 15% aqueous sodium hydroxide (1.6 mL) and water (4.8 mL) were carefully and sequentially added. The resulting mixture was filtered through Celite® and the filtrate was concentrated to give the title compound (1.65 g; 94%) as a clear oil. [MH]⁺=212/214.

Step C

To a boiling solution of the title compound from Step B above (1.13 g) in methanol (2.3 mL) was added a hot solution of commercially available N-acetyl-L-leucine (924 mg) in methanol (3 mL). The solution was allowed to cool to room temperature, which afforded a white precipitate. The solid was separated from the supernatant and washed with methanol (2 mL). The solid was recrystalized two times from methanol. To the resulting solid were added 10% aqueous sodium hydroxide (20 mL) and diethyl ether (20 mL). Once the solid was dissolved, the organic layer was separated and the aqueous layer was washed with diethyl ether. The combined organic layers were dried (MgSO₄), filtered and concentrated to give the title compound (99 mg; 18%) as a clear oil. [MH]⁺=212/214.

Step D

To a solution of the title compound from Step C above (300 mg), di-tert-butyl dicarbonate (370 mg) and triethylamine (237 μL) in tetrahydrofuran (10 mL) was allowed to stir for 16 h at room temperature. The solution was concentrated and the remaining residue was purified by chromatography (silica, hexanes/ethyl acetate) to give the title compound (460 mg; >99%) as a clear oil. [(M-isobutene)H]⁺=256/258, [MNa]⁺=334/336.

Step E

A mixture of the title compound from Step D above (460 mg), tetrakis triphenylphosphinepalladium (89 mg), zinc cyanide (200 mg) in N,N-dimethylformamide (5 mL) under an atmosphere of argon in a sealed vial was allowed to stir for 18 h at 110° C. The mixture was allowed to cool to room temperature before diethyl ether (20 mL) and water (20 mL) were added. The separated aqueous layer was washed with diethyl ether (4×10 mL). The combined organic layers were washed with water (3×10 mL) and brine (10 mL), dried (MgSO₄), filtered and concentrated. The resulting residue was purified by chromatography (silica, hexanes/ethyl acetate) to afford the title compound (170 mg; 47%) as a clear oil. [MH]⁺=259, [MNa]⁺=281.

Step F

To the title compound from Step E above (170 mg) was added a 4M solution of hydrochloric acid in dioxane (2 mL). The resulting solution was allowed to stir for 3 h at room temperature at which time a precipitate had formed. The mixture was concentrated to give 1(S)-amino-indan-5-carbonitrile hydrochloride (128 mg; >99%). [M-Cl]⁺=159.

### Preparative Example 2

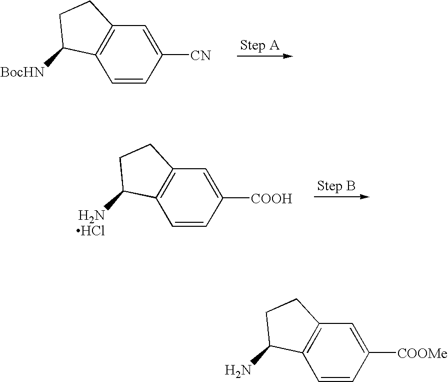 Step A

(5-Cyano-indan-1(S)-yl)-carbamic acid tert-butyl ester (1.0 g) was suspended in 6N hydrochloric acid (50 mL) and heated to 110-112° C. for 20 h upon which the solution became homogeneous. The solvent was removed under reduce pressure to give the intermediate. [M-Cl]⁺=178.

Step B

The intermediate from Step A above was dissolved in anhydrous MeOH (150 mL) and saturated with anhydrous hydrogen chloride gas. The reaction mixture was then heated to reflux for 20 h. After cooling to room temperature, the solvent was removed under reduced 5 pressure to give an oil. The oil was taken up in dichloromethane and washed with saturated NaHCO₃. The organic phase was separated and dried over MgSO₄, filtered and concentrated to give 1(S)-amino-indan-5-carboxylic acid methyl ester (0.66 g, 89% over two steps) as an oil which slowly crystallized into a light brown solid.

### Preparative Example 3

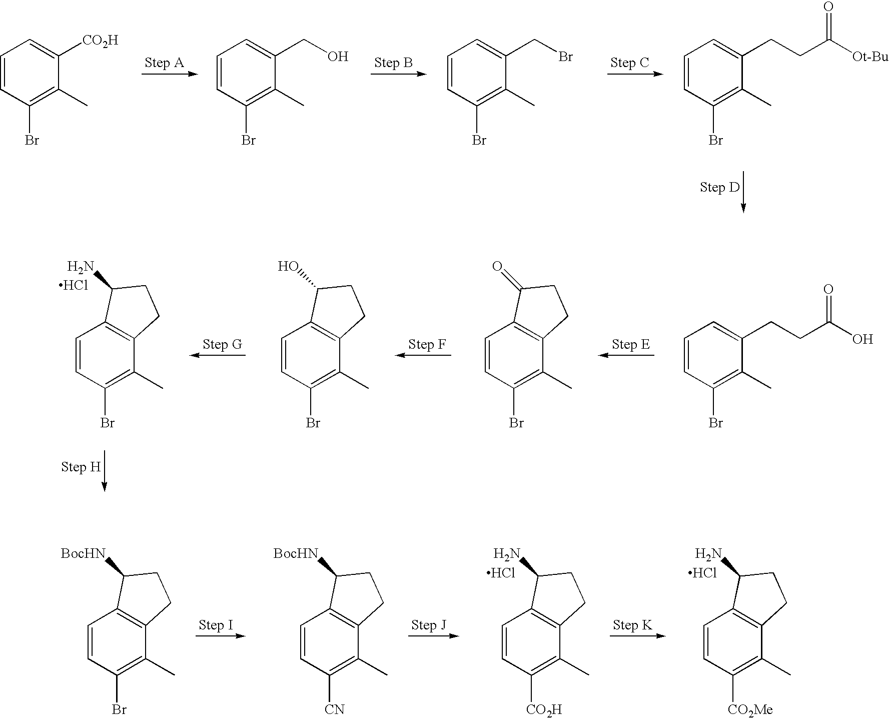 Step A

3-Bromo-2-methyl-benzoic acid (20.0 g) was dissolved in anhydrous THF (200 mL) under nitrogen and the reaction vessel was cooled to 0° C. in an ice bath. To this cooled solution was added BH₃.THF complex (1M in THF, 140 mL) dropwise over a 3 h period. Once gas evolution had subsided, the reaction mixture was warmed to room temperature and stirred for an additional 12 h. The mixture was then poured into 1N hydrochloric acid (500 mL) cooled with ice and then extracted with Et₂O (3×150 mL). The organic extracts were combined, dried over anhydrous MgSO₄, filtered, and then concentrated to afford the intermediate (18.1 g; 97%) as a colourless solid. ¹H-NMR (CDCl₃) δ=2.40 (s, 3H), 4.70 (s, 2H), 7.10 (t, 1H) 7.30 (d, 1H), 7.50 (d, 1H).

Step B

The intermediate from Step A above (18.1 g) was dissolved in anhydrous CH₂Cl₂ (150 mL) under nitrogen and the reaction vessel was cooled to 0° C. in an ice bath. To this cooled solution was added PBr₃ (5.52 mL) over a 10 min period. Once the addition was complete, the reaction mixture was warmed to room temperature and stirred for an additional 12 h. The mixture was cooled in an ice bath and quenched by the dropwise addition of MeOH (20 mL). The organic phase was washed with saturated NaHCO₃ (2×150 mL), dried over anhydrous MgSO₄, filtered, and then concentrated to afford the intermediate (23.8 g; 97%) as viscous oil. ¹H-NMR (CDCl₃) δ=2.50 (s, 3H), 4.50 (s, 2H), 7.00 (t, H), 7.25 (d, 1H) 7.50 (d, 1H).

Step C

t-Butyl acetate (12.7 mL) was dissolved in anhydrous THF (200 mL) under nitrogen and the reaction vessel was cooled to −78° C. in a dry ice/acetone bath. To this cooled solution was added dropwise lithium diispropylamide (1.5M in cyclohexane, 63.0 mL) and the mixture was allowed to stir for an additional 1 h upon which a solution of intermediate from Step B above (23.8 g) was added in THF (30 mL). Once the addition was complete, the reaction mixture was gradually warmed to room temperature over a 12 h period. The mixture was concentrated and the remaining viscous oil was dissolved in Et₂O (300 mL), washed with 0.5N hydrochloric acid (2×100 mL), dried over anhydrous MgSO₄, filtered, and then concentrated to afford the intermediate (21.5 g; 80%) as a pale-yellow viscous oil. ¹H-NMR (CDCl₃) δ=1.50 (s, 9H), 2.40 (s, 3H), 2.50 (t, 2H), 3.00 (t, 2H), 7.00 (t, 1H), 7.25 (d, 1H), 7.50 (d, 1H).

Step D

The intermediate from Step C above (21.5 g) was combined with polyphosphoric acid (250 g) and placed in a 140° C. oil bath for 10 min while mixing the thick slurry occasionally with a spatula. To this mixture was then added ice water (1 L) and the mixture was stirred for 2 h. The mixture was then filtered and the solid was washed with H₂O (2×100 mL) and dried to afford the intermediate (16.7 g; 96%). ¹H-NMR (CDCl₃) δ=2.40 (s, 3H), 2.65 (t, 2H), 3.00 (t, 2H), 7.00 (t, 1H), 7.20 (d, 1H), 7.50 (d, 1H).

Step E

The intermediate from Step D above (11.6 g) was dissolved in anhydrous CH₂Cl₂ (100 mL) under nitrogen and the reaction vessel was cooled to 0° C. in an ice bath. To this mixture was added dropwise oxalyl chloride (12.0 mL) and the mixture was stirred for 3 h after which the mixture was concentrated under reduced pressure. The remaining dark residue was dissolved in anhydrous CH₂Cl₂ (300 mL) and to this mixture was added AlCl₃ (6.40 g). Once the addition was complete, the mixture was refluxed for 4 h upon which the mixture was poured into ice water (500 mL) and extracted with CH₂Cl₂ (2×11 mL). The combined extracts were combined, dried over anhydrous MgSO₄, filtered, and then concentrated to afford the intermediate (10.6 g; 98%) as a light brown solid. ¹H-NMR (CDCl₃) δ=2.40 (s, 9H), 2.70 (t, 2H), 3.05 (t, 2H), 7.50 (d, 1H), 7.65 (d, 1H).

Step F

To a cooled solution of (S)-2-methyl-CBS-oxazaborolidine (1M in toluene, 8.6 mL) and borane.methyl sulfide complex (1M in CH₂Cl₂, 43.0 mL) at −20° C. (internal temperature) in CH₂Cl₂ (200 mL) was added a solution of intermediate from Step E above (9.66 g, in 70 mL CH₂Cl₂) over a 10 h period via a syringe pump. After the addition was complete, the mixture was then quenched by the addition of MeOH (100 mL) at −20° C., warmed to room temperature and concentrated. The crude mixture was purified by flash chromatography (10% to 30% Et₂O/CH₂Cl₂ gradient) to afford the intermediate (8.7 g; 90%) as a colourless solid. ¹H-NMR (CDCl₃) δ=2.00 (m, 1H), 2.35 (s, 3H), 2.50 (m, 1H), 2.90 (m, 1H), 3.10 (m, 1H), 5.25 (m, 1H), 7.20 (d, 1H), 7.50 (d, 1H).

Step G

To a −78° C. cooled solution of intermediate from step F above (8.7 g) in CH₂Cl₂ (200 mL) under nitrogen was added triethylamine (15.9 mL) followed by methanesulfonyl chloride (4.5 mL). This mixture was stirred for 90 min and then NH₃ (˜150 mL) was condensed into the mixture using a dry ice/acetone cold finger at a rate of ˜3 mL/minute. After stirring at −78° C. for an additional 2 h, the mixture was gradually warmed to room temperature allowing the NH₃ to evaporate from the reaction mixture. 1N NaOH (200 mL) was added and the aqueous layer was extracted with CH₂Cl₂ (2×100 mL). The combined extracts were dried over anhydrous MgSO₄, filtered, and then concentrated to afford crude material as a light brown oil. This oil was dissolved in Et₂O (200 mL) and hydrogen chloride (4M in dioxane, 10 mL) was added and the precipitate was collected and dried to give the intermediate (9.0 g; 90%). [M-NH₃Cl]⁺=209/211.

Step H

The intermediate from Step G above (5.2 g) was mixed in dry CH₂Cl₂ (50 mL) and cooled to 0° C. and to this cooled solution was added di-tert-butyl dicarbonate (5.0 g) followed by Et₃N (9.67 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid. ¹H-NMR (CDCl₃, free base) δ=1.80 (m, 1H), 2.30 (s, 3H), 2.60 (m, 1H), 2.80 (m, 1H), 2.90 (m, 1H), 4.30 (t, 1H), 7.00 (d, 1H), 7.40 (m, H).

Step I

The intermediate from Step H above (7.2 g), zinc(II) cyanide (5.2 g) and Pd(PPh₃)₄ (2.6 g) were combined under nitrogen and anhydrous DMF (80 mL) was added. The yellow mixture was heated to 100° C. for 18 h and then concentrated under reduced pressure to afford crude material which was purified by flash chromatography (20% CH₂Cl₂/EtOAc) to give the intermediate (4.5 g; 75%) as an off-white solid. ¹H-NMR (CDCl₃) δ=1.50 (s, 3H), 1.90 (m, 1H), 2.40 (s, 3H), 2.70 (m, 1H), 2.80 (m, H), 2.95 (m, 1H), 4.75 (m, 1H), 5.15 (m, 1H), 7.20 (d, 1H), 7.50 (d, 1H).

Step J

The intermediate from Step I above (1.0 g) was suspended in 6N hydrochloric acid (20 mL) and heated to 100° C. for 12 h upon which the solution become homogeneous. The solvent was removed under reduce pressure to give the intermediate (834 mg; quantitative) as a colourless solid. [M-NH₃Cl]⁺=175.

Step K

The intermediate from Step J above (1.0 g) was dissolved in anhydrous MeOH (20 mL) and cooled to 0° C. and anhydrous hydrogen chloride was bubbled through this solution for 2-3 min. The reaction mixture was then heated to reflux for 12 h. After cooling to room temperature, the solvent was removed under reduced pressure to give 1(S)-amino-4-methyl-indan-5-carboxylic acid methyl ester hydrochloride (880 mg; quantitative) as a colourless solid. [M-NH₃Cl]⁺=189.

### Preparative Example 4

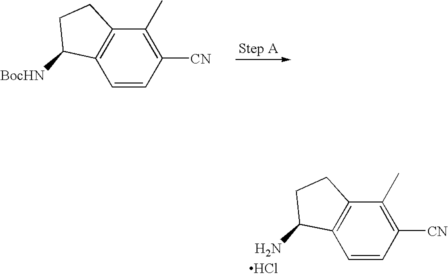 Step A

To (5-cyano-4-methyl-indan-1(S)-carbamic acid tert-butyl ester (108 mg) was added a solution of hydrogen chloride (4M in dioxane, 2 mL) and the resulting solution was allowed to stir at 22° C. for 6 h at which time a precipitate had formed. The mixture was concentrated to give the title compound (83 mg, >99%) as a colourless powder. [M-NH₃Cl]⁺=156.

### Preparative Example 5

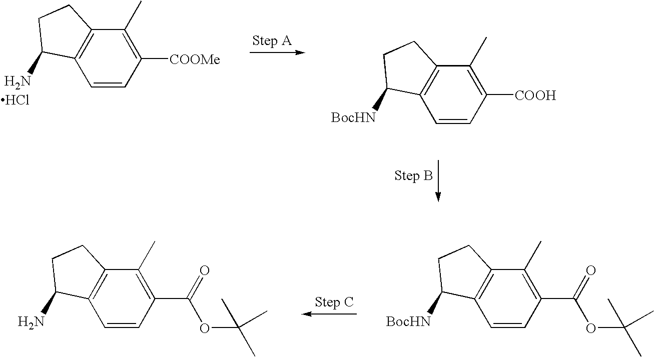 Step A

1(S)-Amino4-methyl-indan-5-carboxylic acid methyl ester hydrochloride (1.5 g) was mixed in dry CH₂Cl₂ (50 mL) and cooled to 0° C. and to this cooled solution was added di-tert-butyl dicarbonate (1.6 g) followed by Et₃N (1 mL). After stirring for 3 h, the mixture was concentrated and redissolved in Et₂O (250 mL). This solution was washed with saturated NaHCO₃ (100 mL) and brine (100 mL). The organic layer was dried over anhydrous MgSO₄, filtered, and concentrated to afford the intermediate (7.28 g; 97%) as a colourless solid which was dissolved in tetrahydrofuran (60 mL). To the mixture was added a 1M aqueous LiOH solution (60 mL) and the mixture was stirred at 50° C. for 2 h. The mixture was concentrated to dryness and redissolved in water, acidified to pH=5 with hydrochloric acid and extracted with ethyl acetate. The organic layer was dried (MgSO₄) and concentrated to afford the intermediate as colourless solid (1.87 g). [MNa]⁺=314.

Step B

To a solution of the title compound from Step A above (1.87 g) in dry toluene (15 mL) was added Di-tert-butoxymethyl dimethylamine (6.2 mL) at 80° C. At this temperature the mixture was stirred for 3 h. After cooling to room temperature the mixture was concentrated and purified by column chromatography (silica, dichloromethane) to afford the intermediate (820 mg; 38%) as a colourless solid. [MNa]⁺=370.

Step C

To a solution of the title compound from Step B above (820 mg) in tert-butyl acetate (40 mL) was added sulfuric acid (0.65 mL) at room temperature. The mixture was stirred for 5 h and concentrated to dryness. The residue was dissolved ethyl acetate and washed with a saturated solution of sodium hydrogen carbonate and brine. After drying (MgSO₄) 1(S)-amino-4-methyl-indan-5-carboxylic acid tert-butyl ester (640 mg; 99%) was obtained as a colourless solid. [M-NH₂]⁺=231.

### Preparitive Example 6

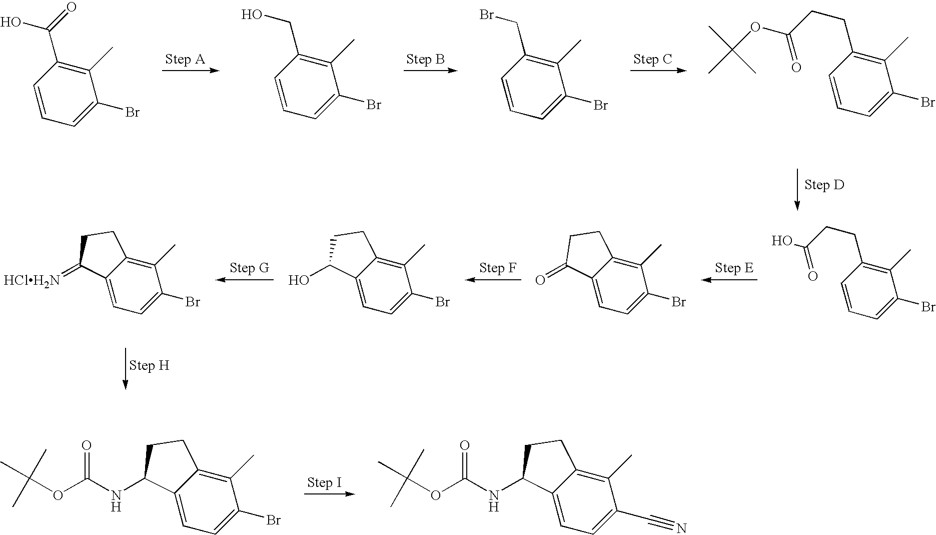 Step A

Under a nitrogen atmosphere a 1M solution of BH₃.THF complex in THF (140 mL) was added dropwise over a 3 h period to an ice cooled solution of commercially available 3-bromo-2-methyl-benzoic acid (20.0 g) in anhydrous THF (200 mL). Once gas evolution had subsided, the cooling bath was removed and mixture stirred at room temperature for 12 h. The mixture was then poured into a mixture of 1N aqueous HCl (500 mL) and ice and then extracted with Et₂O (3×150 mL). The combined organic phases were dried (MgSO₄), filtered and concentrated to afford the title compound as a colorless solid (18.1 g, 97%). ¹H-NMR (CDCl₃) δ=7.50 (d, 1H), 7.30 (d, 1H), 7.10 (t, 1H), 4.70 (s, 2H), 2.40 (s, 3H).

Step B

Under a nitrogen atmosphere PBr₃ (5.52 mL) was added over a 10 min period to an ice cooled solution of the title compound from Step A above (18.1 g) in anhydrous CH₂Cl₂ (150 mL). The cooling bath was removed and mixture stirred at room temperature for 12 h. The mixture was cooled (0-5° C.), quenched by dropwise addition of MeOH (20 mL), washed with saturated aqueous NaHCO₃ (2×150 mL), dried (MgSO₄), filtered and concentrated to afford the title compound as a viscous oil (23.8 g, 97%). ¹H-NMR (CDCl₃) δ=7.50 (d, 1H), 7.25 (d, 1H), 7.00 (t, 1H), 4.50 (s, 2H), 2.50 (s, 3H).

Step C

Under a nitrogen atmosphere a 1.5M solution of lithium diispropylamide in cyclohexane (63 mL) was added dropwise to a cooled (−78° C., acetone/dry ice) solution of ^{t}BuOAc in anhydrous THF (200 mL). The mixture was stirred at −78° C. for 1 h, then a solution of the title compound from Step B above (23.8 g) in THF (30 mL) was added and the mixture was stirred for 12 h while warming to room temperature. The mixture was concentrated, diluted with Et₂O (300 mL), washed with 0.5N aqueous HCl (2×100 mL), dried (MgSO₄), filtered and concentrated to afford the title compound as a pale-yellow viscous oil (21.5 g, 80%). ¹H-NMR (CDCl₃) δ=7.50 (d, 1H), 7.25 (d, 1H), 7.00 (t, 1H), 3.00 (t, 2H), 2.50 (t, 2H), 2.40 (s, 3H), 1.50 (s, 9H).

Step D

A mixture of the title compound from Step C above (21.5 g) and polyphosphoric acid (250 g) was placed in a preheated oil bath (140° C.) for 10 min while mixing the thick slurry occasionally with a spatula. The oil bath was removed, ice and H₂O (1 L) was added and the mixture was stirred for 2 h. The precipitate was isolated by filtration, washed with H₂O (2×100 mL) and dried to afford the title compound (16.7 g, 96%). ¹H-NMR (CDCl₃) δ=7.50 (d, 1H), 7.20 (d, 1H), 7.00 (t, 1H), 3.00 (t, 2H), 2.65 (t, 2H), 2.40 (s, 3H).

Step E

Under a nitrogen atmosphere oxalyl chloride (12.0 mL) was added dropwise to an ice cooled solution of the title compound from Step D above (11.6 g) in anhydrous CH₂Cl₂ (100 mL). The resulting mixture was stirred for 3 h and then concentrated. The remaining dark residue was dissolved in anhydrous CH₂Cl₂ (300 mL) and AlCl₃ (6.40 g) was added. The mixture was heated to reflux for 4 h, cooled and poured into ice water (500 mL). The aqueous phase was separated and extracted with CH₂Cl₂ (2×100 mL). The combined organic phases were dried (MgSO₄), filtered and concentrated to afford the title compound as a light brown solid (10.6 g, 98%). ¹H-NMR (CDCl₃) δ=7.65 (d, 1H), 7.50 (d, 1H), 3.05 (t, 2H), 2.70 (t, 2H), 2.40 (s, 3H).

Step F

Using a syringe pump, a solution of the title compound from Step E above (9.66 g) in anhydrous CH₂Cl₂ (70 mL) was added over a 10 h period to a cooled (−20° C., internal temperature) mixture of a 1M solution of (S)-(−)-2-methyl-CBS-oxazaborolidine in toluene (8.6 mL) and a 1M solution of BH₃.Me₂S complex in CH₂Cl₂ (43.0 mL) in CH₂Cl₂ (200 ml). The mixture was then quenched at −20° C. by addition of MeOH (100 mL), warmed to room temperature, concentrated and purified by flash chromatography (silica, Et₂O/CH₂Cl₂) to afford the title compound as a colorless solid (8.7 g, 90%). ¹H-NMR (CDCl₃) δ=7.50 (d, 1H), 7.20 (d, 1H), 5.25 (m, 1H), 3.10 (m, 1H), 2.90 (m, 1H), 2.50 (m, 1H), 2.35 (s, 3H), 2.00 (m, 1H).

Step G

Under a nitrogen atmosphere NEt₃ (15.9 mL) and methanesulfonyl chloride (4.5 mL) were added subsequently to a cooled (−78° C., acetone/dry ice) solution of the title compound from Step F above (8.7 g) in anhydrous CH₂Cl₂ (200 mL). The mixture was stirred at −78° C. for 90 min, then NH₃ (˜150 mL) was condensed into the mixture using a dry ice condenser at a rate of ˜3 mL/min and stirring at −78° C. was continued for 2 h. Then the mixture was gradually warmed to room temperature allowing the NH₃ to evaporate. 1N aqueous NaOH (200 mL) was added, the organic phase was separated and the aqueous phase was extracted with CH₂Cl₂ (2×100 mL). The combined organic phases were dried (MgSO₄), filtered and concentrated. The remaining light brown oil was dissolved in Et₂O (200 mL) and a 4M solution of HCl in 1,4-dioxane (10 mL) was added. The formed precipitate was collected and dried to give the title compound (9.0 g, 90%). [M-NH₃Cl]⁺=209/211.

Step H

To an ice cooled solution of the title compound from Step G above (5.2 g) in anhydrous CH₂Cl₂ (50 mL) were subsequently added di-tert-butyl dicarbonate (5.0 g) and NEt₃ (9.67 mL). The resulting mixture was stirred for 3 h, concentrated, diluted with Et₂O (250 mL), washed with saturated aqueous NaHCO₃ (100 mL) and saturated aqueous NaCl (100 mL), dried (MgSO₄), filtered and concentrated to afford the title compound as a colorless solid (7.28 g, 97%). ¹H-NMR (CDCl₃, free base) δ=7.40 (m, H), 7.00 (d, 1H), 4.30 (t, 1H) 2.90 (m, 1H), 2.80 (m, 1H), 2.60 (m, 1H), 2.30 (s, 3H), 1.80 (m, 1H).

Step I

Under a nitrogen atmosphere a mixture of the title compound from Step H above (7.2 g), Zn(CN)₂ (5.2 g) and Pd(PPh₃)₄ (2.6 g) in anhydrous DMF (80 mL) was heated to 100° C. for 18 h, concentrated and purified by flash chromatography (silica, CH₂Cl₂/EtOAc) to afford the title compound as an off-white solid (4.5 g, 75%). ¹H-NMR (CDCl₃) δ=7.50 (d, 1H), 7.20 (d, 1H), 5.15 (m, 1H), 4.75 (m, 1H), 2.95 (m, 1H), 2.80 (m, 1H), 2.70 (m, 1H), 2.40 (s, 3H), 1.90 (m, 1H), 1.50 (s, 9H).

### Preparative Example 7

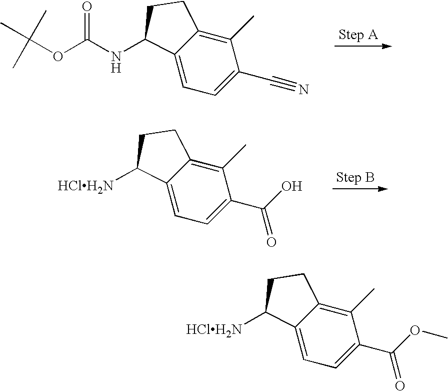 Step A

The title compound from the Preparative Example 1, Step I (1.0 g) was suspended in 6N aqueous HCl (20 mL), heated to 100° C. for 12 h and concentrated to give the title compound as a colorless solid. (834 mg, >99%). [M−NH₃Cl]⁺=175.

Step B

Anhydrous HCl gas was bubbled through an ice cooled solution of the title compound from Step A above (1.0 g) in anhydrous MeOH (20 mL) for 2-3 min. The cooling bath was removed, the mixture was heated to reflux for 12 h, cooled to room temperature and concentrated to give the title compound as a colorless solid (880 mg, 83%). [M−NH₃Cl]⁺=189.

### Preparative Example 8

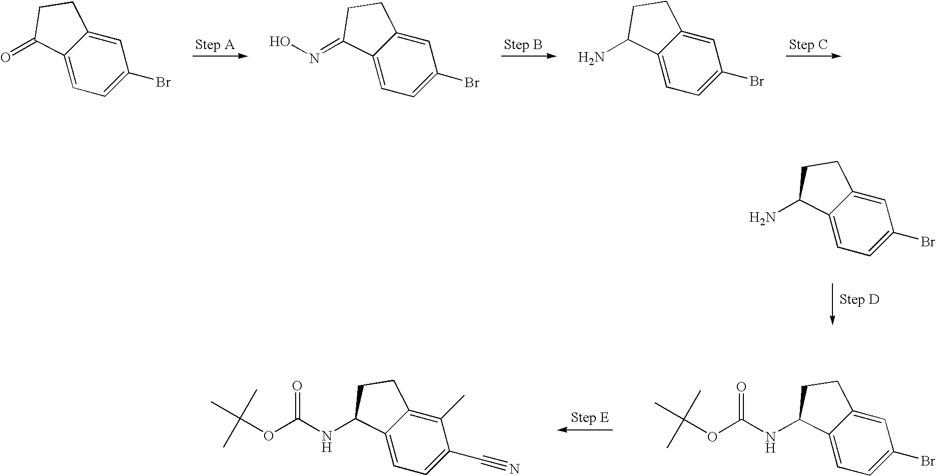 Step A

A mixture of commercially available 5-bromo-indan-1-one (1.76 g), hydroxylamine hydrochloride (636 mg) and NaOAc (751 mg) in MeOH (40 mL) was stirred at room temperature for 16 h and then diluted with H₂O (100 mL). The formed precipitate was collected by filtration, washed with H₂O (3×20 mL) and dried to afford the title compound as a colorless solid (1.88 g, >99%). [MH]⁺=226/228.

Step B

Under an argon atmosphere a 1M solution of LiAlH₄ in Et₂O (42.4 mL) was slowly added to a cooled (−78° C., acetone/dry ice) solution of the title compound from Step A above (1.88 g) in Et₂O (20 mL). Then the cooling bath was removed and the mixture was heated to reflux for 5 h. The mixture was cooled (0-5° C.) and H₂O (1.6 nmL), 15% aqueous NaOH (1.6 mL) and H₂O (4.8 mL) were carefully and sequentially added. The resulting mixture was filtered through a plug of celite® and concentrated to give the title compound as a clear oil (1.65 g, 94%). [MH]⁺=212/214.

Step C

To a boiling solution of the title compound from Step B above (1.13 g) in MeOH (2.3 mL) was added a hot solution of commercially available N-acetyl-L-leucine (924 mg) in MeOH (3 mL). The solution was allowed to cool to room temperature, which afforded a white precipitate. The precipitate was collected by filtration, washed with MeOH (2 mL) and recrystalized from MeOH (2×). The obtained solid was dissolved in a mixture of 10% aqueous NaOH (20 mL) and Et₂O (20 mL), the organic phase was separated and the aqueous phase was extracted with Et₂O. The combined organic phases were dried (MgSO₄), filtered and concentrated to give the title compound as a clear oil (99 mg, 18%). [MH]⁺=212/214.

Step D

To a solution of the title compound from Step C above (300 mg) in THF (10 mL) were subsequently added di-tert-butyl dicarbonate (370 mg) and NEt₃ (237 μL). The resulting mixture was stirred at room temperature for 16 h, concentrated and purified by chromatography (silica, hexanes/EtOAc) to afford the title compound as a clear oil (460 mg, >99%). [MNa]⁺=334/336.

Step E

Under an argon atmosphere a mixture of the title compound from Step D above (460 mg), Zn(CN)₂ (200 mg) and Pd(PPh₃)₄ (89 mg) in anhydrous DMF (5 mL) was heated in a sealed vial to 110° C. for 18 h. The mixture was cooled to room temperature and diluted with Et₂O (20 mL) and H₂O (20 mL). The organic phase was separated and the aqueous phase was extracted with Et₂O (4×10 mL). The combined organic phases were washed with H₂O (3×10 mL) and saturated aqueous NaCl (10 mL), dried (MgSO₄), filtered, concentrated and purified by chromatography (silica, hexanes/EtOAc) to afford the title compound as a clear oil (170 mg, 47%). [MH]⁺=259.

### Preparative Example 9

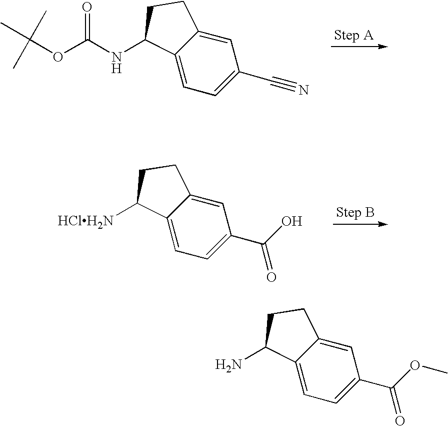 Step A

The title compound from the Preparative Example 3, Step E (1.0 g) was suspended in 6N aqueous HCl (50 mL), heated under closed atmosphere to 110-112° C. for 20 h and concentrated to give the title compound (827 mg, >99%). [M−Cl]⁺=178.

Step B

The title compound from Step A above (827 mg) was dissolved in anhydrous MeOH (150 mL) and saturated with anhydrous HCl gas. The resulting mixture was heated to reflux for 20 h, cooled to room temperature and concentrated. The remaining oil was taken up in CH₂Cl₂ and washed with saturated aqueous NaHCO₃, dried (MgSO₄), filtered and concentrated to give the title compound as an oil which slowly crystallized into a light brown solid (660 mg, 89%). [MH]⁺=192.

### Preparative Example 10

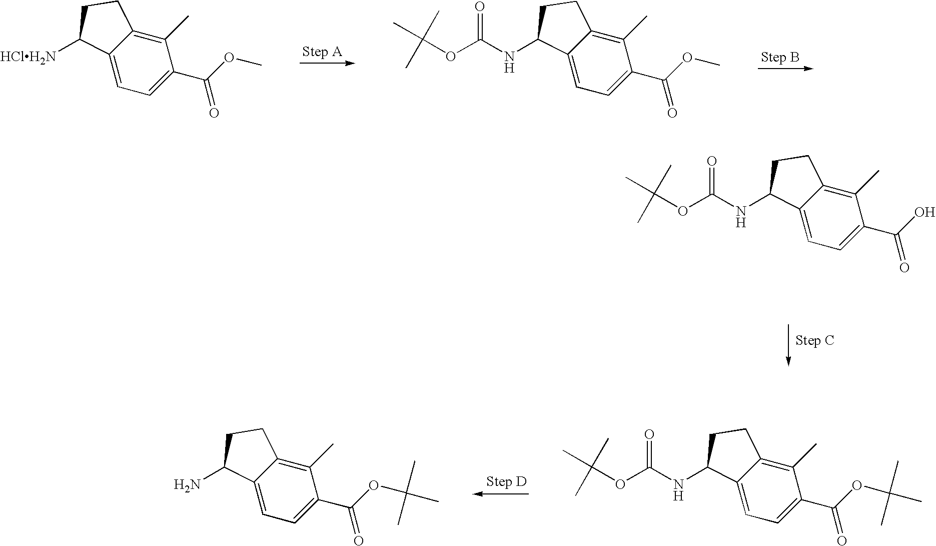 Step A

To an ice cooled solution of the title compound from the Preparative Example 2, Step B (5.94 g) in dry CH₂Cl₂ (50 mL) were subsequently added di-tert-butyl dicarbonate (1.6 g) and NEt₃ (1 mL). The mixture was stirred for 3 h, concentrated, diluted with Et₂O (250 mL), washed with saturated aqueous NaHCO₃ (100 mL) and saturated aqueous NaCl (100 nmL), dried (MgSO₄), filtered and concentrated to afford the title compound as a colorless solid (7.28 g, 97%). [MNa]⁺=328.

Step B

To a mixture of the title compound from Step A above (7.28 g) in THF (60 mL) was added 1M aqueous LiOH (60 mL). The mixture was stirred at 50° C. for 2 h, concentrated, diluted with H₂O, adjusted to pH 5 with HCl and extracted with EtOAc. The combined organic phases were dried (MgSO₄), filtered and concentrated to afford the title compound as colorless solid (1.87 g, 27%). [MNa]⁺=314.

Step C

At 80° C. N,N-dimethylformamide di-tert-butyl acetal (6.2 mL) was added to a solution of the title compound from Step B above (1.87 g) in dry toluene (15 mL). The mixture was stirred at 80° C. for 3 h, cooled to room temperature, concentrated and purified by chromatography (silica, CH₂Cl₂) to afford the title compound as a colorless solid (820 mg, 38%). [MNa]⁺=370.

Step D

To a solution of the title compound from Step C above (820 mg) in ^{t}BuOAc (40 mL) was added concentrated H₂SO₄ (0.65 mL). The resulting mixture was stirred at room temperature for 5 h, concentrated, diluted with EtOAc, washed with saturated aqueous NaHCO₃ and saturated aqueous NaCl, dried (MgSO₄), filtered and concentrated to afford the title compound as a colorless solid (640 mg, 99%). [M—NH₂]⁺=231.

### Preparative Example 11

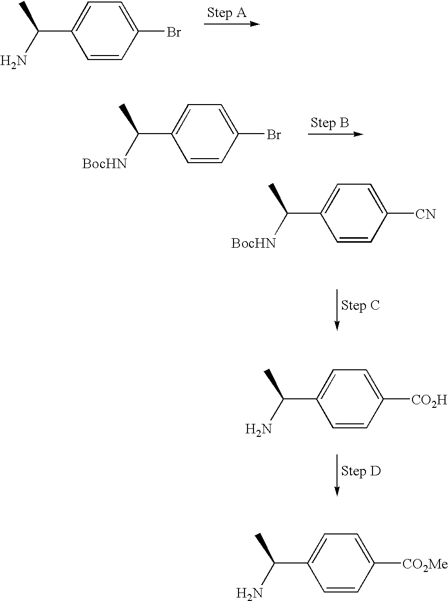 Step A

Commercially obtained (S)-(−)-1-(4-bromophenyl)ethylamine (2.0 g, 10.1 mmol) was dissolved in 50 mL dry tetrahydrofuran (THF) and cooled to 0° C. and to this cooled solution was added di-t-butyl dicarbonate (2.0 g, 9.1 mmol) dissolved in 3.0 mL of methylene chloride (CH₂Cl₂) followed by Et₃N (2.8 mL, 20.1 mmol). The solution was allowed to warm to room temperature. After stirring for 3 hours, the mixture was concentrated and re-dissolved in 100 mL methylene chloride (CH₂Cl₂). This solution was washed with IN HCl (2×50 mL) and saturated NaHCO₃ (1×50 mL). The CH₂Cl₂ layer was dried over anhydrous MgSO₄, filtered, and concentrated to afford 2.5 g of the Boc protected product in 92% yield as a white solid.

¹H-NMR δ (CDCl₃) 1.35 (br. s, 12H), 4.72 (br. s, 2H), 7.17 (d, 2H), 7.43 (d, 2H).

Step B

The Boc protected product from Step A (4.0 g, 13.3 mmol), ZnCN₂ (3.0 g, 24.4 mmol), and Pd[PPh₃]₄ (1.5 g, 1.3 mmol) were combined under nitrogen and anhydrous dimethylformamide (25 mL) was added. The yellow mixture was heated to 100° C. for 18 h and then concentrated under reduced pressure to afford crude cyano compound which was purified by flash chromatography (20% hexane/CH2Cl2) to give 2.0 g of the desired cyano containing compound as an oil in 60% yield.

¹H-NMR δ (CDCl₃) 0.89-1.62 (br. m, 12H), 4.81 (br. s, 2H), 7.42 (d, 2H), 7.65 (d, 2H).

MH⁺=247

Step C

The cyano compound (2.0 g, 8.1 mmol) was suspended in 6N HCl (50 mL) and heated to 100-105° C. for 20 hours upon which the solution becomes homogeneous. The solvent was removed under reduce pressure to give 1.8 g of the amino acid as the hydrochloride salt in quantitative yield as a white solid.

Step D

The hydrochloride salt of the amino acid (1.0 g, 4.9 mmol) was dissolved in anhydrous MeOH (150 mL) saturated with anhydrous HCl gas. The reaction mixture was then heated to reflux for 20 hours. After cooling to room temperature, the solvent was removed under reduced pressure to give a solid. The solid was taken up in methylene chloride (CH₂Cl₂) and washed with saturated NaHCO₃. The organic was separated and dried over MgSO₄, filtered and concentrated to give 0.31 g of 4-(1(S)-amino-ethyl)-benzoic acid methyl ester in 35% yield as an oil which slowly crystallized into a light brown solid. MH⁺=180

### Preparative Example 12

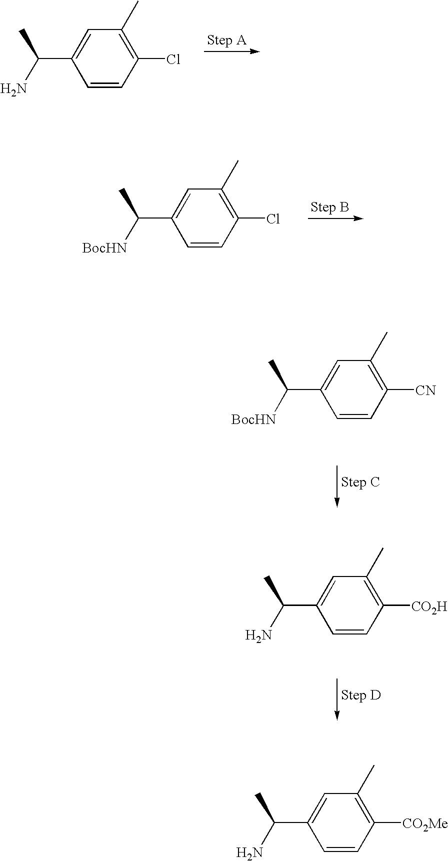 Step A

Commercially available (S)-1-(4-chloro-3-methylophenyl)ethylamine (1.5 mmol) was dissolved in 10 mL dry Tetrahydrofuran (THF) and cooled to 0° C. and to this cooled solution was added di-t-butyl dicarbonate (1.5 mmol) dissolved in 1.0 mL of metheylene chloride (CH₂Cl₂) followed by Et₃N (2.8 mL, 5 mmol). The solution was allowed to warm to room temperature. After stirring for 3 hours, the mixture was concentrated and re-dissolved in 100 mL methylene chloride (CH₂Cl₂). This solution was washed with IN HCl (2×50 muL) and saturated NaHCO₃ (1×50 mL). The CH₂Cl₂ layer was dried over anhydrous MgSO₄, filtered, and concentrated to afford the Boc protected product.

Step B

If to the Boc protected amine product (1 mmol) was added ZnCN₂ (2 mmol), Pd[PPh₃]₄ (0.1 mmol) and anhydrous dimethylformamide (6 mL) and the yellow mixture heated to 100° C. for 18 h and then purified by flash chromatography (20% hexane/CH2Cl2) one would get the desired cyano containing compound.

Step C

If the cyano containing compound (0.5 mmol) was suspended in 6N HCl (10 mL) and heated to 100-105° C. for 20 hours until the solution becomes homogeneous and the solvent removed under reduce pressure one would get the amino acid as the hydrochloride salt.

Step D

If the hydrochloride salt of the amino acid (0.5 mmol) was dissolved in anhydrous MeOH (50 mL) saturated with anhydrous HCl gas and then heated to reflux for 20 hours one would get the 4-(1(S)-amino-ethyl)-2-methyl-benzoic acid methyl ester.

### Preparative Example 13

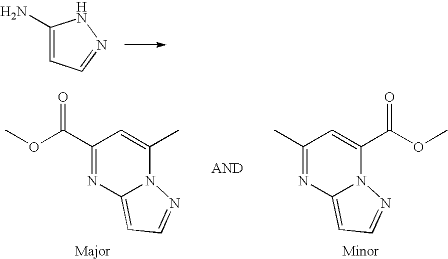

To a solution of commercially available 1H-pyrazol-5-amine (86.4 g) in MeOH (1.80 L) was added commercially available methyl acetopyruvate (50.0 g). The mixture was heated to reflux for 5 h and then cooled to room temperature overnight. The precipitated yellow needles were collected by filtration and the supernatant was concentrated at 40° C. under reduced pressure to ˜⅔ volume until more precipitate began to form. The mixture was cooled to room temperature and the precipitate was collected by filtration. This concentration/precipitation/filtration procedure was repeated to give 3 batches. This material was combined and recrystallized from MeOH to give the major isomer, methyl 7-methyl-pyrazolo[1,5-a]pyrimidine-5-carboxylate (81.7 g, 72%). [MH]⁺=192.

### Preparative Example 14

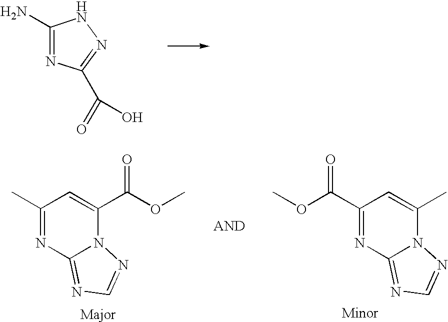

A mixture of commercially available 5-amino-1H-[1,2,4]triazole-3-carboxylic acid (20.3 g) and methyl acetopyruvate (20.0 g) in glacial AcOH (250 mL) was heated to 95° C. for 3 h. The mixture was concentrated and diluted with saturated aqueous NaHCO₃ (200 mL) and CH₂Cl₂ (500 mL). The organic phase was separated, dried (MgSO₄), filtered and concentrated to give a pale orange mixture of regioisomers (80:20, 21.3 g, 80%). Recrystallization of the crude material from hot THF (110 mL) afforded the major isomer, 5-methyl-[1,2,4]triazolo[1,5-a]pyrimidine-7-carboxylic acid methyl ester (13.0 g, 49%). [MH]⁺=193. The supernatant was concentrated and purified by chromatography (silica, hexanes/EtOAc) to afford the minor isomer, 7-methyl-[1,2,4]triazolo[1,5-a]pyrimidine-5-carboxylic acid methyl ester. [MH]⁺=193.

### Preparative Example 15

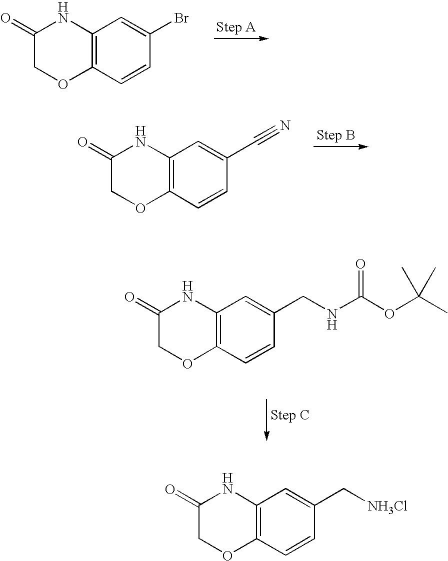 Step A

A degassed suspension of commercially available 6-Bromo-4H-benzo[1,4]oxazin-3-one (8.39 g), Zn(CN)₂ (3.46 g) and Pd(PPh₃)₄ (2.13 g) in DMF (70 mL) was stirred in a oil bath (80° C.) overnight. The mixture was cooled to room temperature and then poured into water (500 mL). The precipitate was collected by suction, air dried, washed with pentane, dissolved in CH₂Cl₂/MeOH (1:1), filtered through an silica pad and concentrated to yield a yellow solid (5.68 g, 89%; MH⁺=175).

Step B

To an ice cooled solution of the title compound from Step A above (5.6 g), di-tert-butyl dicarbonate (14.06 g) and NiCl₂.6H₂O (1.53 g) in MeOH, NaBH₄ (8.51 g) was added in portions. The mixture was vigorously stirred for lh at 0° C. and 1 h at room temperature. After the addition of diethylenetriamine (3.5 mL) the mixture was concentrated, diluted with EtOAc, washed subsequently with 1N HCl, saturated aqueous NaHCO₃ and saturated aqueous NaCl, dried (MgSO₄), concentrated to afford the title compound as an off white solid (7.91 g, 88%; M+Na⁺=397).

Step C

The title compound from Step B above (7.91 g) was dissolved in a 4M solution of HCl in 1,4-dioxane (120 mL), stirred for 14 h, concentrated, suspended in Et₂O, filtered and dried to afford the title compound as an off-white solid (5.81 g, 96%; M−NH₃Cl⁺=162).

### Preparative Example 16

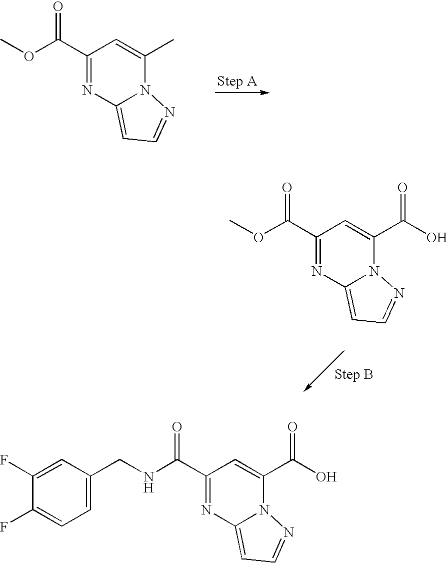 Step A

A mixture of 7-methyl-pyrazolo[1,5-a]pyrimidine-5-carboxylic acid methyl ester (13 g) and selenium dioxide (17.38 g) in 1,4-dioxane (120 mL) was heated to 130° C. under closed atmosphere for 12 h, cooled and filtered through celite®. To the filtrate were added oxone (20.91 g) and H₂O (120 mL) and the resulting suspension was stirred at room temperature overnight. The mixture was concentrated and then mixed with H₂O and 5% MeOH in CH₂Cl₂. The undissolved solid was filtered, washed with 5% MeOH in CH₂Cl₂ and dried to give pyrazolo[1,5-a]pyrimidine-5,7-dicarboxylic acid 5-methyl ester (5 g, 33%). [MH]⁺=222.

Step B

Pyrazolo[1,5-a]pyrimidine-5,7-dicarboxylic acid 5-methyl ester (664 mg, 3 mmol) and 3-4difluorobenzylamine (1.3 g, 9 mmol) were dissolved in N,N-dimethylformamide (2.5 mL) and heated to 60° C. for 12 h. The solution was cooled down to room temperature and diluted with 1N hydrochloric acid (10 mL). The resulting precipitate was colleted and dried to afford 5-(3,4-difluoro-benzylcarbamoyl)-pyrazolo[1,5-a]pyrimidine-7-carboxylic acid (1 g, yield 99%). MS (M+H): 333.

### Preparative Example 17

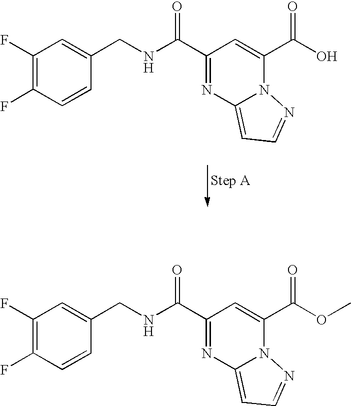 Step A

To a solution of 5-(3,4-Difluoro-benzylcarbamoyl)-pyrazolo[1,5-a]pyrimidine-7-carboxylic acid (350 mg) in MeOH (1 mL) and benzene (3 mL) was added TMSCHN₂ (0.8 mL, 2M in ether). The solution was stirred for 1 h and concentrated. The solution was absorbed onto silica and purified by silica gel chromatography to give 5-(3,4-Difluoro-benzylcarbamoyl)-pyrazolo[1,5-a]pyrimidine-7-carboxylic acid methyl ester (215 mg, 60%). [MH]⁺=347.

### Preparative Example 18

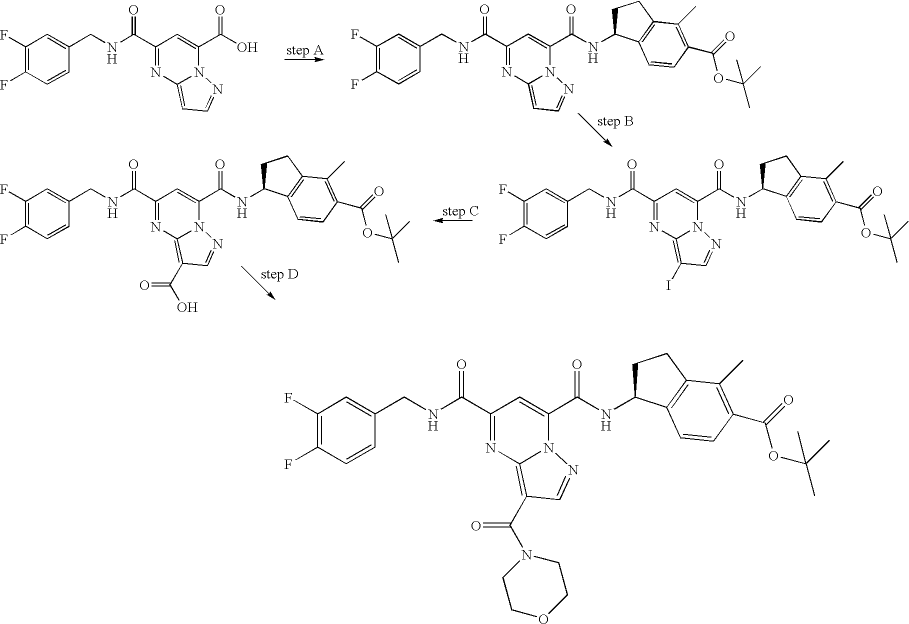 Step A

To a solution of 5-(3,4-Difluoro-benzylcarbamoyl)-pyrazolo[1,5-a]pyrirnidine-7-carboxylic acid (222 mg), and DMF (2 μL) in CH₂Cl₂ (5 mL) at 0° C. was added oxalyl chloride (287 μl). The solution was allowed to warm to 22° C. stirred for 3 h and concentrated. The resulting residue was brought up in CH₂Cl₂ (2.5 mL) and cooled to 0° C. To this cooled solution were added triethyl amine (102 μL) and a solution of (S)-1-amino-4-methyl-indan-5-carboxylic acid tert-butyl ester (165 mg) and triethyl amine (102 μL) in CH₂Cl₂ (1 mL). The resulting solution was stirred at 22° C. for 18 h and absorbed onto silica and purified by silica gel chromatography to give (S)-1-{[5-(3,4-difluoro-benzylcarbamoyl)-pyrazolo[1,5-a]pyrimidine-7-carbonyl]-amino}-4-methyl-indan-5-carboxylic acid tert-butyl ester (309 mg, 81%). [M−H]⁻=560.4.

Step B

A solution of (S)-1-{[5-(3,4-difluoro-benzylcarbamoyl)-pyrazolo[1,5-a]pyrimidine-7-carbonyl]-amino}-4-methyl-indan-5-carboxylic acid tert-butyl ester (309 mg) and N-iodosuccinimide (147 mg) in chloroform (5 mL) was stirred at 70° C. for 1 h. The solution was absorbed onto silica and purified by silica gel chromatography to give (S)-1-{[5-(3,4-Difluoro-benzylcarbamoyl)-3-iodo-pyrazolo[1,5-a]pyrimidine-7-carbonyl]-amino}-4-methyl-indan-5-carboxylic acid tert-butyl ester (365 mg, 97%). [M−H]⁻=686.4.

Step C

A mixture of (S)-1-{[5-(3,4-Difluoro-benzylcarbamoyl)-3-iodo-pyrazolo[1,5-a]pyrimidine-7-carbonyl]-amino}-4-methyl-indan-5-carboxylic acid tert-butyl ester (188 mg), Pd(OAc)₂ (4.6 mg), 1,1′-bis(diphenylphosphino)ferrocene (32.2 mg), potassium acetate (110 mg) in DMSO (1.5 mL) under 1 atm of carbon monoxide was stirred at 60° C. for 18 h. EtOAc was added and the organic layer was washed twice with 1N HCl, once with brine, dried over MgSO₄, filtered, absorbed onto silica and purified by silica gel chromatography to give (S)-7-(5-tert-butoxycarbonyl-4-methyl-indan-1-ylcarbamoyl)-5-(3,4-difluoro-benzylcarbamoyl)-pyrazolo[1,5-a]pyrimidine-3-carboxylic acid (150 mg, 85%), [M−H]⁻=604.5.

Step D

To a solution of (S)-7-(5-tert-butoxycarbonyl-4-methyl-indan-1-ylcarbamoyl)-5-(3,4-difluoro-benzylcarbamoyl)-pyrazolo[1,5-a]pyrimidine-3-carboxylic acid (8 mg), and DMF (1 μL) in CH₂Cl₂ (0.3 mL) at 0° C. was added oxalyl chloride (5 μl). The solution was allowed to warm to 22° C. stirred for 3 h and concentrated. The resulting residue was brought up in CH₂Cl₂ (0.2 mL) and cooled to 0° C. To this cooled solution were added triethyl amine (4 μL) and a solution of morpholine (4 μL) in CH₂Cl₂ (0.2 mL). The resulting solution was stirred at 22° C. for 18 h and absorbed onto silica and purified by silica gel chromatography to give (S)-1-{[5-(3,4-difluoro-benzylcarbamoyl)-3-(morpholine-4-carbonyl)-pyrazolo[1,5-a]pyrimidine-7-carbonyl]-amino}-4-methyl-indan-5-carboxylic acid tert-butyl ester (6.4 mg, 73%). [M−H]⁻=673.6.

Preparative Example 19

Following a similar procedure as that described in Preparative Example 18, step A except using the amine indicated in table below, the following compound was prepared.

| Prep. | | | 1. Yield |
|---|---|---|---|
| Ex. # | amine | product | 2. [M − H]⁻ |
| 19 | 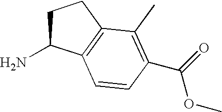 | 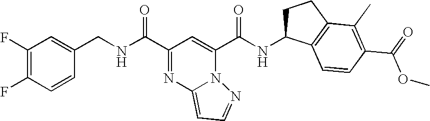 | 1. 56% 2. 518.6 |

### Preparative Example 20-22

Following a similar procedure as that described in Preparative Example 18, step B except using the amide indicated in table below, the following compounds were prepared.

| Prep. | | | 1. Yield |
|---|---|---|---|
| Ex. # | amide | product | 2. [M]⁺ |
| 20 | 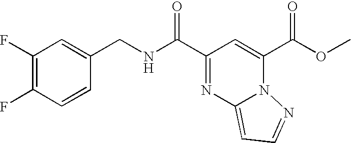 | 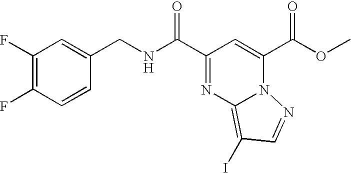 | 1. 97% 2. M + H⁺ = 473 |
| 21 | 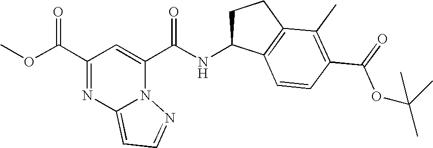 | 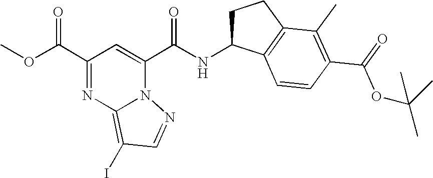 | 1. 100% 2. M + Na⁺ = 599 |
| 22 | 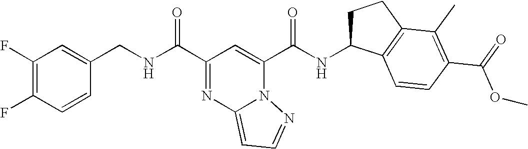 | 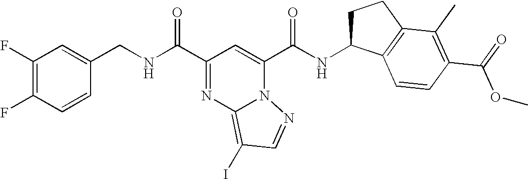 | 1. 78% 2. M − H⁻ = 644.2 |

### Preparative Example 23-24

Following a similar procedure as that described in Preparative Example 18, step C except using the iodides indicated in table below, the following compounds were prepared.

| Prep. | | | 1. Yield |
|---|---|---|---|
| Ex. # | iodide | product | 2. [M − H]⁻ |
| 23 | 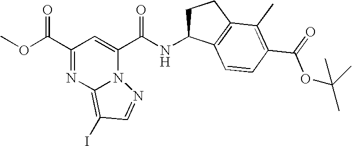 | 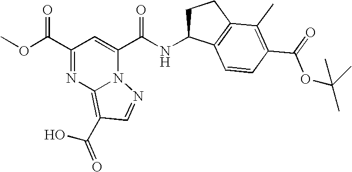 | 1. 88% 2. 588.4 |
| 24 | 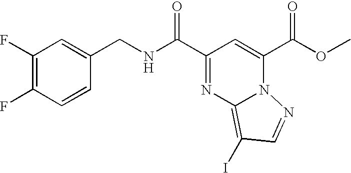 | 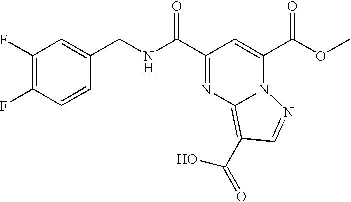 | 1. 100% 2. 389 |

### Preparative Example 25-26

Following a similar procedure as that described in Preparative Example 18, step D except using the acids and amines indicated in table below, the following compounds were prepared.

| Prep. | | | 1. Yield |
|---|---|---|---|
| Ex. # | Acid; amine | product | 2. [M − H]⁻ |
| 25 | 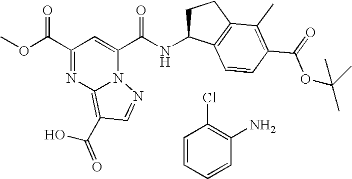 | 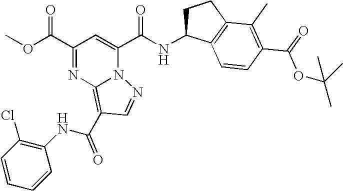 | 1. 67% 2. 602.3 |
| 26 | 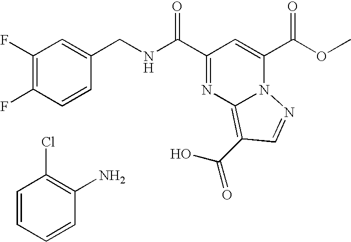 | 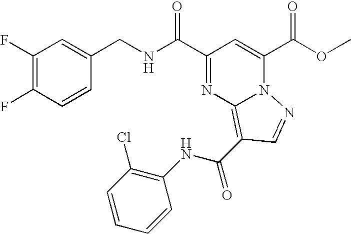 | 1. 42% 2. 598 |

### Preparative Example 27-31

Following a similar procedure as that described in Preparative Example 18, step D except using amines indicated in table below and (S)-5-(3,4-Difluoro-benzylcarbamoyl)-7-(5-methoxycarbonyl-4methyl-indan-1-ylcarbamoyl)-pyrazolo[1,5-a]pyrimidine-3-carboxylic acid, the following compounds were prepared.

| Prep. | | | 1. Yield |
|---|---|---|---|
| Ex. # | amine | product | 2. [M − H]⁻ |
| 27 | 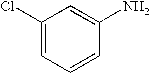 | 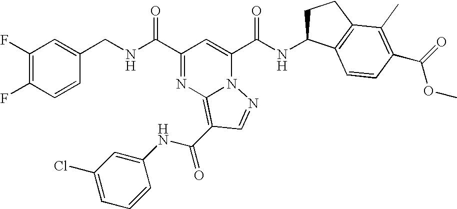 | 1. 90% 2. 671.3 |
| 28 | 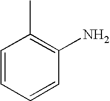 | 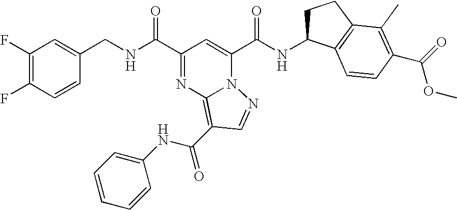 | 1. 87% 2. 651.5 |
| 29 | 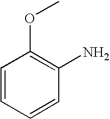 | 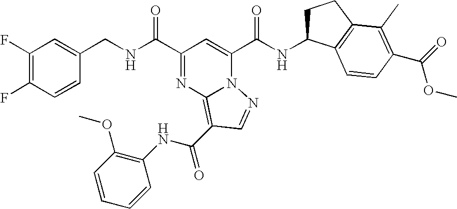 | 1. 78% 2. 667.4 |
| 30 | 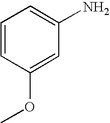 | 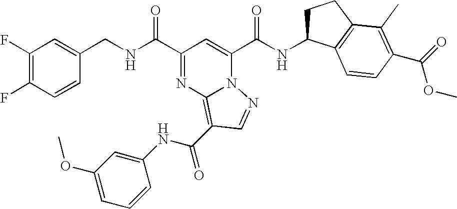 | 1. 65% 2. 667.4 |
| 31 | 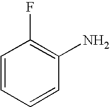 | 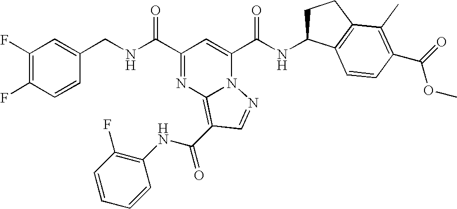 | 1. 99% 2. 655.3 |

### Preparative Example 32

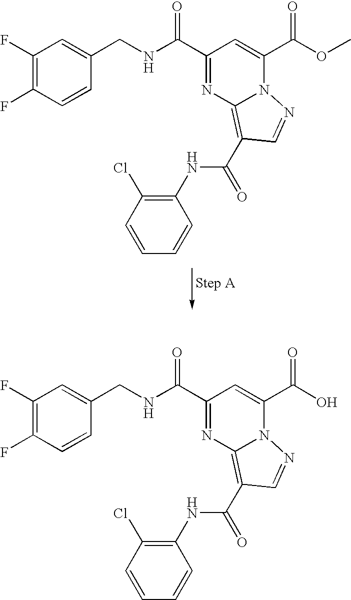 Step A

To a solution of 3-(2-chloro-phenylcarbamoyl)-5-(3,4-difluoro-benzylcarbamoyl)-pyrazolo[1,5-a]pyrimidine-7-carboxylic acid methyl ester (155 mg), in THF (5 mL) and MeOH (1 mL) at 0° C. was added aqueous LiOH (0.5 mL, 1N). The solution was allowed to warm to 22° C. stirred for 1 h and neutralized with aqueous NaHSO₄.(0.3 mL, 2M) The resulting residue was concentrated to get rid of THF and MeOH. The resulting precipitate was collected to give 3-(2-chloro-phenylcarbamoyl)-5-(3,4-difluoro-benzylcarbamoyl)-pyrazolo[1,5-a]pyrimidine-7-carboxylic acid (150 mg, 99%). [MH]⁺=486.

### Preparative Example 33

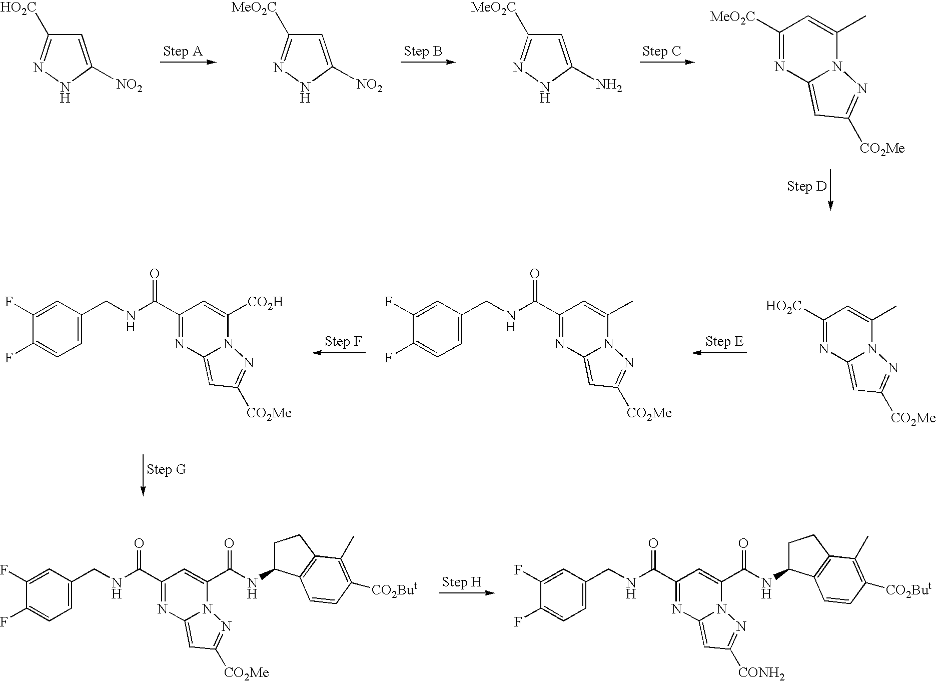 Step A

5-Nitro-1H-pyrazole-3-carboxylic acid (1.57 g, 10 mmol) in methanol (25 mL) was added sulfuric acid (1 g, 10 mmol) and heated at 160° C. for 12 mins in microwave. The solution was concentrated to dryness after being cooled down. The crude product methyl 5-nitro-1H-pyrazole-3-carboxylate was pure enough to use without further purification. MS (M+H): 172.

Step B

To methyl 5-nitro-1H-pyrazole-3-carboxylate (1.45 g, 6.3 mmol) in methanol (25 mL) was added palladium on carbon (106 mg, 0.1 mmol), hydrogenated for 2 h at 25 psi. The reaction 10 mixture was filtered through a bed of celite and concentrated to give desired product, methyl 3-amino-1H-pyrazole 5-carboxylate as white solid (1.25 g, yield, 88%). MS (M+H): 142.

Step C

Methyl 3-amino-1H-pyrazole 5-carboxylate (325 mg, 2.3 mmol) and methyl acetoacetate (330mg, 2.3 mmol) in methanol (10 mL) were heated to reflux for 2 h and cooled down. The resulting precipitate was collected to give white solid product 7-Methyl-pyrazolo[1,5-a]pyrimidine-2,5-dicarboxylic acid dimethyl ester (356 mg, yield 62%). MS (M+H): 250.

Step D

To a solution of methyl-pyrazolo[1,5-a]pyrimidine-2,5-dicarboxylic acid dimethyl ester (229 mg, 0.92 mmol) in dioxane (10 mL) and methanol (2 mL) was added a solution of sodium hyroxide (1N 1 mL). The solution was stirred overnight, acidified, and filter the white precipitate to afford the crude product monoacid (177 mg, 38%). MS (M+H): 236.

Step E

To a mixture of the monoacid and diacid (172 mg), DMF (0.1 mL) and CH₂Cl₂ (2.5 mL) at 0° C. was added oxalyl chloride (180 μL, 2.2 mmol). The ice bath was removed and the mixture was stirred for 45 min and concentrated. The resulting residue was brought up in CH₂Cl₂ (2.5 mL) and added 3,4-difluorobenzylamine (114 mg, 0.8 mmol) and triethylamine (210 μL, 1.5 mmol) in CH₂Cl₂ (1 mL). The resulting mixture was stirred for 16 h and concentrated. The crude product was purified by silica gel chromatography to give the product, 5-(3,4-difluoro-benzylcarbamoyl)-7-methyl-pyrazolo[1,5-a]pyrimidine-2-carboxylic acid methyl ester (171 mg, yield, 65%). MS (M+H): 361.

Step F

The mixture of above ester (151 mg, 0.42 mmol) in dioxane (5 mL) was added selenium dioxide (116 mg, 1.05 mmol) and heated to reflux overnight. After it was cooled down and filter through a bed of celite, the resulting clear yellow solution was added oxone (646 mg, 1.05 mmol) and stirred for 24 h. The solution was filtered and concentrated to dryness. The crude product, 5-(3,4-difluoro-benzylcarbamoyl)-pyrazolo[1,5-a]pyrimidine-2,7-dicarboxylic acid 2-methyl ester, was utilized without further purification. MS (M+H): 391.

Step G

To a mixture of the 5-(3,4difluoro-benzylcarbamoyl)-pyrazolo[1,5-a]pyrimidine-2,7-dicarboxylic acid 2-methyl ester (0.48 mmol), DMF (0.1 mL) and CH₂Cl₂ (5 mL) at 0° C. was added oxalyl chloride (100 μL, 1.3 mmol). The ice bath was removed and the mixture was stirred for 45 min and concentrated. The resulting residue was brought up in CH₂Cl₂ (5 mL) and added [(S)-1-amino-4-methyl-indan-5-carboxylic acid tert-butyl ester (104 mg, 0.42 mmol) and triethylamine (140 μL, 1 mmol) in CH₂Cl₂ (2 mL). The resulting mixture was stirred for 16 h and concentrated. The crude product was purified by silica gel chromatography to give the diamide, [(S)-7-(5-tert-butoxycarbonyl-4methyl-indan-1-ylcarbamoyl)]-5-(3,4-difluoro-benzyl carbamoyl)-pyrazolo[1,5-a]pyrimidine-2-carboxylic acid methyl ester (58 mg, yield, 10%). MS (M+Na): 642.

Step H

[(S)-7-(5-tert-butoxycarbonyl-4-methyl-indan-1-ylcarbamoyl)]-5-(3,4-difluoro-benzylcarbamoyl)-pyrazolo[1,5-a]pyrimidine-2-carboxylic acid methyl ester (5 mg, 0.08 mmol) in ammonia methanol solution (7N, 2 mL) was heated to 65° C. overnight, concentrated and purified by silica gel chromatography to give (S)-1-{[2-carbamoyl-5-(3,4-difluoro-benzylcarbamoyl)-pyrazolo[1,5-a]pyrimidine-7-carbonyl]-amino}-4-methyl-indan-5-carboxylic acid tert-butyl ester (4.5 mg, yield 90%). MS (M+H): 605.

### Preparative Example 34

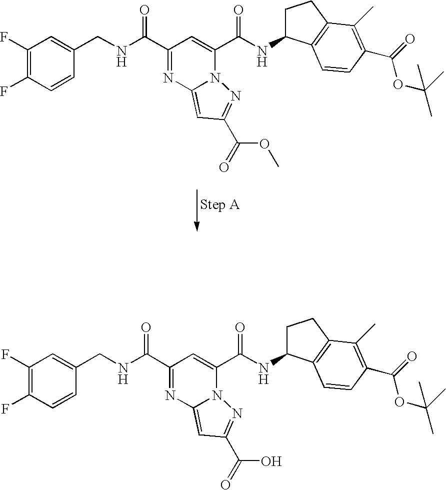 Step A

The mixture of [(S)-7-(5-tert-butoxycarbony-4-methy-indan-1-ylcarbamoyl)]-5-(3,4difluoro-benzyicarbamoyl)-pyrazolo[1,5-a]pyrimnidine-2-carboxylic acid methyl ester (25 mg, 0.04 mmol), trimethyltin hydroxide (18.2 mg, 0.1 mmol) in 1,2-dichloroethane (2 mL) was heated to reflux for overnight and concentrated. The crude product was washed witb hydrochloric acid and dried to give yellow solid (S)-7-(5-tert-butoxycarbonyl-4-methy-indan-1-ylcarbamoyl)-5-(3,4-difluoro-benzyl carbamoyl)-pyrazolo[1,5-a]pyrirnidine-2-carboxylic acid (21.5 mg, yield, 86%). MS (M+H): 606.

### Preparative Example 35

Following a similar procedure as that described in Preparative Example 34 except using the ester indicated in table below, the following compound was prepared.

| Prep. | | | 1. Yield |
|---|---|---|---|
| Ex. # | ester | product | 2. [M − H]⁻ |
| 35 | 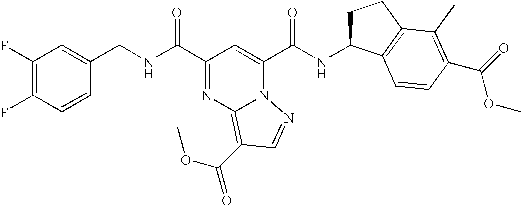 | 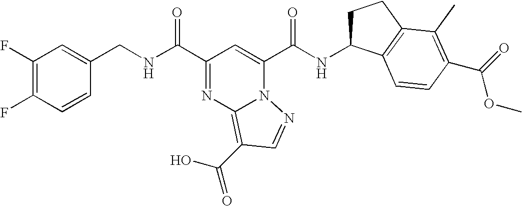 | 1. 90% 2. 564.3 |

### Preparative Example 36

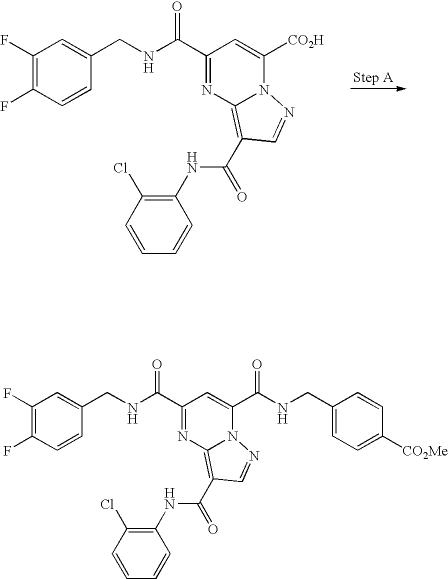 Step A

To a mixture of the 3-(2-chloro-phenylcarbamoyl)-5-(3,4-difluoro-benzylcarbamoyl)-pyrazolo[1,5-a]pyrimidine-7-carboxylic acid (23 mg, 0.05 mmol), DMF (0.1 mL) and CH₂Cl₂ (2.5 mL) at 0° C. was added oxalyl chloride (12 μL, 0.15 mmol). The ice bath was removed and the mixture was stirred for 45 min and concentrated. The resulting residue was brought up in CH₂Cl₂ (2.5 mL) and added 3,4-difluorobenzylamine (15 mg, 0.075 mmol) and triethylamine (21 μL, 0.15 mmol) in CH₂Cl₂ (1 mL). The resulting mixture was stirred for 16 h and concentrated. The crude product was purified by silica gel chromatography to give the product, 4-({[3-(2-chloro-phenylcarbamoyl)-5-(3,4-difluoro-benzylcarbamoyl)-pyrazolo[1,5-a]pyrimidine-7-carbonyl]-amino}-methyl)-benzoic acid methyl ester (6 mg, yield, 19%). MS (M+H): 633.

### Preparative Example 37-38

If one followed a similar procedure as described in Preparative Example 36 except using the amines indicated in table below, the following compounds could be prepared.

| Prep. | | |
|---|---|---|
| Ex. # | amine | product |
| 37 | 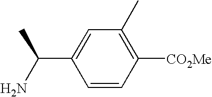 | 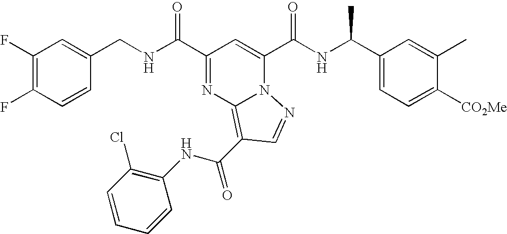 |
| 38 | 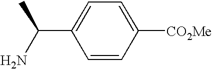 | 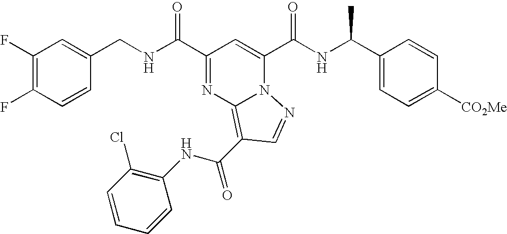 |

### Preparative Example 39

Following a similar procedure as that described in Preparative Example 36 except using the amine indicated in table below, the following compounds were prepared.

| Prep. | | | 1. Yield |
|---|---|---|---|
| Ex. # | amine | product | 2. MH⁺ |
| 39 | 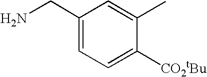 | 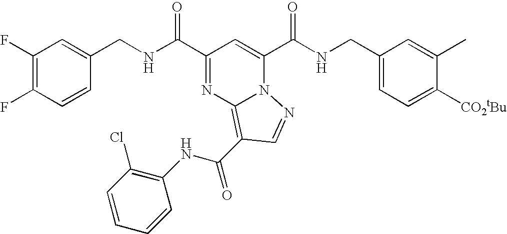 | 1. 36% 2. 689 |

### Preparative Example 40

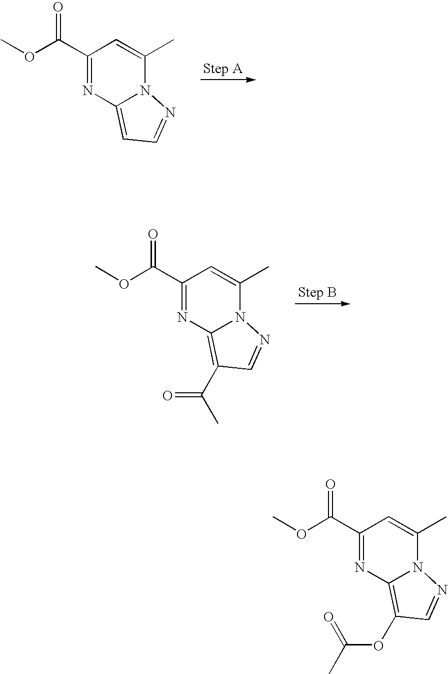 Step A

To a solution of the major isomer of the title compound from the Preparative Example 13 (2.0 g) in CH₂Cl₂ (20 mL) were added acetyl chloride (3.0 mL) and SnC₄ (10.9 g). The resulting mixture was heated to reflux overnight, cooled and quenched with H₂O (10 mL). The aqueous phase was separated and extracted with CH₂Cl₂ (2×). The combined organic phases were concentrated and purified by chromatography (silica, cyclohexane/EtOAc) to afford the title compound (1.2 g, 49%). [MH]⁺=234.

Step B

Trifluoroacetic anhydride (4.6 mL) was added dropwise to an ice cooled suspension of urea hydrogen peroxide (5.8 g) in CH₂Cl₂ (40 mL). The mixture was stirred for 30 min, then a solution of the title compound from Step A above (1.8 g) in CH₂Cl₂ (20 mL) was added and the mixture was stirred at room temperature overnight. NaHSO₃ (1.0 g) was added and the resulting mixture was diluted with saturated aqueous NaHCO₃ (40 mL). The aqueous phase was separated and extracted with CH₂Cl₂. The combined organic phases were concentrated and purified by chromatography (silica, cyclohexane/EtOAc) to afford 3-acetoxy-7-methyl-pyrazolo[5-a]pyrimidine-5-carboxylic acid methyl ester (500 mg, 26%). ¹H-NMR (CDCl₃) δ=8.40 (s, 1H), 7.47 (d, 1H), 4.03 (s, 3H), 2.84 (d, 3H), 2.42 (s, 3H).

### Preparative Example 41

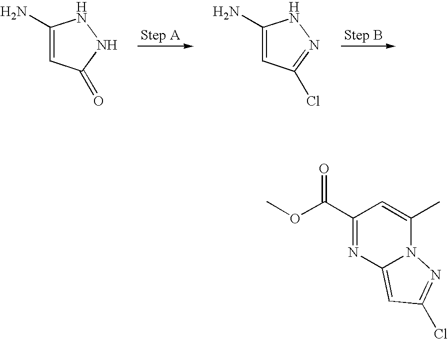 Step A

A mixture of commercially available 5-aminopyrazolone (5 g) and POCl₃ (50 mL) was heated to 210° C. for 5 h, concentrated and quenched with MEOH (10 mL) at 0° C. Purification by chromatography (silica, hexanes/EtOAc) afforded the desired product (293 mg, 5%). [MH]⁺=118.

Step B

A mixture of the title compound from Step A above (117 mg) and methyl acetopyruvate (144 mg) in MeOH (5 mL) was heated to reflux for 2 h and then cooled to 0° C. The formed precipitate was collected by filtration to give 2-chloro-7-methyl-pyrazolo[1,5-a]pyrimidine-5-carboxylic acid methyl ester (200 mg, 89%). [MH]⁺=226.

### Preparative Example 42

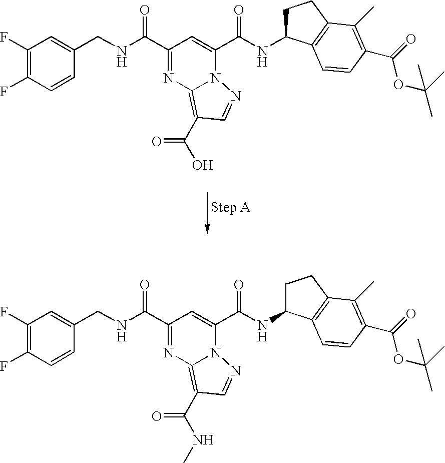 Step A

To a solution of (S)-7-(5-tert-butoxycarbonyl-4-methyl-indan-1-ylcarbamoyl)-5-(3,4-difluoro-benzylcarbamoyl)-pyrazolo[1,5-a]pyrimidine-3-carboxylic acid (8 mg), and DMF (1 μL) in CH₂Cl₂ (0.3 mL) at 0° C. was added oxalyl chloride (5 μl). The solution was allowed to warm to 22° C. stirred for 3 h and concentrated. The resulting residue was brought up in CH₂Cl₂ (0.2 mL) and cooled to 0° C. To this cooled solution were added triethyl amine (4 μL) and a solution of methylam ine hydrochloroide salt (3 mg) and triethylamine (7 μL) in CH₂Cl₂ (0.2 mL). The resulting solution was stirred at 22° C. for 18 h and absorbed onto silica and purified by silica gel chromatography to give (S)-1-{[5-(3,4-difluoro-benzylcarbamoyl)-3-methylcarbamoyl-pyrazolo[1,5-a]pyrimidine-7-carbonyl]-amino}-4-methyl-indan-5-carboxylic acid tert-butyl ester (5.3 mg, 66%). [M−H]⁻=617.5.

### Preparative Example 43

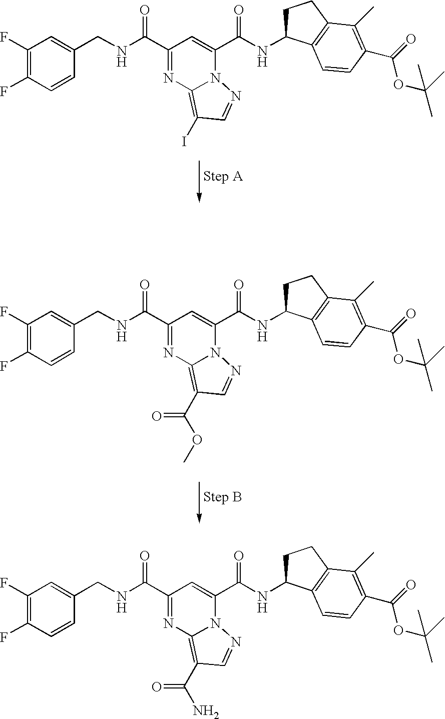 Step A

A mixture of (S)-1-{[5-(3,4-Difluoro-benzylcarbamyl)-3-iodo-pyrazolo[1,5-a]pyrimidine-7-carbonyl]-amino}-4-methyl-indan-5-carboxylic acid tert-butyl ester (393 mg), Pd(PPh₃)₄ (66 mg), and triethylamine (800 μL) in DMSO (1.5 mL) and MeOH (1.5 mL) under 1 atm of carbon monoxide was stirred at 80° C. for 18 h. 1N HCl was added and the aqueous layer was washed three times with EtOAc. The organic layers were combined and washed twice with 1N HCl and once with brine, dried over MgSO₄, filtered, absorbed onto silica and purified by silica gel chromatography to give (S)-7-(5-tert-butoxycarbonyl4methyl-indan-1-ylcarbamoyl)-5-(3,4-difluoro-benzylcarbamoyl)-pyrazolo[1,5-a[pyrmdine-3-carboxylic acid methyl ester (195 mg, 55%), [M−H]⁻=618.4

Step B

A solution of (S)-7-(5-tert-butoxycarbonyl-4-methyl-indan-1-ylcarbamoyl)-5-(3,4-difluoro-benzylcarbamoyl)-pyrazolo[1,5-a]pyrimidine-3-carboxylic acid methyl ester (15 mg) in 7M ammonia in MeOH was stirred at 70° C. for three days in a sealed vial. The solution was concentrated and purified by preparatory plate to give (S)-1-{[3-carbamoyl-5-(3,4-difluoro-benzylcarbamoyl)-pyrazolo[1,5-a]pyrimidine-7-carbonyl]-amino }-4-methyl-indan-5-carboxylic acid tert-butyl ester (2.5 mg, 17%). [M−H]⁻=603.5.

### Preparative Example 44

Following a similar procedure as that described in Preparative Example 43, step A except using the iodide indicated in table below, the following compound was prepared.

| Prep. | | | 1. Yield |
|---|---|---|---|
| Ex. # | ester | product | 2. [M − H]⁻ |
| 44 | 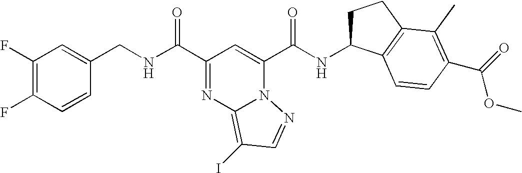 | 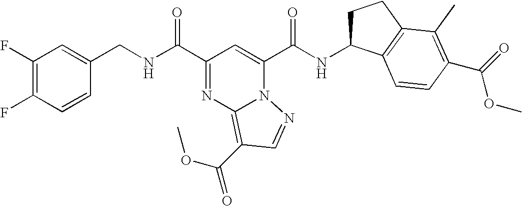 | 1. 98% 2. 576.4 |

### Preparative Example 45

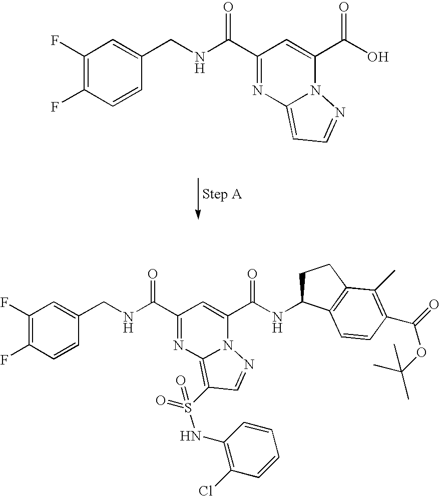 Step A

A mixture of 5-(3,4-Difluoro-benzylcarbamoyl)-pyrazolo[1,5-a]pyrimidine-7-carboxylic acid (168 mg) in chlorosulfonic acid (2 mL) was stirred at 90° C. for 2 h. The solution was cooled and cautiously poured onto ice (15 g). Once the ice had melted the precipitate was collected by filtration and dried on vacuum. The resulting solid was mixed with 2-chloroaniline (100 μL) and chloroform (5 mL) and stirred at 70° C. for 18 h. The solution was purified by silica gel chromatography to give a residue (9 mg) that contained 3-(2-chloro-phenylsulfamoyl)-5-(3,4-difluoro-benzylcarbamoyl)-pyrazolo[1,5-a]pyrimidine-7-carboxylic acid. [M−H]⁻=520.5. To the residue (9 mg) and DMF (1 μL) in CH₂Cl₂ (0.2 mL) at 0° C. was added oxalyl chloride (8 μl). The solution was allowed to warm to 22° C. stirred for 3 h and concentrated. The resulting residue was brought up in CH₂Cl₂ (0.2 mL) and cooled to 0° C. To this cooled solution were added triethyl amine (4 μL) and a solution of (S)-1-amino-4-methyl-indan-5-carboxylic acid tert-butyl ester (5 mg) and triethylamine (4 μL) in CH₂Cl₂ (0.2 mL). The resulting solution was stirred at 22° C. for 18 h and purified by preparatory plate to give 1-{[3-(2-Chloro-phenylsulfamoyl)-5-(3,4-difluoro-benzylcarbamoyl)-pyrazolo[1,5-a]pyrimidine-7-carbonyl]-amino}-4-methyl-indan-5-carboxylic acid tert-butyl ester (3 mg, 0.8%). [M−H]⁻=749.4.

### Preparative Example 46

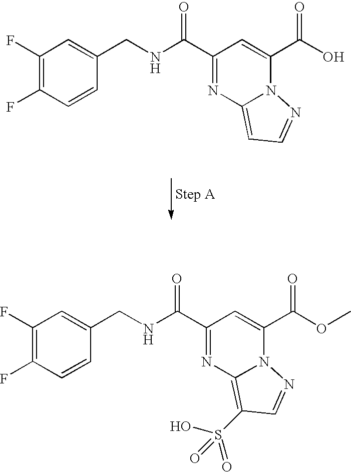 Step A

A mixture of 5-(3,4-Difluoro-benzylcarbamoyl)-pyrazolo[1,5-alpyrimidine-7-carboxylic acid (50 mg) and chlorosulfonic acid (0.5 mL) was stirred at 90° C. for 1 h. The solution was cooled and cautiously poured onto ice (5 g). Once the ice had melted the precipitate was collected by filtration and dried on vacuum. The resulting solid was added to a premixed solution of acetyl chloride (100 μL) in MeOH (1 mL) and stirred at 40° C. for 1 h and concentrated to give 5-(3,4-difluoro-benzylcarbamoyl)-3-sulfo-pyrazolo[1,5-a]pyrimidine-7-carboxylic acid methyl ester (42 mg, 65%). [M−H]⁻=425.3.

### Preparative Example 47

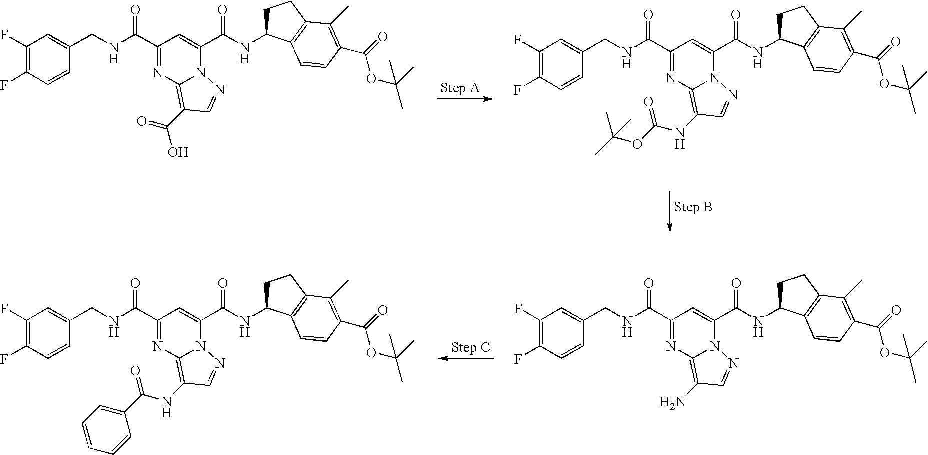 Step A

To a mixture of (S)-7-(5-tert-butoxycarbonyl-4-methyl-indan-1-ylcarbamoyl)-5-(3,4-difluoro-benzylcarbamoyl)-pyrazolo[1,5-a]pyrimidine-3-carboxylic acid (150 mg), and DMF (2 μL) in CH₂Cl₂ (2.5 mL) at 0° C. was added oxalyl chloride (108 μl). The solution was allowed to warm to 22° C. stirred for 2 h and concentrated. The resulting residue was brought up in acetone (1.5 mL) and cooled to 0° C. To this cooled solution was added a solution of sodium azide (100 mg) in water (0.5 mL). The ice bath was removed and the resulting solution was stirred at 22° C. for 1 h. Water (5 mL) was added and the aqueous layer was washed three times with toluene (3×5 mL). The organic layers were combined, dried over MgSO₄, filtered and concentrated. The resulting residue and 4 molecular sieves (100 mg) was brought up in toluene (1 mL) and tert-butanol (1 mL) and stirred at 100° C. for 1.5 h. The mixture was filtered and the supernatant was absorbed onto silica and purified by silica gel chromatography to give (S)-1-{[3-tert-butoxycarbonylamino-5-(3,4-difluoro-benzylcarbamoyl)-pyrazolo[1,5-a]pyrimidine-7-carbonyl]-amino}-4-methyl-indan-5-carboxylic acid tert-butyl ester (88 mg, 52%). [M−H]⁻=675.6.

Step B

A solution of (S)-1-{[3-tert-butoxycarbonylamino-5-(3,4-difluoro-benzylcarbamoyl)-pyrazolo[1,5-a]pyrimidine-7-carbonyl]-amino }-4-methyl-indan-5-carboxylic acid tert-butyl ester (88 mg) in tert-butylacetate (1 mL) and sulfuric acid (35 μl) was stirred for 1.5 h. A saturated solution of sodium bicarbonate (4 mL) and EtOAc (2 mL) were added and the mixture stirred for 1 h. The aqueous layer was separated and washed twice with EtOAc and twice with CH₂Cl₂. The combined organic layers were dried over MgSO₄, filtered and absorbed onto silica gel and purified by silica gel chromatography to give (S)-1-{[3-amino-5-(3,4-difluoro-benzylcarbamoyl)-pyrazolo[1,5-a]pyrimidine-7-carbonyl]-amino}-4-methyl-indan-5-carboxylic acid tert-butyl ester (36 mg, 50%). [M−H]⁺=577.2.

Step C

To a solution of benzoyl chloride (3 μL) in CH₂Cl₂ (100 μL) at 0° C. were added triethylamine (6 mL) and a solution of (S)-1-([3-amino-5-(3,4-difluoro-benzylcarbamoyl)-pytazolo[1,5-a]pyrimidine-7-carbonyl]-amino}-4-methyl-indan-5-carboxylic acid tert-butyl ester (12 mg) in CH₂Cl₂ (100 μL). The solution was allowed to warm to 22° C. and stirred for 18 h and concentrated. The residue was purified by preparatory plate to give (S)-1-{[3-benzoylamino-5-(3,4-difluoro-benzylcarbamoyl)-pyrazolo[1,5-a]pyrimidine-7-carbonyl]-amino }-4-methyl- indan-5-carboxylic acid tert-butyl ester (11.2 mg, 79%). [M−H]⁻=679.6.

### Preparative Examiple 48

Following a similar procedure as that described in Preparative Example 47, step C, except using the chloride in table below, the following compounds were prepared.

| Prep. | | | 1. Yield |
|---|---|---|---|
| Ex. # | chloride | product | 2. [M − H]⁻ |
| 48 | 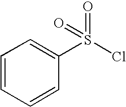 | 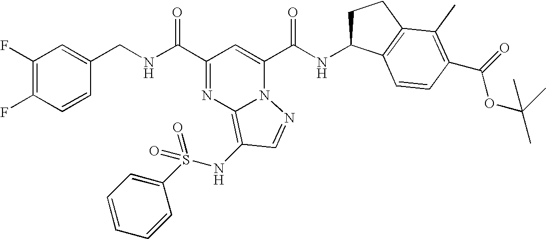 | 1. 21% 2. 715.5 |

### Preparative Example 49

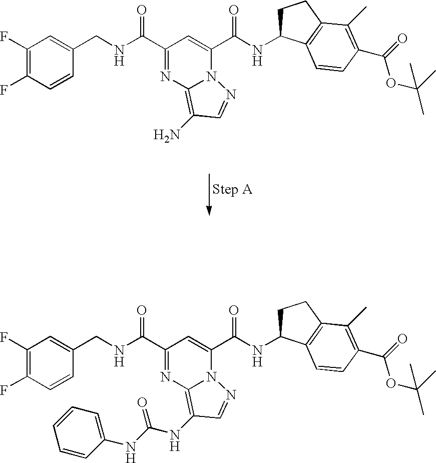 Step A

A solution of (S)-1-{[3-amino-5-(3,4-difluoro-benzylcarbamoyl)-pyrazolo[1,5-a]pyrimidine-7-carbonyl]-amino)-4-methyl-indan-5-carboxylic acid tert-butyl ester (12 mg) and phenylisocyanate (3 μL) in CH₂Cl₂ (200 μL) was stirred for three days and concentrated. The residue was purified by silica gel chromatography to give 1-{[5-(3,4-difluoro-benzylcarbamoyl)-3-(3-phenyl-ureido)-pyrazolo[1,5-a]pyrimidine-7-carbonyl]-amino}-4-methyl-indan-5-carboxylic acid tert-butyl ester (11 mg, 76%). [M−H]⁻=694.5.

### Preparative Example 50

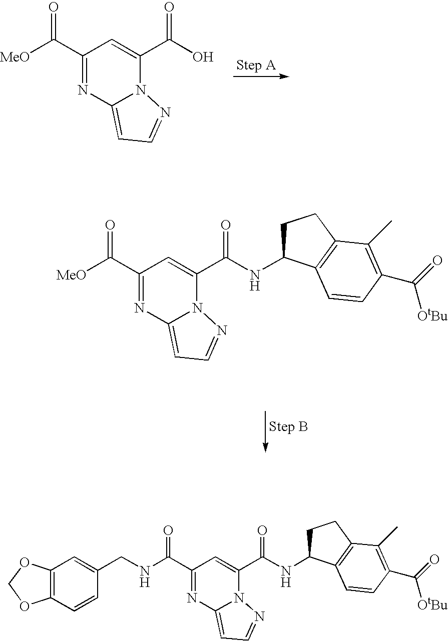 Step A

Pyrazolo[1,5-a]pyrimidine-5,7-dicarboxylic acid 5-methyl ester (100 mg) was treated with oxylyl chloride (116 μL) and DMF (2 drops) in methylene chloride (4 mL) for 1 h. The reaction mixture was concentrated under reduced pressure and redissloved in methylene chloride (4 mL). (S)-1-Amino4methyl-indan-5-carboxylic acid tert-butyl ester (133 mg) and triethylamine (19 μL) were added to the mixture and stirred for 15 h before it was concentrated and purified by column chromatography (silica, hexane/EtOAc) to afford (S)-7-(5-tert-butoxycarbonyl-4-methyl-indan-1-ylcarbamoyl)-pyrazolo[1,5-a]pyrimidine-5-carboxylic acid methyl ester (164 mg,81%). [MH]⁺=451.0.

Step B

(S)-7-(5-tert-butoxycarbonyl-4-methyl-indan-1-ylcarbamoyl)-pyrazolo[1,5-a]pyrimidine-5-carboxylic acid methyl ester (20 mg) and piperonylamine (20 mg) was dissolved in DMF (2 mL). The mixture was stirred in microwave at 150° C. for 10 min and concentrated under reduced pressure. The residue was purified by column chromatography to afford title compound. (5 mg,18%). [MH]⁺=570.2.

### Preparative Example 51-64

Following a similar procedure as that described in Preparative Example 27, step B, except using the amine in table below and (S)-7-(5-tert-Butoxycarbonyl-4-methyl-indan-1-ylcarbamoyl)-3-(2-chloro-phenylcarbamoyl)-pyrazolo[1,5-a]pyrimidine-5-carboxylic acid methyl ester, the following compounds were prepared.

| Prep. | | | |
|---|---|---|---|
| Ex. | | | 1. Yield |
| # | amine | product | 2. [M − H]⁻ |
| 51 | NH₂Me | 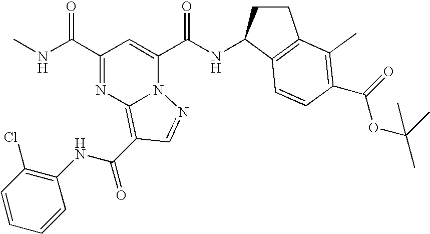 | 1. 100% 2. 601.5 |
| 52 | 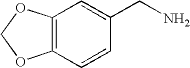 | 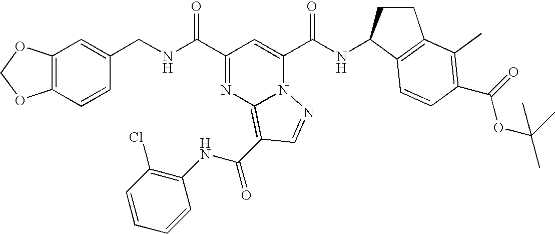 | 1. 65% 2. 721.4 |
| 53 | 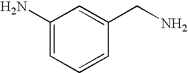 | 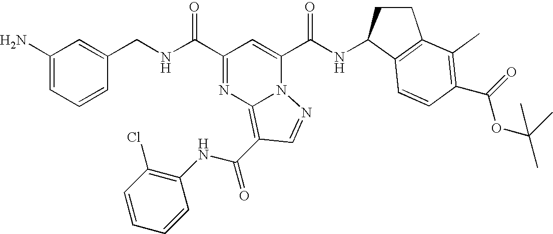 | 1. 48% 2. 692.6 |
| 54 | 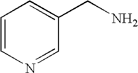 | 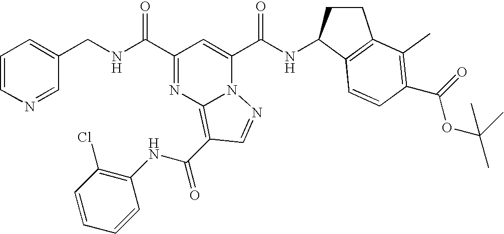 | 1. 37% 2. 678.6 |
| 55 | 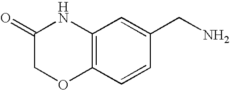 | 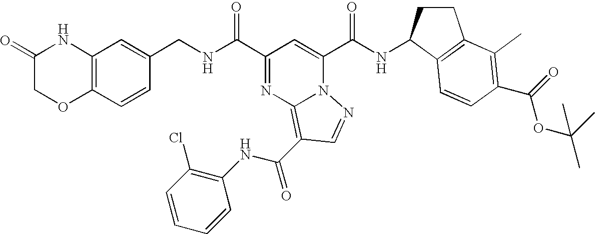 | 1. 63% 2. 683.5 |
| 56 | 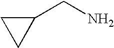 | 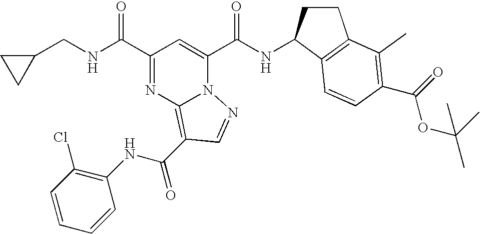 | 1. 67% 2. 641.5 |
| 57 | 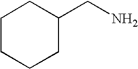 | 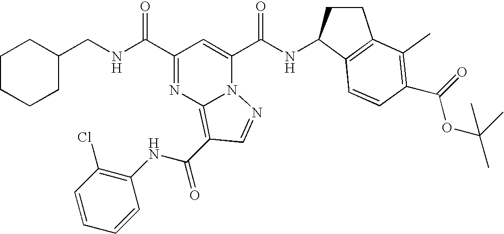 | 1. 63% 2. 683.5 |
| 58 | 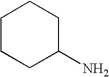 | 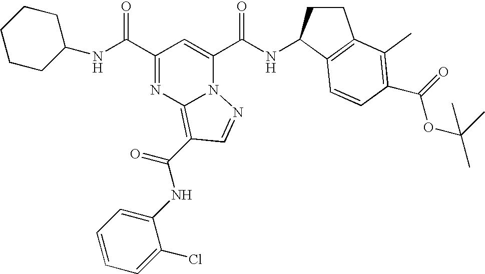 | 1. 73% 2. 669.5 |
| 59 | 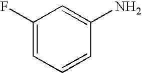 | 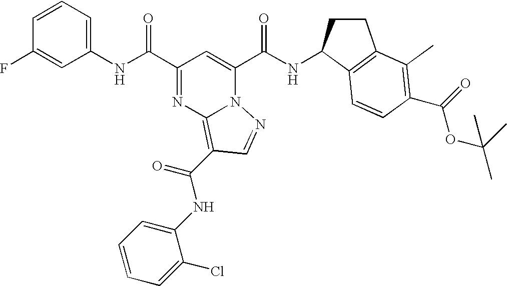 | 1. 68% 2. 681.4 |
| 60 | 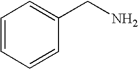 | 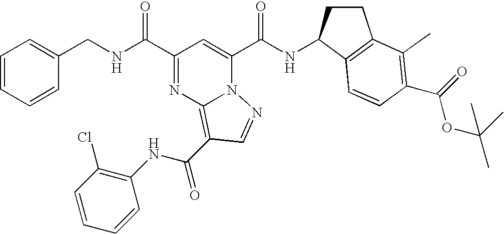 | 1. 62% 2. 677.5 |
| 61 | 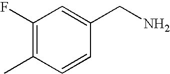 | 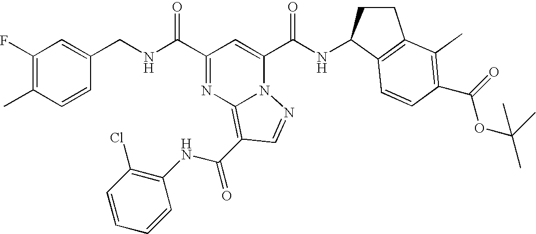 | 1. 70% 2. 709.5 |
| 62 | 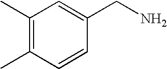 | 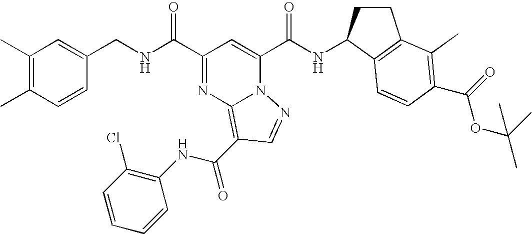 | 1. 68% 2. 705.5 |
| 63 | 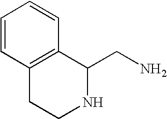 | 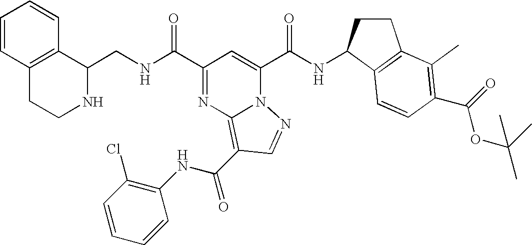 | 1. 42% 2. 732.7 |
| 64 | 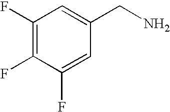 | 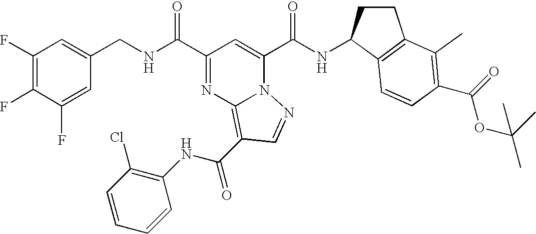 | 1. 17% 2. 731.4 |

### Example 1

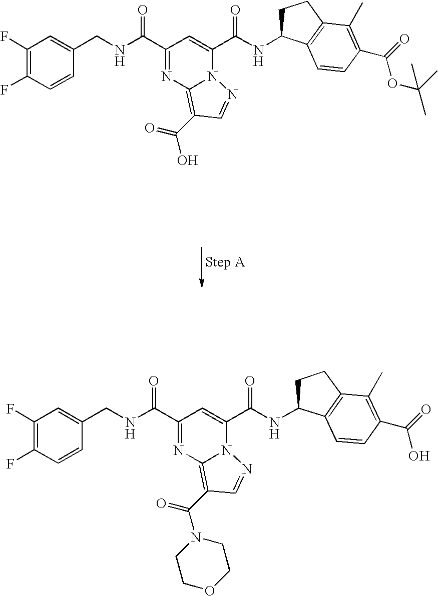 Step A

To a solution of (S)-7-(5-tert-butoxycarbonyl4-methyl-indan-1-ylcarbainoyl)-5-(3,4-difluoro-benzylcarbamoyl)-pyrazolo[1,5-a]pyrimidine-3-carboxylic acid (8 mg), and DMF (1 μL) in CH₂Cl₂ (0.3 mL) at 0° C. was added oxalyl chloride (5 μl). The solution was allowed to warm to 22° C. stirred for 3 h and concentrated. The resulting residue was brought up in CH₂Cl₂ (0.2 mL) and cooled to 0° C. To this cooled solution were added triethyl amine (4 μL) and a solution of morpholine (4 μL) in CH₂Cl₂ (0.2 mL). The resulting solution was stirred at 22° C. for 18 h and absorbed onto silica and purified by silica gel chromatography to give (S)-1-{[5-(3,4-difluoro-benzylcarbamoyl)-3-(morpholine4-carbonyl)-pyrazolo[1,5-a]pyrimidine-7-carbonyl]-amino)}4-methyl-indan-5-carboxylic acid tert-butyl ester. To a solution of (S)-1-{[5-(3,4-difluoro-benzylcarbamoyl)-3-(morpholinercarbonyl)-pyrazolo[1,5-a]pyriridine-7-carbonyl]-amino}4-methyl-indan-5-carboxylic acid tert-butyl ester in CH₂Cl₂ (0.06 mL) at 0° C. was added trifluoroacetic acid (0.06 mL) and this solution stirred for 1 h and was concentrated. The resulting solid was washed 3 times with Et₂O (0.2 mL) to give 1-{[5-(3,4-Difluoro-benzylcarbamoyl)-3-(morpholine4-carbonyl)-pyrazolo[1,5-a]pyrimidine-7-carbonyl]-amino}4-methyl-indan-5-carboxylic acid (3.2 mg, 60%). [M−H]⁻=617.4

### Example 2-20

Following a similar procedure as described in example 1 except using the amines as indicated in the table below, the following compounds were prepared.

| | | | 1. Yield |
|---|---|---|---|
| Ex. # | amine | product | 2. [M − H]⁻ |
| 2 | 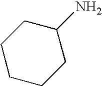 | 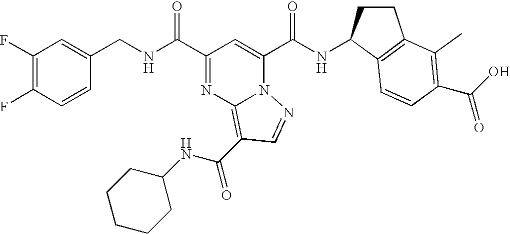 | 1. 85% 2. 629.4 |
| 3 | 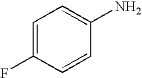 | 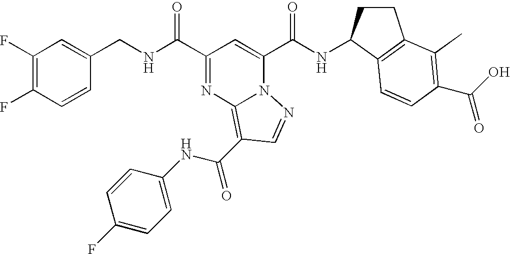 | 1. 83% 2. 641.3 |
| 4 | 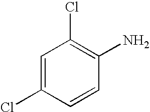 | 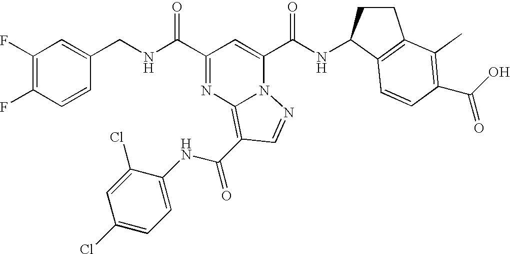 | 1. 80% 2. 691.3 |
| 5 | 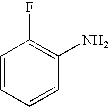 | 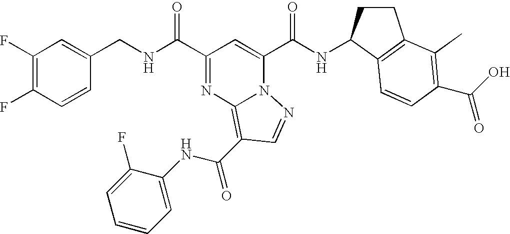 | 1. 53% 2. 641.3 |
| 6 | 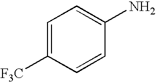 | 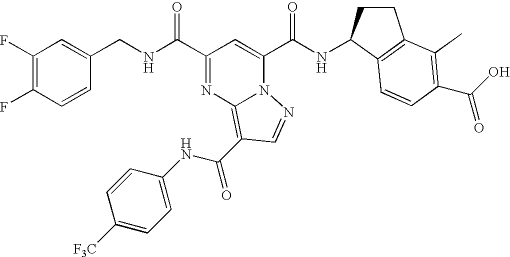 | 1. 35% 2. 691.3 |
| 7 | 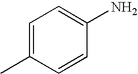 | 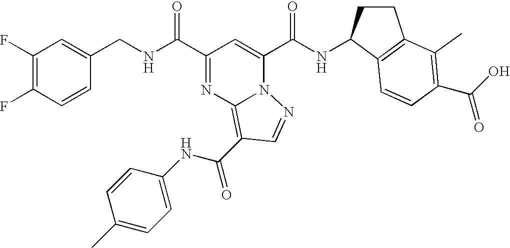 | 1. 76% 2. 637.3 |
| 8 | 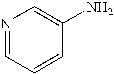 | 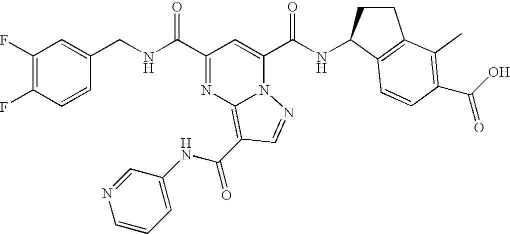 | 1. 67% 2. 624.4 |
| 9 | 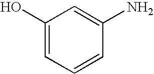 | 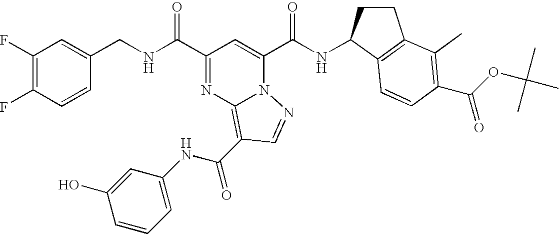 | 1. 65% 2. 639.4 |
| 10 | 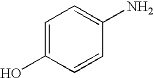 | 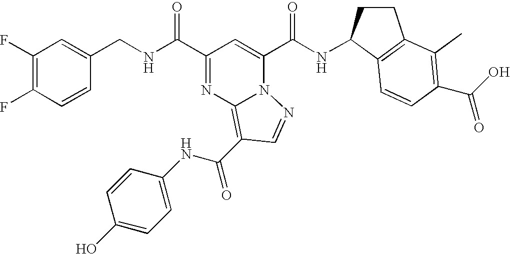 | 1. 70% 2. 639.3 |
| 11 | 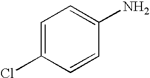 | 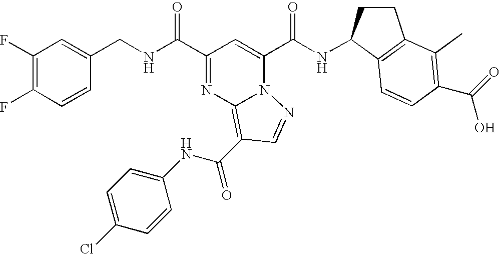 | 1. 42% 2. 623 |
| 12 | 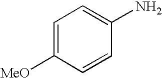 | 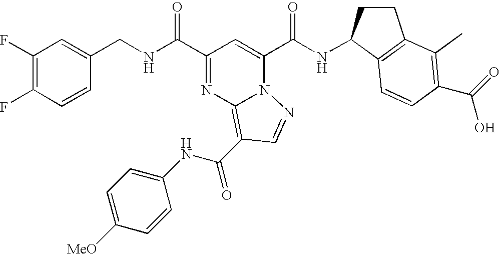 | 1. 45% 2. 653 |
| 13 | 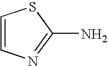 | 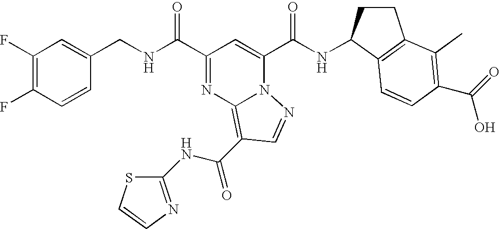 | 1. 36% 2. 630 |
| 14 | 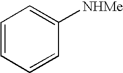 | 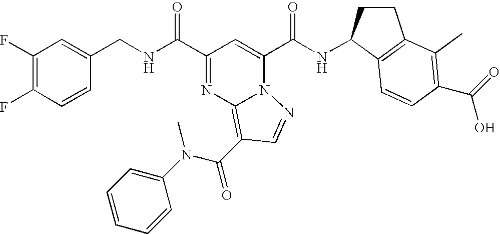 | 1. 32% 2. 637 |
| 15 | 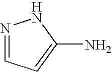 | 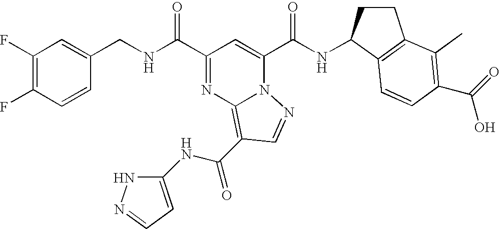 | 1. 39% 2. 613 |
| 16 | 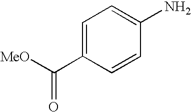 | 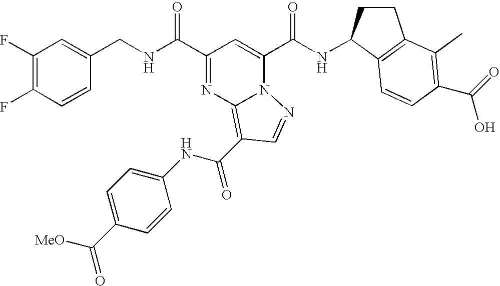 | 1. 8% 2. 681 |
| 17 | 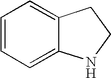 | 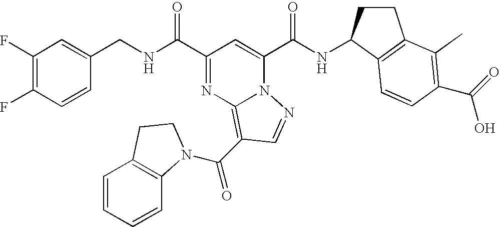 | 1. 74% 2. 649 |
| 18 | 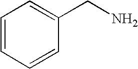 | 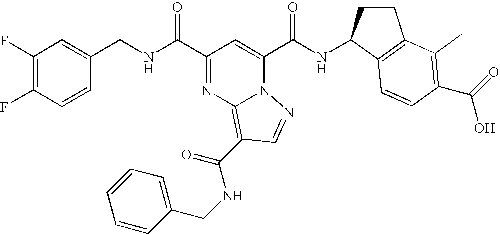 | 1. 72% 2. 637.5 |
| 19 | 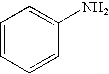 | 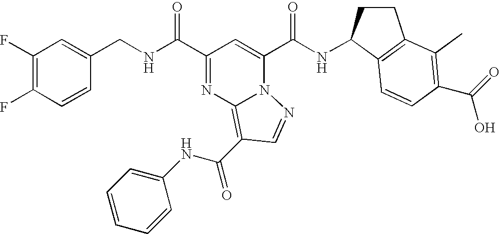 | 1. 17% 2. 623 |
| 20 | 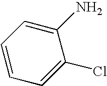 | 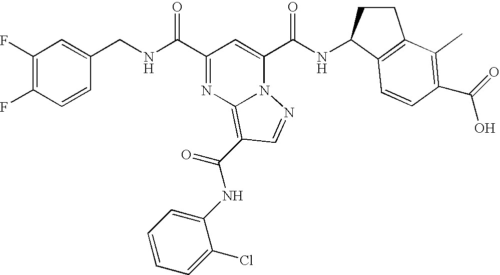 | 1. 65% 2. 657.2 |

### Emample 21

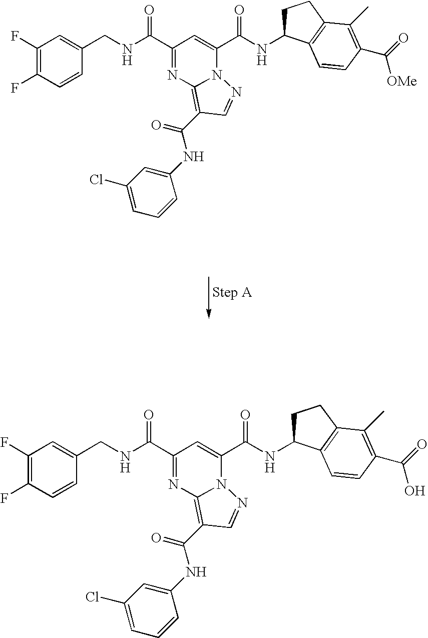 Step A

1-{[3-(3-chloro-phenylcarbamoyl)-5-(3,4-difluoro-benzylcarbamoyl)-pyrazolo[1,5-a]pyriridine-7-carbonyl]-amino}-4-methyl-indan-5-carboxylic acid methyl ester (16 mg) and aluminum bromide (20 mg) were dissolved in tetrahydrothiophene (1 mL) and stirred for 24 h. The mixture was concentrated and purified by silica gel chromatograph (silica, CH₂Cl_{2/}MeOH) to yield 1-{[3-(3-chloro-phenylcarbamoyl)-5-(3,4-difluoro-benzylcarbamoyl)-pyrazolo[1,5-a]pyrimidine-7-carbonyl]-amino})methyl-indan-5-carboxylic acid (6.3 mg, 40%). [M−H]⁻=657.3.

### Example 22-25

Following a similar procedure as described in example 21 except using esters as indicated in the table below, the following compounds were prepared.

| | | | 1. Yield |
|---|---|---|---|
| Ex. # | ester | product | 2. [M − H]⁻ |
| 22 | 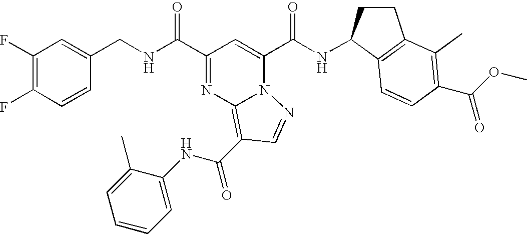 | 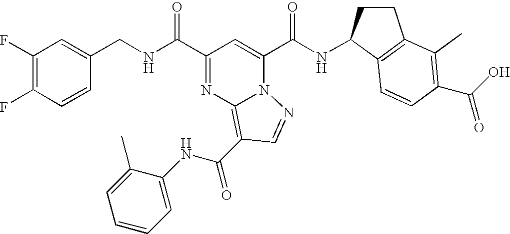 | 1. 55% 2. 637.4 |
| 23 | 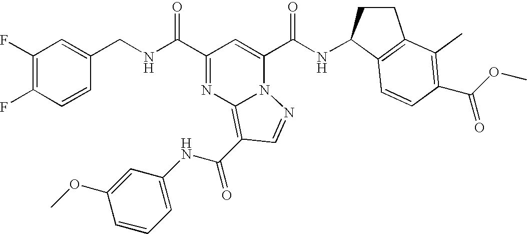 | 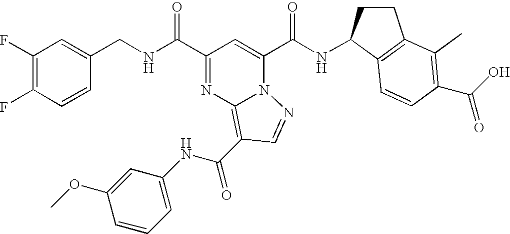 | 1. 40% 2. 653.3 |
| 24 | 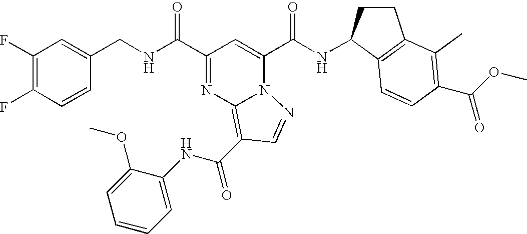 | 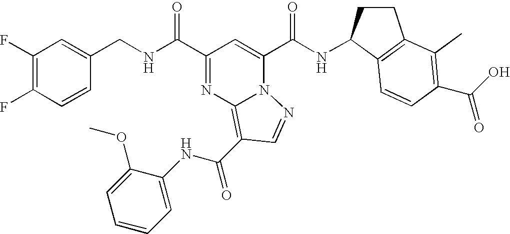 | 1. 34% 2. 653.4 |
| 25 | 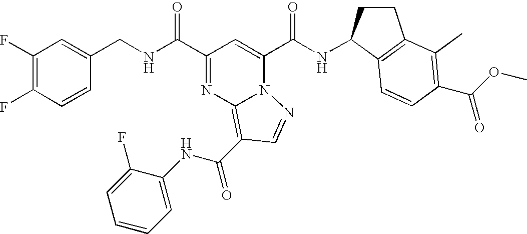 | 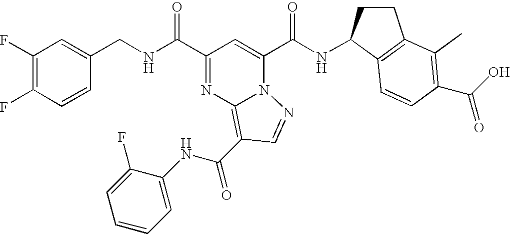 | 1. 40% 2. 641.3 |

### Example 26

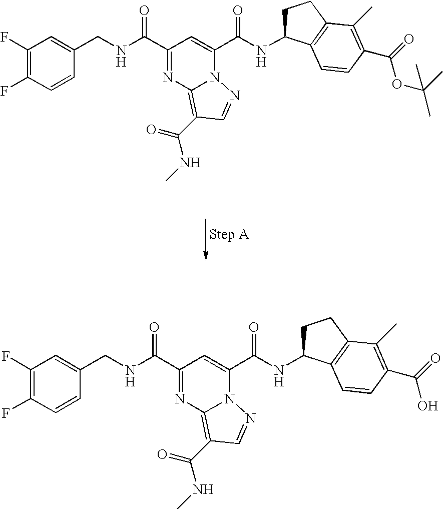

To a solution of (S)-1-{[5-(3,sdifluoro-benzylcarbarnoyl)-3-methylcarbarnoyl-pyrazolo[1,5-a]pyrimnidine-7-carbonyl]-amino}-4-methyl-indan-5-carboxylic acid tert-butyl ester (5.3 mg) in CH₂Cl₂ (0.06 mL) at 0° C. was added trifluoroacetic acid (0.06 mL) and this solution stirred for 1 h and was concentrated. The resulting solid was washed 3 times with Et₂O (0.2 mL) to give (S)-1-{[5-(3,4-difluoro-benzylcarbamoyl)-3-methylcarbamoyl-pyrazolo[1,5-a]pyrirnidine-7-carbonyl]-amnino}-4-methyl-indan-5-carboxylic acid (3.6 mg, 99%). [M−H⁻=561.4

### Example 27-47

Following a similar procedure as described in example 26 except using esters as indicated in the table below, the following compounds were prepared.

| | | | 1. Yield |
|---|---|---|---|
| Ex. # | ester | product | 2. [M − ]⁻ |
| 27 | 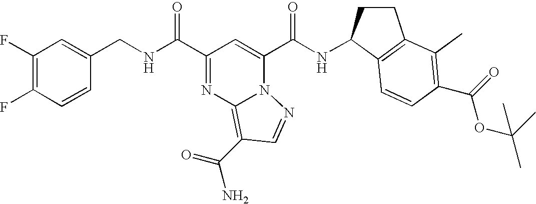 | 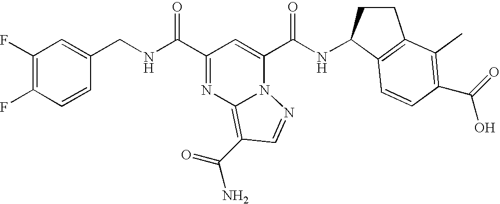 | 1. 40% 2. 547.4 |
| 28 | 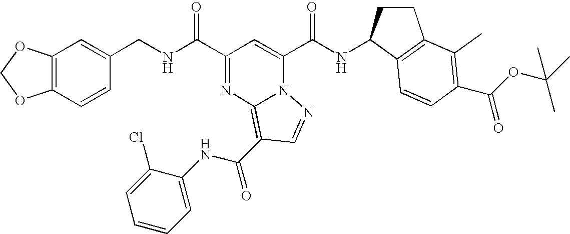 | 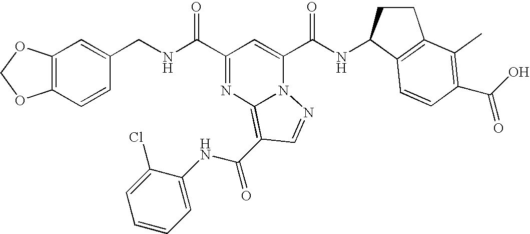 | 1. 94% 2. 665.3 |
| 29 | 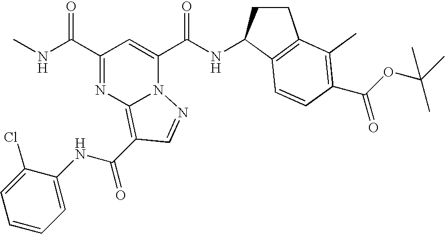 | 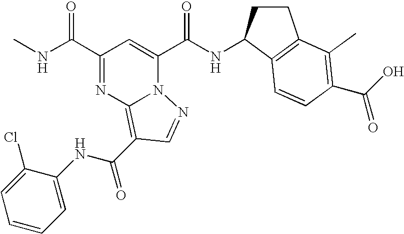 | 1. 100% 2. 601.5 |
| 30 | 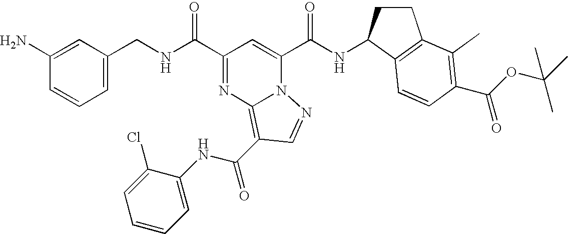 | 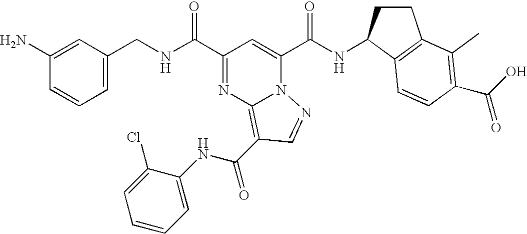 | 1. 100% 2. 636.5 |
| 31 | 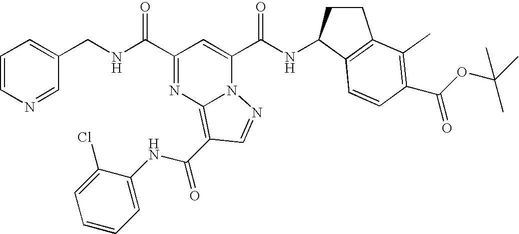 | 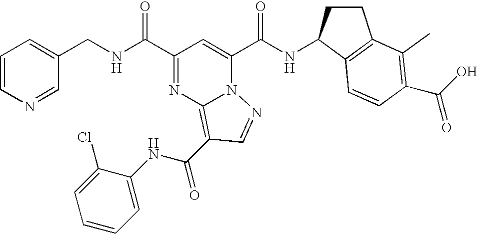 | 1. 100% 2. 622.5 |
| 32 | 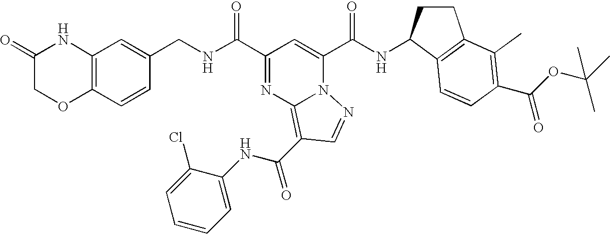 | 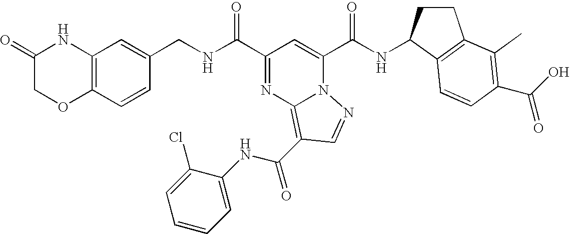 | 1. 100% 2. 692.3 |
| 33 | 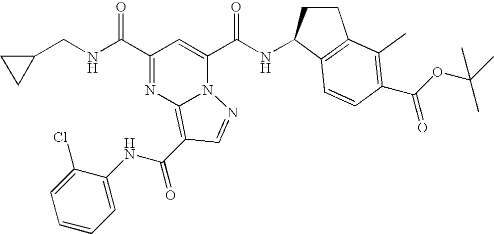 | 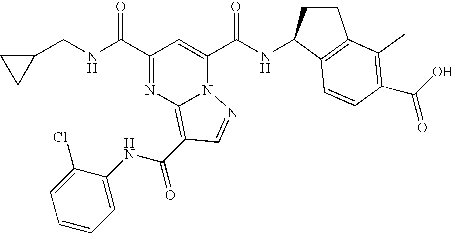 | 1. 100% 2. 585.4 |
| 34 | 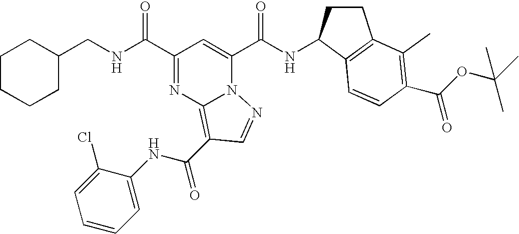 | 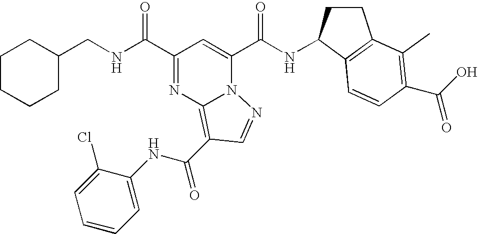 | 1. 94% 2. 627.3 |
| 35 | 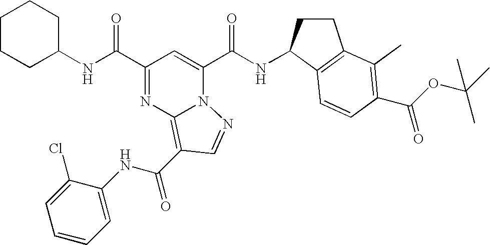 | 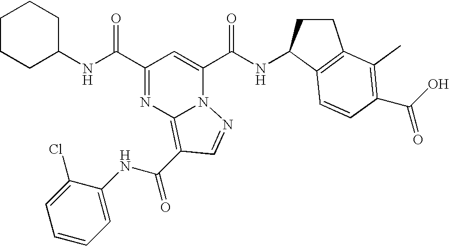 | 1. 100% 2. 613.4 |
| 36 | 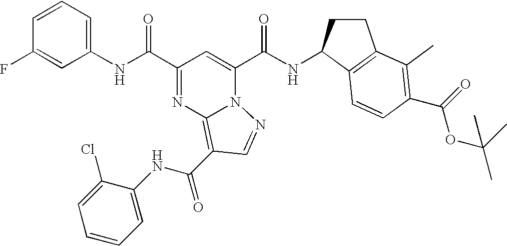 | 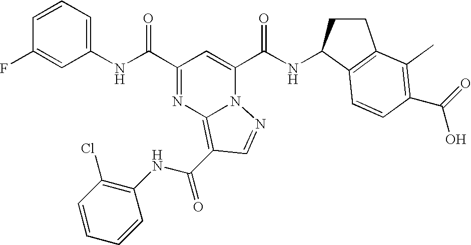 | 1. 100% 2. 625.5 |
| 37 | 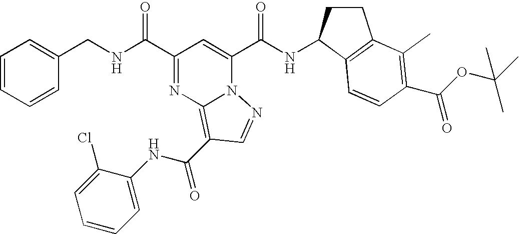 | 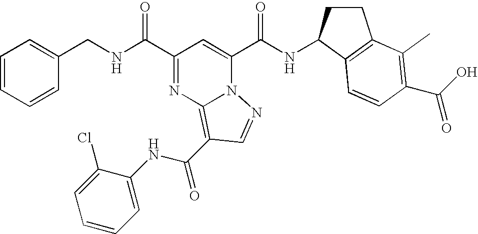 | 1. 86% 621.3 |
| 38 | 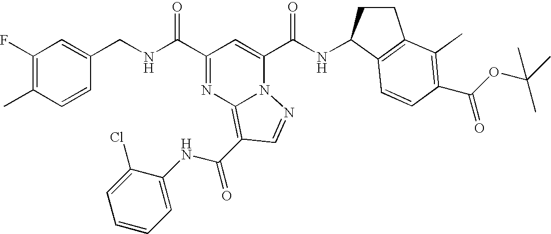 | 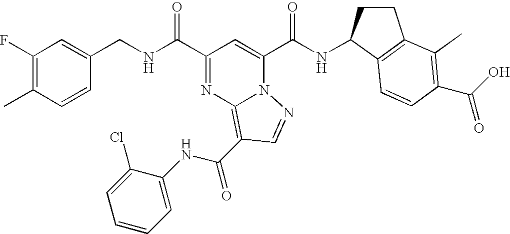 | 1. 79% 2. 653.3 |
| 39 | 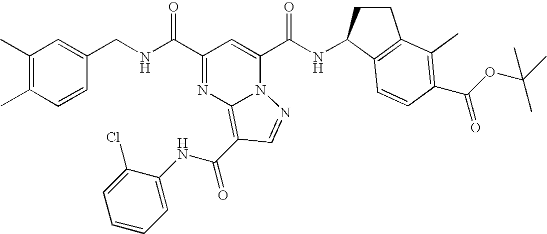 | 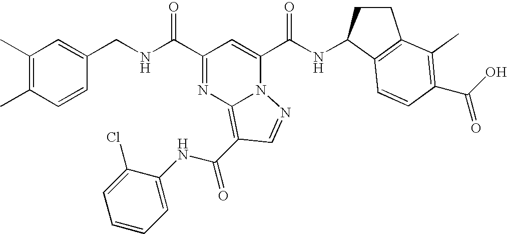 | 1. 68% 2. 649.3 |
| 40 | 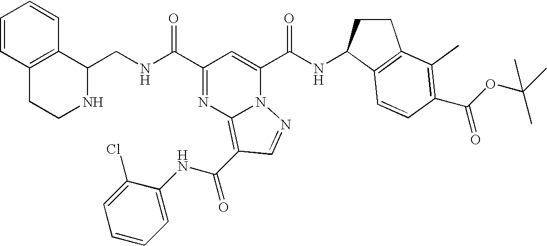 | 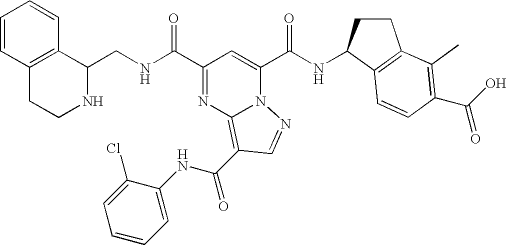 | 1. 100% 2. 676.5 |
| 41 | 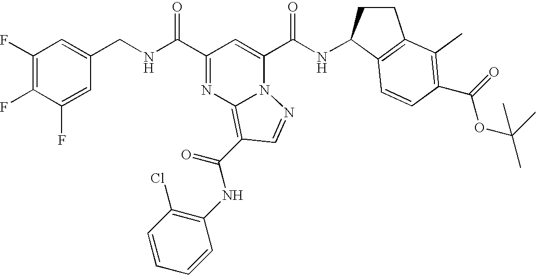 | 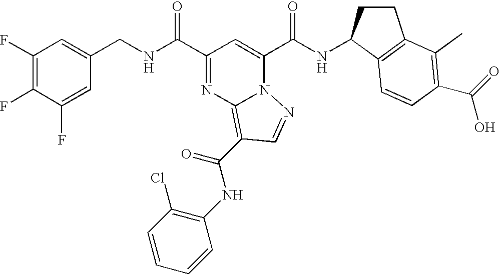 | 1. 50% 2. 675.4 |
| 42 | 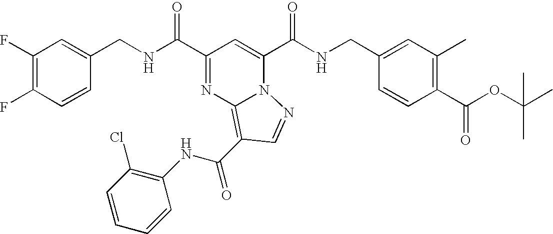 | 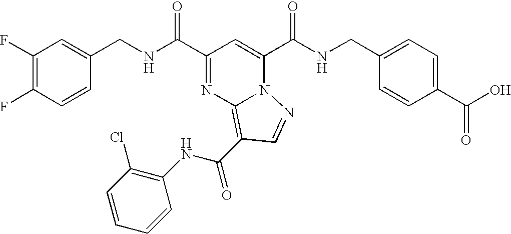 | 1. 99% 2. 631 |
| 43 | 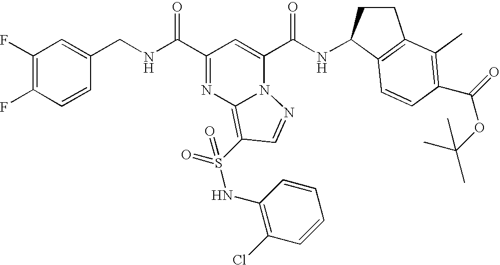 | 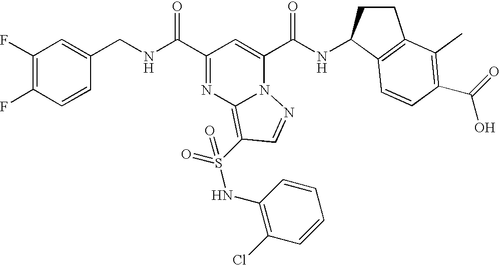 | 1. 25% 2. 693.4 |
| 44 | 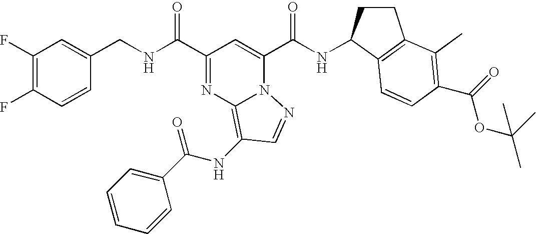 | 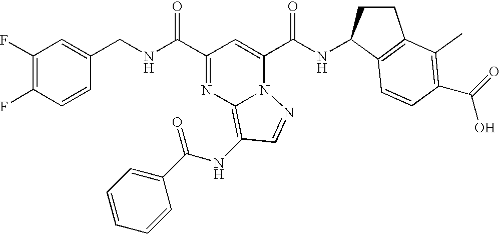 | 1. 98% 2. 623.5 |
| 45 | 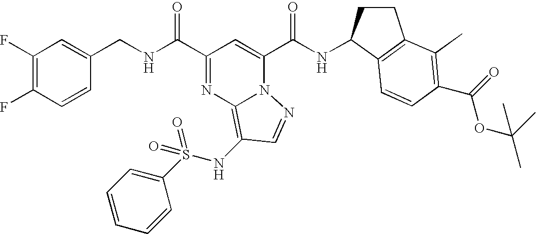 | 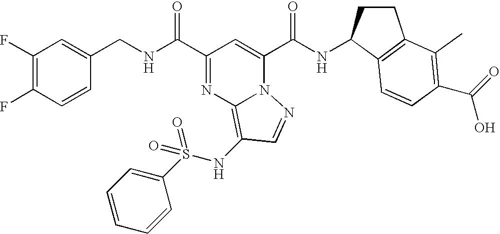 | 1. 63% 2. 659.5 |
| 46 | 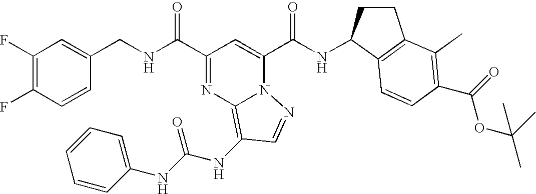 | 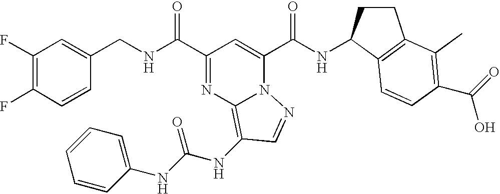 | 1. 94% 2. 638.5 |
| 47 | 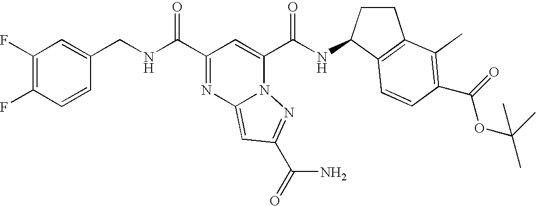 | 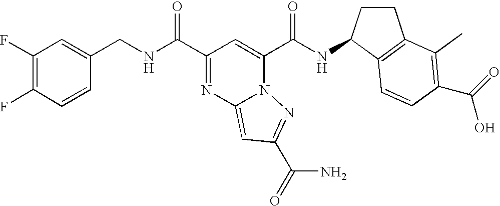 | 1. 99% 2. 547 |

### Example 48-50

Following a similar procedure as described in example 1 except using the amines and acids as indicated in the table below, the following compounds were prepared.

| | | | 1. Yield |
|---|---|---|---|
| Ex. # | Ester; amine | product | 2. [M − H]⁻ |
| 48 | 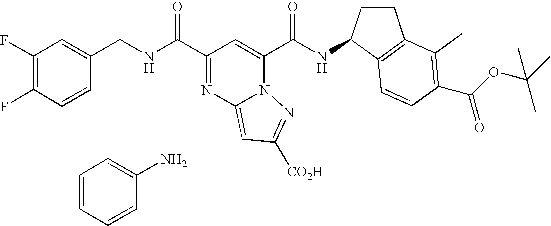 | 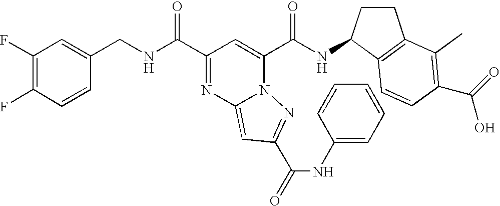 | 1. 99% 2. 623 |
| 49 | 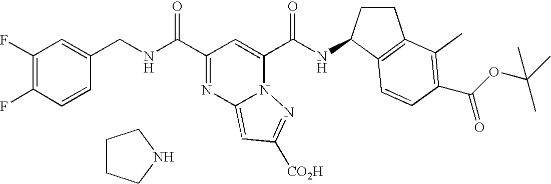 | 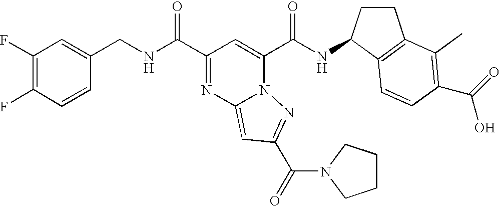 | 1. 99% 2. 601 |
| 50 | 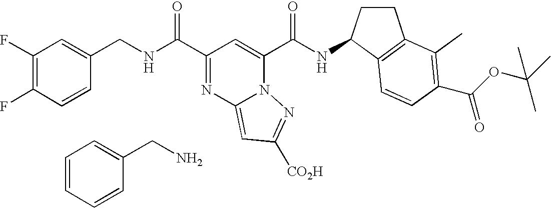 | 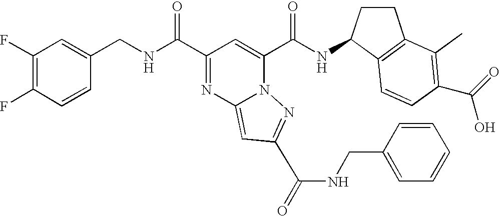 | 1. 99% 2. 637 |

### Example 51

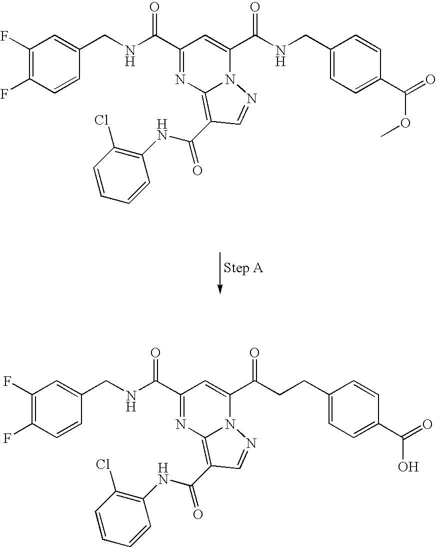

4-({[3-(2-Chloro-phenylcarbamoyl)-5-(3,4-difiuoro-benzylcarbamoyl)-pyrazolo[1,5-a]pyrimidine-7-carbonyl]-amino}-methyl)-benzoic acid methyl ester (6 mg) and trimethyltin hydroxide (6 mg) in dichloroethane (0.2 mL) was stirred at 90° C. for 18 h and concentrated. The crude product was purified by silica gel chromatography to give 4-({[3-(2-chloro-phenylcarbamoyl)-5-(3,4-difluoro-benzylcarbamoyl)-pyrazolo[1,5-a]pyrimidine-7-carbonyl]-amino}-methyl)-benzoic acid, (4 mg, 64%). [M−H]⁻=617.

### Example 52-53

If one followed a similar procedure as described in Preparative Example 51 except using the esters indicated in table below, the following compounds could be prepared.

| Ex. # | Ester | product |
|---|---|---|
| 52 | 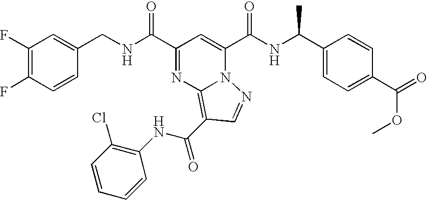 | 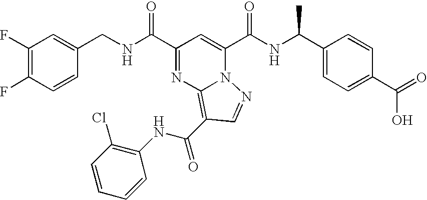 |
| 53 | 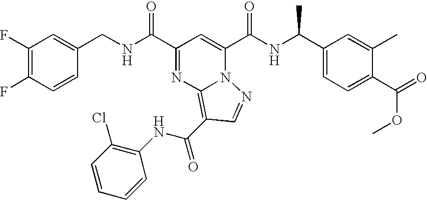 | 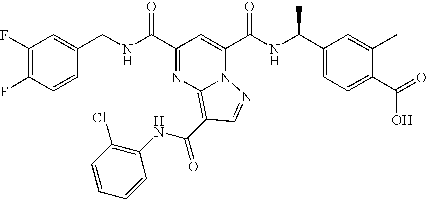 |

### Example 54

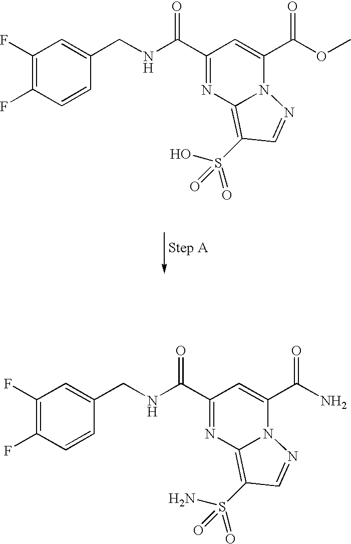 Step A

To a solution of 5-(3,4-difluoro-benzylcarbamoyl)-3-sulfo-pyrazolo[1,5-a]pyrimidine-7-carboxylic acid methyl ester (20 mg), and DMF (2 μL) in CH₂Cl₂ (0.4 mL) at 0° C. was added oxalyl chloride (20 μl). The solution was allowed to warm to 22° C. stirred for 3 h and concentrated. The resulting residue was brought up in CH₂Cl₂ (0.4 mL) and cooled to −78° C. To this cooled solution was condensed ammonia (1 mL). The cold bath was removed and he resulting solution was stirred and allowed to warm up to 22° C. over 18 h and absorbed onto silica and purified by silica gel chromatography to give 3-sulfamoyl-pyrazolo[1,5-a]pyrimidine-5,7-dicarboxylic acid 7-amide 5-(3,4-difluoro-benzylamide) (3.3 mg, 31%). [MH]⁺=411.0.

### Example 55-67

If one were to follow a similar procedure as described in Example 1, except using the amines and acids listed in the table below, the following compounds would be obtained.

| Ex. # | acid, amine | product |
|---|---|---|
| 55 | 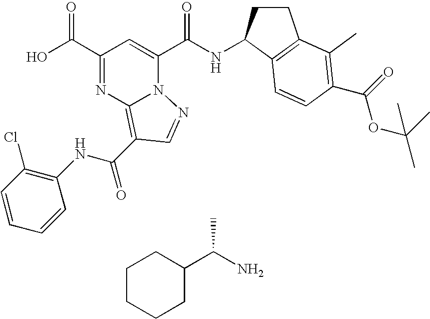 | 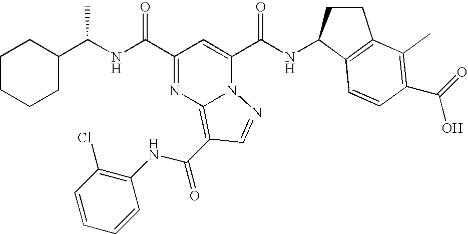 |
| 56 | 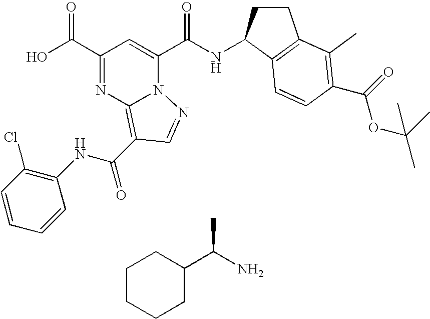 | 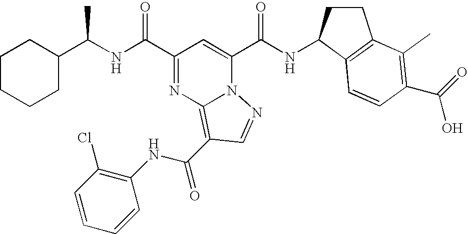 |
| 57 | 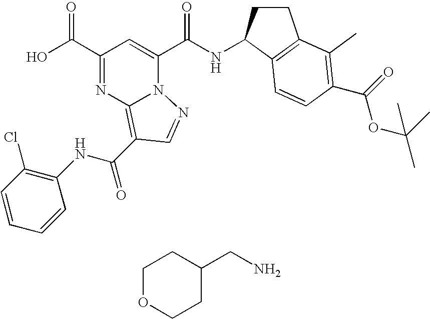 | 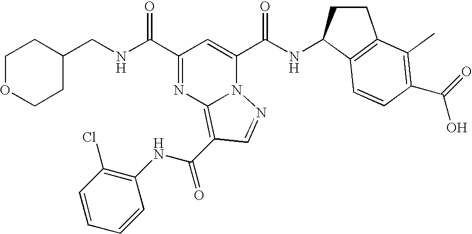 |
| 58 | 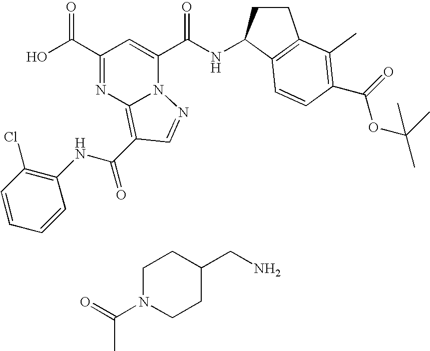 | 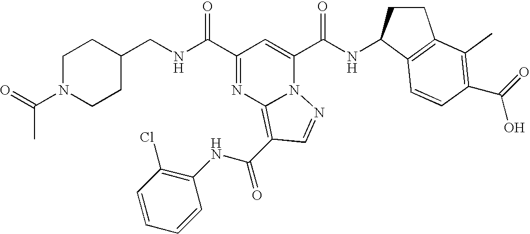 |
| 59 | 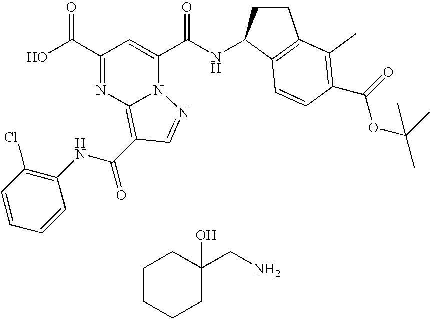 | 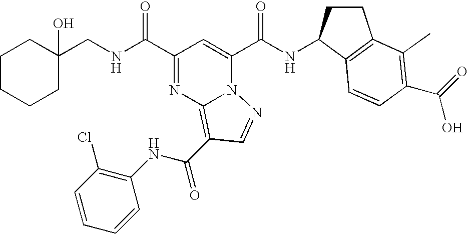 |
| 60 | 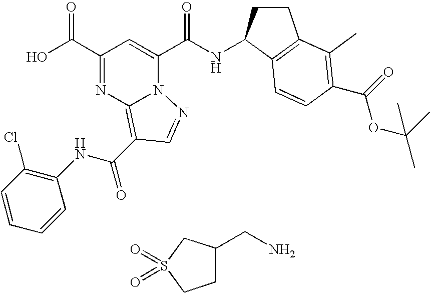 | 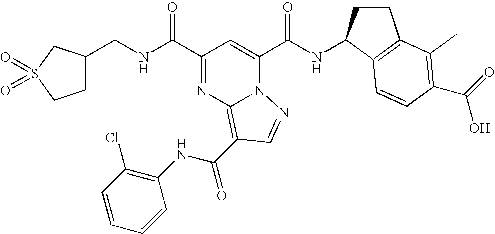 |
| 61 | 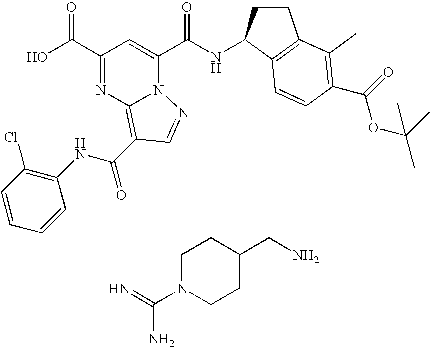 | 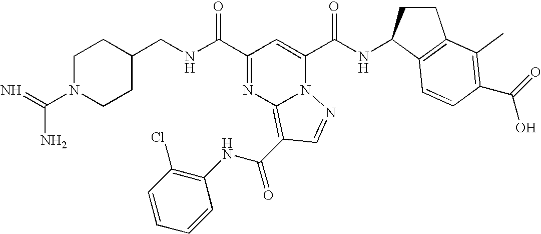 |
| 62 | 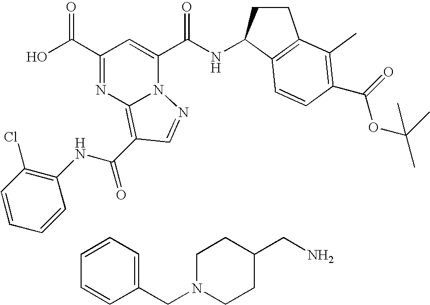 | 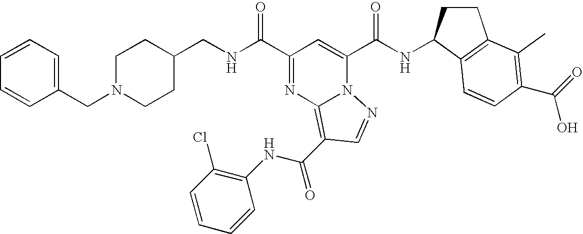 |
| 63 | 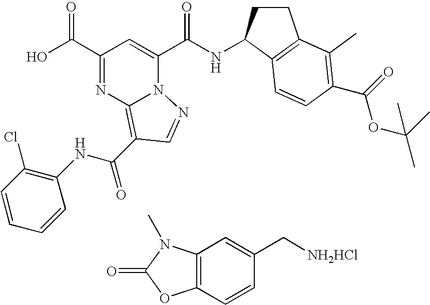 | 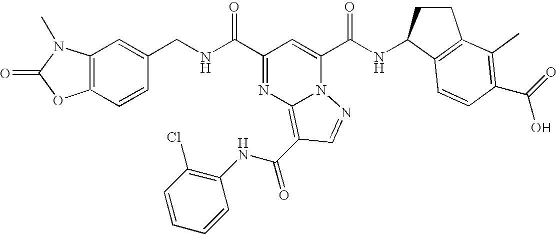 |
| 64 | 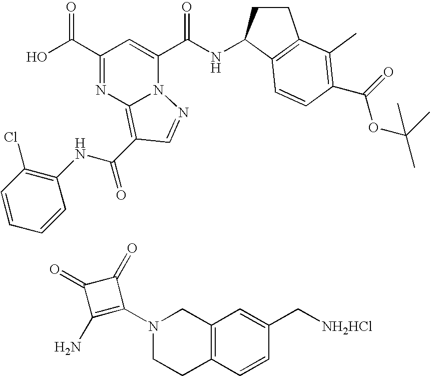 | 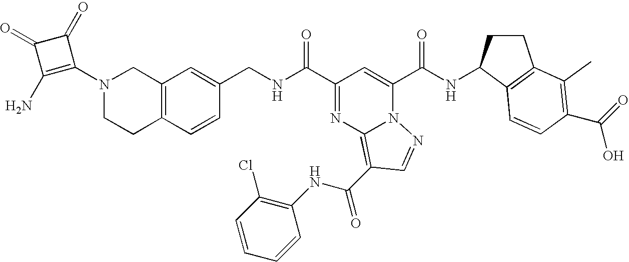 |
| 65 | 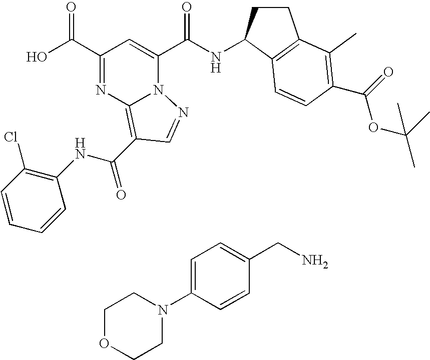 | 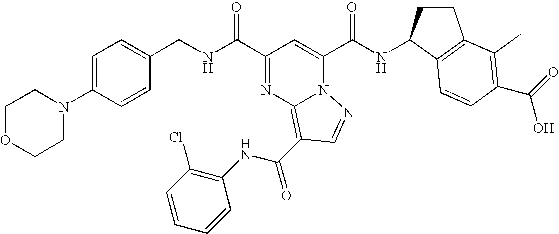 |
| 66 | 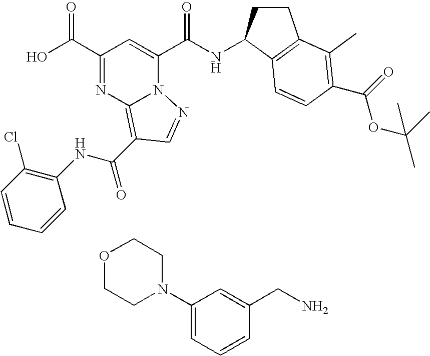 | 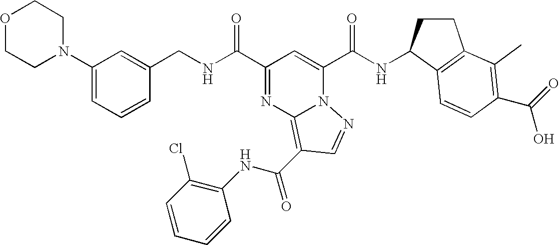 |
| 67 | 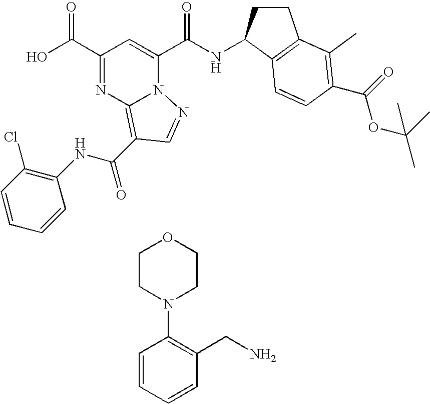 | 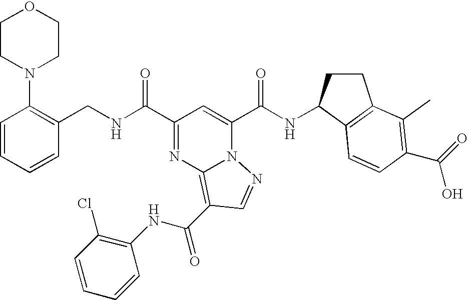 |

### Example 1700

### Assay for Determining Aggrecanase-1 (ADAMTS-4) Inhibition

The typical assay for aggrecanase-1 activity is carried out in assay buffer comprised of 50 M Tris, pH 7.5, 150 mM NaCl, 5 mM CaCl₂ and 0.05% Brij-35. Different concentrations of tested compounds are prepared in assay buffer in 50 μL aliquots. 10 μL of a 75 nM stock solution of aggrecanase-1 (Invitek) is added to the compound solution. The mixture of enzyme and compound in assay buffer is thoroughly mixed. The reaction is started by addition of 40 μL of a 250 nM stock solution of aggrecan-IGD substrate (Invitek) and incubation at 37° C. for exact 15 min. The reaction is stopped by addition of EDTA and the samples are analysed by using aggrecanase ELISA (Invitek, InviLISA, Cat. No. 30510111) according to the protocol of the supplier. Shortly: 100 μL of each proteolytic reaction are incubated in a pre-coated micro plate for 90 min at room temperature. After 3 times washing, antibody-peroxidase conjugate is added for 90 min at room temperature. After 5 times washing, the plate is incubated with TMB solution for 3 min at room temperature. The peroxidase reaction is stopped with sulfurous acid and the absorbance is red at 450 nm. The IC₅₀ values are calculated from the absorbance signal corresponding to residual aggrecanase activity.

### Example 1701

### Assay for Determining MMP-3 Inhibition

The typical assay for MMP-3 activity is carried out in assay buffer comprised of 50 mM MES, pH 6.0, 10 mM CaCl₂ and 0.05% Brij-35. Different concentrations of tested compounds are prepared in assay buffer in 50 μL aliquots. 10 μL of a 100 nM stock solution of the catalytic domain of MMP-3 enzyme (Biomol, Cat. No. SE-109) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of NFF-3 fluorescent substrate (Calbiochem, Cat. No. 480455). The time-dependent increase in fluorescence is measured at the 330 nm excitation and 390 nm emission by automatic plate multireader. The IC₅₀ values are calculated from the initial reaction rates

### Example 1702

### Assay for Determining MMP-8 Inhibition

The typical assay for MMP-8 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of activated MMP-8 enzyme (Calbiochem, Cat. No. 444229) is added to the compound solution. The mixture of enzyme and compound in assay buffer is thoroughly mixed and incubated for 10 min at room temperature. Upon the completion of incubation, the assay is started by addition of 40 μL of a 10 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### Example 1703

### Assay for Determining MMP-12 Inhibition

The typical assay for MMP-12 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of the catalytic domain of MMP-12 enzyme (Biomol, Cat. No. SE-138) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of OmniMMP fluorescent substrate (Biomol, Cat. No. P-126). The time-dependent increase in fluorescence is measured at the 320 nm excitation and 390 nm emission by automatic plate multireader at 37° C. The IC₅₀ values are calculated from the initial reaction rates.

### Example 1704

### Assay for Determining MMP-13 Inhibition

The typical assay for MMP-13 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 50 nM stock solution of catalytic domain of MMP-13 enzyme (produced by Alantos) is added to the compound solution. The mixture of enzyme and compound in assay buffer is thoroughly mixed and incubated for 10 min at room temperature. Upon the completion of incubation, the assay is started by addition of 40 μL of a 12.5 μM stock solution of MMP-13 fluorescent substrate (Calbiochem, Cat. No. 444235). The time-dependent increase in fluorescence is measured at the 320 nm excitation and 390 nm emission by automatic plate multireader. The IC₅₀ values are calculated from the initial reaction rates.

### Example 1705

### Assay for Determining ADAMTS-5 Inhibition

The typical assay for ADAMTS-5 activity is carried out in assay buffer comprised of 50 mM Tris, pH 7.5, 150 mM NaCl, 5 mM CaCl₂ and 0.05% Brij-35. Different concentrations of tested compounds are prepared in assay buffer in 50 μL aliquots. 10 μL of a 75 nM stock solution of ADAMTS-5 (Invitek) is added to the compound solution. The mixture of enzyme and compound in assay buffer is thoroughly mixed. The reaction is started by addition of 40 μL of a 250 nM stock solution of aggrecan-IGD substrate (Invitek) and incubation at 37° C. for exact 15 min. The reaction is stopped by addition of EDTA and the samples are analysed by using aggrecanase ELISA (Invitek, InviLISA, Cat. No. 30510111) according to the protocol of the supplier. Shortly: 100 μL of each proteolytic reaction are incubated in a pre-coated micro plate for 90 min at room temperature. After 3 times washing, antibody-peroxidase conjugate is added for 90 min at room temperature. After 5 times washing, the plate is incubated with TMB solution for 3 min at room temperature. The peroxidase reaction is stopped with sulfurous acid and the absorbance is red at 450 nm. The IC₅₀ values are calculated from the absorbance signal corresponding to residual aggrecanase activity.

What is claimed is:

1. A compound having Formula (I): 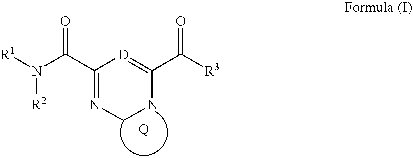
wherein:
R¹ is selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted one or more times by R⁹, or
wherein R¹ is optionally substituted by one R¹⁶ group and optionally substituted by one or more R⁹ groups;
R² is selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is NR²⁰R²¹;
R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰OR¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)-NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰OR¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from the group consisting of hydrogen, alkyl, C(O)NR¹⁰OR¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{NR}¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C⁰-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰—(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂-(C₀-C₆)-alkyl-aryl, S(O)₂(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from the group consisting of hydrogen, alkyl, alkenyl, alkynyl, cycloalkyl, cycloalkylalkyl, heterocycloalkyl, bicycloalkyl, heterobicycloalkyl, spiroalkyl, spiroheteroalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, bicycloalkyl, heterobicycloalkyl, spiroalkyl, spiroheteroalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 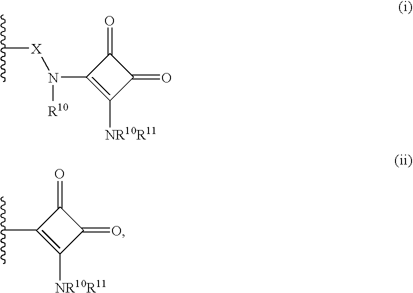
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from the group consisting of hydrogen and alkyl, wherein alkyl is optionally substituted one or more times;
R²¹ is a bicyclic or tricyclic fused ring system, wherein at least one ring is partially saturated, and
wherein R²¹ is optionally substituted one or more times, or
wherein R²¹ is optionally substituted by one or more R⁹ groups;
R²² is selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)OR¹⁰, C(O)NR¹⁰R¹¹, SO₂R¹⁰, SO₂NR¹⁰R¹¹ and fluoroalkyl, wherein alkyl, cycloalkyl, alkoxy, alkenyl, alkynyl, and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁸⁰ and R⁸¹ in each occurrence are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹, —C(R¹⁰OR¹¹)C(R¹⁰R¹¹)—, —CH₂—W¹— and 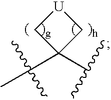
Q is a 5- or 6-membered ring selected from the group consisting of aryl and heteroaryl, wherein aryl and heteroaryl are optionally substituted one or more times with R⁴;
D is a member selected from the group consisting of CR²² and N;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2;
w is independently selected from 0-4;
x is selected from 0 to 2;
y is selected from 1 and 2; and
N-oxides, pharmaceutically acceptable salts, prodrugs, formulation, polymorphs, racemic mixtures and stereoisomers thereof.

2. The compound of claim 1, selected from the group consisting of: 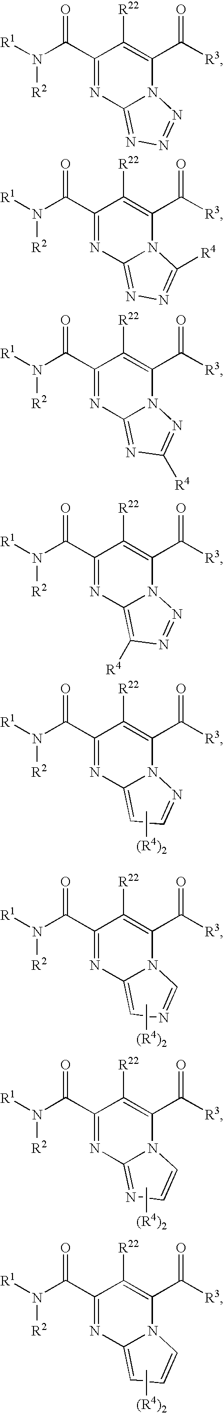 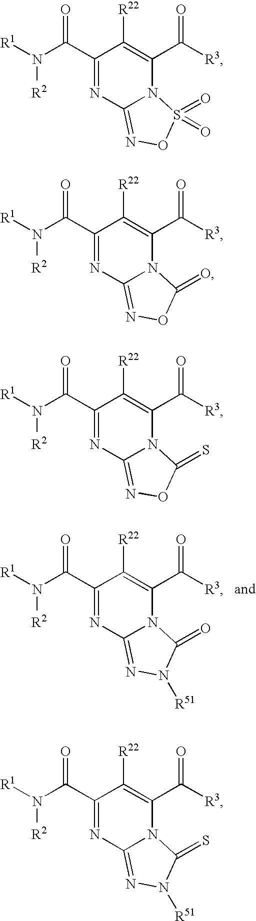
wherein:
R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

3. The compound of claim 2, selected from the group consisting of:

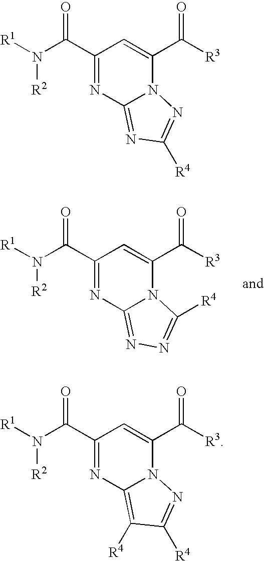

4. The compound of claim 3, selected from the group consisting of:

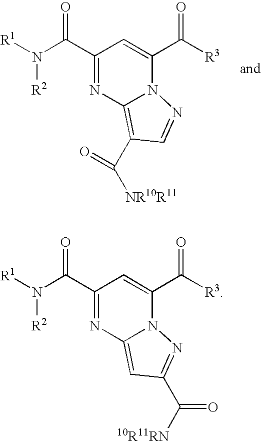

5. The compound of claim 4, selected from th e group consisting of: 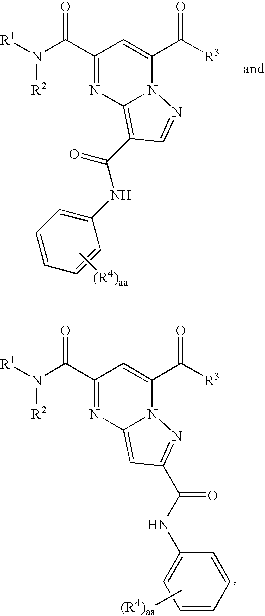
wherein:
aa is selected from 0-5.

6. The compound of claim 2, wherein R³ is selected from the group consisting of: 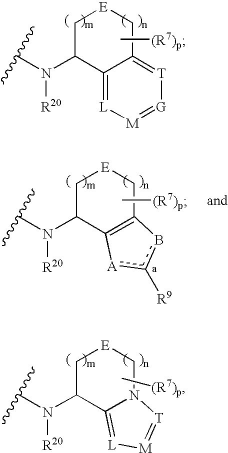
wherein:
R⁷ is independently selected from the group consisting of hydrogen, alkyl, cycloalkyl, halo, R⁴ and NR¹⁰R¹¹, or optionally two R⁷ groups together at the same carbon atom form ═O, ═S or ═NR¹⁰;
A and B are independently selected from the group consisting of CR⁹, CR⁹R¹⁰, NR¹⁰, N, O and S(O)ₓ;
G, L, M and T are independently selected from the group consisting of CR⁹ and N;
m and n are independently selected from 0-3, provided that:
(1) when E is present, m and n are not both 3;
(2) when E is —CH₂—W¹—, m and n are not 3; and
(3) when E is a bond, m and n are not 0; and
p is selected from 0-6;
wherein the dotted line represents a double bond between one of: carbon “a” and A, or carbon “a” and B.

7. The compound according to claim 6, wherein R³ is selected from the group consisting of: 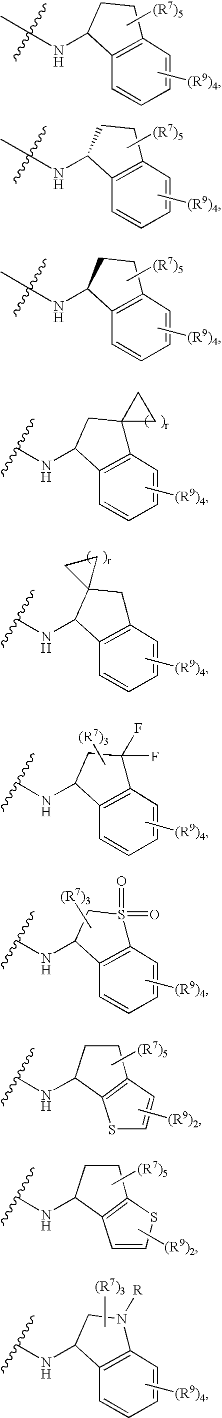 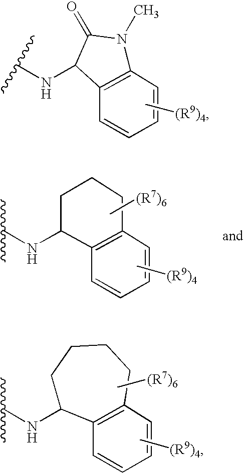
wherein:
R is selected from the group consisting of C(O)NR¹⁰R¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and
r is selected from 1-6.

8. The compound according to claim 6, wherein R³ is selected from the group consisting of:

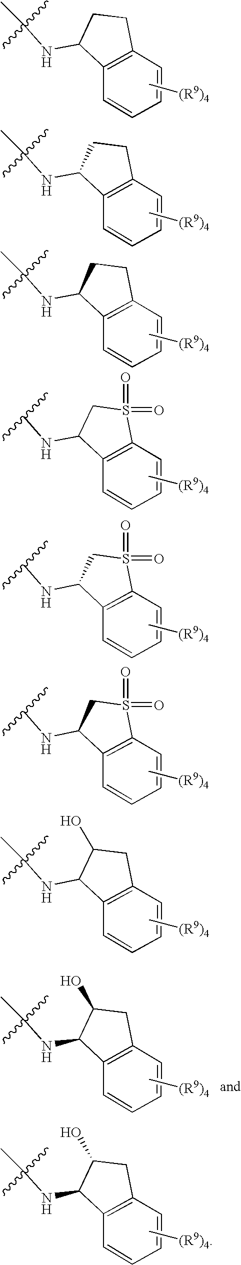

9. The compound according to claim 8, wherein R⁹ is selected from the group consisting of: 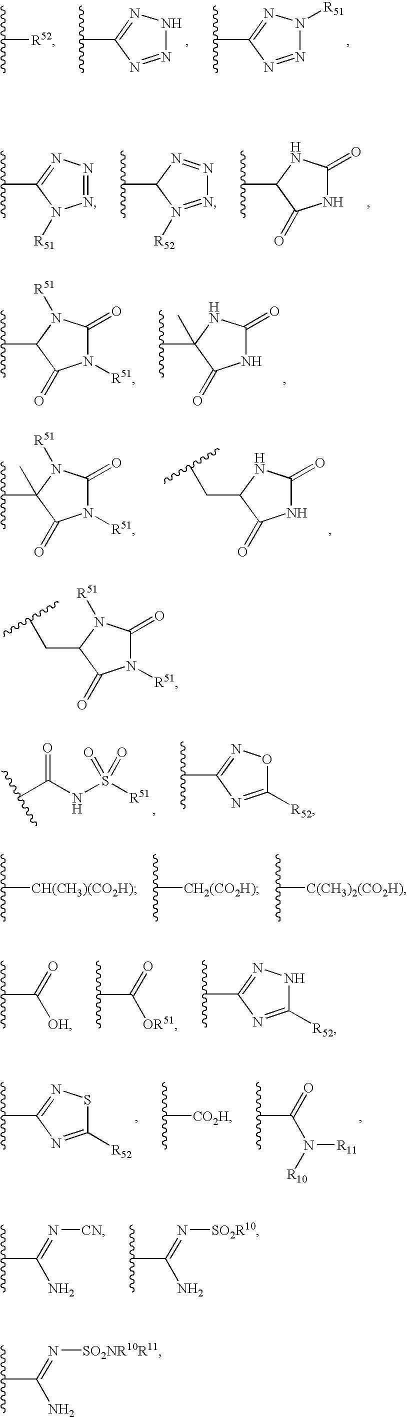 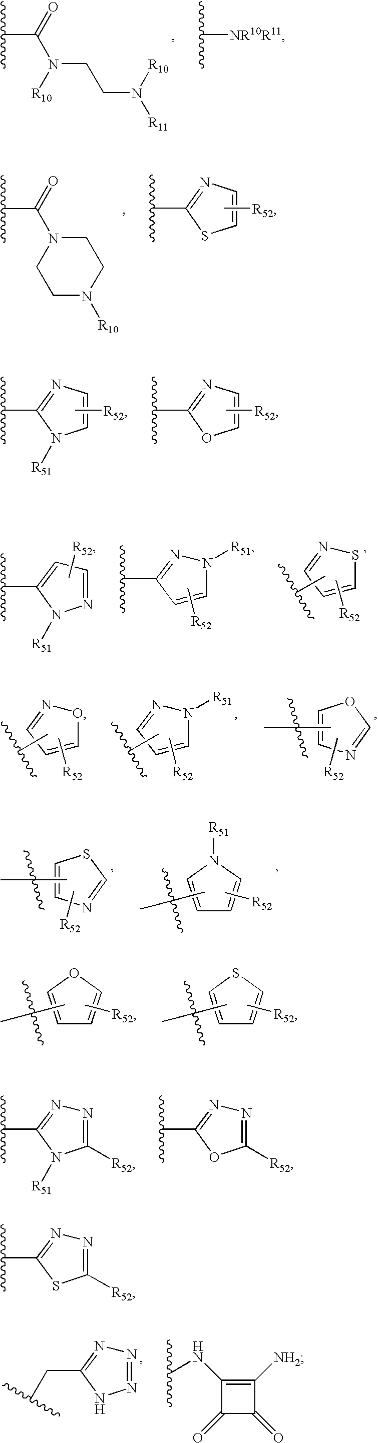 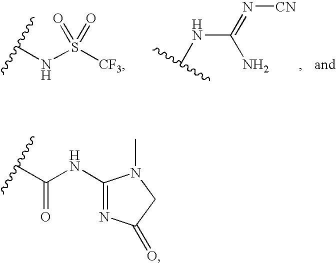
wherein:
R⁵² is selected from the group consisting of hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylakyl, cycloalkyalkyl, heteroarylalkyl, and halo are optionally substituted one or more times.

10. The compound according to claim 8, wherein R³ is

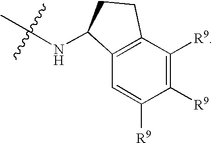

11. The compound according to claim 10, wherein R³ is selected from the group consisting of: 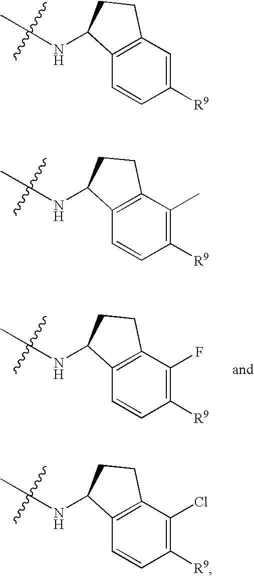
wherein:
R⁹ is selected from the group consisting of hydrogen, fluoro, halo, CN, alkyl, CO₂H,

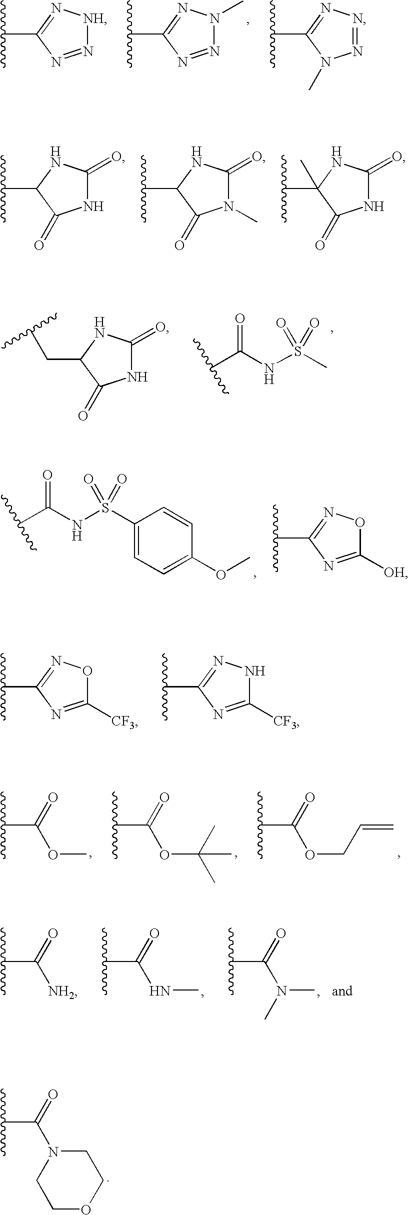

12. The compound according to claim 2, wherein R¹ is selected from the group consisting of: 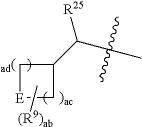
wherein:
ab is selected from the integer (2×ac)+(2×ad)+1;
ac is selected from 1-5;
ad is selected from 0-5;
optionally two R⁹ groups together at the same carbon atom form ═O, ═S or ═NR¹⁰; and
R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times.

13. The compound according to claim 12, wherein R¹ is selected from the group consisting of:

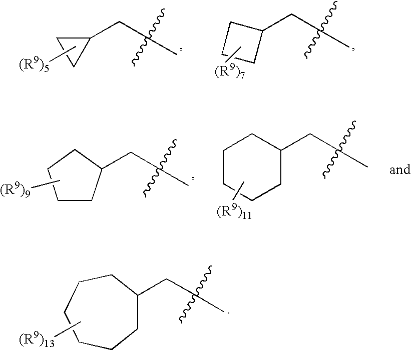

14. The compound according to claim 13, wherein R¹ is selected from the group consisting of:

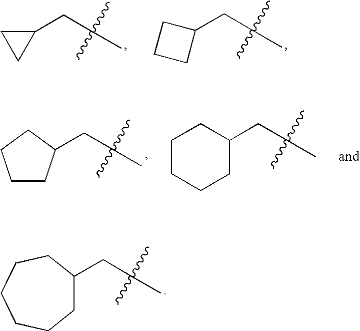

15. The compound according to claim 2, wherein R¹ is selected from the group consisting of: 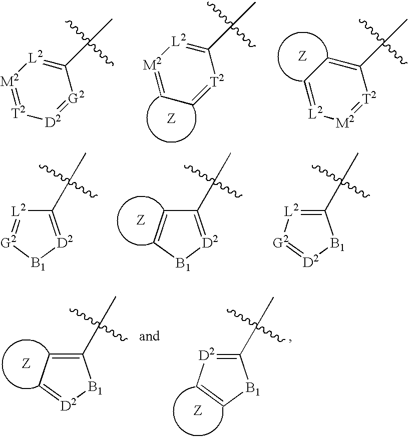
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
B₁ is selected from the group consisting of NR¹⁰, O and S(O)ₓ;
D², G², L², M² and T² are independently selected from the group consisting of CR⁹, CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

16. The compound according to claim 15, wherein R¹ is selected from the group consisting of: 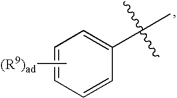
wherein:
ad is selected from 0-5.

17. The compound according to claim 16, wherein R¹ is selected from the group consisting of:

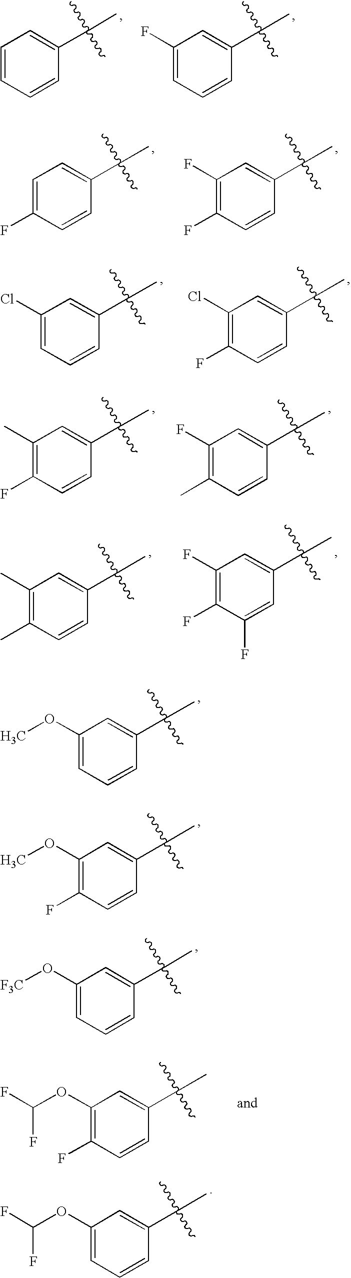

18. The compound according to claim 2, wherein R¹ is selected from the group consisting of: 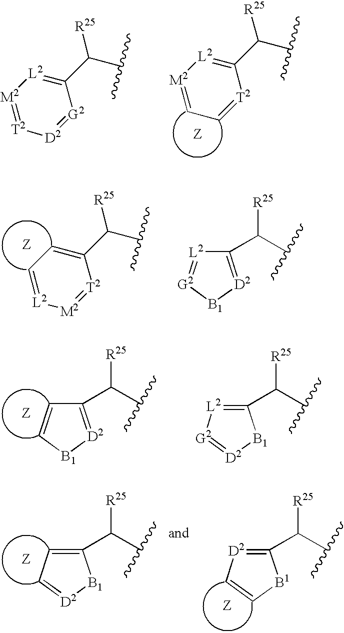
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
B₁ is selected from the group consisting of NR¹⁰, O and S(O)ₓ;
D², G², L², M² and T² are independently selected from the group consisting of CR⁹, CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

19. The compound according to claim 18, wherein R¹ is selected from the group consisting of:

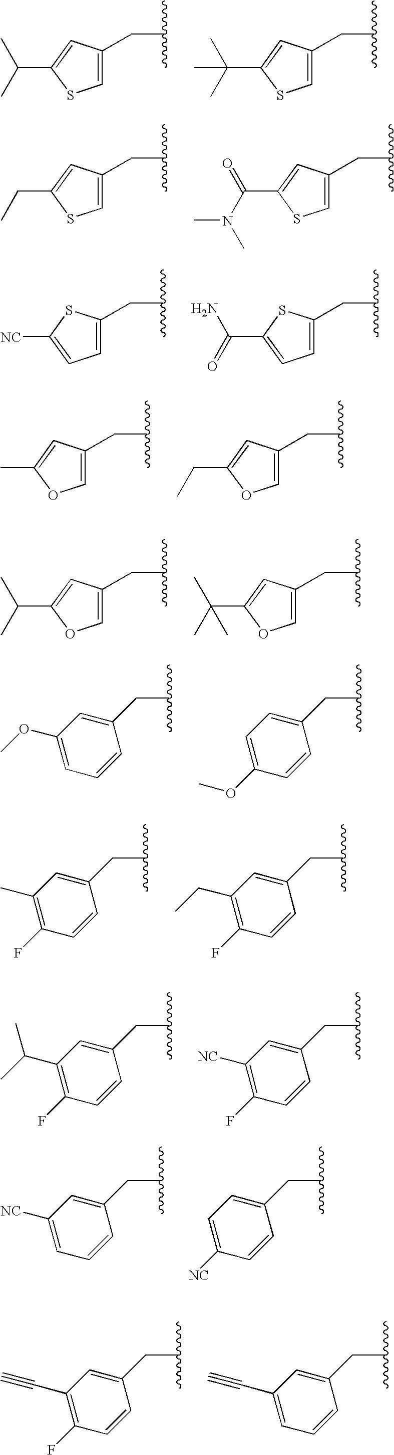

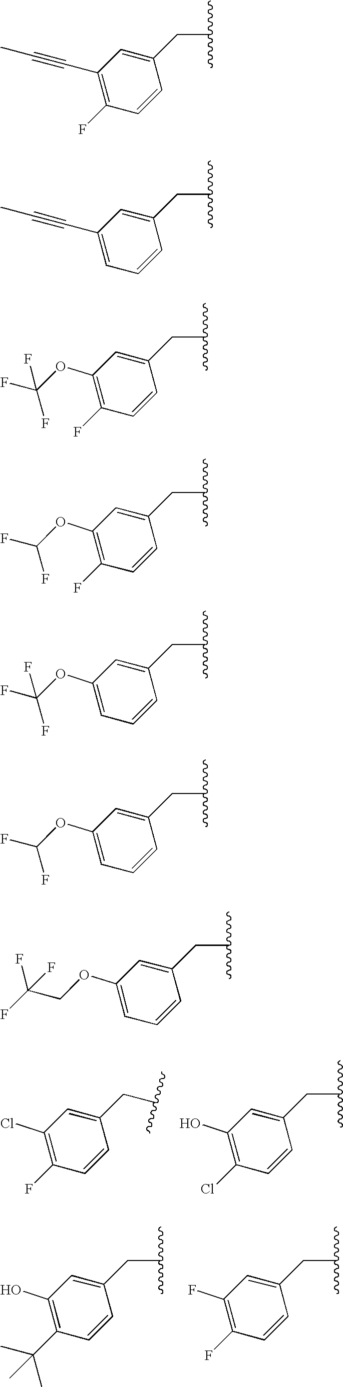

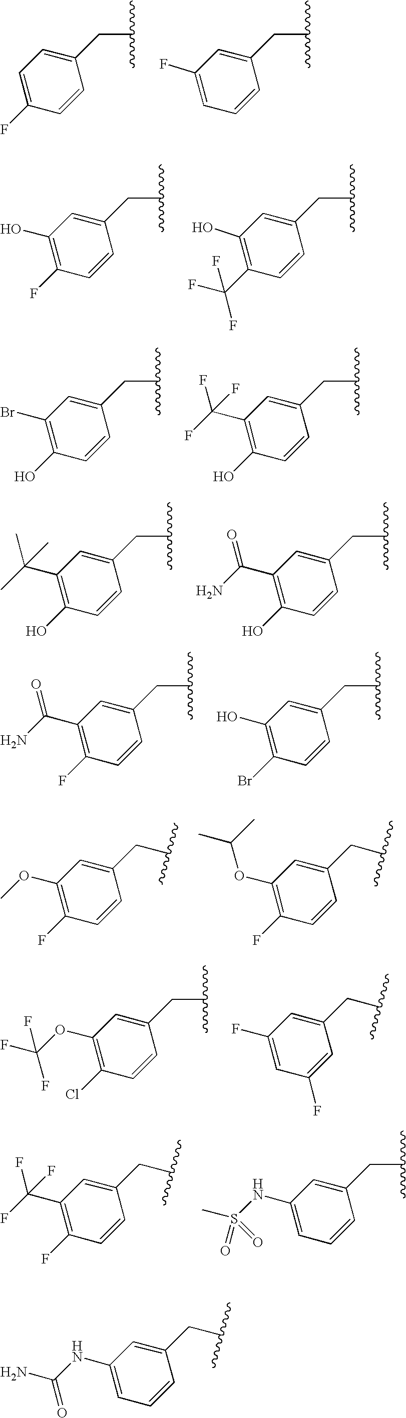

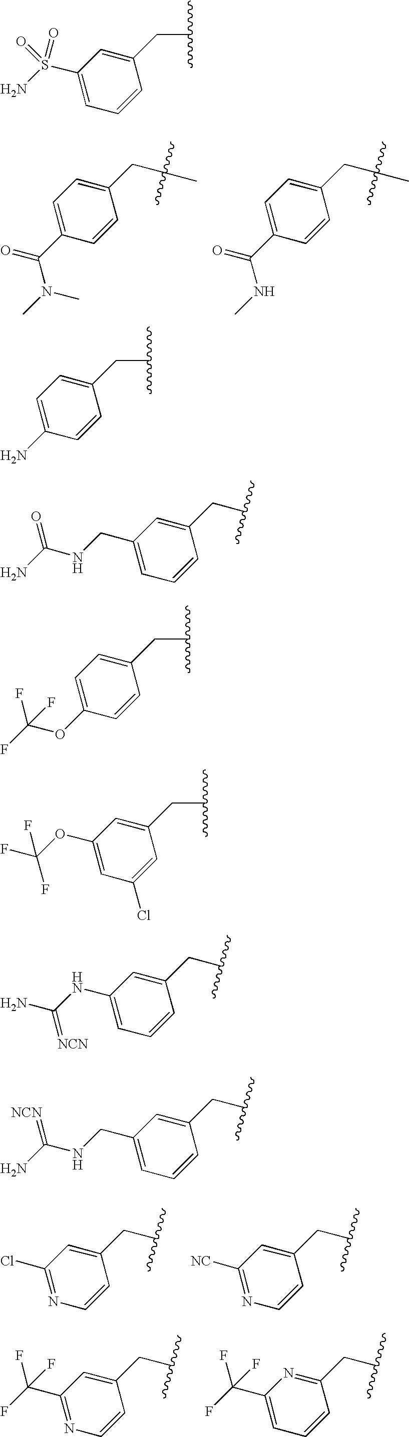

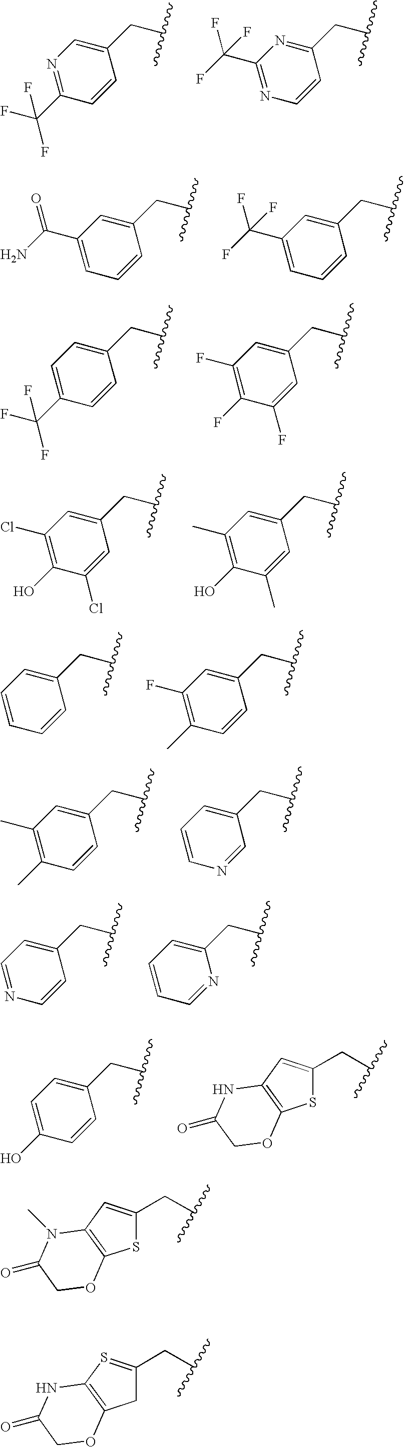

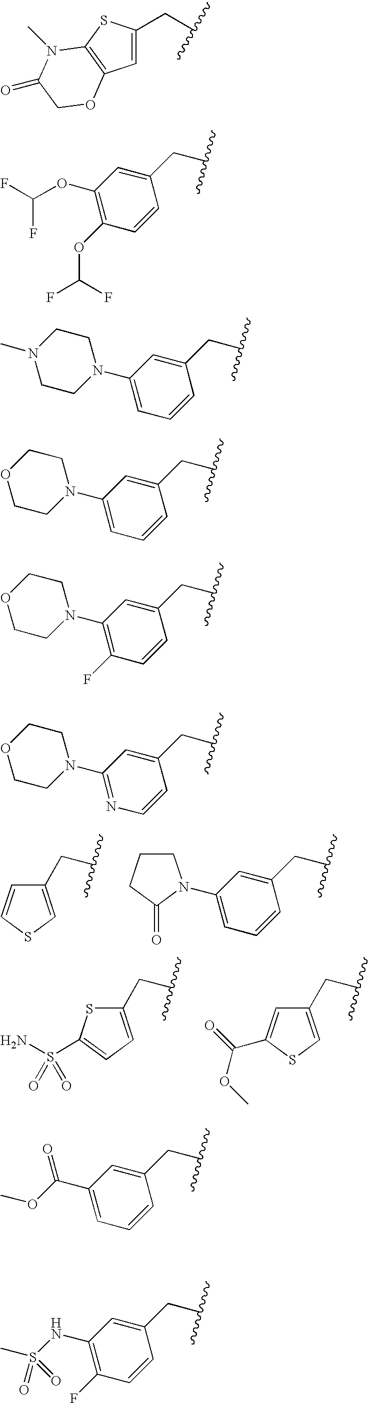

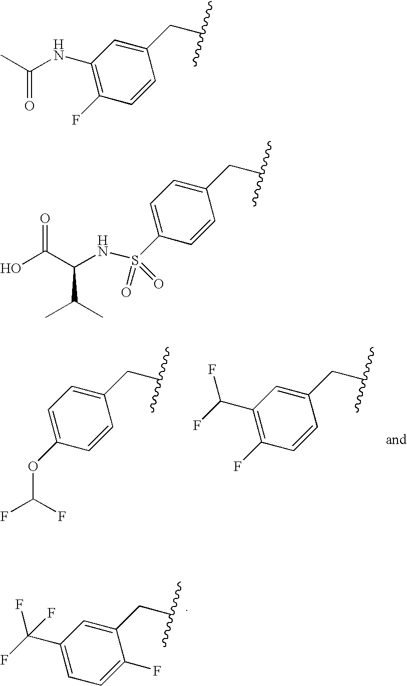

20. The compound of claim 2, wherein R¹ is selected from the group consisting of: 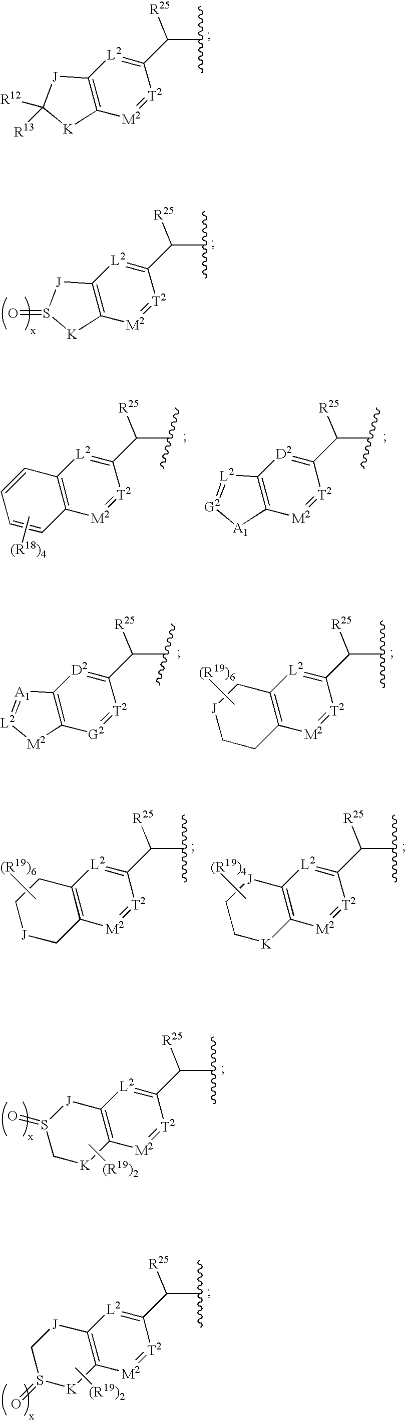 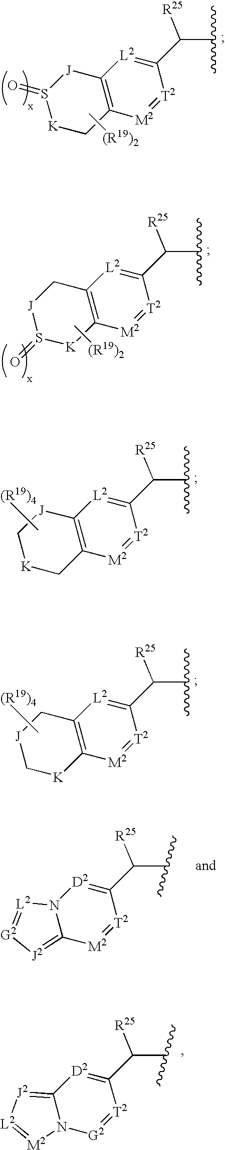
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O and S(O)ₓ; and
D², G², J², L², M² and T² are independently selected from the group consisting of CR⁹, CR¹⁸ and N.

21. The compound of claim 20, wherein R¹ is selected from the group consisting of:

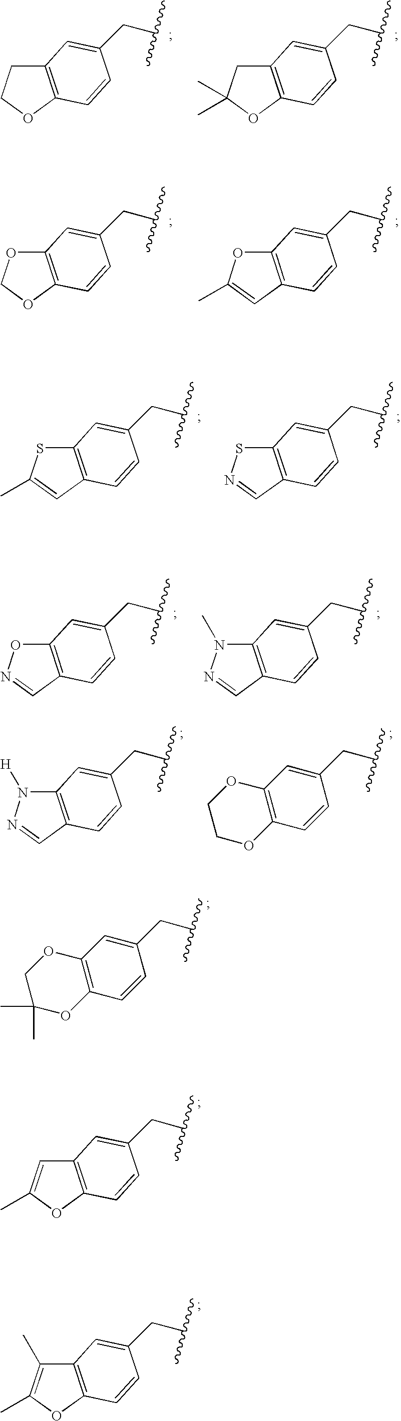

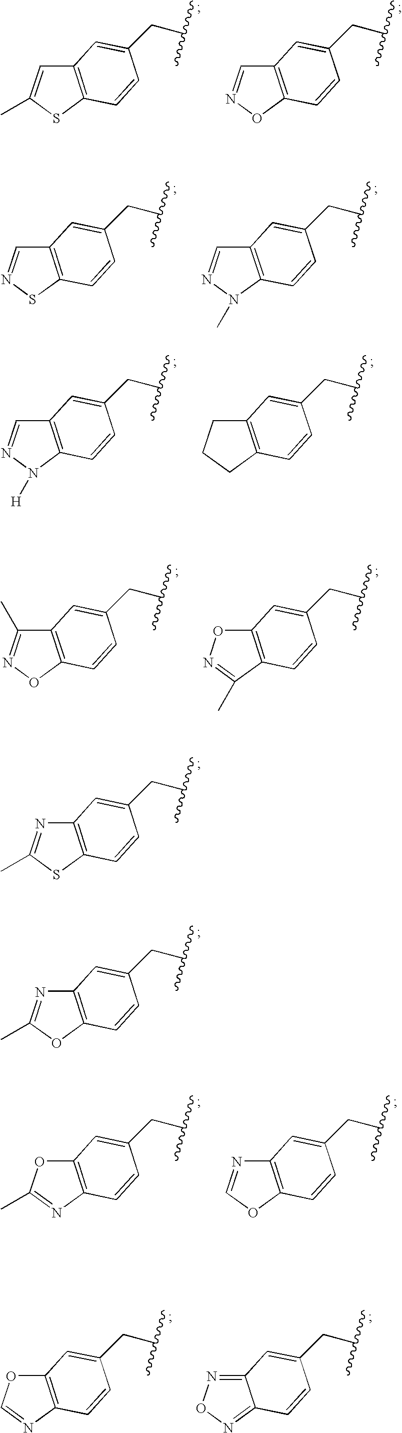

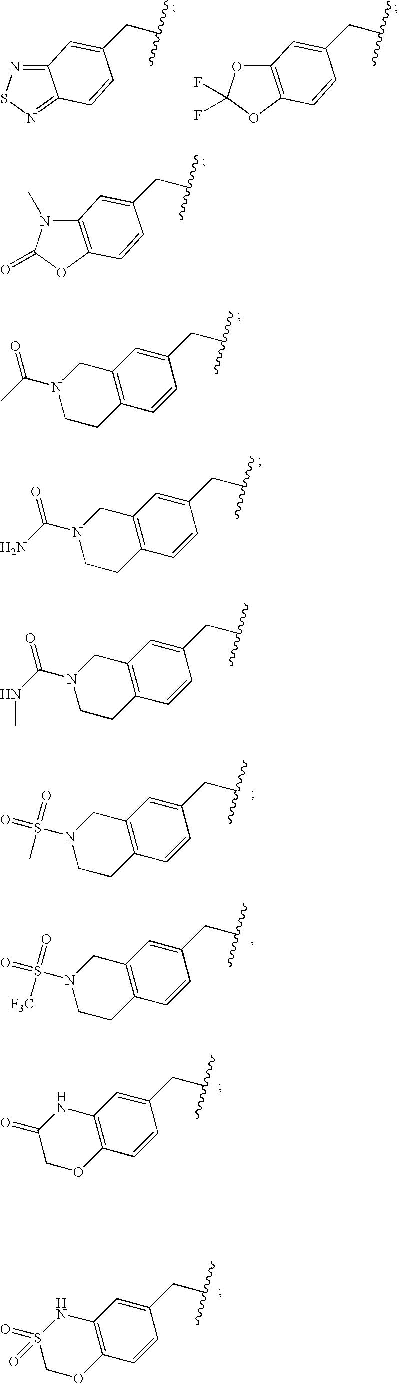

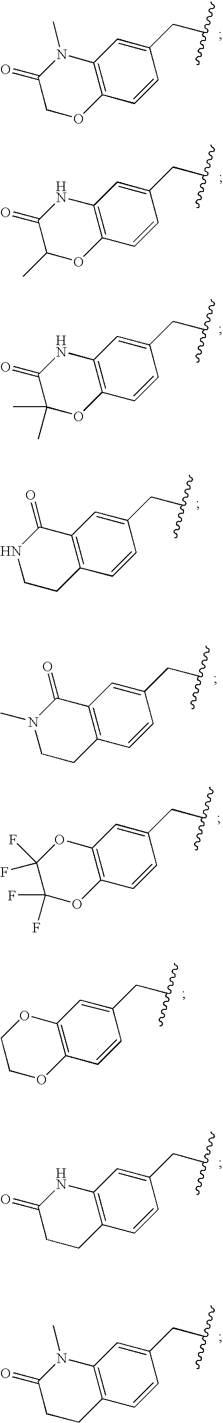

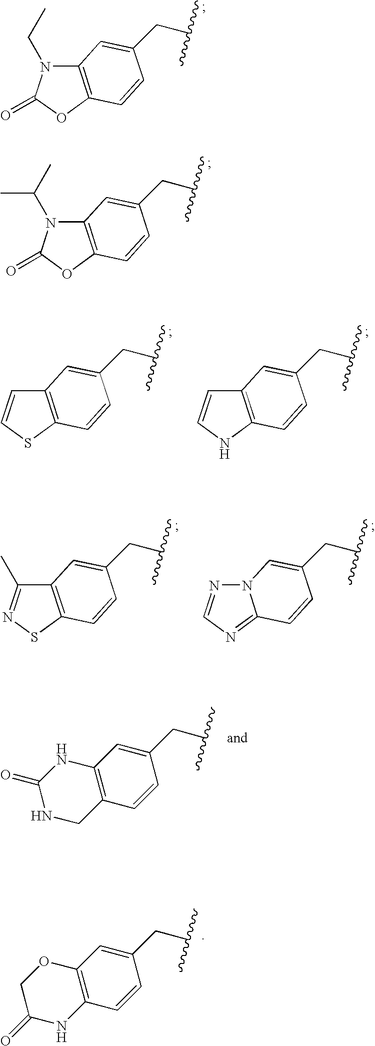

22. A compound having Formula (II): 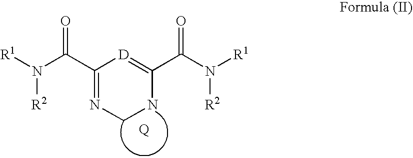
wherein:
R¹ in each occurrence is independently selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted one or more times by R⁹, or
wherein R¹ is optionally substituted by one R¹⁶ group and optionally substituted by one or more R⁹ groups;
R² in each occurrence is independently selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C⁶)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-S(O)_{y}NR¹⁰R¹¹, (C₀-C⁶)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹⁰, O—(C₀-C₆)-alkyl-aryl and O—(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆) -alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alky -OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C6)-alkyl-C(O)NR¹⁰R¹¹, (C₀-C₆)-alkyl-C(O)NR¹⁰SO₂R¹¹, C(O)NR¹⁰-(C₀-C₆)-alkyl-heteroaryl, C(O)NR¹⁰—(C₀-C₆)-alkyl-aryl, S(O)₂NR¹⁰—(C₀-C₆)-alkyl-aryl, S(O)₂NR¹⁰—(C₀-C₆)-alyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆)-alkyl-C(O)OR¹⁰, S(O)ₓ—(C₀-C6)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from the group consisting of hydrogen, alkyl, alkenyl, alkynyl, cycloalkyl, cycloalkylalkyl, heterocycloalkyl, bicycloalkyl, heterobicycloalkyl, spiroalkyl, spiroheteroalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, bicycloalkyl, heterobicycloalkyl, spiroalkyl, spiroheteroalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 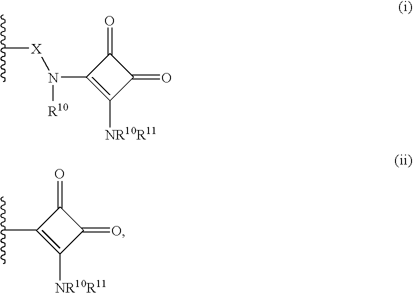
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²² is selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)OR¹⁰ , C(O)NR¹⁰R¹¹, SO₂R¹⁰, SO₂NR¹⁰R¹¹and fluoroalkyl, wherein alkyl, cycloalkyl, alkoxy, alkenyl, alkynyl, and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁸⁰ and R⁸¹ in each occurrence are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ , —NH, and —N(alkyl) and which is optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰)S(═O)₂, S(═O)₂N(R¹⁰), C═N—OR¹¹—C(R¹⁰R¹¹)C(R¹⁰R¹¹)—, —CH₂—W¹— and 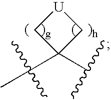
Q is a 5- or 6-membered ring selected from the group consisting of aryl and heteroaryl, wherein aryl and heteroaryl are optionally substituted one or more times with R⁴;
D is a member selected from the group consisting of CR²² and N;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2;
w is independently selected from 0-4;
x is selected from 0 to 2;
y is selected from 1 and 2; and
N-oxides, pharmaceutically acceptable salts, prodrugs, formulation, polymorphs, racemic mixtures and stereoisomers thereof.

23. The compound of claim 22, selected from the group consisting of: 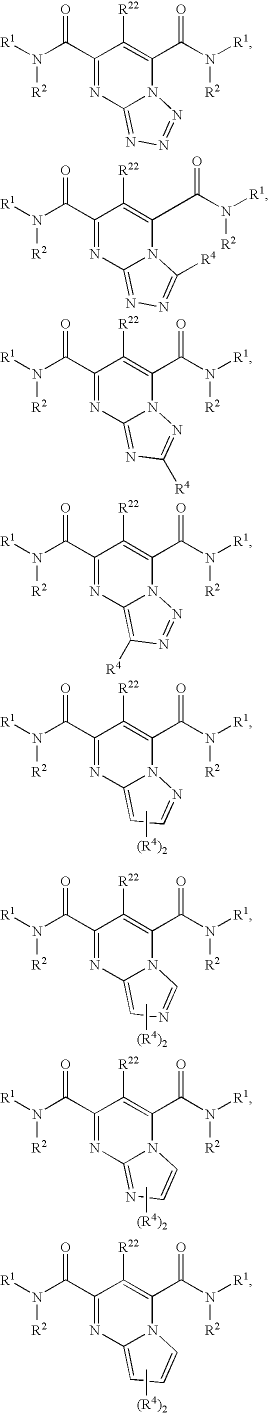 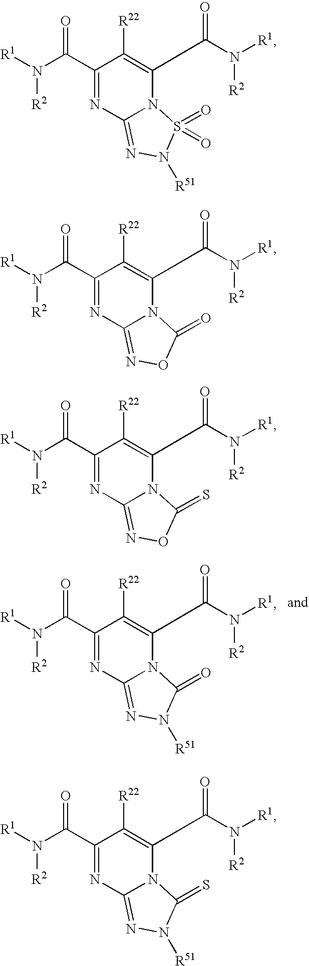
wherein:
R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

24. The compound of claim 23, selected from the group consisting of:

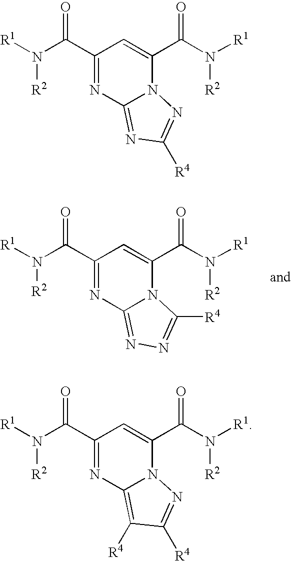

25. The compound of claim 24, selected from the group consisting of:

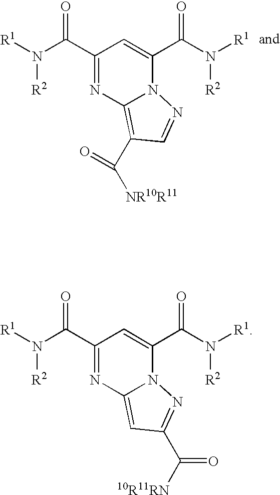

26. The compound of claim 25, selected from the group consisting of: 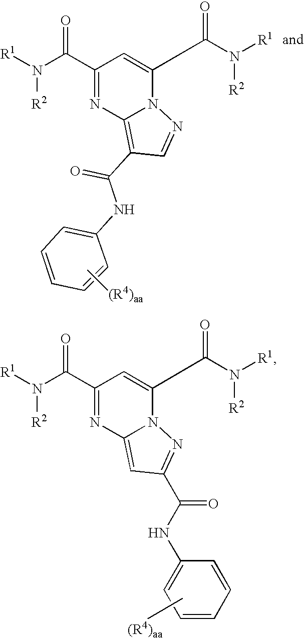
wherein:
aa is selected from 0-5.

27. The compound according to claim 23, wherein one R¹ is selected from the group consisting of: 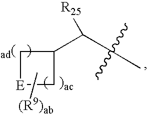
wherein:
ab is selected from the integer (2×ac)+(2×ad)+1;
ac is selected from 1-5;
ad is selected from 0-5;
optionally two R⁹ groups together at the same carbon atom form ═O, ═S or ═NR¹⁰; and
R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times.

28. The compound according to claim 27, wherein one R¹ is selected from the group consisting of:

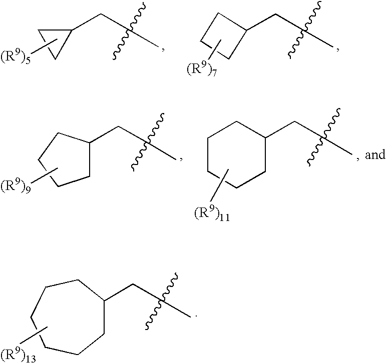

29. The compound according to claim 28, wherein one R¹ is selected from the group consisting of:

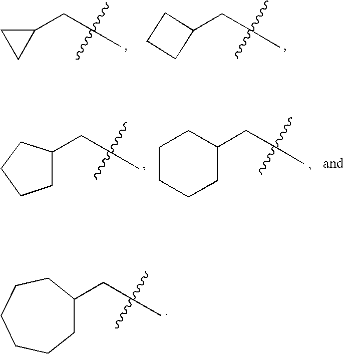

30. The compound according to claim 23, wherein one R¹ is selected from the group consisting of: 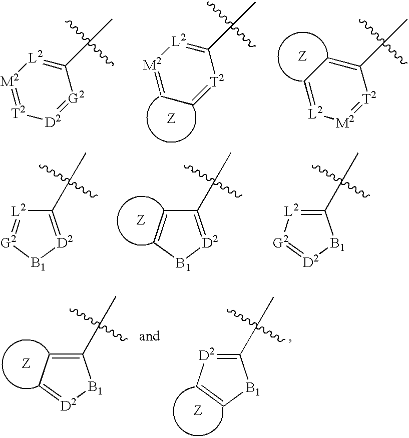
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
B₁ is selected from the group consisting of NR¹⁰, O and S(O)ₓ;
D², G², L², M² and T² are independently selected from the group consisting of CR⁹, CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

31. The compound according to claim 30, wherein R¹ is selected from the group consisting of: 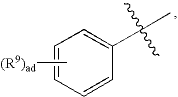
wherein:
ad is selected from 0-5.

32. The compound according to claim 31, wherein R¹ is selected from the group consisting of:

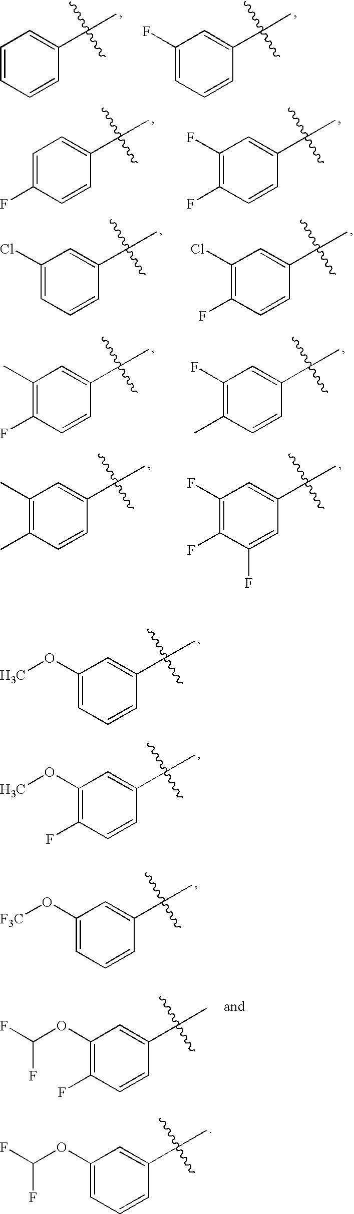

33. The compound of claim 23, wherein at least one R¹ is selected from the group consisting of: 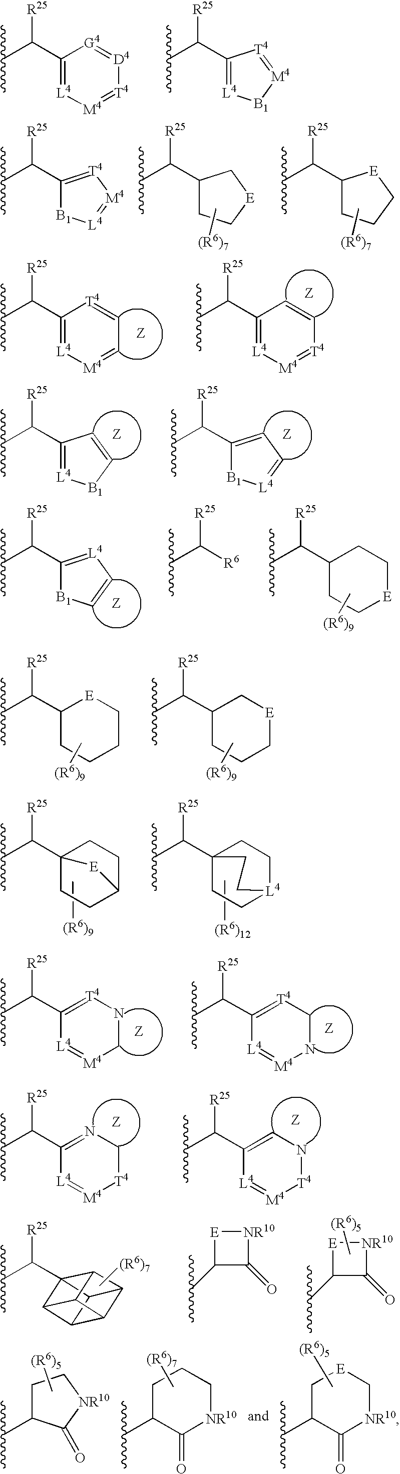
wherein:
R⁶ is independently selected from the group consisting of R⁹, alkenyl, alkynyl, cycloalkyl, heterocycloalkyl, bicycloalkyl, heterobicycloalkyl, spiroalkyl, spiroheteroalkyl, aryl, heteroaryl, C(O)OR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C6)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl- NR¹⁰C(═N—NO₂)NR¹⁰R¹¹, (C₀-C₆)-alkyl-C(═N—NO₂)NR¹⁰R¹¹, (C₀-C₆)-alkyl-C(O))OR¹⁰, (C₀-C6)-alkyl-C(O)NR¹⁰R¹¹, (C₀-C₆)-alkyl-C(O)NR¹⁰SO₂R¹¹, C(O)NR¹⁰-(C₀-C₆)-alkyl-heteroaryl, C(O)NR¹⁰—(C₀-C6)-alkyl-aryl, S(O)₂NR¹⁰—(C₀-C₆)-alkyl-aryl, S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆) -alkyl-heteroaryl, (C₀-C₆)-alkyl-C(O)—NR¹¹—CN, O—(C₀-C₆)-alkyl-C(O)NR¹⁰R¹¹, S(O)ₓ—(C₀-C₆) -alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰—C(O)OR¹⁰, (C₀-C₆)-alkyl-NR¹⁰—C(O)—NR¹⁰R¹¹, (C₀-C₆)-alkyl-NR¹⁰—S(O)_{y}NR¹⁰R¹¹, (C₀-C₆)-alkyl-NR¹⁰—S(O)_{y}R¹¹, O—(C₀-C₆)-alkyl-aryl and O—(C₀-C₆)-alkyl-heteroaryl, wherein each R⁶ group is optionally substituted by one or more R¹⁴ groups;
R⁹ is independently selected from the group consisting of hydrogen, alkyl, halo, CHF₂, CF₃, OR¹⁰, NR¹⁰R¹¹, NO₂, and CN, wherein alkyl is optionally substituted one or more times;
R²⁵ is selected from the group consisting of hydrogen, alkyl, cycloalkyl, CO₂R¹⁰, C(O)NR¹⁰R¹¹ and haloalkyl, wherein alkyl, cycloalkyl, and haloalkyl are optionally substituted one or more times;
R³⁰ is selected from the group consisting of alkyl and (C₀-C₆)-alkyl-aryl, wherein alkyl and aryl are optionally substituted;
B₁ is selected from the group consisting of NR¹⁰, O and S(O)ₓ;
D⁴, G⁴, L⁴, M⁴, and T⁴ are independently selected from CR⁶ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalky, aryl and heteroaryl are optionally substituted one ore more times.

34. The compound of claim 33, wherein at least one R¹ is selected from the group consisting of:

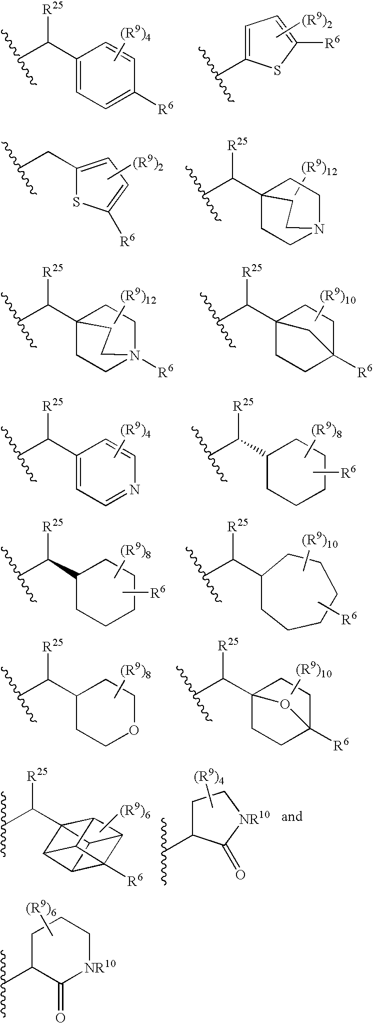

35. The compound of claim 34, wherein:
R⁶ is selected from the group consisting of hydrogen, halo, CN, OH, CH₂OH, CF₃, CHF₂, OCF₃, OCHF₂, COCH₃, SO₂CH₃, SO₂CF₃, SO₂NH₂, SO₂NHCH₃, SO₂N(CH₃)₂, NH₂, NHCOCH₃, N(COCH₃)₂, NHCONH₂, NHSO₂CH₃, alkoxy, alkyl, cycloalkyl, heterocycloalkyl, bicycloalkyl, CO₂H, 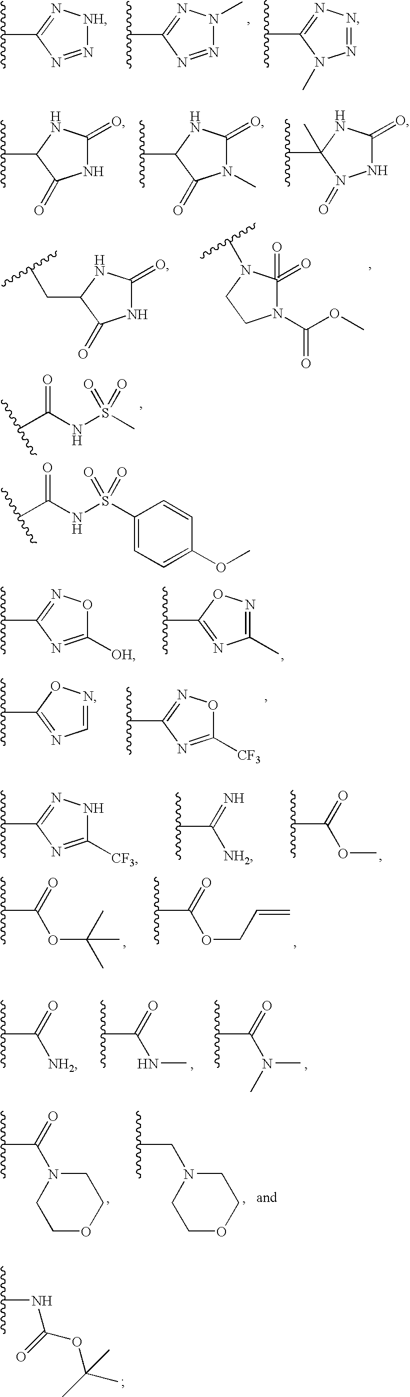
R⁹ is independently selected from the group consisting of hydrogen, fluoro, chloro, CH₃, CF₃, CHF₂, OCF₃, and OCHF₂;
R²⁵ is selected from the group consisting of hydrogen, CH₃, COOCH₃, COOH, and CONH₂.

36. The compound of claim 35, wherein at least one R¹ is selected from the group consisting of:

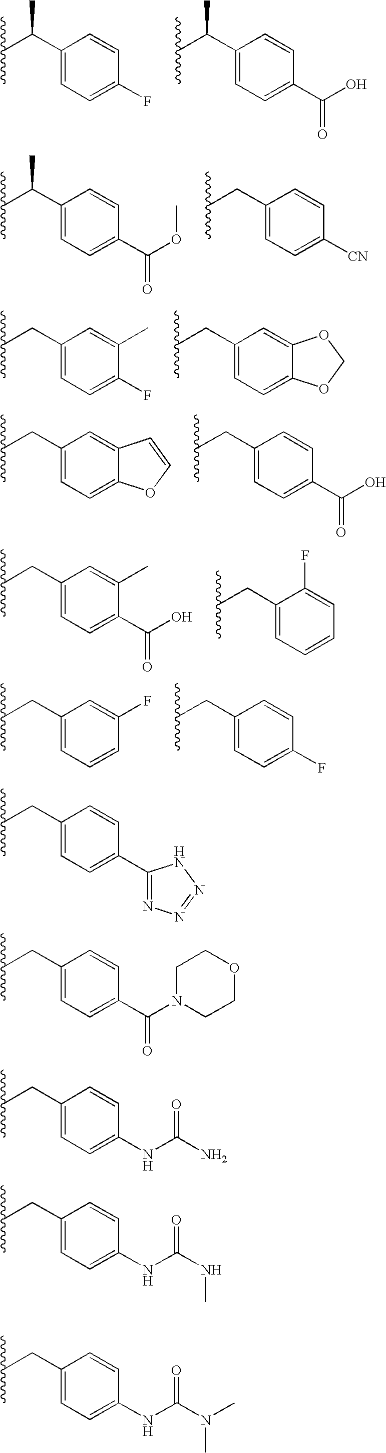

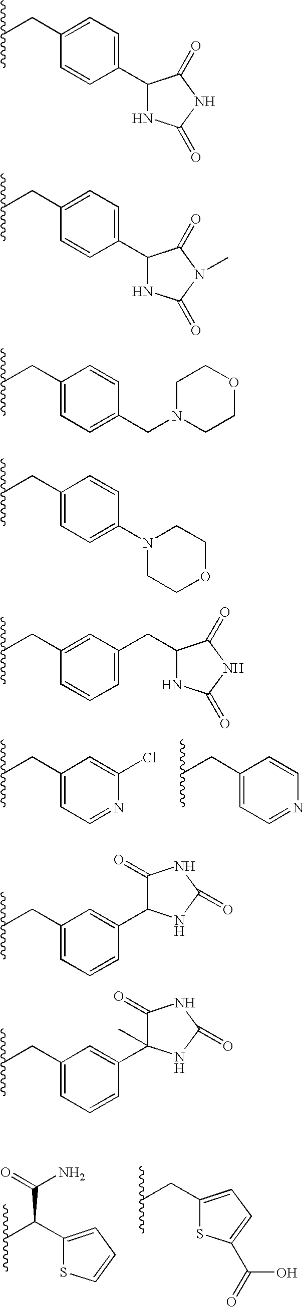

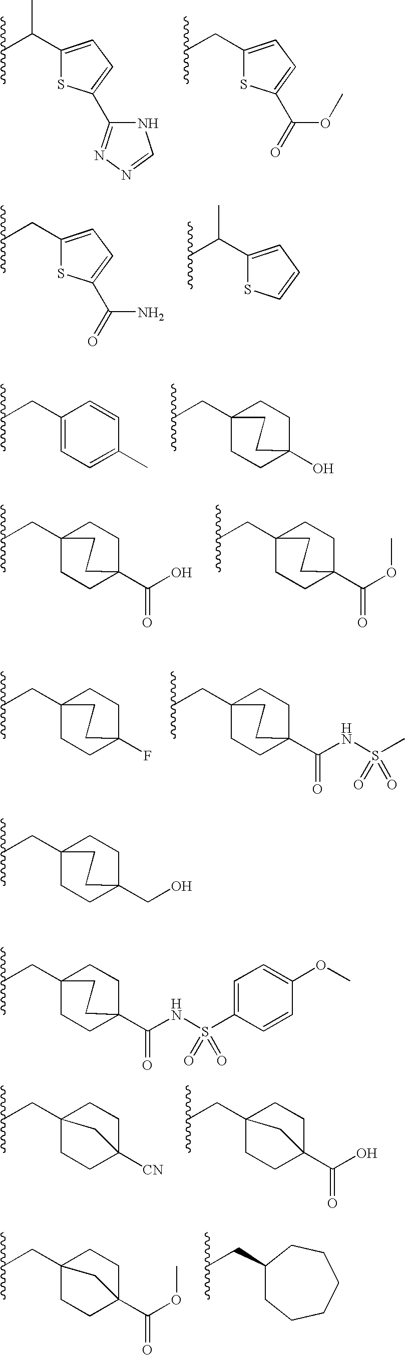

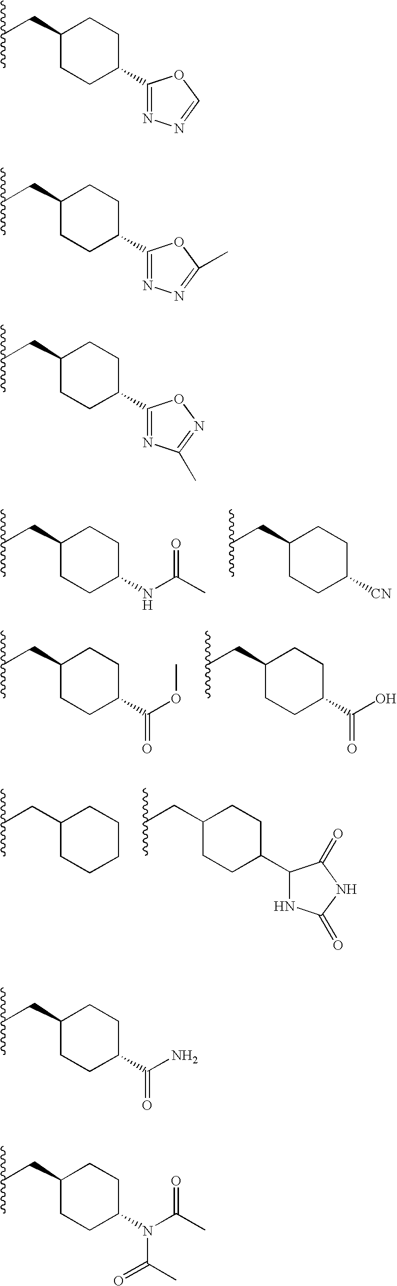

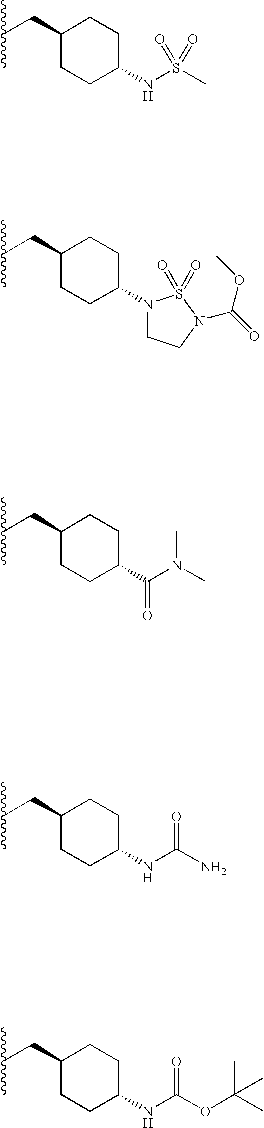

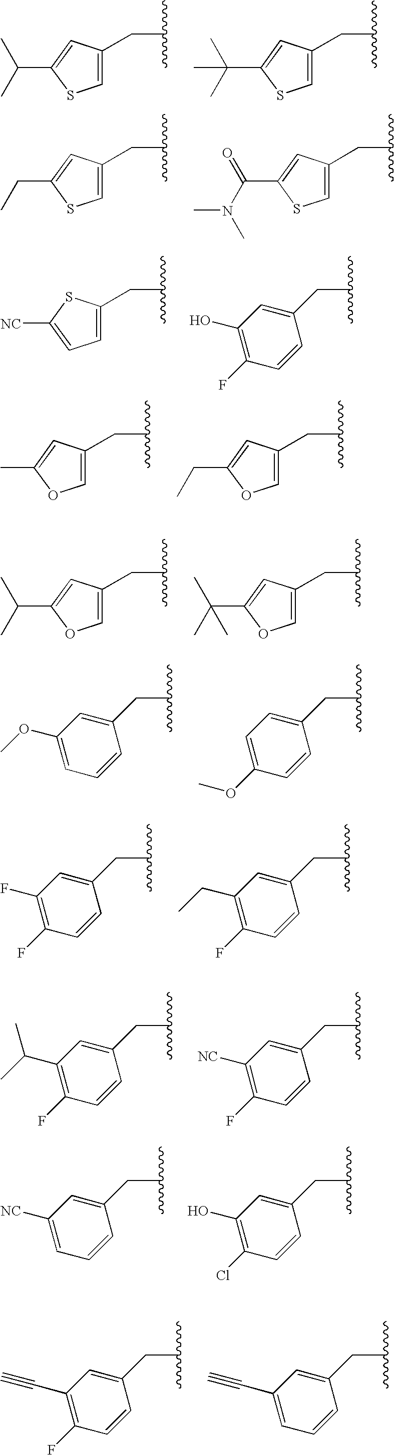

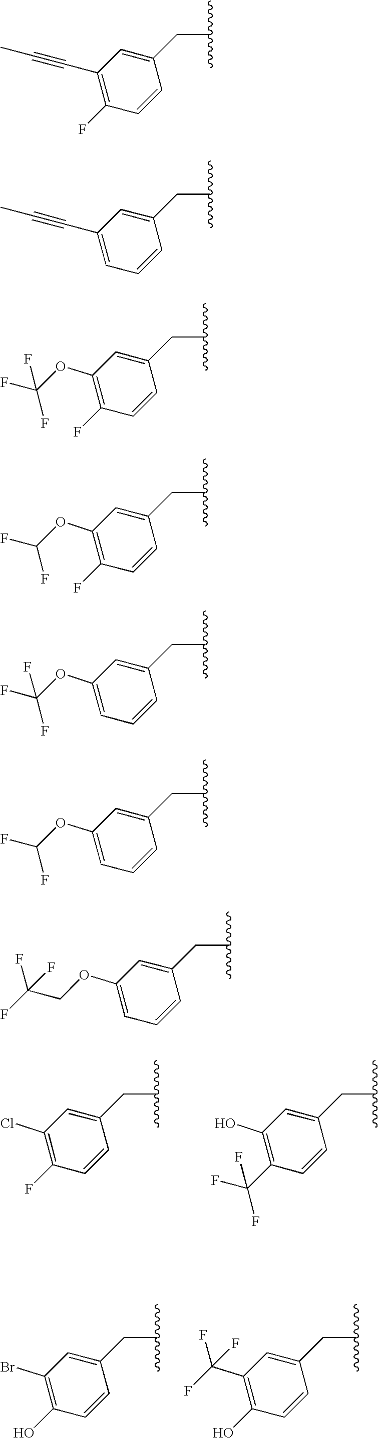

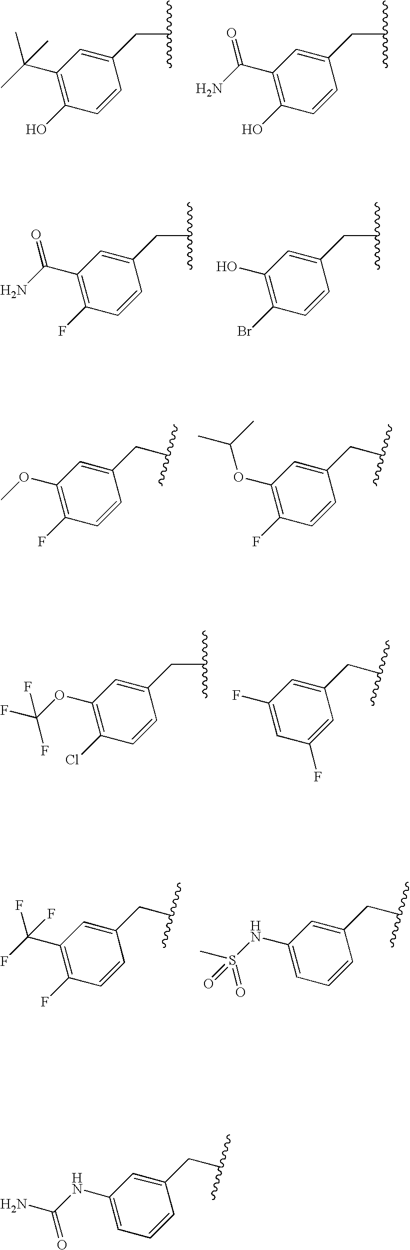

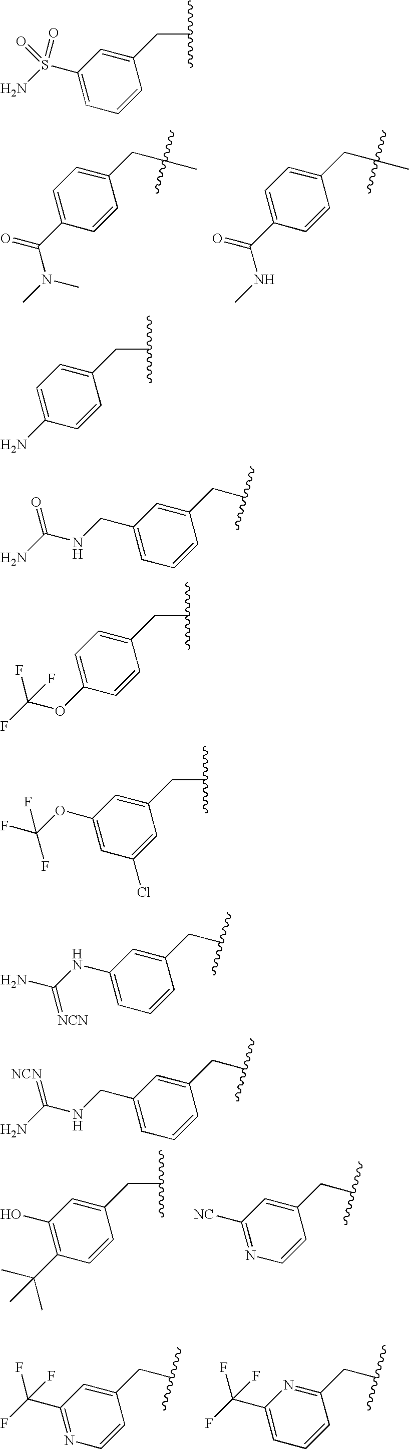

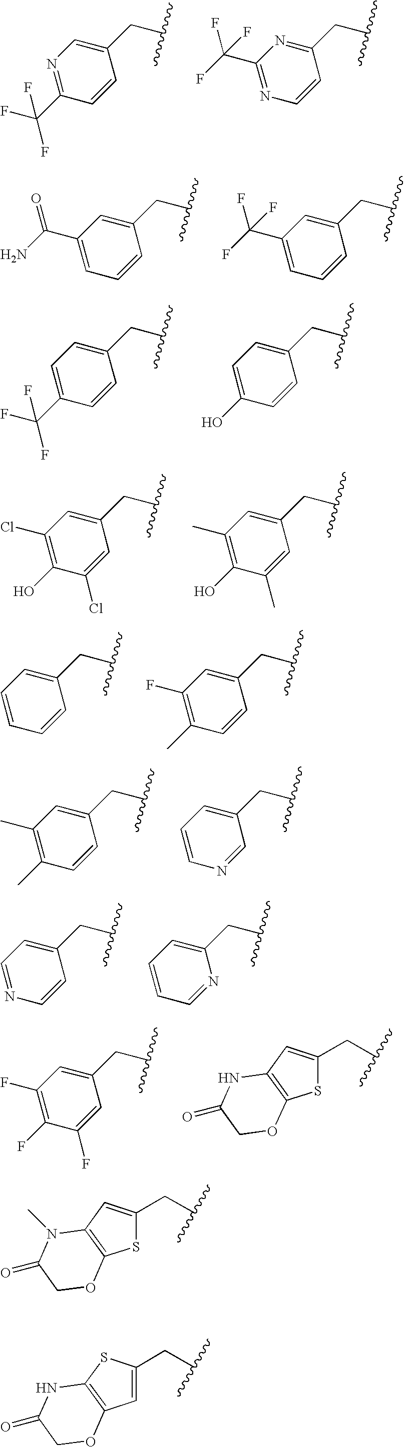

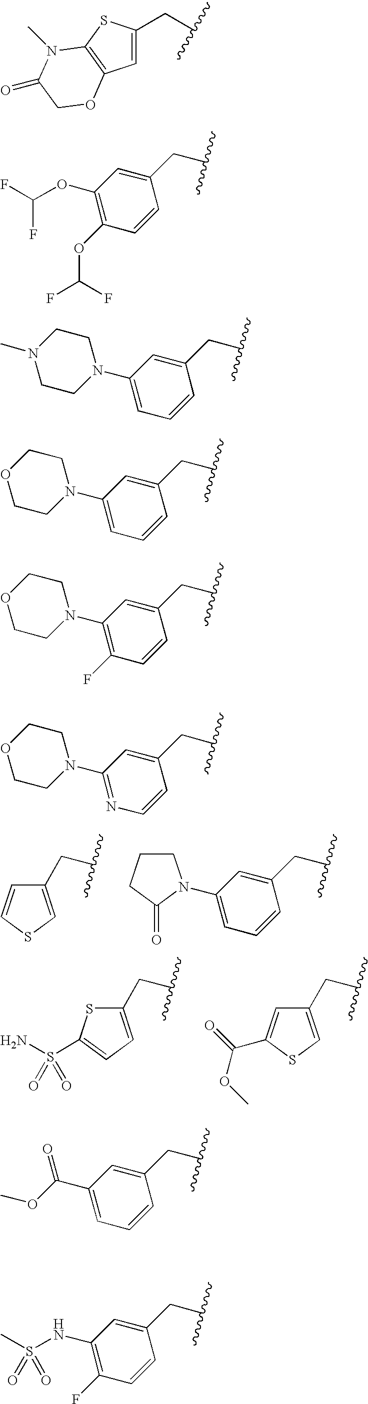

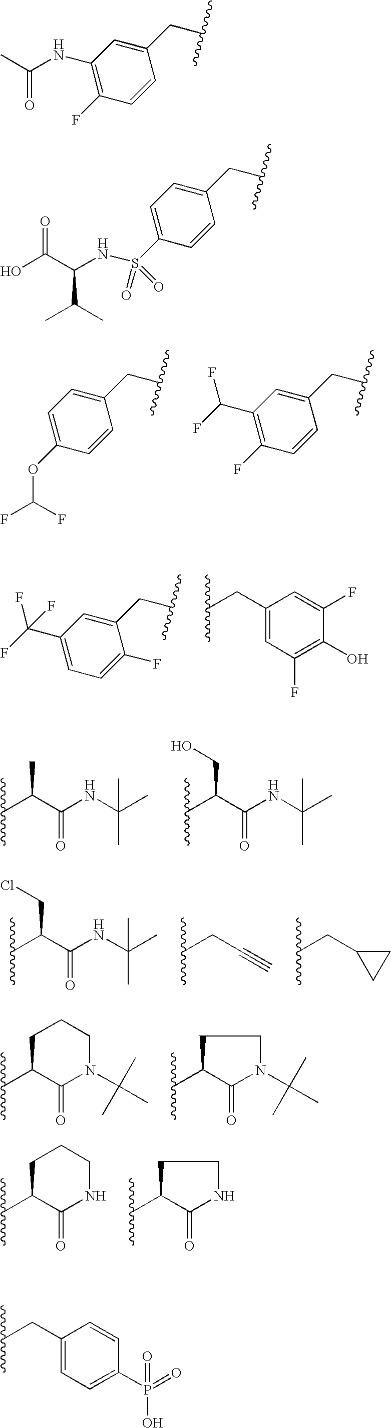

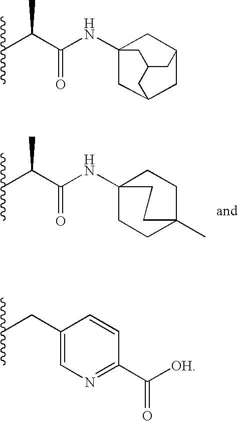

37. The compound of claim 23, wherein at least one R¹ is selected from the group consisting of: 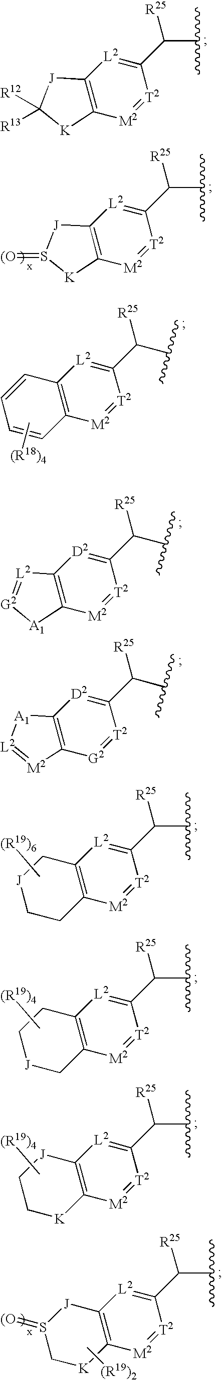 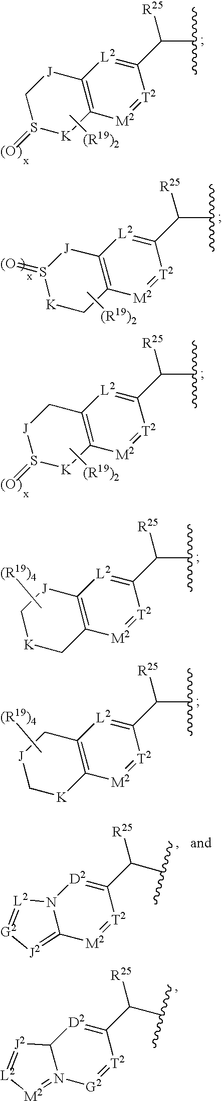
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalksyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R11, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O and S(O)ₓ; and
D², G², J², L², M² and T² are independently selected from the group consisting of CR⁹, CR¹⁸ and N.

38. The compound of claim 37, wherein at least one R¹ is selected from the group consisting of:

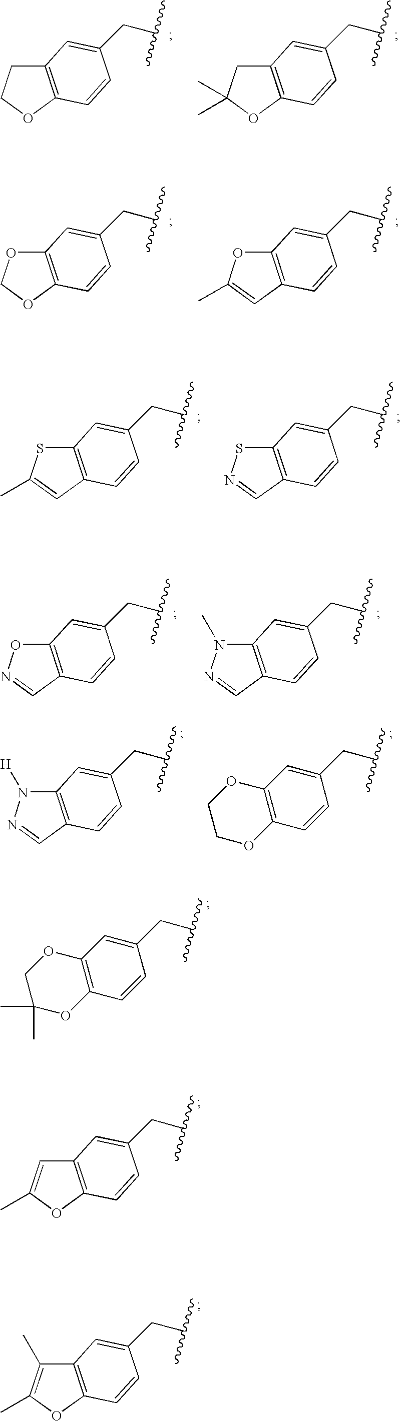

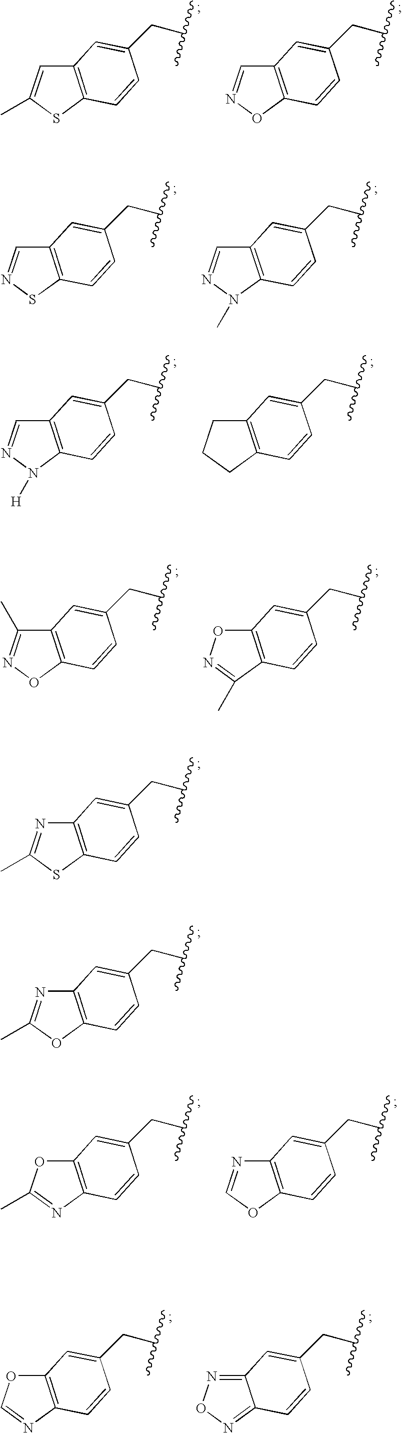

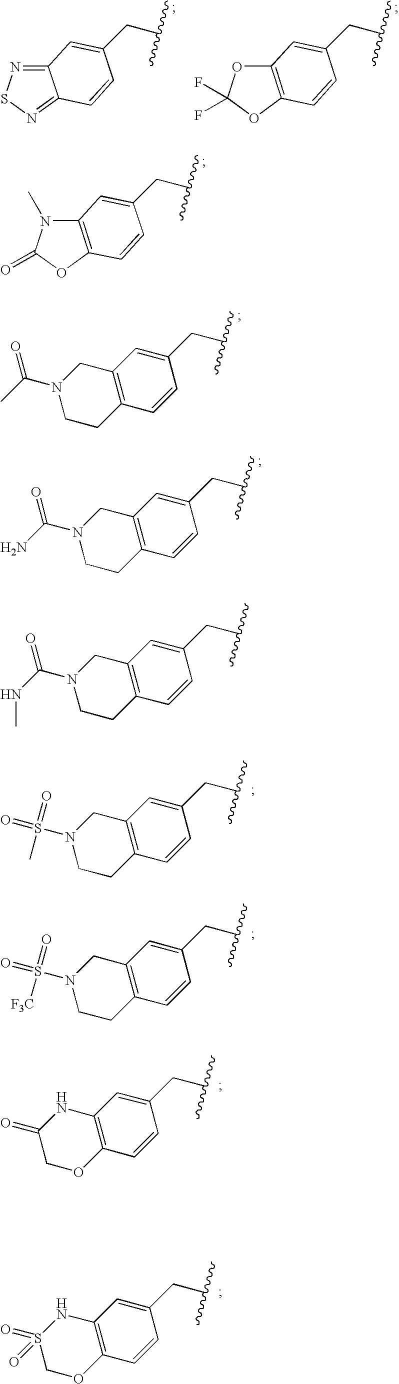

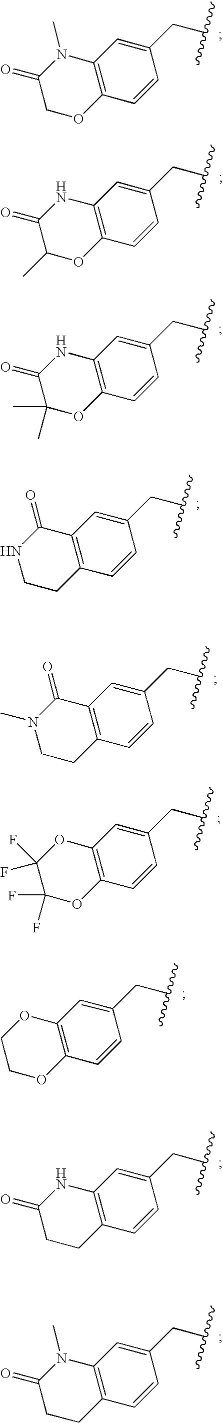

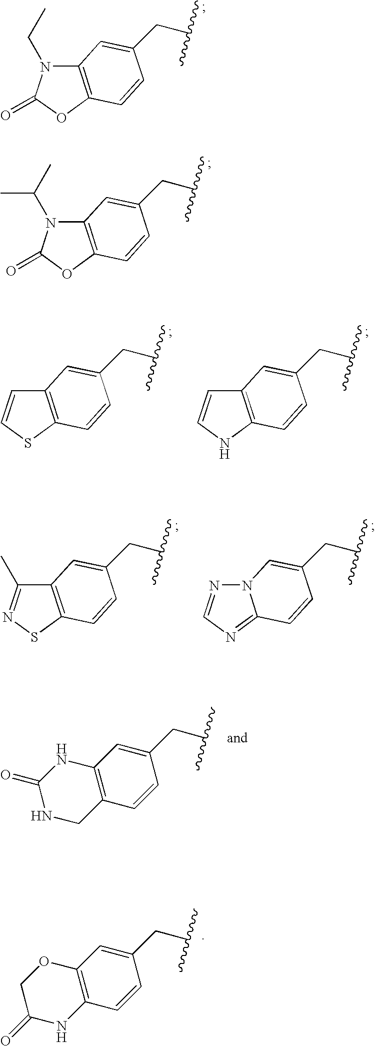

39. A compound having Formula (III) 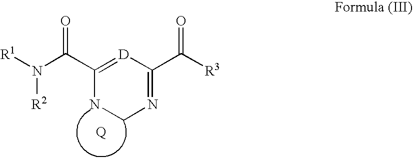
wherein:
R¹ is selected from the group consisting of hydrogen, alkyl, alkenyl, alkynyl, cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl,
wherein R¹ is optionally substituted one or more times, or
wherein R¹ is optionally substituted one or more times by R⁹, or
wherein R¹ is optionally substituted by one R¹⁶ group and optionally substituted by one or more R⁹ groups;
R² is selected from the group consisting of hydrogen and alkyl, wherein alkyl is optionally substituted one or more times or R¹ and R² when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R³ is NR²⁰R²¹;
R⁴ in each occurrence is independently selected from the group consisting of R¹⁰, hydrogen, alkyl, cycloalkyl, heterocycloalkyl, aryl, heteroaryl, halo, haloalkyl, CF₃, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O),OR¹⁰, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, (C₀-C₆)-alkyl-C(O)—NR¹¹—CN, O-(C ₀-C₆)-alkyl-C(O)NR¹⁰R¹¹, S(O)ₓ-(C₀-C₆)-alkyl-C(O)OR¹⁰, S(O)ₓ-(C₀-C₆)-alkyl-C(O)NR¹⁰R¹¹, (C₀-C₆)-alkyl-C(O)NR¹⁰-(C₀-C₆)-alkyl-NR¹⁰R¹¹, (C₀-C₆)-alkyl-NR¹⁰-C(O)R¹⁰, (C₀-C₆)-alkyl-NR¹⁰-C(O)OR¹⁰, (C₀-C₆)-alkyl-NR¹⁰-C(O)—NR¹⁰R¹¹, (C₀-C₆)-alkyl-NR¹⁰-S(O)_{y}NR¹⁰R¹¹, (C₀-C₆)-alkyl-NR¹⁰-S(O)_{y}R¹⁰, O-(C₀-C₆)-alkyl-aryl and O-(C₀-C₆)-alkyl-heteroaryl,
wherein each R⁴ group is optionally substituted one or more times, or
wherein each R⁴ group is optionally substituted by one or more R¹⁴ groups;
R⁵ in each occurrence is independently selected from the group consisting of hydrogen, alkyl, C(O)NR¹⁰R¹¹, aryl, arylalkyl, SO₂NR¹⁰R¹¹ and C(O)OR¹⁰, wherein alkyl, aryl and arylalkyl are optionally substituted one or more times;
R⁹ in each occurrence is independently selected from the group consisting of R¹⁰, hydrogen, alkyl, cycloalkyl, heterocycloalkyl, aryl, heteroaryl, halo, CHF₂, CF₃, OR¹⁰, SR¹⁰, COOR¹⁰, CH(CH₃)CO₂H, (C₀-C₆)-alkyl-COR¹⁰, (C₀-C₆)-alkyl-OR¹⁰, (C₀-C₆)-alkyl-NR¹⁰R¹¹, (C₀-C₆)-alkyl-NO₂, (C₀-C₆)-alkyl-CN, (C₀-C₆)-alkyl-S(O)_{y}OR¹⁰, (C₀-C₆)-alkyl-P(O)₂OH, (C₀-C₆)-alkyl-S(O)_{y}NR¹⁰R¹¹, (C₀-C₆)-alkyl-NR¹⁰CONR¹¹SO₂R³⁰, (C₀-C₆)-alkyl-S(O)ₓR¹⁰, (C₀-C₆)-alkyl-OC(O)R¹⁰, (C₀-C₆)-alkyl-OC(O)NR¹⁰R¹¹, (C₀-C₆)-alkyl-C(═NR¹⁰)NR¹⁰R¹¹, (C₀-C₆)-alkyl-NR¹⁰C(═NR¹¹)NR¹⁰R¹¹, (C₀-C₆)-alkyl-NR¹⁰C(═N—CN)NR¹⁰R¹¹, (C₀-C₆)-alkyl-C(═N—CN)NR¹⁰R¹¹, (C₀-C₆)-alkyl-NR¹⁰C(═N—NO₂)NR¹⁰R¹¹, (C₀-C₆)-alkyl-C(═N—NO₂)NR¹⁰R¹¹, (C₀-C₆)-alkyl-C(O)OR¹⁰, (C₀-C₆)-alkyl-C(O)NR¹⁰R¹¹, (C₀-C₆)-alkyl-C(O)NR¹⁰SO₂R¹¹, C(O)NR¹⁰-(C₀-C₆)-alkyl-heteroaryl, C(O)NR¹⁰—(C₀-C₆)-alkyl-aryl, S(O)₂NR¹⁰—(C₀-C₆)-alkyl-aryl,S(O)₂NR¹⁰—(C₀-C₆)-alkyl-heteroaryl, S(O)₂NR¹⁰-alkyl, S(O)₂—(C₀-C₆)-alkyl-aryl, S(O)₂—(C₀-C₆)-alkyl-heteroaryl, (C₀-C₆)-alkyl-C(O)—NR¹¹—CN, O—(C₀-C₆)-alkyl-C(O)NR¹⁰R₁₁, S(O)ₓ—(C₀-C₆)-alkyl-C(O)OR¹⁰, S(O)ₓ—(C₀-C₆)-alkyl-C(O)NR¹⁰R¹¹, (C₀-C₆)-alkyl-C(O)NR¹⁰—(C₀-C₆)-alkyl-NR¹⁰R¹¹, (C₀-C₆)-alkyl-NR¹⁰—C(O)R¹⁰, (C₀-C₆)-alkyl-NR¹⁰-C(O)OR¹⁰, (C₀-C₆)-alkyl-NR¹⁰-C(O)—NR¹⁰R¹¹, (C₀-C₆)-alkyl-NR¹⁰-S(O)_{y}NR¹⁰R¹¹, (C₀-C₆)-alkyl-NR¹⁰-S(O)_{y}R¹¹, O—(C₀-C₆)-alkyl-aryl and O-(C₀-C₆)-alkyl-heteroaryl,
wherein each R⁹ group is optionally substituted, or
wherein each R⁹ group is optionally substituted by one or more R¹⁴ groups;
R¹⁰ and R¹¹ in each occurrence are independently selected from the group consisting of hydrogen, alkyl, alkenyl, alkynyl, cycloalkyl, cycloalkylalkyl, heterocycloalkyl, bicycloalkyl, heterobicycloalkyl, spiroalkyl, spiroheteroalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, bicycloalkyl, heterobicycloalkyl, spiroalkyl, spiroheteroalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted one or more times, or R¹⁰ and R¹¹ when taken together with the nitrogen to which they are attached complete a 3- to 8-membered ring containing carbon atoms and optionally containing a heteroatom selected from O, S(O)ₓ, or NR⁵⁰ and which is optionally substituted one or more times;
R¹⁴ is independently selected from the group consisting of hydrogen, alkyl, arylalkyl, cycloalkylalkyl, heteroarylalkyl, heterocyclylalkyl and halo, wherein alkyl, arylalkyl, cycloalkylalkyl, heteroarylalkyl and heterocyclylalkyl are optionally substituted one or more times;
R¹⁶ is selected from the group consisting of cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, heterocycloalkyl fused heteroarylalkyl, (i) and (ii): 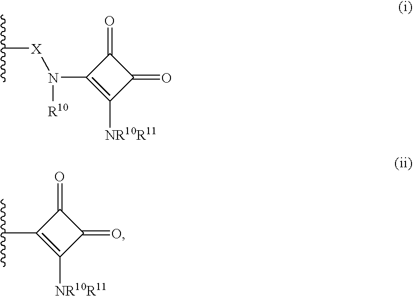
wherein cycloalkyl, heterocycloalkyl, bicycloalkyl, heterobicycloalkyl, spiroalkyl, spiroheteroalkyl, aryl, heteroaryl, cycloalkyl fused aryl, heterocycloalkyl fused aryl, cycloalkyl fused heteroaryl, heterocycloalkyl fused heteroaryl, cycloalkylalkyl, heterocycloalkylalkyl, bicycloalkylalkyl, heterobicycloalkylalkyl, spiroalkylalkyl, spiroheteroalkylalkyl, arylalkyl, heteroarylalkyl, cycloalkyl fused arylalkyl, heterocycloalkyl fused arylalkyl, cycloalkyl fused heteroarylalkyl, and heterocycloalkyl fused heteroarylalkyl are optionally substituted one or more times;
R²⁰ is selected from the group consisting of hydrogen and alkyl, wherein alkyl is optionally substituted one or more times;
R²¹ is a bicyclic or tricyclic fused ring system, wherein at least one ring is partially saturated, and
wherein R²¹ is optionally substituted one or more times, or
wherein R²¹ is optionally substituted by one or more R⁹ groups;
R²² is selected from the group consisting of hydrogen, hydroxy, halo, alkyl, cycloalkyl, alkoxy, alkenyl, alkynyl, NO₂, NR¹⁰R¹¹, CN, SR¹⁰, SSR¹⁰, PO₃R¹⁰, NR¹⁰NR¹⁰R¹¹, NR¹⁰N═CR¹⁰R¹¹, NR¹⁰SO₂R¹¹, C(O)OR¹⁰, C(O)NR¹⁰R¹¹, SO₂R¹⁰, SO₂NR¹⁰R¹¹ and fluoroalkyl, wherein alkyl, cycloalkyl, alkoxy, alkenyl, alkynyl, and fluoroalkyl are optionally substituted one or more times;
R³⁰ is selected from the group consisting of alkyl and (C₀-C₆)-alkyl-aryl, wherein alkyl and aryl are optionally substituted;
R⁵⁰ in each occurrence is independently selected from the group consisting of hydrogen, alkyl, aryl, heteroaryl, C(O)R⁸⁰, C(O)NR⁸⁰R⁸¹, SO₂R⁸⁰ and SO₂NR⁸⁰R⁸¹, wherein alkyl, aryl, and heteroaryl are optionally substituted one or more times;
R⁸⁰ and R⁸¹ in each occurrence are independently selected from the group consisting of hydrogen, alkyl, cycloalkyl, cycloalkylalkyl, heterocycloalkyl, fluoroalkyl, heterocycloalkylalkyl, haloalkyl, alkenyl, alkynyl, aryl, heteroaryl, arylalkyl, heteroarylalkyl and aminoalkyl, wherein alkyl, cycloalkyl, cycloalkylalkyl, heterocycloalkyl, fluoroalkyl, heterocycloalkylalkyl, alkenyl, alkynyl, aryl, heteroaryl, arylalkyl, heteroarylalkyl and aminoalkyl are optionally substituted, or R⁸⁰ and R⁸¹ when taken together with the nitrogen to which they are attached complete a 3- to 8-membered ring containing carbon atoms and optionally a heteroatom selected from O, S(O)ₓ, —NH, and —N(alkyl) and which is optionally substituted one or more times;
E is selected from the group consisting of a bond, CR¹⁰R¹¹, O, NR⁵, S, S═O, S(═O)₂, C(═O), N(R¹⁰)(C═O), (C═O)N(R¹⁰), N(R¹⁰S(═O)₂, S(═O)₂N(R¹⁰), C═N—OR¹¹, —C(R¹⁰R¹¹)C(R¹⁰R¹¹)—, —CH₂—W¹— and 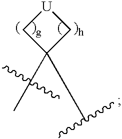
Q is a 5- or 6-membered ring selected from the group consisting of aryl and heteroaryl, wherein aryl and heteroaryl are optionally substituted one or more times with R⁴;
D is a member selected from the group consisting of CR²² and N;
U is selected from the group consisting of C(R⁵R¹⁰), NR⁵, O, S, S═O and S(═O)₂;
W¹ is selected from the group consisting of O, NR⁵, S, S═O, S(═O)₂, N(R¹⁰)(C═O), N(R¹⁰)S(═O)₂ and S(═O)₂N(R¹⁰);
X is selected from the group consisting of a bond and (CR¹⁰R¹¹)_{w}E(CR¹⁰R¹¹)_{w};
g and h are independently selected from 0-2;
w is independently selected from 0-4;
x is selected from 0 to 2;
y is selected from 1 and 2; and
N-oxides, pharmaceutically acceptable salts, prodrugs, formulation, polymorphs, racemic mixtures and stereoisomers thereof.

40. The compound of claim 39, selected from the group consisting of: 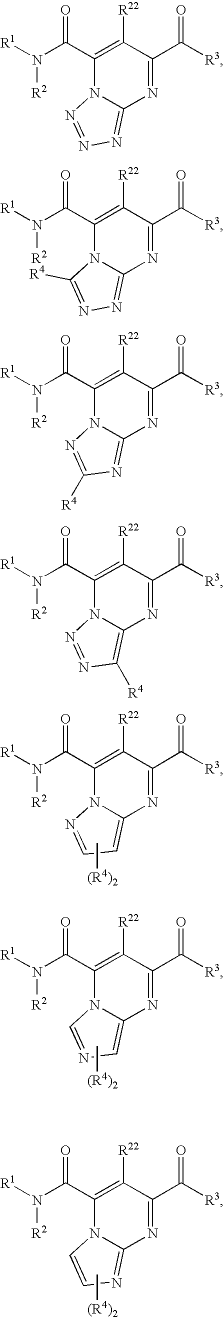 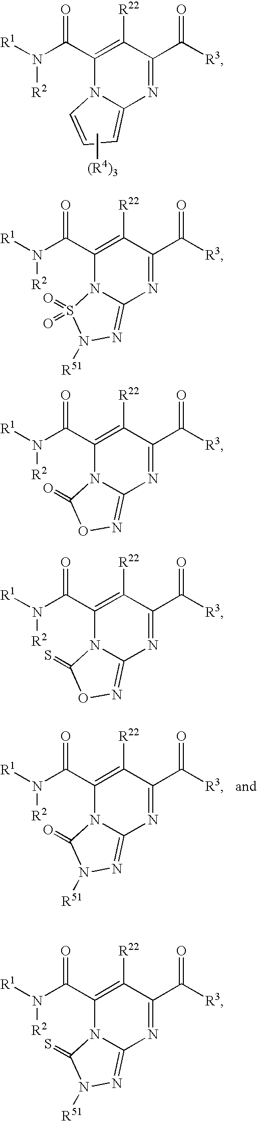
wherein:
R⁵¹ is independently selected from the group consisting of hydrogen, alkyl, aryl, heteroaryl, arylalkyl, cycloalkylalkyl, heteroarylalkyl and haloalkyl, wherein alkyl, aryl, heteroaryl, arylalkyl, cycloalkylalkyl, heteroarylalkyl and haloalkyl are optionally substituted one or more times.

41. The compound of claim 40, selected from the group consisting of:

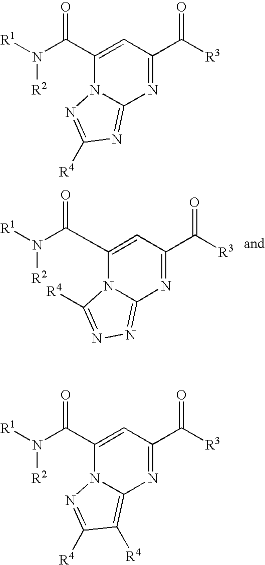

42. The compound of claim 41, selected from the group consisting of:

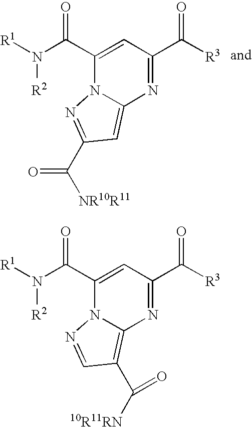

43. The compound of claim 42, selected from the group consisting of: 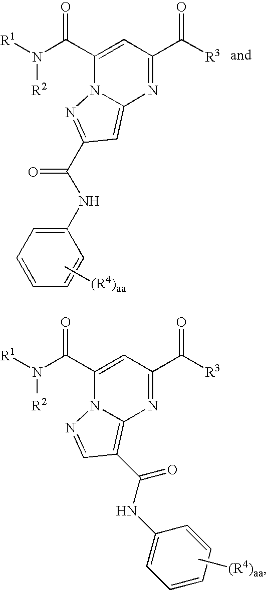
wherein:
aa is selected from 0-5.

44. The compound of claim 40, wherein R³ is selected from the group consisting of: 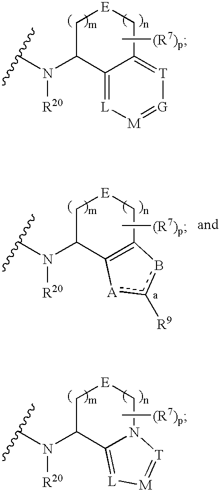
wherein:
R⁷ is independently selected from the group consisting of hydrogen, alkyl, cycloalkyl, halo, R⁴ and NR¹⁰R¹¹, or optionally two R⁷ groups together at the same carbon atom form ═O, ═S or ═NR¹⁰;
A and B are independently selected from the group consisting of CR⁹, CR⁹R¹⁰, NR¹⁰, N, O and S(O)ₓ;
G, L, M and T are independently selected from the group consisting of CR⁹ and N;
m and n are independently selected from 0-3, provided that:
(1) when E is present, m and n are not both 3;
(2) when E is —CH₂—W¹—, m and n are not 3; and
(3) when E is a bond, m and n are not 0; and
p is selected from 0-6;
wherein the dotted line represents a double bond between one of: carbon “a” and A, or carbon “a” and B.

45. The compound according to claim 44, wherein R³ is selected from the group consisting of: 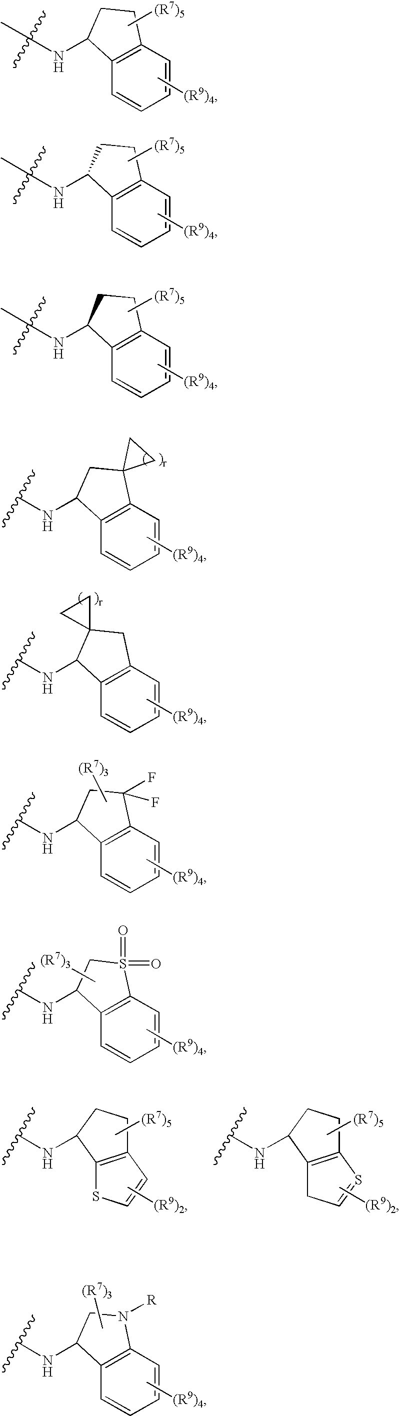 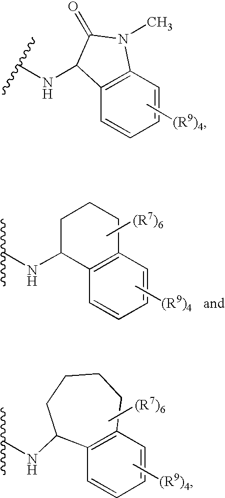
wherein:
R is selected from the group consisting of C(O)NR¹⁰OR¹¹, COR¹⁰, SO₂NR¹⁰R¹¹, SO₂R¹⁰, CONHCH₃ and CON(CH₃)₂, wherein C(O)NR¹⁰R¹¹, COR¹⁰, SO₂NR¹⁰R¹¹, SO₂R¹⁰, CONHCH₃ and CON(CH₃)₂ are optionally substituted one or more times; and
r is selected from 1-6.

46. The compound according to claim 45, wherein R³ is selected from the group consisting of:

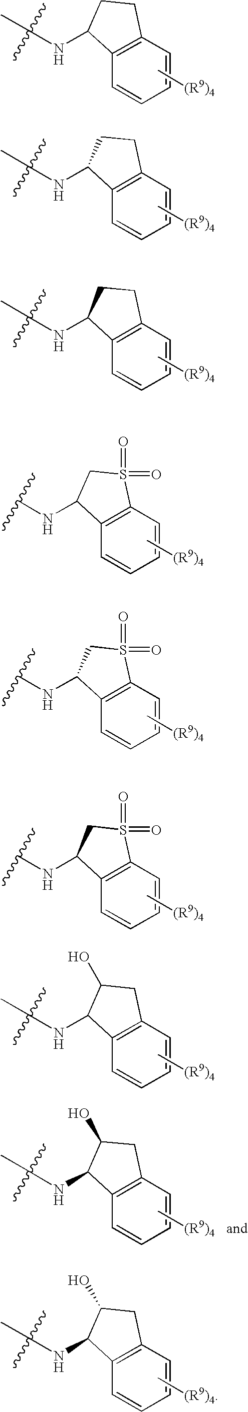

47. The compound according to claim 46, wherein R⁹ is selected from the group consisting of: 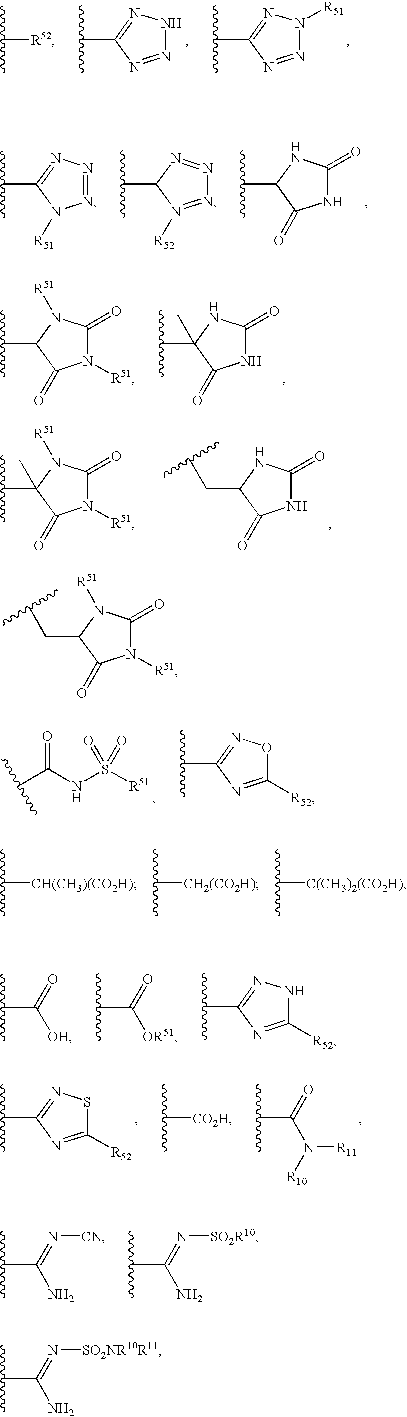 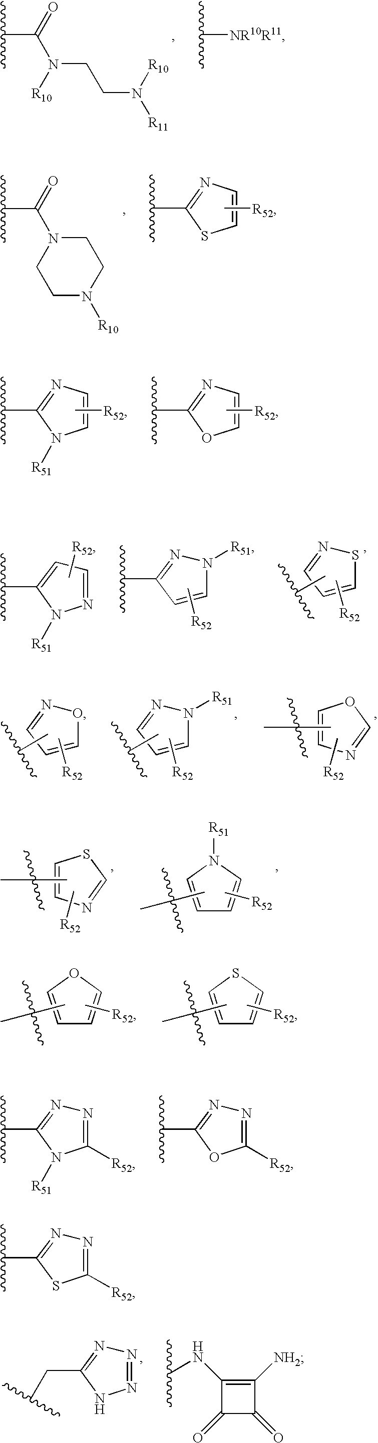 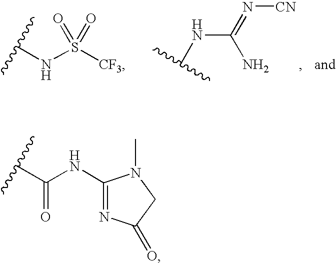
wherein:
R⁵² is selected from the group consisting of hydrogen, halo, CN, hydroxy, alkoxy, fluoroalkoxy, alkyl, aryl, heteroaryl, arylalkyl, cycloalkylalkyl, heteroarylalkyl, haloalkyl, C(O)NR¹⁰R¹¹ and SO₂NR¹⁰R¹¹, wherein alkoxy, fluoroalkoxy, alkyl, aryl, heteroaryl, arylalkyl, cycloalkylalkyl, heteroarylalkyl, and haloalkyl are optionally substituted one or more times.

48. The compound according to claim 46, wherein R³ is

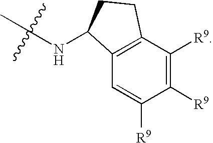

49. The compound according to claim 48, wherein R³ is selected from the group consisting of: 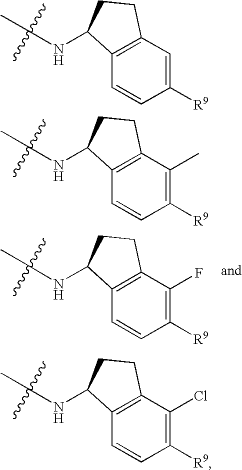
wherein:
R⁹ is selected from the group consisting of hydrogen, fluoro, halo, CN, alkyl, CO₂H,

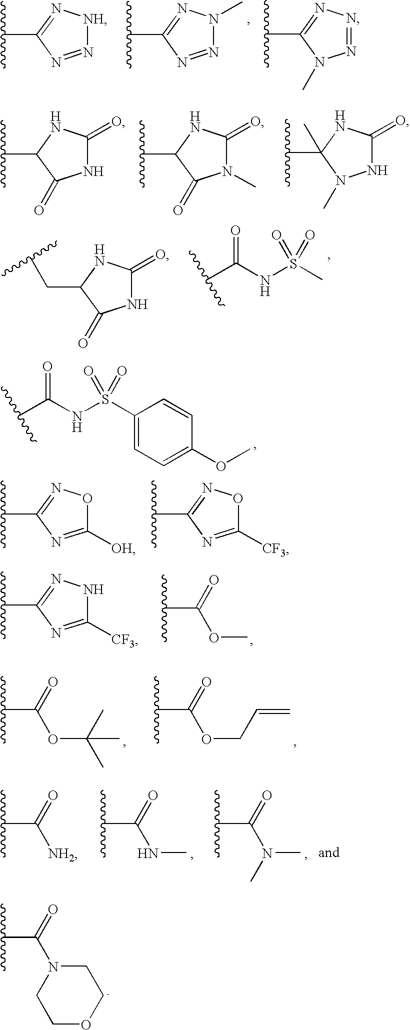

50. The compound according to claim 40, wherein R¹ is selected from the group consisting of: 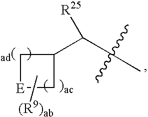
wherein:
ab is selected from the integer (2×ac)+(2×ad)+1;
ac is selected from 1-5;
ad is selected from 0-5;
optionally two R⁹ groups together at the same carbon atom form ═O, ═H or ═NR¹⁰; and
R²⁵ is selected from the group consisting of hydrogen, alkyl, cycloalkyl, CO₂R¹⁰, C(0)NR¹⁰R¹¹ and haloalkyl, wherein alkyl, cycloalkyl, and haloalkyl are optionally substituted one or more times.

51. The compound according to claim 50, wherein R¹ is selected from the group consisting of:

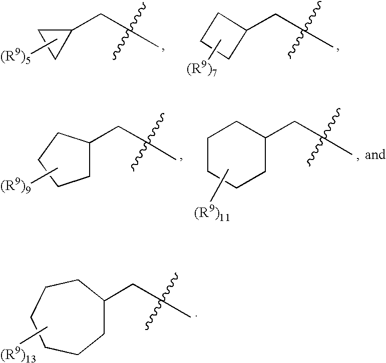

52. The compound according to claim 51, wherein R¹ is selected from the group consisting of:

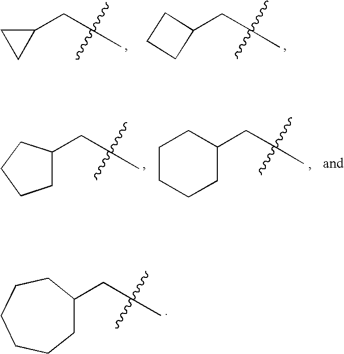

53. The compound according to claim 40, wherein R¹ is selected from the group consisting of: 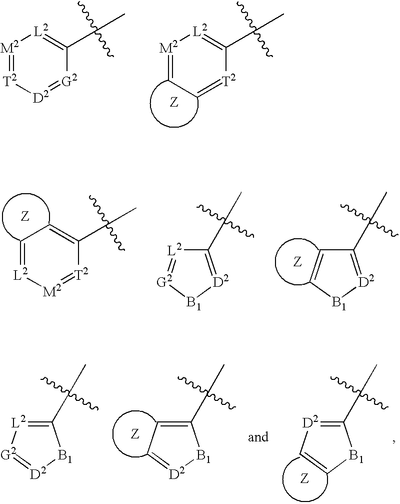
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰OR¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
B₁, is selected from the group consisting of NR¹⁰, O and S(O)ₓ;
D², G², L², M² and T² are independently selected from the group consisting of CR⁹, CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

54. The compound according to claim 53, wherein R¹ is selected from the group consisting of: 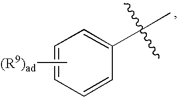
wherein:
ad is selected from 0-5.

55. The compound according to claim 54, wherein R¹ is selected from the group consisting of:

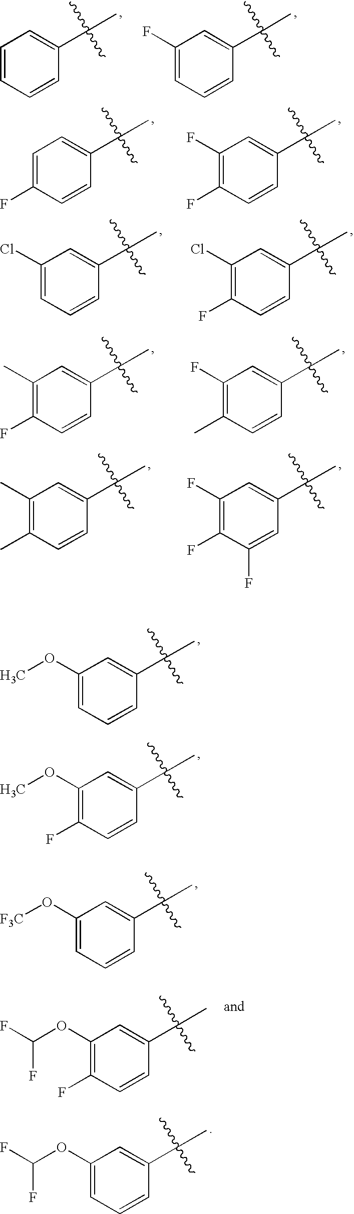

56. The compound according to claim 40, wherein R¹ is selected from the group consisting of: 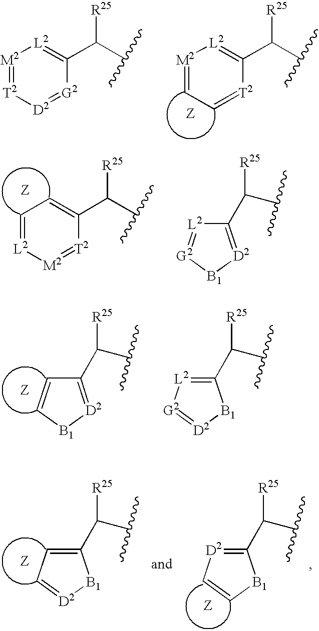
wherein:
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰ , OCF₃, OCHF₂, NR¹⁰ CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, heteroaryl are optionally substituted one or more times;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
B₁ is selected from the group consisting of NR¹⁰, O and S(O)ₓ;
D², G², L², M² and T² are independently selected from the group consisting of CR⁹, CR¹⁸ and N; and
Z is a 5- to 8-membered ring selected from the group consisting of cycloalkyl, heterocycloalkyl, or a 5- to 6-membered ring selected from the group consisting of aryl and heteroaryl, wherein cycloalkyl, heterocycloalkyl, aryl and heteroaryl are optionally substituted one or more times.

57. The compound according to claim 56, wherein R¹ is selected from the group consisting of:

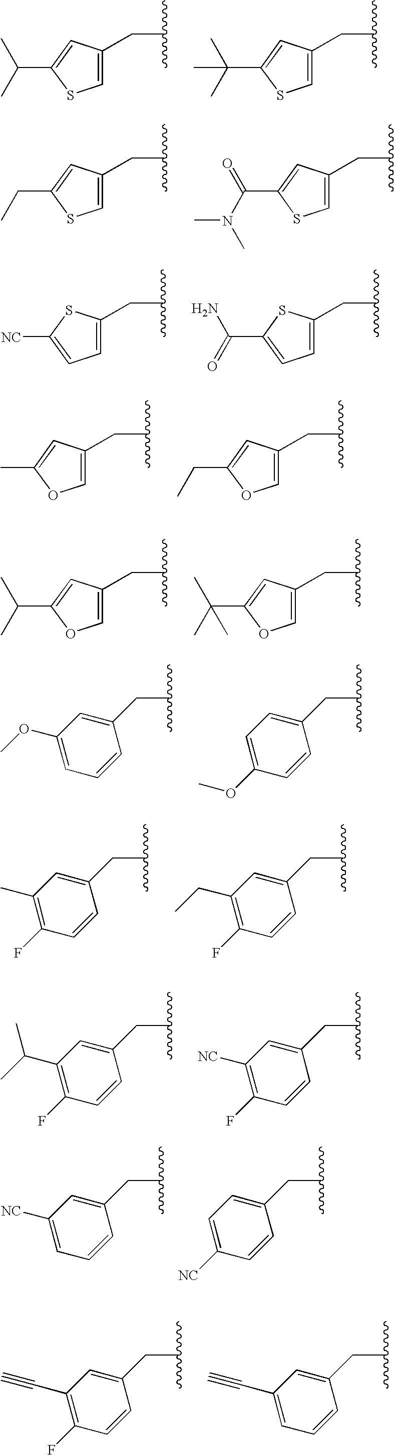

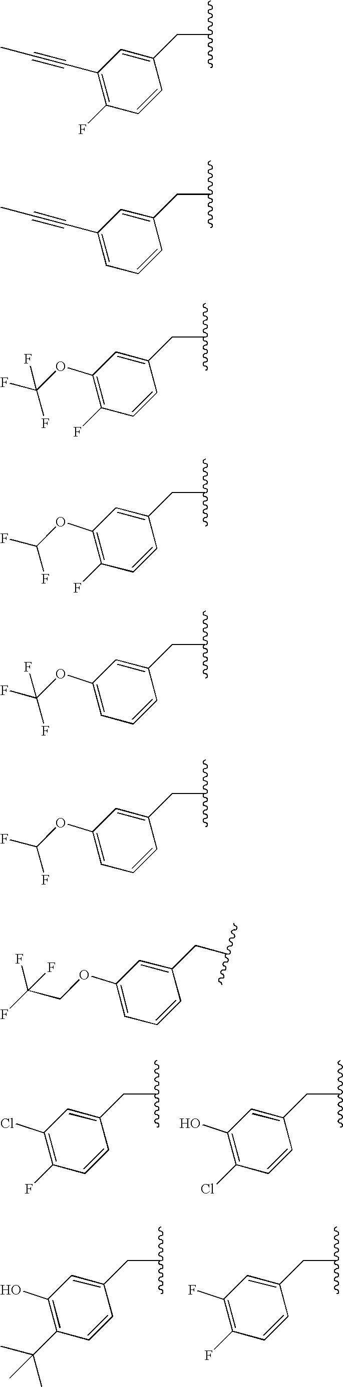

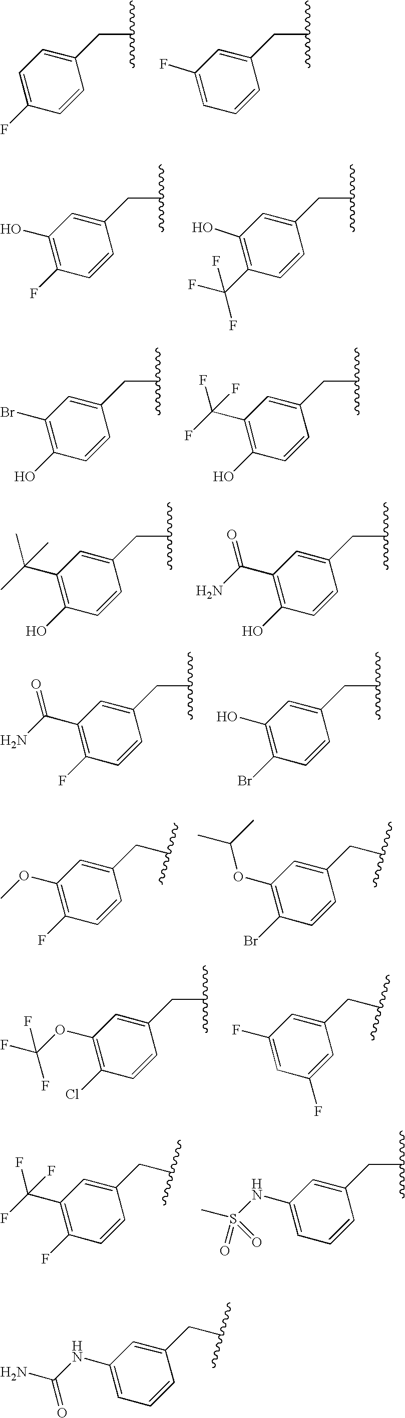

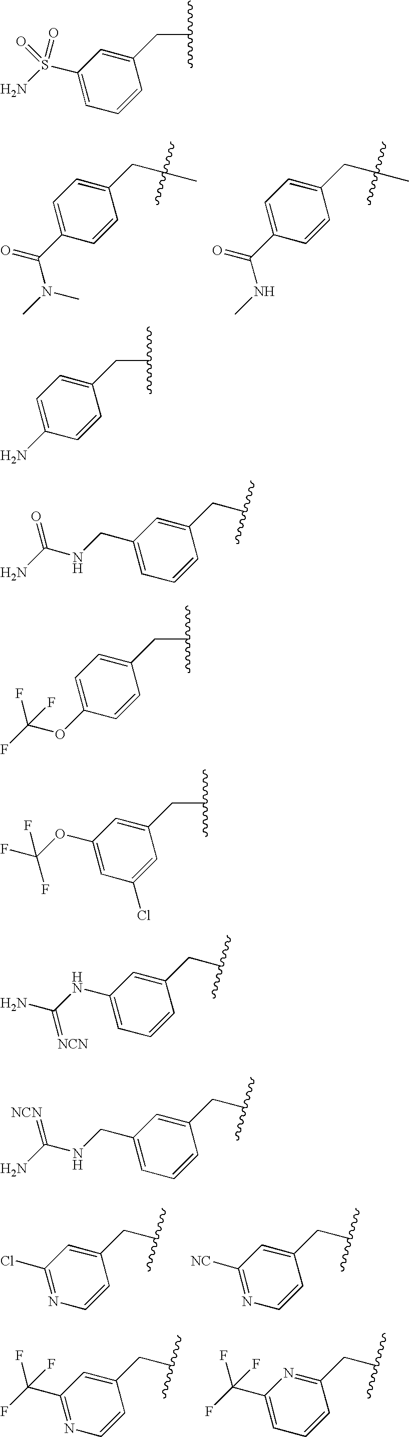

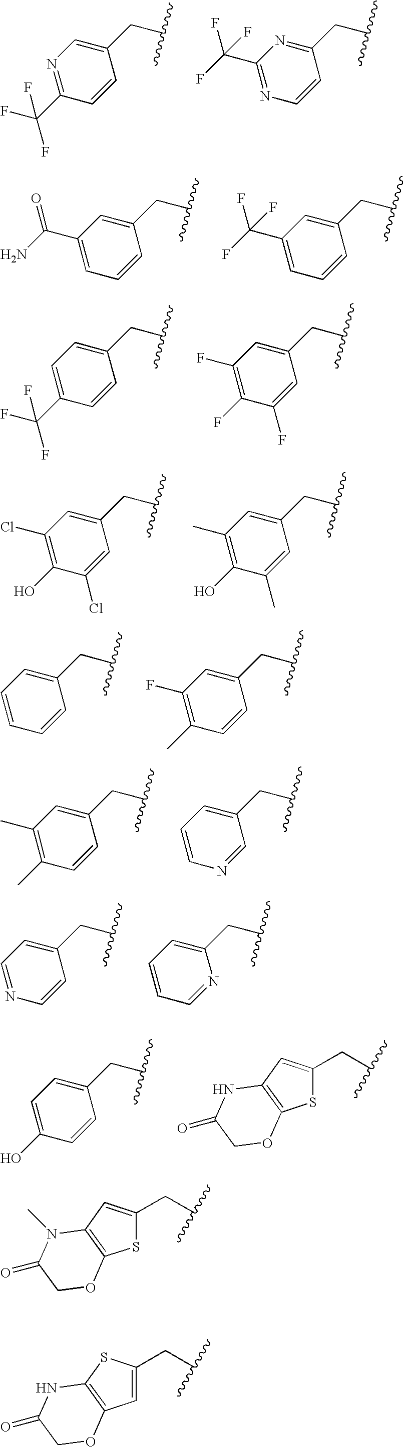

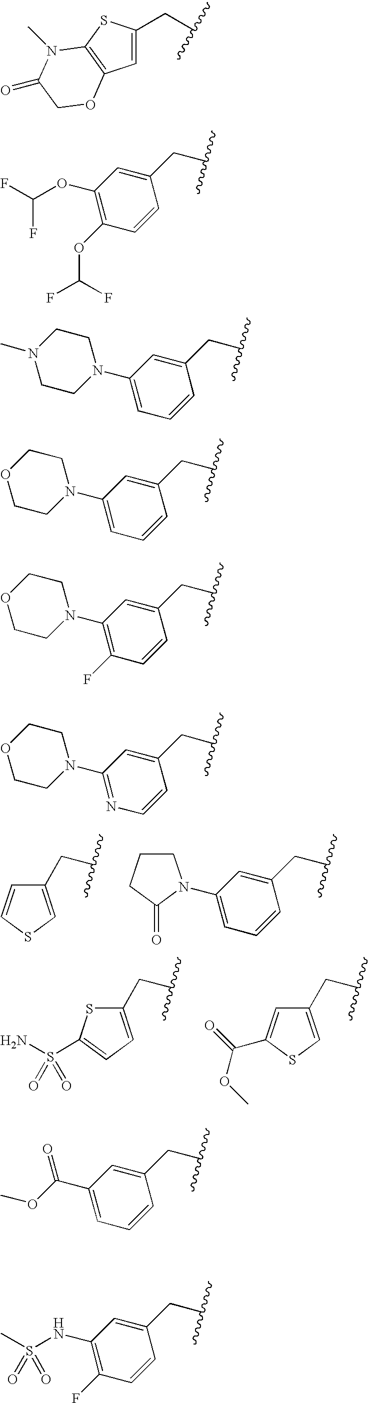

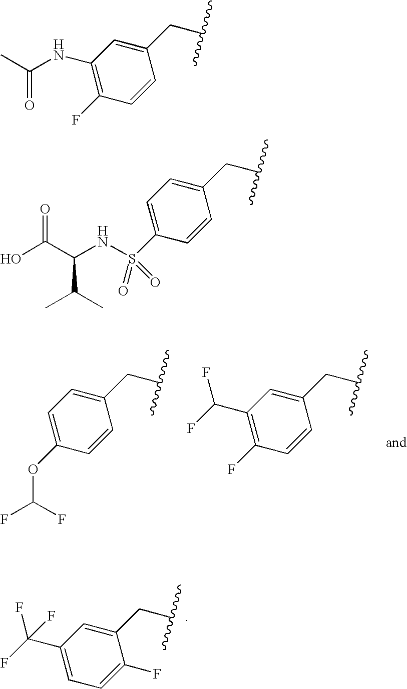

58. The compound of claim 40, wherein R¹ is selected from the group consisting of: 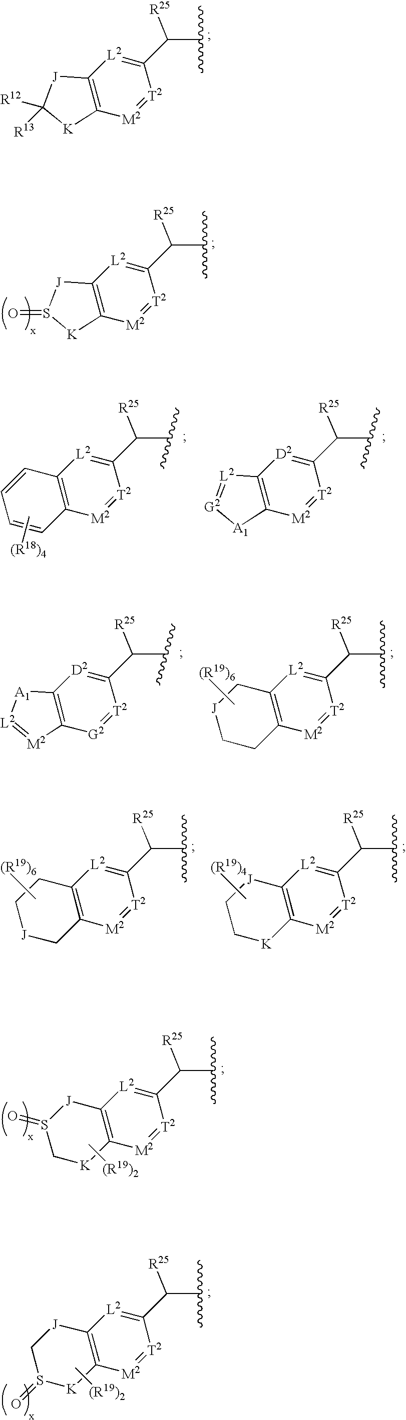 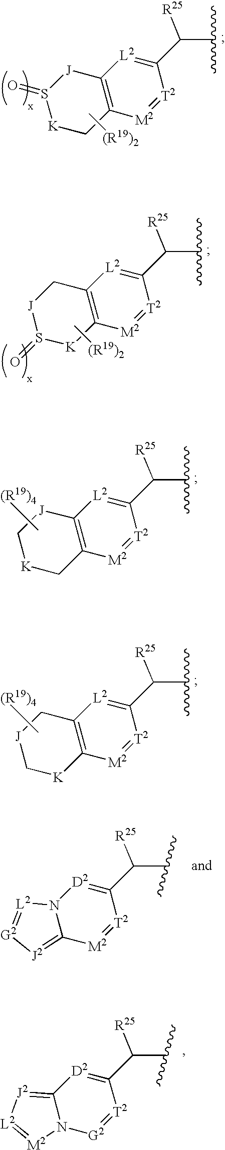
wherein:
R¹² and R¹³ are independently selected from the group consisting of hydrogen, alkyl and halo, wherein alkyl is optionally substituted one or more times, or optionally R¹² and R¹³ together form ═O, ═S or ═NR¹⁰;
R¹⁸ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times;
R¹⁹ is independently selected from the group consisting of hydrogen, alkyl, haloalkyl, cycloalkyl, heterocycloalkyl, alkynyl, aryl, heteroaryl, OH, halo, CN, C(O)NR¹⁰R¹¹, CO₂R¹⁰, OR¹⁰, OCF₃, OCHF₂, NR¹⁰CONR¹⁰R¹¹, NR¹⁰COR¹¹, NR¹⁰SO₂R¹¹, NR¹⁰SO₂NR¹⁰R¹¹, SO₂NR¹⁰R¹¹ and NR¹⁰R¹¹, wherein alkyl, haloalkyl, cycloalkyl, heterocycloalkyl, alkynyl, aryl, and heteroaryl are optionally substituted one or more times, or optionally two R¹⁹ groups together at one carbon atom form ═O, ═S or ═NR¹⁰;
R²⁵ is selected from the group consisting of hydrogen, alkyl, cycloalkyl, C(O)NR¹⁰R¹¹ and haloalkyl, wherein alkyl, cycloalkyl, and haloalkyl are optionally substituted one or more times;
J and K are independently selected from the group consisting of CR¹⁰R¹⁸, NR¹⁰, O and S(O)ₓ;
A₁ is selected from the group consisting of NR¹⁰, O and S(O)ₓ; and
D², G², J², L², M² and T² are independently selected from the group consisting of CR⁹, CR¹⁸ and N.

59. The compound of claim 58, wherein R¹ is selected from the group consisting of:

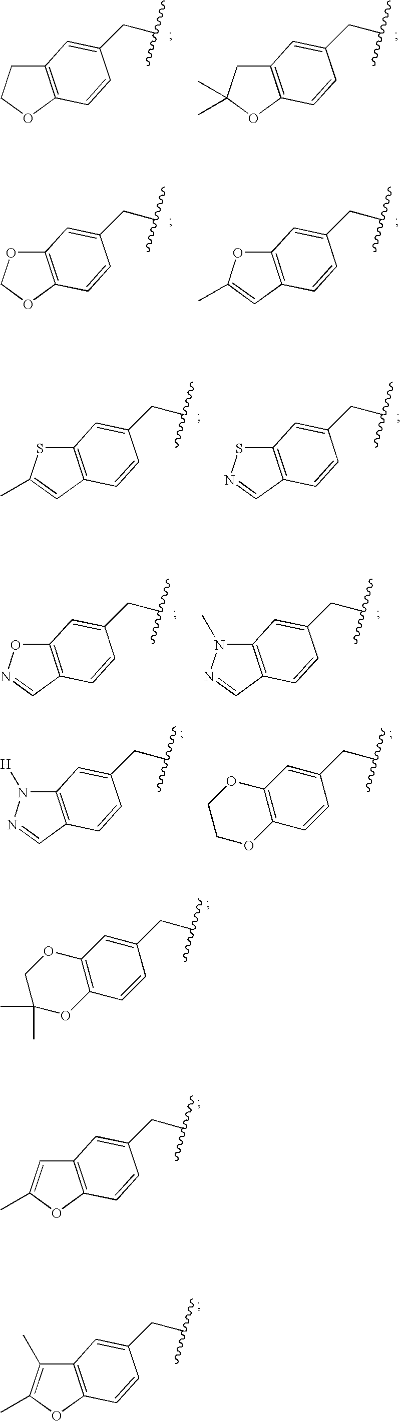

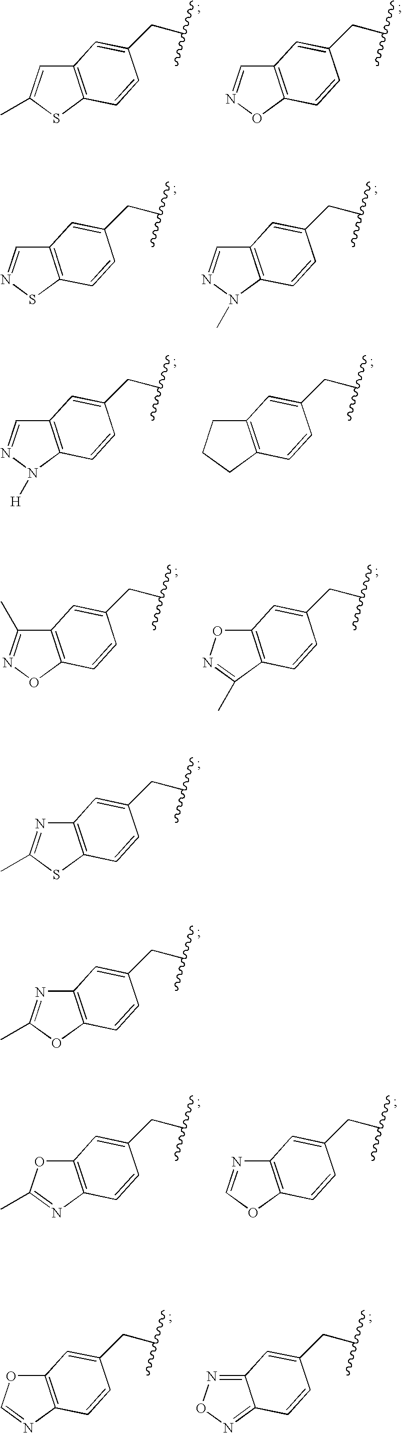

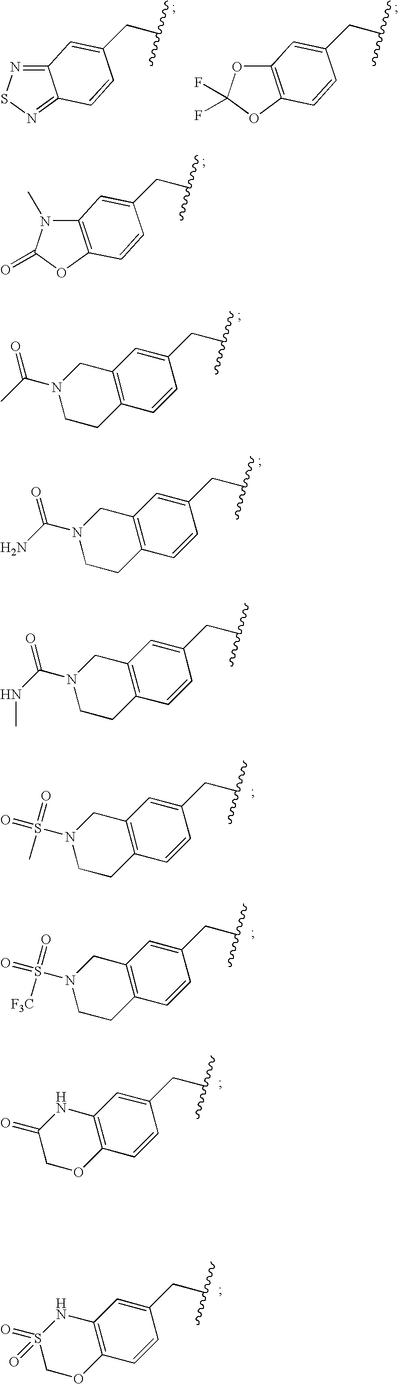

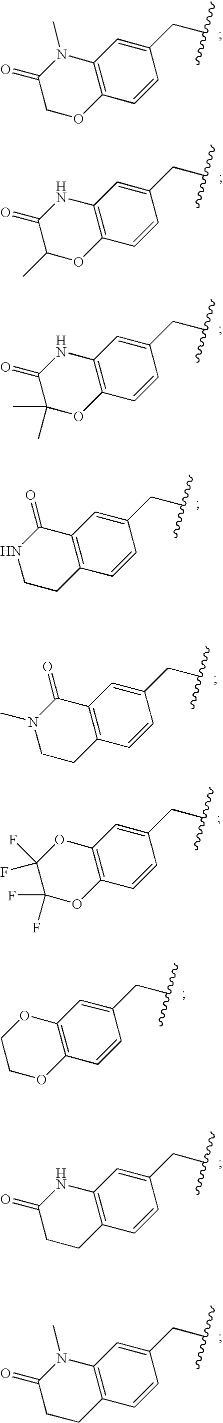

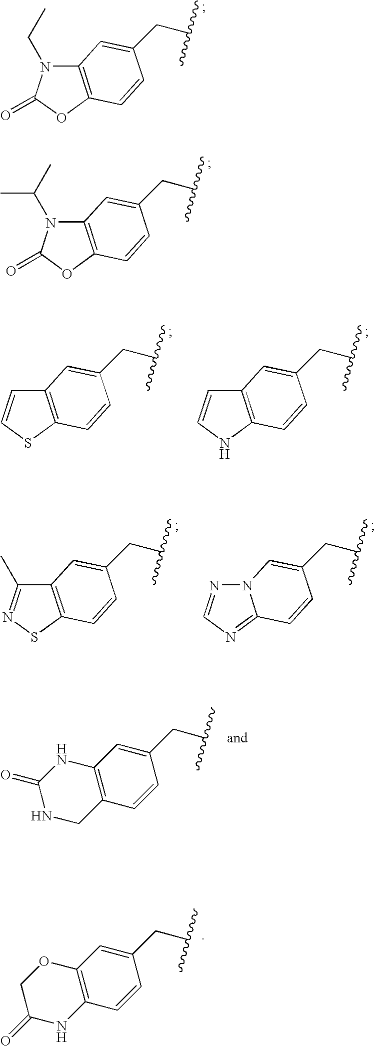

60. A compound selected from the group consisting of: 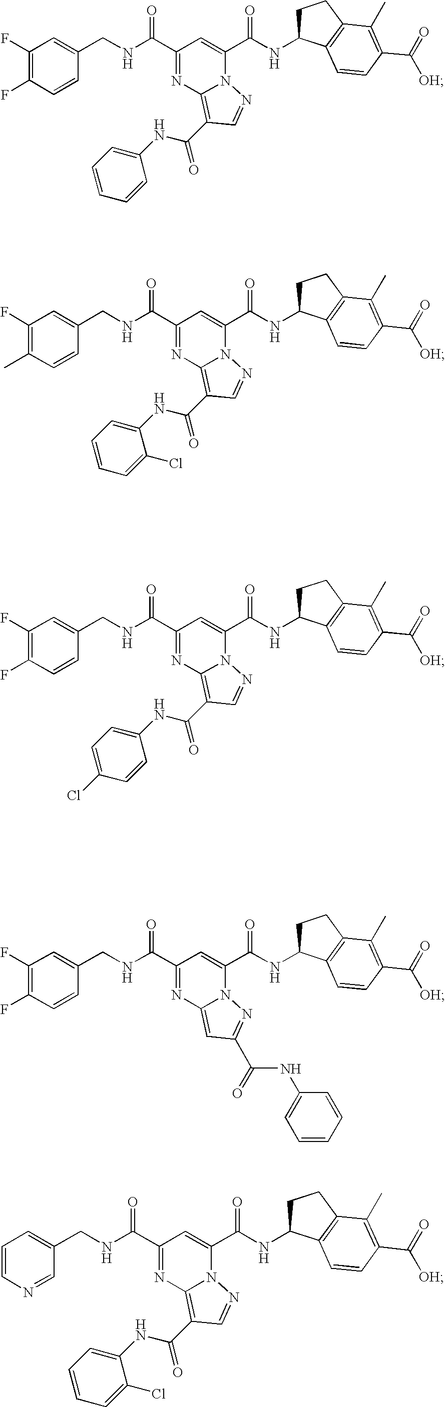 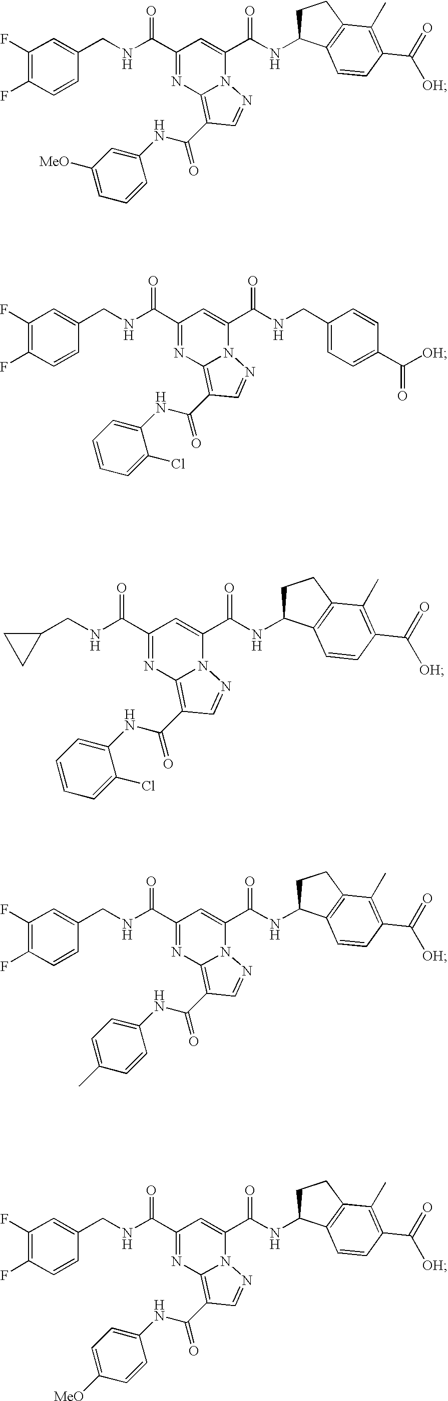 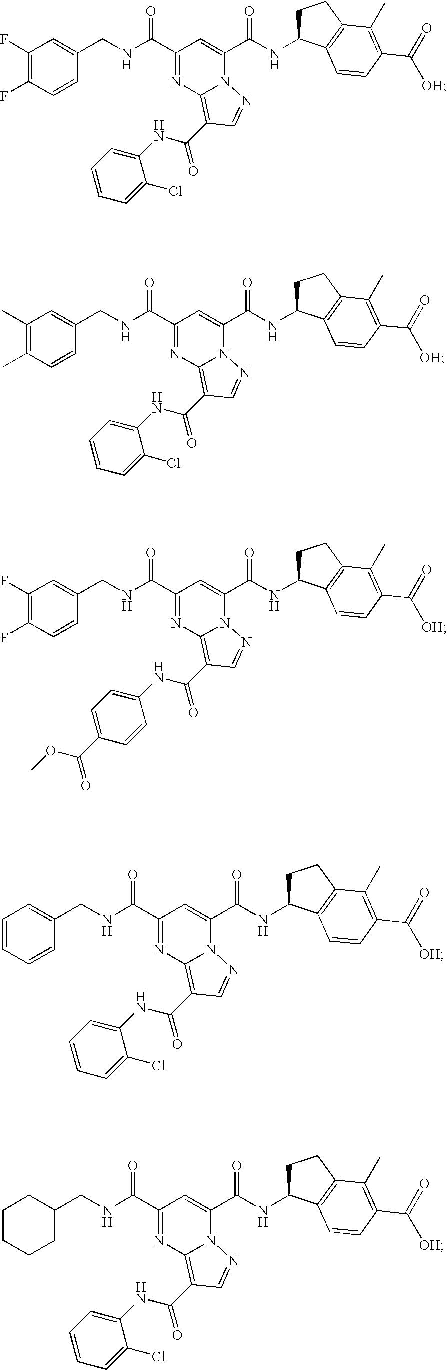 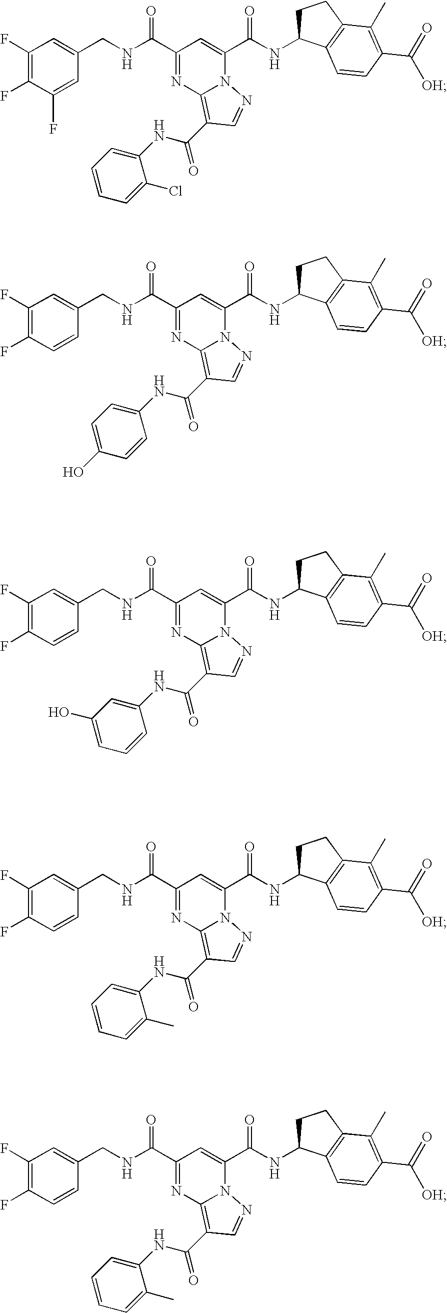 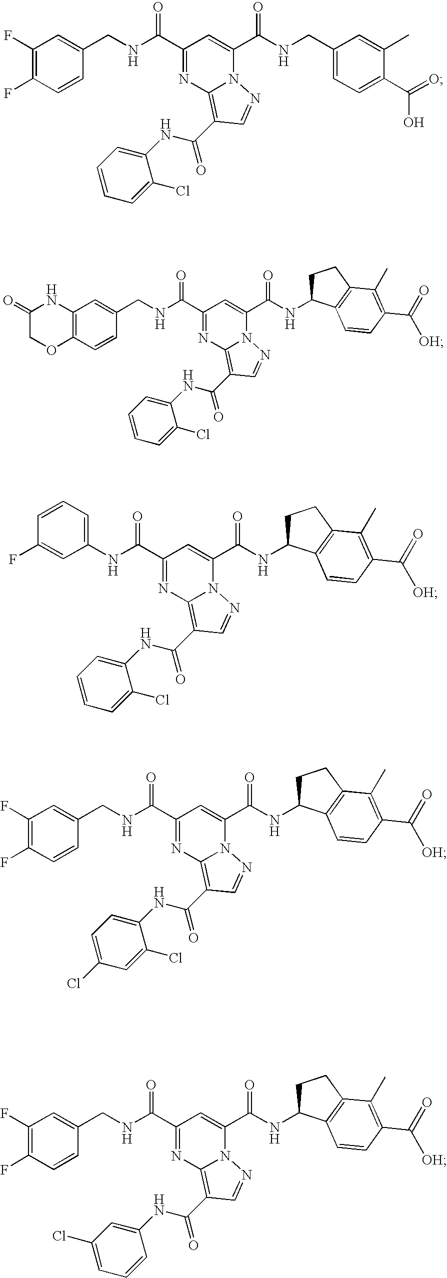 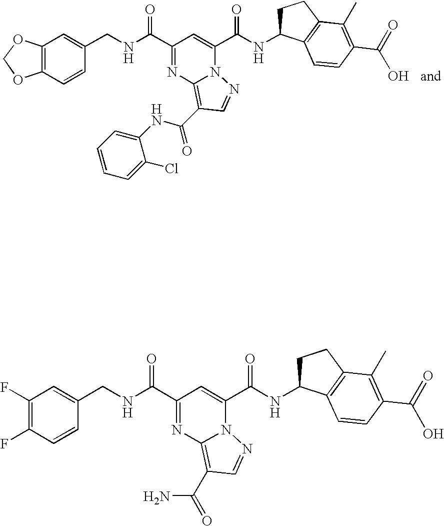
or a pharmaceutically acceptable salt thereof.

61. A compound selected from the group consisting of: 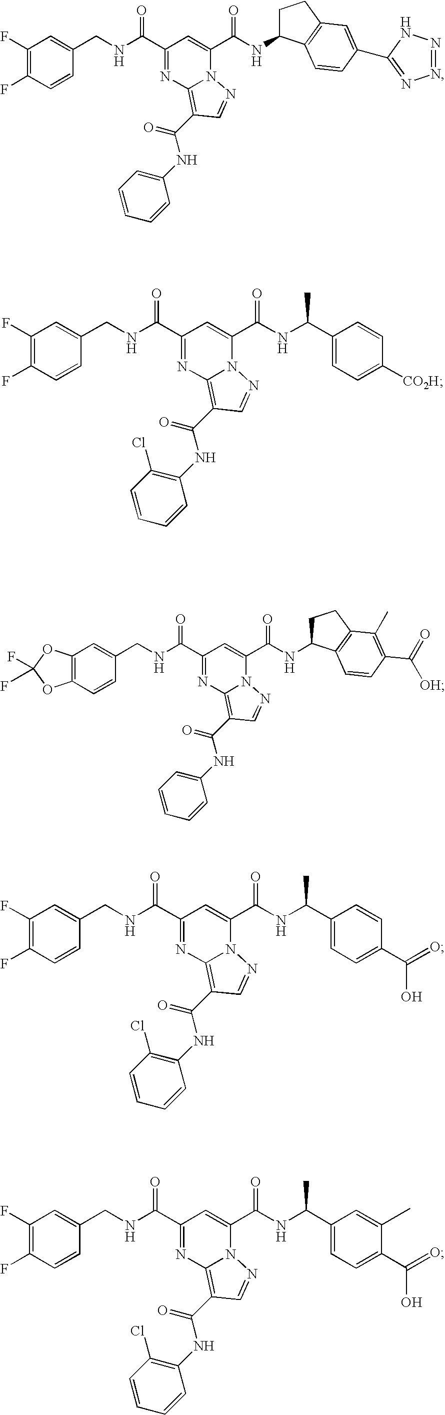 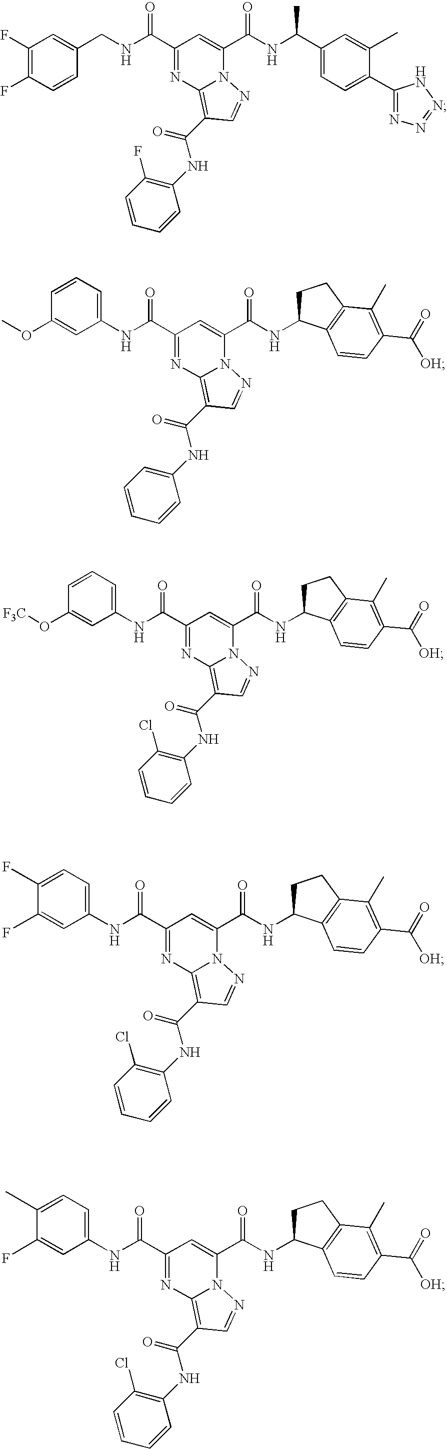 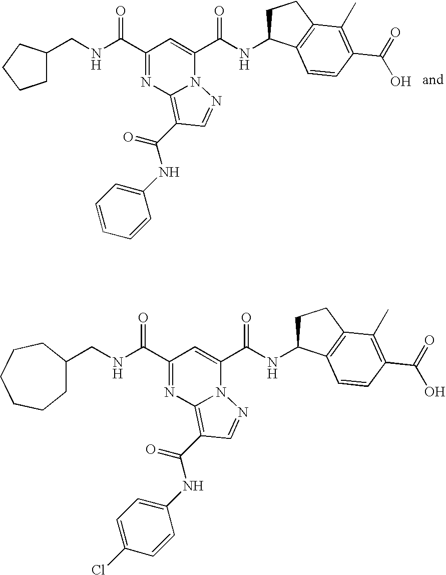
or a pharmaceutically acceptable salt thereof.

62. A compound selected from the group consisting of: 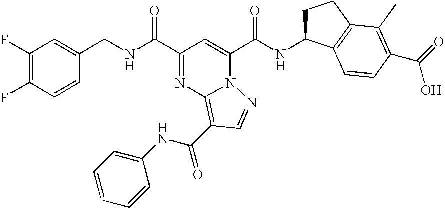
or a pharmaceutically acceptable salt thereof.

63. The compound of claim 22, having the structure: 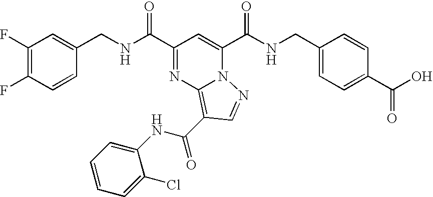
or a pharmaceutically acceptable salt thereof.

64. The compound of claim 1, having the structure: 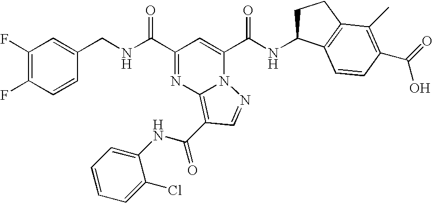
or a pharmaceutically acceptable salt thereof.

65. The compound of claim 22, having the structure: 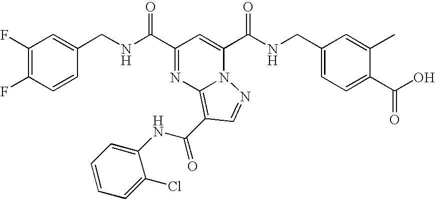
or a pharmaceutically acceptable salt thereof.

66. The compound of claim 1, having the structure: 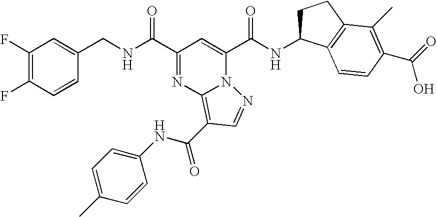
or a pharmaceutically acceptable salt thereof.

67. A compound of claim 1, having the structure: 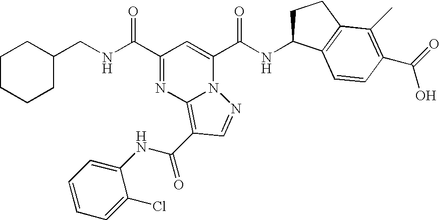
or a pharmaceutically acceptable salt thereof.

68. The compound of claim 1, having the structure: 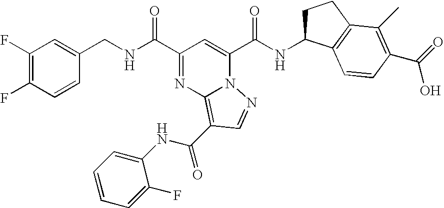
or a pharmaceutically acceptable salt thereof.

69. The compound of claim 1, having the structure: 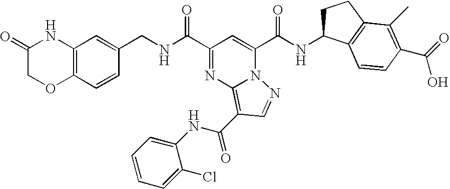
or a pharmaceutically acceptable salt thereof.

70. The compound of claim 1, having the structure: 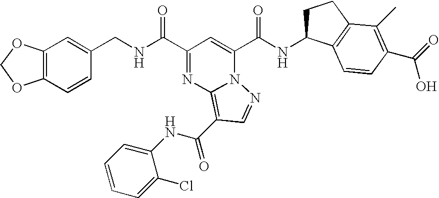
or a pharmaceutically acceptable salt thereof.

71. The compound of claim 1, having the structure: 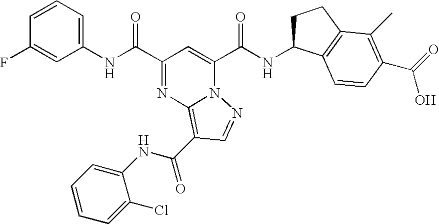
or a pharmaceutically acceptable salt thereof.

72. The compound of claim 1, having the structure: 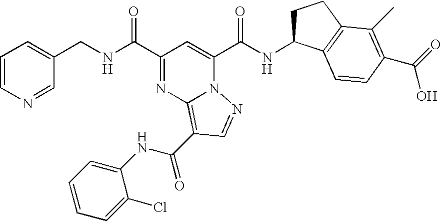
or a pharmaceutically acceptable salt thereof.

73. The compound of claim 1, having the structure: 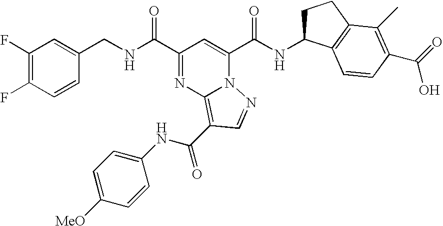
or a pharmaceutically acceptable salt thereof.

74. A compound of claim 1, having the structure: 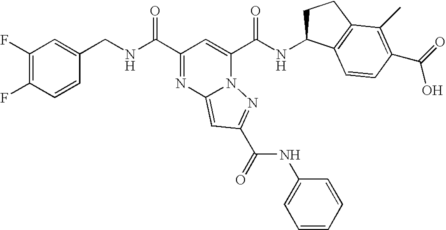
or a pharmaceutically acceptable salt thereof.

75. A compound of claim 1, having the structure: 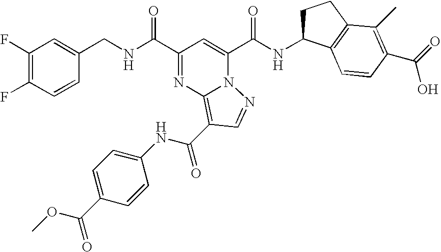
or a pharmaceutically acceptable salt thereof.

76. A pharmaceutical composition comprising an effective amount of the compound of claim 1 and a pharmaceutically acceptable carrier.

77. A pharmaceutical composition comprising an effective amount of the compound of claim 22 and a pharmaceutically acceptable carrier.

78. A pharmaceutical composition comprising an effective amount of the compound of claim 39 and a pharmaceutically acceptable carrier.

79. A method of inhibiting a metalloprotease enzyme, comprising administering a compound of claim 1.

80. The method of claim 79, wherein said metalloprotease enzyme is selected from the group MMP-3, MMP-8, MMP-12, MMP-13, ADAMTS-4 and ADAMTS-5 enzymes.

81. The method of claim 80, wherein said metalloprotease enzyme is the ADAMTS-4 enzyme.

82. A method of inhibiting a metalloprotease enzyme, comprising administering a compound of claim 22.

83. The method of claim 82, wherein said metalloprotease enzyme is selected from the group MMP-3, MMP-8, MMP-12, MMP-13, ADAMTS-4 and ADAMTS-5 enzymes.

84. The method of claim 83, wherein said metalloprotease enzyme is the ADAMTS-4 enzyme.

85. A method of inhibiting a metalloprotease enzyme, comprising administering a compound of claim 39.

86. The method of claim 82, wherein said metalloprotease enzyme is selected from the group MMP-3, MMP-8, MMP-12, MMP-13, ADAMTS-4 and ADAMTS-5 enzymes.

87. The method of claim 86, wherein said metalloprotease enzyme is the ADAMTS-4 enzyme.

88. A method of treating a metalloprotease mediated disease, comprising administering to a subject in need of such treatment an effective amount of a compound of claim 1.

89. The method of claim 88, wherein said metalloprotease mediated disease is selected from the a MMP-3 mediated disease, a MMP-8 mediated disease, a MMP-12 mediated disease, a MMP-13 mediated disease, a ADAMTS-4 mediated disease and a ADAMTS-5 mediated disease.

90. The method of claim 89, wherein said metalloprotease mediated disease is a ADAMTS-4 mediated disease.

91. A method of treating a metalloprotease mediated disease, comprising administering to a subject in need of such treatment an effective amount of a compound of claim 22.

92. The method of claim 91, wherein said metalloprotease mediated disease is selected from the a MMP-3 mediated disease, a MMP-8 mediated disease, a MMP-12 mediated disease, a MMP-13 mediated disease, a ADAMTS-4 mediated disease and a ADAMTS-5 mediated disease.

93. The method of claim 92, wherein said metalloprotease mediated disease is a ADAMTS-4 mediated disease.

94. A method of treating a metalloprotease mediated disease, comprising administering to a subject in need of such treatment an effective amount of a compound of claim 39.

95. The method of claim 94, wherein said metalloprotease mediated disease is selected from the a MMP-3 mediated disease, a MMP-8 mediated disease, a P-12 mediated disease, a MMP-13 mediated disease, a ADATFS-4 mediated disease and a ADAMTS-5 mediated disease.

96. The method of claim 95, wherein said metalloprotease mediated disease is a ADAMTS-4 mediated disease.

97. The method according to claim 88, wherein the disease is rheumatoid arthritis.

98. The method according to claim 88, wherein the disease is osteoarthritis.

99. The method according to claim 88, wherein the disease is inflammatory disorders.

100. The method according to claim 88, wherein the disease is atherosclerosis.

101. The method according to claim 88, wherein the disease is multiple sclerosis.

102. The method according to claim 91, wherein the disease is rheumatoid arthritis.

103. The method according to claim 91, wherein the disease is osteoarthritis.

104. The method according to claim 91, wherein the disease is inflammatory disorders.

105. The method according to claim 91, wherein the disease is atherosclerosis.

106. The method according to claim 91, wherein the disease is multiple sclerosis.

107. The method according to claim 94, wherein the disease is rheumatoid arthritis.

108. The method according to claim 94, wherein the disease is osteoarthritis.

109. The method according to claim 94, wherein the disease is inflammatory disorders.

110. The method according to claim 94, wherein the disease is atherosclerosis.

111. The method according to claim 94, wherein the disease is multiple sclerosis.

112. A pharmaceutical composition comprising:
a) an effective amount of a compound according to claim 1;
b) a pharmaceutically acceptable carrier; and
c) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

113. A pharmaceutical composition comprising:
a) an effective amount of a compound according to claim 22;
b) a pharmaceutically acceptable carrier; and
c) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

114. A pharmaceutical composition comprising:
a) an effective amount of a compound according to claim 39;
b) a pharmaceutically acceptable carrier; and
c) a member selected from the group consisting of: (a) a disease modifying antirheumatic drug; (b) a nonsteroidal anti-inflammatory drug; (c) a COX-2 selective inhibitor; (d) a COX-1 inhibitor; (e) an immunosuppressive; (f) a steroid; (g) a biological response modifier; and (h) a small molecule inhibitor of pro-inflammatory cytokine production.

115. A pharmaceutical composition comprising at least one compound selected from the group consisting of: 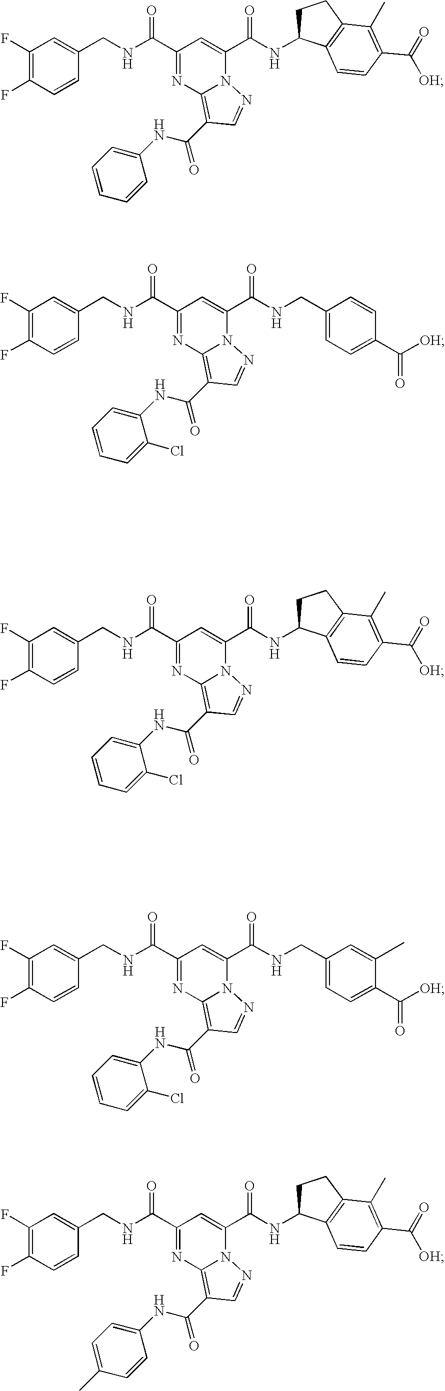 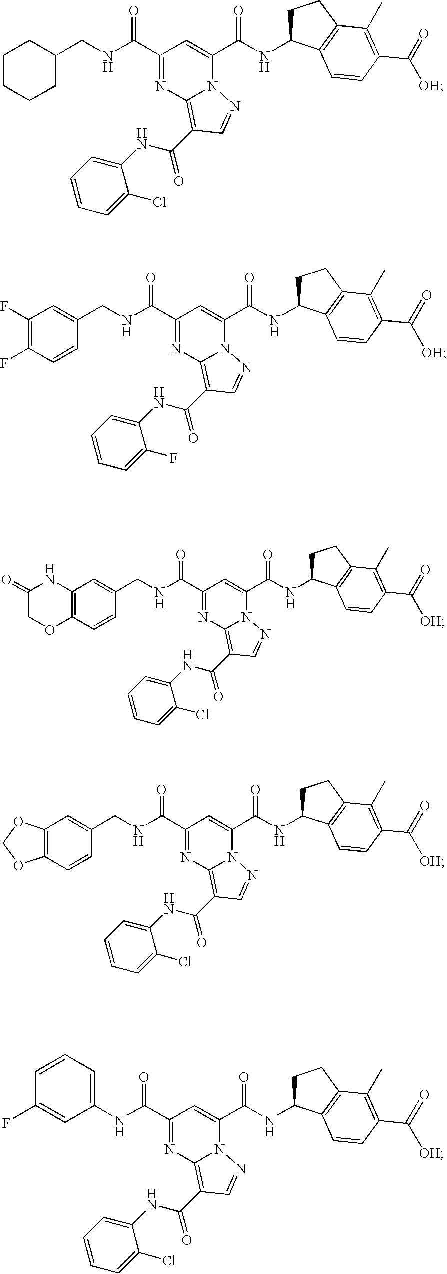 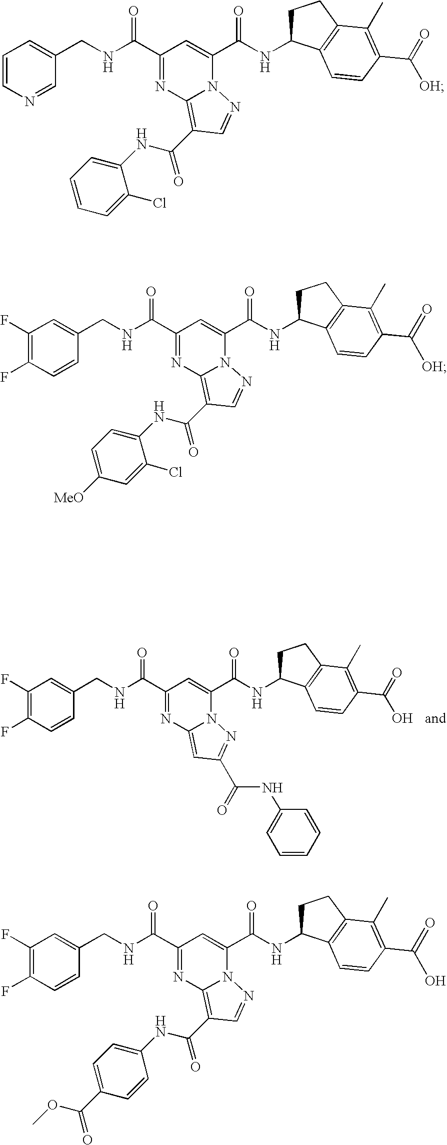
or a pharmaceutically acceptable salt thereof.
